(19) United States
(12) Patent Application Publication
(10) Pub. No.: US 2021/0345618 A1
(43) Pub. Date: Nov. 11, 2021

(54) METHODS AND COMPOSITIONS FOR IMPROVING PHOSPHATE SOLUBILIZATION

(71) Applicant: Pivot Bio, Inc., Berkeley, CA (US)

(72) Inventors: Sarah Bloch, Berkeley, CA (US); Karsten Temme, Berkeley, CA (US); Alvin Tamsir, Berkeley, CA (US); Douglas Higgins, Berkeley, CA (US); Austin Davis-Richardson, Berkeley, CA (US); Rosemary Clark, Berkeley, CA (US); Shayin Gottlieb, Berkeley, CA (US); James Gerard Lorigan, Berkeley, CA (US)

(21) Appl. No.: 17/278,022

(22) PCT Filed: Sep. 19, 2019
(86) PCT No.: PCT/US2019/052003

§ 371 Date: Mar. 19, 2021

Related U.S. Application Data
(60) Provisional application No. 62/734,777, filed on Sep. 21, 2018.

(51) Int. Cl. A01N 63/20; C12N 15/01; A01N 63/30; C12N 1/14; C12N 1/20

(52) U.S. Cl. CPC A01N 63/20; C12N 15/01; C12N 1/20; C12N 1/14; A01N 63/30

(57) ABSTRACT

Methods and systems are provided for increasing the phosphate solubilization activity of a microbe. Microbes identified through the use of the disclosed methods and systems are also provided. The methods include the steps of and the systems include instructions for identifying a coding sequence associated with phosphate solubilization, altering the coding sequence associated with phosphate solubilization, functionally linking the altered coding sequence associated with phosphate solubilization to a promoter, and introducing the altered coding sequence associated with phosphate solubilization under the control of the promoter into the microbe. Alterations to the coding sequence can include, e.g., codon randomization of native codons, deletion of coding sequence, and insertion of regulatory sequences.

### CROSS REFERENCE TO RELATED APPLICATIONS

This application claims the benefit of U.S. Provisional Patent Application No. 62/734,777, filed Sep. 21, 2018, the contents of which is hereby incorporated in its entirety.

### SEQUENCE LISTING

The instant application contains a Sequence Listing which has been submitted electronically in ASCII format and is hereby incorporated by reference in its entirety. Said ASCII copy, created on Sep. 19, 2019, is named 47736-711_601_SL.txt and is 344,652 bytes in size.

### BACKGROUND

Plants are linked to the microbiome via a shared metabolome. A multidimensional relationship between a particular crop trait and the underlying metabolome is characterized by a landscape with numerous local maxima. Optimizing from an inferior local maximum to another representing a better trait by altering the influence of the microbiome on the metabolome may be desirable for a variety of reasons, such as for crop optimization. Economically, environmentally, and socially sustainable approaches to agriculture and food production are required to meet the needs of a growing global population. By 2050 the United Nations' Food and Agriculture Organization projects that total food production must increase by 70% to meet the needs of the growing population, a challenge that is exacerbated by numerous factors, including diminishing freshwater resources, increasing competition for arable land, rising energy prices, increasing input costs, and the likely need for crops to adapt to the pressures of a drier, hotter, and more extreme global climate.

Phosphorus is a key nutrient in crop production. Phosphorus is commonly plentiful in agricultural soils; however, the vast majority is tied up in insoluble forms that are unavailable to crop plants. In some soils, insoluble organic phosphorus and insoluble inorganic phosphorus can comprise up to 90% of total soil phosphorus.

### SUMMARY

One aspect of the disclosure provides a method of increasing the phosphate solubilization activity of a microbe by isolating a coding sequence associated with phosphate solubilization from the microbe; codon randomizing the coding sequence associated with phosphate solubilization; functionally linking the codon randomized coding sequence associated with phosphate solubilization to a promoter; and reintroducing the codon randomized coding sequence associated with phosphate solubilization under the control of the promoter into the microbe.

Provided herein are engineered microbes comprising an alteration in a gene associated with phosphate solubilization, wherein the gene associated with phosphate solubilization is native to the microbe, whereby the engineered microbe solubilizes phosphates at a greater capacity or rate as compared to a non-engineered microbe of the same species.

In some embodiments, the engineered microbe comprises an alteration in a gene associated with phosphate solubilization, wherein the engineered microbe is a non-intergeneric engineered microbe, and wherein the engineered microbe solubilizes phosphates at a greater capacity or rate as compared to a non-engineered microbe of the same species.

In some embodiments, the gene associated with phosphate solubilization is a non-specific acid phosphatase gene. In further embodiments, the non-specific acid phosphatase gene comprises phoC, napA, napD, napE, acpA, appA, or a functional variant thereof, or any combination thereof.

In some embodiments, the gene associated with phosphate solubilization is a phytase gene. In further embodiments, the phytase gene is appA, phy, or a functional variant thereof, or any combination thereof.

In some embodiments, the gene associated with phosphate solubilization is a gluconic acid biosynthetic gene. In further embodiments, the gluconic acid biosynthetic gene is pqqA, pqqB, pqqC, pqqD, pqqE, gcd, gabY, or a functional variant thereof, or any combination thereof.

In some embodiments, the gene associated with phosphate solubilization is a gluconic acid transporter, a gluconate dehydrogenase, a glucose dehydrogenase, or a functional variant thereof, or any combination thereof. In further embodiments, the engineered microbe comprises altered expression of the gene associated with phosphate solubilization as compared to a microbe of the same species which lacks the alteration in the gene.

In some embodiments, the alteration in a gene associated with phosphate solubilization comprises an insertion of a regulatory element. In further embodiments, the regulatory element is a constitutive promoter. In some embodiments, the regulatory element is an inducible promoter. In some embodiments, the regulatory element is a tissue-specific promoter. In some embodiments, the regulatory element is derived from a microbe of the same species as the engineered microbe. In some embodiments, the regulatory element is derived from a microbe of the same genus as the engineered microbe. In some embodiments, the regulatory element is derived from a microbe of a different species than the engineered microbe. In some embodiments, the regulatory element is derived from a microbe of a different genus than the engineered microbe.

In some embodiments, the alteration in a gene associated with phosphate comprises codon optimization. In some embodiments, the alteration in a gene associated with phosphate comprises codon randomization. In some embodiments, the alteration in a gene associated with phosphate comprises a reduction in gene function. In some embodiments, the alteration in a gene associated with phosphate comprises a loss of function mutation. In some embodiments, the alteration in a gene associated with phosphate comprises gene deletion.

In some embodiments, the engineered microbe is an engineered bacterium. In further embodiments, the engineered bacterium is selected from the group consisting of: Alcaligenes spp., Aerobactor aerogenes, Achromobacter spp., Actinomadura oligospora, Agrobacterium spp., Azospirillum brasilense, Bacillus spp., Bacillus circulans, Bacillus cereus, Bacillus fusiformis, Bacillus pumils, Bacillus megaterium, Bacillus mycoides, Bacillus polymyxa, Bacillus coagulans, Bacillus chitinolyticus, Bacillus subtilis, Bradyzhizobium spp., Brevibacterium spp., Citrobacter spp., Pseudomonas spp., Pseudomonas putida, Pseudomonas striata, Pseudomonas fluorescens, Pseudomonas calcis, Flavobacterium spp., Nitrosomonas spp., Erwinia spp., Mirococcus spp., Escherichia intermedia, Enterobacter asburiae, Serratia phosphoticum, Nitrobacter spp., Thiobacillus ferroxidans, Thiobacillus thiosidans, Rhizobium meliloti, and Xanthomonas spp.

In some embodiments, the engineered microbe is an engineered fungus. In further embodiments, engineered fungus is selected from the group consisting of: Aspergillus awamori, Aspergillus niger, Aspergillus tereus, Aspergillus flavus, Aspergillus nidulans, Aspergillus foetidus, Aspergillus wentii, Fusarium oxysporum, Alternaria teneius, Achrothcium spp., Penicillium bilaiae, Penicillum digitatum, Penicillum lilacinium, Penicillum balaji, Penicillum funicolosum, Cephalosporium spp., Cladosprium spp., Curvularia lunata, Cunnighamella spp., Candida spp., Chaetomium globosum, Humicola inslens, Humicola lanuginose, Helminthosporium spp., Paecilomyces fusisporous, Pythium spp., Phoma spp., Populospora mytilina, Myrothecium roridum, Morteirella spp., Micromonospora spp., Oidendendron spp., Rhizoctonia solani, Rhizopus spp., Mucor spp., Trichoderma viridae, Torula thermophila, Schwanniomyces occidentalis, and Sclerotium rolfsii.

In some embodiments, the engineered microbe is an engineered yeast. In some embodiments, the engineered microbe is a biocontrol microbe. In some embodiments, the engineered microbe expresses a bacterial toxin. In some embodiments, the engineered microbe further comprises an alteration in a gene associated with nitrogen fixation or assimilation, and wherein the engineered microbe excretes fixed nitrogen at a greater capacity or rate than a non-engineered microbe of the same species. In some embodiments, the engineered microbe fixes nitrogen.

Also provided herein are engineered microbes comprising an alteration in a gene selected from the group consisting of a non-specific acid phosphatase, a phytase, a pqq biosynthetic gene, a gluconic acid transporter, a gluconate dehydrogenase, a glucose dehydrogenase, or a functional variant thereof, or any combination thereof, wherein the engineered microbe solubilizes phosphates at a greater capacity or rate as compared to a non-engineered microbe of the same species.

In some embodiments, the alteration comprises an alteration in a gene selected from the group consisting of phoC, napD, napE, acpA, appA, pqqA, pqqB, pqqC, pqqD, pqqE, gcd, or a functional variant thereof, or any combination thereof. In some embodiments, the alteration comprises codon optimization of one or more codons in the gene. In some embodiments, the alteration comprises codon randomization of one or more codons in the gene. In some embodiments, the engineered microbe comprises altered expression of the gene as compared to a non-engineered microbe of the same species.

In some embodiments, the alteration comprises an insertion of a regulatory element. In some embodiments, the regulatory element is a constitutive promoter. In some embodiments, the regulatory element is an inducible promoter. In some embodiments, the regulatory element is a tissue-specific promoter. In some embodiments, the regulatory element is derived from a microbe of the same species as the engineered microbe. In some embodiments, the regulatory element is derived from a microbe of the same genus as the engineered microbe. In some embodiments, the regulatory element is derived from a microbe of a different species than the engineered microbe. In some embodiments, the regulatory element is derived from a microbe of a different genus than the engineered microbe.

Also provided herein are engineered microbes comprising an alteration in a gene selected from the group consisting of a pqq gene, gabY, gcd, or a functional variant thereof, or any combination thereof, wherein the alteration comprises codon alteration, and wherein the engineered microbe solubilizes phosphates at a greater capacity or rate as compared to a non-engineered microbe of the same species.

In some embodiments, the engineered microbe solubilizes phosphate in the presence of at least about 12 mM of soluble phosphate. In some embodiments, the engineered microbe does not contain any DNA elements derived from an organism of a different genus.

Also provided herein are methods of solubilizing phosphate, the method comprising contacting insoluble phosphate with an engineered microbe described herein.

Also provided herein are methods of increasing an amount of soluble phosphate in soil, the method comprising contacting soil comprising insoluble phosphate with an engineered microbe described herein.

In some embodiments, the insoluble phosphate is organic phosphate. In some embodiments, wherein the insoluble phosphate is inorganic phosphate.

Also provided herein are methods of producing an engineered microbe with improved phosphate solubilization activity, the method comprising: (a) altering a codon usage of a native coding sequence associated with phosphate solubilization to yield a codon altered coding sequence associated with phosphate solubilization; (b) functionally linking the codon altered coding sequence associated with phosphate solubilization to a promoter; and (c) introducing the promoter and the codon altered coding sequence associated with phosphate solubilization into a microbe to produce the improved microbe.

In some embodiments, the native coding sequence is identified from a microbe of the same species as the improved microbe. In some embodiments, altering codon usage of the native coding sequence comprises codon randomization. In some embodiments, altering codon usage of the native coding sequence comprises codon optimization.

In some embodiments, the engineered microbe is able to solubilize at least 5% more phosphate as compared to a non-engineered microbe of the same species. In some embodiments, the engineered microbe is able to solubilize at least 10% more phosphate as compared to a non-engineered microbe of the same species. In some embodiments, the engineered microbe is able to solubilize at least 15% more phosphate as compared to a non-engineered microbe of the same species. In some embodiments, the engineered microbe is able to solubilize at least 50% more phosphate as compared to a non-engineered microbe of the same species. In some embodiments, the engineered microbe is able to solubilize at least 90% more phosphate as compared to a non-engineered microbe of the same species.

In some embodiments, the amount of phosphate solubilized is measured by a modified ascorbic acid method.

Also provided herein are methods of increasing an amount of soluble phosphate in soil, the method comprising contacting soil comprising insoluble phosphate with an engineered microbe, wherein the engineered microbe has decreased function of a gad gene, a gntT gene, or a functional variant thereof, or any combination thereof.

In some embodiments, the decreased function of a gad gene, a gntT gene, or a functional variant thereof, or any combination thereof, is caused by deletion of the gad gene, the gntT gene, or the functional variant thereof, or any combination thereof. In some embodiments, the gad gene is gad1 or gad2.

Also provided herein are engineered microbes comprising a codon altered alkaline phosphatase gene selected from the group consisting of phoA, phoC, and phoD, wherein the engineered microbe solubilizes phosphates at a greater capacity or rate as compared to a non-engineered microbe of the same species.

In some embodiments, the codon altered alkaline phosphatase gene is codon randomized. In some embodiments, the codon altered alkaline phosphatase gene is codon is codon optimized.

Also provided herein are methods of increasing an amount of phosphorous in a plant, the method comprising contacting the plant with engineered microbes, the engineered microbes comprising at least one genetic variation in a gene associated with phosphorous solubilization.

In some embodiments, the engineered microbes are engineered non-intergeneric microbes. In some embodiments, contacting the plant with engineered non-intergeneric microbes comprises applying the engineered non-intergeneric microbes into soil in which seeds of the plant are planted. In some embodiments, contacting the plant with engineered non-intergeneric microbes comprises applying the engineered non-intergeneric microbes into furrows in which seeds of the plant are planted. In some embodiments, contacting the plant with engineered non-intergeneric microbes comprises coating the engineered non-intergeneric microbes onto a seed of the plant. In some embodiments, the plant is an agricultural crop plant selected from the group consisting of sorghum, canola, tomato, strawberry, barley, rice, corn, and wheat.

In some embodiments, the engineered non-intergeneric microbes colonize at least a root of the plant such that the engineered non-intergeneric microbes are present in the plant in an amount of at least 10⁵ colony forming units per gram fresh weight of tissue. In some embodiments, the engineered non-intergeneric microbes solubilize organic phosphorous. In some embodiments, the engineered non-intergeneric microbes solubilize inorganic phosphorous. In some embodiments, the engineered non-intergeneric microbes excrete a phosphate-solubilizing product. Engineered non-intergeneric microbes, wherein the engineered non-intergeneric microbes, in planta, solubilize at least 1% of phosphorous in a plant. In some embodiments, the engineered non-intergeneric microbes are bacteria. In some embodiments, the engineered non-intergeneric microbes are fungi.

Also provided herein are bacterial phosphorous solubilization systems comprising nucleic acids encoding: at least one operon comprising a plurality of coding sequences for a set of polypeptides encoded by genes collectively associated with phosphate solubilization, wherein at least one of the plurality of coding sequences comprises at least one non-native codon; a heterologous promoter region that directs expression of the at least one operon; and a heterologous transcriptional controller coding sequence that encodes a protein that directs expression of the at least one operon of the solubilization system, wherein the protein binds directly or indirectly to the heterologous promoter region.

Also provided herein are methods comprising: (a) providing a plurality of microbial species that are associated with a target plant of interest; (b) assaying the plurality of microbial species for a colonization metric and an ability to solubilize phosphates; (c) selecting a candidate microbial species from the plurality of assayed microbial species; (d) characterizing, in the candidate microbial species, a gene selected from the group consisting of: a non-specific acid phosphatase, a phytase, a gluconic acid biosynthetic gene, a gluconic acid transporter, a gluconate dehydrogenase, and a glucose dehydrogenase; (e) introducing one or more targeted genetic variations into the candidate microbial species; (f) confirming integration of the one or more targeted genetic variations at a target genomic locus; and (g) repeating steps (d) and (e) one or more times, until the candidate microbial species has acquired an improved ability to solubilize phosphates.

In some embodiments, the one or more targeted genetic variations are non-intergeneric genetic variations. In some embodiments, the one or more targeted genetic variations are non-intergeneric genetic variations, and step (f) further comprises confirming an absence of transgenic sequence. In some embodiments, step (b) comprises assaying the plurality of microbial species for transcriptionally active genes under metabolically relevant environmental conditions.

In some embodiments, step (e) comprises: (a) transforming the candidate microbial species with a transformation plasmid comprising: (i) a selection marker, (ii) a counterselection marker, (iii) a DNA fragment comprising a genetic variation to be introduced into the candidate microbial species at a target genomic locus in one or more genomic pathways, or sets of genes, that are associated with phosphate solubilization, and homology arms to the target genomic locus flanking the genetic variation, and (iv) a plasmid backbone; (b) selecting for the candidate microbial species that has undergone an initial homologous recombination such that the genetic variation is integrated into the target genomic locus based on the presence of the selection marker in a genome of the candidate microbial species; and (c) selecting for the candidate microbial species that has the genetic variation integrated into the target genomic locus, and has undergone an additional homologous recombination that loops-out the plasmid backbone, based on the absence of the counterselection marker.

In some embodiments, the DNA fragment comprises a nonintergeneric genetic variation.

In some embodiments, step (f) comprises sequencing a portion of a genome of the candidate microbial species. In some embodiments, step (f) comprises confirming absence of transgenic sequence from a transformation plasmid.

In some embodiments, step (b) comprises assaying the plurality of microbial species for the colonization metric under greenhouse or lab-based conditions. In some embodiments, step (b) comprises assaying the plurality of microbial species for the colonization metric under field conditions. In some embodiments, step (b) comprises assaying the plurality of microbial species for the colonization metric under (i) greenhouse or lab-based conditions and (ii) field conditions. In some embodiments, the colonization metric assayed in step (b) comprises spatial colonization patterns, temporal colonization dynamics, density of colonization, or combinations thereof.

In some embodiments, assaying the plurality of microbial species for transcriptionally active genes under metabolically relevant environmental conditions occurs under greenhouse or lab-based conditions. In some embodiments, assaying the plurality of microbial species for transcriptionally active genes under metabolically relevant environmental conditions occurs under greenhouse or lab-based conditions and comprises measuring a transcriptomic profile of the microbial species. In some embodiments, assaying the plurality of microbial species for transcriptionally active genes under metabolically relevant environmental conditions occurs under greenhouse or lab-based conditions and comprises measuring a transcriptomic activity of genes associated with the ability to solubilize phosphates. In some embodiments, assaying the plurality of microbial species for transcriptionally active genes under metabolically relevant environmental conditions occurs under greenhouse or lab-based conditions and comprises measuring a transcriptomic activity of regulatory gene sequences. In some embodiments, assaying the plurality of microbial species for transcriptionally active genes under metabolically relevant environmental conditions occurs under greenhouse or lab-based conditions and comprises measuring a transcriptomic activity of promoter sequences. In some embodiments, the plurality of microbial species for transcriptionally active genes under metabolically relevant environmental conditions occurs under greenhouse or lab-based conditions and comprises measuring a transcriptomic activity of promoter sequences in the presence of insoluble phosphates. In some embodiments, assaying the plurality of microbial species for transcriptionally active genes under metabolically relevant environmental conditions occurs under greenhouse or lab-based conditions and comprises measuring a transcriptomic activity of promoter sequences in the presence of insoluble phosphate, wherein the transcriptomic activity of the promoter sequences is measured by quantifying the expression of a regulated gene. In some embodiments, assaying the plurality of microbial species for transcriptionally active genes under metabolically relevant environmental conditions occurs under field conditions. In some embodiments, assaying the plurality of microbial species for transcriptionally active genes under metabolically relevant environmental conditions occurs under field conditions and comprises measuring a transcriptomic profile of the microbial species. In some embodiments, assaying the plurality of microbial species for transcriptionally active genes under metabolically relevant environmental conditions occurs under field conditions and comprises measuring a transcriptomic activity of genes associated with the ability to solubilize phosphates. In some embodiments, assaying the plurality of microbial species for transcriptionally active genes under metabolically relevant environmental conditions occurs under field conditions and comprises measuring a transcriptomic activity of regulatory gene sequences. In some embodiments, assaying the plurality of microbial species for transcriptionally active genes under metabolically relevant environmental conditions occurs under field conditions and comprises measuring a transcriptomic activity of promoter sequences. In some embodiments, assaying the plurality of microbial species for transcriptionally active genes under metabolically relevant environmental conditions occurs under field conditions and comprises measuring a transcriptomic activity of promoter sequences in the presence of soluble phosphates. In some embodiments, assaying the plurality of microbial species for transcriptionally active genes under metabolically relevant environmental conditions occurs under field conditions and comprises measuring a transcriptomic activity of promoter sequences in the presence of soluble phosphates, wherein the transcriptomic activity of the promoter sequences is measured by quantifying the expression of a regulated gene. In some embodiments, assaying the plurality of microbial species for transcriptionally active genes under metabolically relevant environmental conditions occurs in vitro. In some embodiments, assaying the plurality of microbial species for transcriptionally active genes under metabolically relevant environmental conditions occurs in vitro and comprises measuring a transcriptomic profile of the microbial species. In some embodiments, assaying the plurality of microbial species for transcriptionally active genes under metabolically relevant environmental conditions occurs in vitro and comprises measuring a transcriptomic activity of genes associated with the microbial species ability to solubilize phosphates. In some embodiments, assaying the plurality of microbial species for transcriptionally active genes under metabolically relevant environmental conditions occurs in vitro and comprises measuring a transcriptomic activity of regulatory gene sequences. In some embodiments, assaying the plurality of microbial species for transcriptionally active genes under metabolically relevant environmental conditions occurs in vitro and comprises measuring a transcriptomic activity of promoter sequences. In some embodiments, assaying the plurality of microbial species for transcriptionally active genes under metabolically relevant environmental conditions occurs in vitro and comprises measuring a transcriptomic activity of promoter sequences in soluble-phosphate-depleted and soluble-phosphate-replete conditions. In some embodiments, assaying the plurality of microbial species for transcriptionally active genes under metabolically relevant environmental conditions occurs in vitro and comprises measuring a transcriptomic activity of promoter sequences in soluble-phosphate-depleted and soluble-phosphate-replete conditions, wherein the transcriptomic activity of the promoter sequences is measured by quantifying the expression of a regulated gene. In some embodiments, assaying the plurality of microbial species for colonization metrics, comprises growing the plurality of microbial species in intimate association with a target plant. In some embodiments, assaying the plurality of microbial species for colonization metrics, comprises growing the plurality of microbial species in intimate association with a target plant under greenhouse or lab-based conditions. In some embodiments, assaying the plurality of microbial species for colonization metrics, comprises growing the plurality of microbial species in intimate association with a target plant under field conditions. In some embodiments, assaying the plurality of microbial species for transcriptionally active genes under metabolically relevant environmental conditions, comprises growing the plurality of microbial species in intimate association with a target plant. In some embodiments, assaying the plurality of microbial species for transcriptionally active genes under metabolically relevant environmental conditions, comprises growing the plurality of microbial species in intimate association with a target plant under greenhouse or lab-based conditions. In some embodiments, assaying the plurality of microbial species for transcriptionally active genes under metabolically relevant environmental conditions, comprises growing the plurality of microbial species in intimate association with a target plant under field conditions.

In some embodiments, step (b) comprises assaying the plurality of microbial species for ability to solubilize phosphate under greenhouse or lab-based conditions. In some embodiments, step (b) comprises assaying the plurality of microbial species for phosphate solubilization activity in a phosphate solubilization assay.

In some embodiments, the transformation plasmid is a suicide plasmid.

Also provided herein are methods for the rational improvement of plant-associated microbes to solubilize phosphate, the method comprising: (a) providing a plurality of microbial species; (b) assaying the plurality of microbial species for a colonization metric and an ability to solubilize phosphate; (c) selecting a candidate microbial species from the plurality of assayed microbial species; (d) introducing one or more targeted genetic variations into the candidate microbial species at a target genomic locus in a gene selected from the group consisting of: a non-specific acid phosphatase, a phytase, a gluconic acid biosynthetic gene, a gluconic acid transporter, a gluconate dehydrogenase, a glucose dehydrogenase, and any combination thereof; (e) confirming integration of the genetic variation at the target genomic locus; and (f) repeating steps (d)-(e) one or more times, until the candidate microbial species has acquired an improved ability to solubilize phosphate.

In some embodiments, step (b) comprises assaying transcriptionally active genes under metabolically relevant environmental conditions. In some embodiments, in step d), the one or more targeted genetic variations comprise full gene deletions, partial gene deletions, promoter insertions, single base pair changes, and combinations thereof. In some embodiments, the one or more targeted genetic variations are non-intergeneric genetic variations, and step (f) further comprises confirming absence of any transgenic genetic sequence. In some embodiments, step (e) comprises sequencing a portion of the genome of the candidate microbial species.

Also provided herein are methods for the rational improvement of plant-associated microbes to solubilize phosphate, comprising: (a) providing a plurality of microbial species; (b) assaying the plurality of microbial species for colonization metrics and an ability to solubilize phosphate; (c) selecting a candidate microbial species from the plurality of assayed microbial species; (d) introducing two or more targeted genetic variations into the candidate microbial species at two or more target genomic loci, in one or more genes selected from the group consisting of: a non-specific acid phosphatase, a phytase, a gluconic acid biosynthetic gene, a gluconic acid transporter, a gluconate dehydrogenase, a glucose dehydrogenase, and any combination thereof; and (e) confirming introduction of the genetic variations at the target genomic loci.

In some embodiments, step (b) comprises assaying transcriptionally active genes under metabolically relevant environmental conditions. In some embodiments, in step (d), the genetic variations are selected from the group consisting of: full gene deletions, partial gene deletions, promoter insertions, single base pair changes, and combinations thereof. In some embodiments, the one or more targeted genetic variations are non-intergeneric genetic variations, and wherein step (f) further comprises confirming absence of any transgenic genetic sequence. In some embodiments, step (e) comprises sequencing the genome of the candidate microbial species.

Also provided herein are computational methods, comprising: (a) accessing a plurality of microbial whole genome sequences; (b) identifying a plurality of regulatory gene sequences that actively regulate transcription of a gene under a metabolically relevant environmental condition; (c) identifying a plurality of genes associated with phosphate solubilization from the group consisting of: non-specific acid phosphatases, phytases, gluconic acid biosynthetic genes, gluconic acid transporters, gluconate dehydrogenases, glucose dehydrogenases, and any combination thereof (d) selecting a regulatory gene sequence and a gene associated with phosphate solubilization from the plurality of regulatory gene sequences and the plurality of genes associated with phosphate solubilization, wherein steps a)-d) occur in silico; and (e) manufacturing, in vivo, a remodeled microbial cell that comprises the selected regulatory gene sequence operably linked to the selected gene associated with phosphate solubilization, thereby improving the expression of the gene associated with phosphate solubilization.

Also provided herein are computational systems for the rational improvement of plant-associated microbes to solubilize phosphate, comprising: (a) one or more processors; and (b) one or more memories operatively coupled to the one or more processors and having instructions stored thereon, that when executed by the one or more processors, cause the system to: (i) access a plurality of microbial whole genome sequences; (ii) identify a plurality of regulatory gene sequences that actively regulate the transcription of a gene under a metabolically relevant environmental condition; (iii) identify a plurality of genes associated with phosphate solubilization selected from the group consisting of non-specific acid phosphatases, phytases, gluconic acid biosynthetic genes, gluconic acid transporters, gluconate dehydrogenases, glucose dehydrogenases, and any combination thereof; and (iv) select a regulatory gene sequence and a gene associated with phosphate solubilization from the plurality of regulatory gene sequences and the plurality of genes associated with phosphate solubilization.

Also provided herein are computational methods for the rational improvement of plant-associated microbes to solubilize phosphate, the method comprising: (a) activating a computer system comprising one or more processors and one or more memories operatively coupled to the one or more processors and comprising instructions stored thereon, thereby causing the one or more processors to execute the instructions, and cause the system to: (i) access a plurality of microbial whole genome sequences; (ii) identify a plurality of regulatory gene sequences that actively regulate transcription of a gene under a metabolically relevant environmental condition; (iii) identify a plurality of genes associated with phosphate solubilization from the group consisting of: non-specific acid phosphatases, phytases, gluconic acid biosynthetic genes, gluconic acid transporters, gluconate dehydrogenases, glucose dehydrogenases, and any combination thereof; (iv) select a regulatory gene sequence and a gene associated with phosphate solubilization from the pluralities; and (b) manufacturing, in vivo, a remodeled microbial cell that comprises the selected regulatory gene sequence operably linked to the selected gene associated with phosphate solubilization, thereby improving the expression of the gene associated with phosphate solubilization.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts colonization of corn roots by phoC1 (19-1237, 19-1235) and phoC2 (19-1234) expressing strains and the corresponding wild-type parent strain (C1019).

FIG. 2A depicts phoC1 (19-1237, 19-1235) transcript levels in the overexpressing strains compared to the wild-type parent strain (C1019).

FIG. 2B depicts phoC2 (19-1234) transcript levels in the overexpressing strains compared to the wild-type parent strain (C1019).

FIG. 3 shows a box plot depicting the results of a soluble phosphate screen performed on the media of 1 wild type and 7 mutant Klebsiella variicola strains.

FIG. 4 shows a box plot depicting the results of a soluble phosphate screen performed on the media of 1 wild type and 3 mutant Rahnella aquatilis strains.

FIG. 5 shows a box plot depicting the results of a soluble phosphate screen performed on the media of 1 wild type and 8 mutant Rahnella aquatilis strains.

### DETAILED DESCRIPTION

Plant-associated microbes have been found which liberate soil organic phosphorus through the expression and release of non-specific acid phosphatases (NSAPs). This disclosure provides methods and compositions for liberating organic and inorganic phosphorus in soil. Additionally, this disclosure provides an isolated phosphate-solubilizing strain of Rahnella aquatilis (CI019) that contains two paralogs of the phoC gene, (SEQ ID NOs:1 and 2), characterized in another bacterium as encoding acid phosphomonoesterases.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism.” An exemplary “intergeneric microorganism” includes a microorganism containing a mobile genetic element, or a regulatory element, which was isolated from a microorganism in a genus different from the recipient microorganism.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism.”

As used herein, “introduced genetic material” means genetic material that is added to, and remains as a component of, the genome of the recipient.

As used herein, a “control sequence” refers to an operator, promoter, silencer, or terminator.

As used herein, “in planta” refers to in, on, associated with, or in the presence of a plant.

In some embodiments, native or endogenous control sequences of genes of the present disclosure are replaced with one or more intrageneric control sequences.

As used herein, “introduced” refers to the introduction by means of modern biotechnology, and not a naturally occurring introduction.

In some embodiments, the bacteria of the present disclosure have been modified such that they are not naturally occurring bacteria.

As used herein, “introduced genetic material” means genetic material that is added to, and remains as a component of, the genome of the recipient.

As used herein, a “constitutive promoter” is a promoter, which is active under most conditions in a given organism. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; and production of compounds that requires ubiquitous activity in the organism. A sequence may be a constitutive promoter in one species but not in another. Non-limiting exemplary constitutive promoters include, tetracycline resistant promoter, T7 promoters and SP6 promoters.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, a “tissue specific” promoter in the context of a bacterium is a promoter that initiates transcription of a gene based on which plant tissue the bacterium is in. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues. This is one of the main reasons for the large number of tissue-specific promoters isolated from particular tissues found in both scientific and patent literature.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see, e.g., the EMBOSS Needle aligner available at www.ebi.ac.uk/Tools/psa/emboss_needle/nucleotide.html, optionally with default settings), the BLAST algorithm (see, e.g., the BLAST alignment tool available at blast.ncbi.nlm.nih.gov/Blast.cgi, optionally with default settings), or the Smith-Waterman algorithm (see, e.g., the EMBOSS Water aligner, available at www.ebi.ac.uk/Tools/psa/emboss water/nucleotide.html, optionally with default settings). Optimal alignment may be assessed using any suitable parameters of a chosen algorithm, including default parameters.

As used herein, the term “about” is used synonymously with the term “approximately.” Illustratively, the use of the term “about” with regard to an amount indicates that values slightly outside the cited values, e.g., plus or minus 0.1% to 10%.

Phosphate-solubilizing microbes may also solubilize inorganic phosphorous. Phosphate-solubilizing microbes release inorganic phosphorous through the production and excretion of organic acids, which acidifies the surrounding soil and increases chelation of phosphate from insoluble mineral complexes. One example is gluconic acid, which is produced and excreted by many phosphate-solubilizing bacteria.

In some cases, this disclosure provides a method for optimizing activity of a gene or pathway in an organism. In some cases, the method involves selecting a gene to optimize, codon randomizing the gene's coding sequence to remove internal regulatory sequences, and linking the codon randomized coding sequence to a promoter and ribosomal binding site (RBS). In some cases, the codon randomization step may comprise generating many different codon randomized sequences and analyzing the sequences, in silica or in vivo, to determine which sequences are most efficiently expressed in the desired organism.

### Organisms

The organism may be a multicellular organism or a single-celled organism. In some cases, the organism is a microbe. In some cases, the organism is a bacterium, an archaea, or a fungus. In some cases, the organism is a plant endophyte. In some cases, the organism is a rhizosphere-associated microbe. In some cases, the organism is capable of colonizing a plant. In some cases, the organism may be in close association with the plant. In some cases the organism may be Rahnella aquatilis, Pantoea cedenensis, Pseudomonas extremorientalis, Rhizobium halophytocola, Rhizobium cellulosilyticum, Enterobacter sacchari, Kosakonia sacchari, Burkholderia gladioli, Burkholderia anthina, Pseudomonas sp., Burkholderia sp., Variovorax paradoxus, Enterobacter xiangfangensis, Burkholderia latens, Pseudomonas monteilii, Pantoea sp., Rhizobium sp., Klebsiella sp., Sphingomonas sanxanigenens, Burkholderia vietnamiensis, Azospirillum hpoferum, Klebsiella oxytoca, Lelliottia sp., Pseudomonas parafulva, or Klebsiella variicola.

In some cases, a phosphate solubilizing microbe as described herein may be a bacterium, a fungus, an actinomycetes, or a cyanobacterium. In certain cases, phosphate solubilizing actinomycetes may be selected from the group consisting of Actinomyces spp., and Streptomyces spp. In various cases, phosphate solubilizing cyanobacteria may be selected from the group consisting of: Anabena spp., Calothrix braunii, Nostoc spp., and Scytonema spp. In some cases, a phosphate solubilizing microbe may be a Glomus fasciculatum microbe.

In certain cases, phosphate solubilizing fungi may be selected from the group consisting of: Aspergillus awamori, Aspergillus niger, Aspergillus tereus, Aspergillus flavus, Aspergillus nidulans, Aspergillus foetidus, Aspergillus wentii, Fusarium oxysporum, Alternaria teneius, Achrothcium spp., Penicillum digitatum, Penicillum lilacinium, Penicillum balaji, Penicillum funicolosum, Cephalosporium spp., Cladosprium spp., Curvularia lunata, Cunnighamella spp., Candida spp., Chaetomium globosum, Humicola inslens Humicola lanuginose, Helminthosporium spp., Paecilomyces fusisporous, Pythium spp., Phoma spp., Populospora mytilina, Myrothecium roridum, Morteirella spp., Micromonospora spp., Oidendendron spp., Rhizoctonia solani, Rhizopus spp., Mucor spp., Trichoderma viridae, Torula thermophila, Schwanniomyces occidentalis, and Sclerotium rolfsii. In various cases, phosphate solubilizing fungi may be selected from the group consisting of: Penicillium bilaiae ATCC 18309, Penicillium bilaiae ATCC 20851, Penicillium bilaiae ATCC 22348, Penicillium bilaiae NRRL 50162, Penicillium bilaiae NRRL 50169, Penicillium bilaiae NRRL 50776, Penicillium bilaiae NRRL 50777, Penicillium bilaiae NRRL 50778, Penicillium bilaiae NRRL 50777, Penicillium bilaiae NRRL 50778, Penicillium bilaiae NRRL 50779, Penicillium bilaiae NRRL 50780, Penicillium bilaiae NRRL 50781, Penicillium bilaiae NRRL 50782, Penicillium bilaiae NRRL 50783, Penicillium bilaiae NRRL 50784, Penicillium bilaiae NRRL 50785, Penicillium bilaiae NRRL 50786, Penicillium bilaiae NRRL 50787, Penicillium bilaiae NRRL 50788, Penicillium bilaiae RS7B-SD1, Penicillium brevicompactum AgRF18, Penicillium canescens ATCC 10419, Penicillium expansum ATCC 24692, Penicillium expansum YT02, Penicillium fellatanum ATCC 48694, Penicillium gaestrivorus NRRL 50170, Penicillium glabrum DAOM 239074, Penicillium glabrum CBS 229.28, Penicillium janthinellum ATCC 10455, Penicillium lanosocoeruleum ATCC 48919, Penicillium radicum ATCC 201836, Penicillium radicum FRR 4717, Penicillium radicum FRR 4719, Penicillium radicum N93/47267, Penicillium raistrickii ATCC 10490, and Pseudomonas jessenii PS06.

In some cases, phosphate solubilizing bacteria may be selected from the group consisting of: Alcaligenes spp., Aerobactor aerogenes, Achromobacter spp., Actinomadura oligospora, Agrobacterium spp., Azospirillum brasilense, Bacillus spp., Bacillus circulans, Bacillus cereus, Bacillus fusiformis, Bacillus pumils, Bacillus megaterium, Bacillus mycoides, Bacillus polymyxa, Bacillus coagulans, Bacillus chitinolyticus, Bacillus subtilis, Bradyzhizobium spp., Brevibacterium spp., Citrobacter spp., Pseudomonas spp., Pseudomonas putida, Pseudomonas striata, Pseudomonas fluorescens, Pseudomonas calcis, Flavobacterium spp., Nitrosomonas spp., Erwinia spp., Mirococcus spp., Escherichia intermedia, Enterobacter asburiae, Serratia phosphoticum, Nitrobacter spp., Thiobacillus ferroxidans, Thiobacillus thiosidans, Rhizobium meliloti, and Xanthomonas spp.

In certain cases, a phosphate solubilizing microbe may also be a biocontrol microbe, for example a microbe with biopesticidal activity. Examples of microbial strains that exhibit biopesticidal activity include, but are not limited to, Acinetobacter, Actinomycetes, Aegerita, Agrobacterium (e.g., A. radiobacter strains such as K1026 and K84), Akanthomyces, Alcaligenes, Alternaria, Aminobacter (e.g., A. aganoensis, A. aminovorans, A. anthyllidis, A. ciceronei, A. lissarensis, A. niigataensis), Ampelomyces (e.g., A. quisqualis strains such as M-10), Anabaena (e.g., A. aequalis, A. afflnis, A. angstumalis angstumalis, A. angstumalis marchita, A. aphanizomendoides, A. azollae, A. bornetiana, A. catenula, A. cedrorum, A. circinalis, A. confervoides, A. constricta, A. cyanobacterium, A. cycadeae, A. cylindrica, A. echinispora, A. felisii, A. flos-aquae flos-aquae, A. flos-aquae minor, A. flos-aquae treleasei, A. helicoidea, A. inaequalis, A. lapponica, A. laxa, A. lemmermannii, A. levanderi, A. limnetica, A. macrospora macrospora, A. macrospora robusta, A. monticulosa, A. nostoc, A. ascillarioides, A. planctonica, A. raciborski, A. scheremetievi, A. sphaerica, A. spiroides crassa, A. spiroides sprroides, A. subcylindrica, A. torulosa, A. unispora, A. variabilis, A. verrucosa, A. viguieri, A. wisconsinense, A. zierlingii), Arthrobacter, Arthrobotrys (e.g., A. aggregata, A. alaskana, A. ameropora, A. anomala, A. apscheronica, A. arthrobotryoides, A. azerbaijanica, A. bakunika, A. botryospora, A. brochopaga, A. chazarica, A. chilensis, A. cladodes, A. calvispora, A. compacta, A. conoides, A. constringens, A. cylindrospora, A. dactyloides, A. deflectans, A. dendroides, A. doliiformis, A. drechsleri, A. elegans, A. ellipsospora, A. entomopaga, A. ferox, A. foliicola, A. fruticulosa, A. globospora, A. hatospora, A. hertziana, A. indica, A. irregularis, A. javanica, A. kirghizica, A. longa, A. longiphora, A. longiramulifera, A. longispora, A. mangrovispora, A. megaspora, A. microscaphoides, A. microspora, A. multisecundaria, A. musiformis, A. nematopaga, A. nonseptata, A. oligospora, A. oudemansii, A, oviformis, A. perpasta, A. polycephala, A. pseudoclavata, A. pyriformis, A. recta, A. robusta, A. rosea, A. scaphoides, A. sclerohypha, A. shahriari, A. shizishanna, A. sinensis, A. soprunovii, A. stilbacea, A. straminicola, A. superba, A. tabrizica, A. venusta, A. vermicola, A. yunnanensis), Ascher sonia, Ascophaera, Aspergillus (e.g., A. flavus strains such as NRRL 21882, A. parasiticus), Aulosira (e.g. A. aenigmatica, A. africana, A. bohemensis, A. bombayensis, A. confluens, A. fertilissima, A. fertilissma var. tenius, A. fritschii, A. godoyana, A. implexa, A. laxa, A. plantonica, A. prolifica, A. pseuodoramosa, A. schauinslandii, A. striata, A. terrestris, A. thermalis), Aureobacterium, Aureobasidium (e.g., A. pullulans strains such as DSM 14940 and DSM 14941), Azobacter, Azorhizobium (e.g., A. caulinodans, A. doebereinerae, A. oxalatiphilum), Azospirillum (e.g., A. amazonense strains such as BR 11140 (SpY2T). A. brasilense strains such as INTA Az-39, AZ39, XOH, BR 11002, BR 11005, Ab-V5 and Ab-V6, A. canadense, A. doebereinerae, A. formosense, A. halopraeferans, A. irakense, A. largimobile, A. lipoferum strains such as BR 11646, A. melinis, A. oryzae, A. picis, A. rugosum, A. thiophilum, A. zeae), Azotobacter (e.g., A. agilis, A. armeniacus, A. sp. AR, A. beijerinckii, A. chroococcum, A. DCU26, A. FA8, A. nigricans, A. paspali, A. salinestris, A. tropicalis, A. vinelandii), Bacillus (e.g., B. amyloliquefaciens strains, B. cereus strains, B. laevolacticus, B. lichenformis, B. macerns, B. firmus, B. mycoides strains such as NRRL B-21664, B. pasteurii, B. pumilus, B. sphaericus, B. subtilis, B. thuringiensis strains such as ATCC 13367, GC-91, NRRL B-21619, ABTS-1857, SAN 401 I, ABG-6305, ABG-6346, AM65-52, SA-12, SB4, ABTS-351, HD-1, EG 2348, EG 7826, EG 7841, DSM 2803, NB-125 and NB-176), Beijerinckia, Beauveria (e.g., B. bassiana strains such as ATCC 26851, ATCC 48023, ATCC 48585, ATCC 74040, ATCC-74250, DSM 12256 and PPM 5339), Beijerinckia, Blastodendrion, Bosea (e.g., B. eneae, B. lathyri, B. lupini, B. massiliensis, B. minatitlanensis, B. robiniae, B. thiooxidans, B. vestrisii), Bradyrhizobium (e.g., B. arachidis, B. bete, B. canariense, B. cytisi, B. daqingense, B. denitriflcans, B. diazoefflciens, B. elkanii strains such as SEMIA 501, SEMIA 587 and SEMIA 5019, B. ganzhouense, B. huanghuauhaiense, B. icense, B. ingae, B. iriomotense, B. japonicum strains such as NRRL B-50586 (also deposited as NRRL B-59565), NRRL B-50587 (also deposited as NRRL B-59566), NRRL B-50588 (also deposited as NRRL B-59567), NRRL B-50589 (also deposited as NRRL B-59568), NRRL B-50590 (also deposited as NRRL B-59569), NRRL B-50591 (also deposited as NRRL B-59570), NRRL B-50592 (also deposited as NRRL B-59571), NRRL B-50593 (also deposited as NRRL B-59572), NRRL B-50594 (also deposited as NRRL B-50493), NRRL B-50608, NRRL B-50609, NRRL B-50610, NRRL B-50611, NRRL B-50612, NRRL B-50726, NRRL B-50727, NRRL B-50728, NRRL B-50729, NRRL B-50730, SEMIA 566, SEMIA 5079, SEMIA 5080, USDA 6, USDA 110, USDA 122, USDA 123, USDA 127, USDA 129 and USDA 532C, B. jicamae, B. lablabi, B. liaoningense, B. manausense, B. neotropicale, B. oligotrophicum, B. ottawaense, B. pachyrhizi, B. paxllaeri, B. retamae, B. rifense, B. valentinum, B. yuanmingense), Burkholderia (e.g., B. acidipaludis, B. ambifaria, B. andropogonis, B. anthina, B. arboris, B. bannensis, B. bryophila, B. caledonica, B. caribensis, B. caryophylli, B. cenocepacua, B. choica, B. cocovenenans, B. contaminans, B. denitriflcans, B. diazotrophica, B. diffusa, B. dilworthii, B. dolosa, B. eburnea, B. endofungorum, B. ferrariae, B. fungorum, B. ginsengisoli, B. gladioli, B. glathei, B. glumae, B. graminis, B. grimmiae, B. heleia, B. hospital, B. humi, B. kururiensis, B. lata, B. latens, B. mallei, B. megapolitana, B. metallica, B. mimosarum, B. multivorans, B. nodosa, B. norimbergensis, B. oklahomensis, B. phenazinium, B. phenoliruptrix, B. phymatum, B. phytofirmans, B. pickettii, B. plantarii, B. pseudomallei, B. pseudomultivorans, B. pyrrocinia, B. rhizoxinica, B. rhynchosiae, B. sabiae, B. sacchari, B. sartisoli, B. sediminicola, B. seminalis, B. silvatlantica, B. singaporensis, B. soli, B. sordidcola, B. sp. strains such as A396, B. sprentiae, B. stabilis, B. symbiotica, B. tellur is, B. terrae, B. terrestris, B. terricola, B. thailandensis, B. tropica, B. tuberum, B. ubonensis, B. udeis, B. unamae, B. vandii, B. vietnamiensis, B. xenovorans, B. zhejiangensis), Brevibacillus, Burkholderia (e.g., B. sp. A396 nov. rinojensis NRRL B-50319), Calonectria, Candida (e.g., C. oleophila such 1-182, C. saitoana), Candidatus (e.g., C. Burkholderia calva, C. Burkholderia crenata, C. Burkholderia hispidae, C. Burkholderia kirkii, C. Burkholderia mamillata, C. Burkholderia nigropunctata, C. Burkholderia rigidae, C. Burkholderia schumannianae, C. Burkholderia verschuerenii, C. Burkholderia virens, C. Phytoplasma allocasuarinae, C. Phytoplasma americanum, C. Phytoplasma asteris, C. Phytoplasma aurantifolia, C. Phytoplasma australiense, C. Phytoplasma balanitae, C. Phytoplasma brasiliense, C. Phytoplasma caricae, C. Phytoplasma castaneae, C. Phytoplasma cocosnigeriae, C. Phytoplasma cocostanzaniae, C. Phytoplasma convolvuli, C. Phytoplasma costaricanum, C. Phytoplasma cynodontis, C. Phytoplasma fragariae, C. Phytoplasma fraxini, C. Phytoplasma graminis, C. Phytoplasma japonicum, C. Phytoplasma luffae, C. Phytoplasma lycopersici, C. Phytoplasma malasianum, C. Phytoplasma mali, C. Phytoplasma omanense, C. Phytoplasma oryzae, C. Phytoplasma palmae, C. Phytoplasma palmicola, C. Phytoplasma phoenicium, C. Phytoplasma pini, C. Phytoplasma pruni, C. Phytoplasma prunorum, C. Phytoplasma pyri, C. Phytoplasma rhamni, C. Phytoplasma rubi, C. Phytoplasma solani, C. Phytoplasma spartii, C. Phytoplasma sudamericanum, C. Phytoplasma tamaricis, C. Phytoplasma trifolii, C. Phytoplasma ulmi, C. Phytoplasma vitis, C. Phytoplasma ziziphi), Chromobacterium (e.g., C. subtsugae NRRL B-30655 and PRAA4-1, C. vaccinia strains such as NRRL B-50880, C. violaceum), Chryseomonas, Clavibacter, Clonostachys (e.g., C. rosea f. catenulata (also referred to as Gliocladium catenulatum) strains such as J1446), Clostridium, Coelemomyces, Coelomycidium, Colletotrichum (e.g., C. gloeosporioides strains such as ATCC 52634), Comomonas, Conidiobolus, Coniothyrium (e.g., C. minitans strains such as CON/M/91-08), Cordyceps, Corynebacterium, Couchia, Cryphonectria (e.g., C. parasitica), Cryptococcus (e.g., C. albidus), Cryptophlebia (e.g., C. leucotreta), Culicinomyces, Cupriavidus (e.g., C. alkaliphilus, C. basilensis, C. campinensis, C. gilardii, C. laharis, C. metallidurans, C. numazuensis, C. oxalaticus, C. pampae, C. pauculus, C. pinatubonensis, C. respiraculi, C. taiwanensis), Curtobacterium, Cydia (e.g., C. pomonella strains such as V03 and V22), Dactylaria (e.g., D. Candida), Delftia (e.g., D. acidovorans strains such as RAY209), Desulforibtio, Desulfovibrio, Devosia (e.g., D. neptuniae), Dilophosphora (e.g., D. alopecuri), Engyodontium, Enter obacter, Entomophaga, Entomophthora, Erynia, Escherichia (e.g., E. intermedia), Eupenicillium, Exiguobacaterium, Filariomyces, Filobasidiella, Flavobacterium (e.g., F. H492 NRRL B-50584), Frankia (e.g., F. alni), Fusarium (e.g., F. laterium, F. oxysporum, F. solani), Gibellula, Gigaspora (e.g. G. margarita), Gliocladium (e.g., G.virens strains such as ATCC 52045 and GL-21), Glomus (e.g., G. aggregatum fl. brasilianum fl. clarumfi. deserticolafi. etunicatum, G. fasciculatum, G. intraradices strains such as RTI-801_G. monosporum, G. mosseae), Gluconobacter, Halospirulina, Harposporium (e.g., H. anguillulae), Hesperomyces, Hirsutella (e.g., H. minnesotensis, H. rhossiliensis, H. thorns onii strains such as ATCC 24874), Hydrogenophage, Hymenoscyphous (e.g., H. ericae), Hymenostilbe, Hypocrella, Isaria (e.g., I. fumosorosea strains such as Apopka-97 (deposited as ATCC 20874)), Klebsiella (e.g., K. pneumoniae, K. oxytoca), Kluyvera, Laccaria (e.g., L. bicolor, L. laccata), Lactobacillus, Lagenidium, Lecanicillium (e.g., L. lecanii strains such as KV01, L. longisporum strains such as KV42 and KV71), Leptolegnia, Lysobacter (e.g., L. antibioticus strains such as 13-1 and HS124, L. enzymogenes strains such as 3.1T8), Massospora, Meristacrum (e.g., M. asterospermum), Mesorhizobium (e.g., M. abyssinicae, M. albiziae, M. alhagi, M. amorphae, M. australicum, M. camelthorni, M. caraganae, M. chacoense, M. ciceri, M. gobiense, M. hawassense, M. huakuii, M. loti, M. mediterraneum, M. metallidurans, M. muleiense, M. opportunistum, M. plurifarium, M. qingshengii, M. robiniae, M. sangaii, M. septentrionale, M. shangrilense, M. shonense, M. silamurunense, M. tamadayense, M. tarimense, M. temperatum, M. thiogangeticum, M. tianshanense), Metarhizium (e.g., M. anisopliae (also referred to as M. brunneum, Metarrhizium anisopliae, and green muscadine) strains such as IMI 330189, FI-985, FI-1045, F52 (deposited as DSM 3884, DSM 3885, ATCC 90448, SD 170, and ARSEF 7711) and ICIPE 69), M. flavoviride strains such as ATCC 32969), Methylobacterium (e.g., M. adhaesivum, M. aerolatum, M. aminovorans, M. aquaticum, M. brachiatum, M. brachythecii, M. bullatum, M. cerastii, M. chloromethanicum, M. dankookense, M. dichloromethanicum, M. extorquens, M. fujisawaense, M. gnaphalii, M. goesingense, M. gossipiicola, M. gregans, M. haplocladii, M. hispanicum, M. iners, M. isbiliense, M. jeotgali, M. komagatae, M. longum, M. lusitanum, M. marchantiae, M. mesophilicum, M. nodulans, M. organophilum, M. oryzae, M. oxalidis, M. persicinum, M. phyllosphaerae, M. platani, M. podarium, M. populi, M. radiotolerans, M. rhodesianum, M. rhodinum, M. salsuginis, M. soli, M. suomiense, M. tardum, M. tarhaniae, M. thiocyanatum, M. thurigiense, M. trifolii, M. variabile, M. zatmanii), Metschnikowia (e.g., M. fructicola), Microbacterium (e.g., M. laevaniformans), Microdochium (e.g., M. dimerum), Microsphaeropsis (e.g., M. ochracea P130A), Microvirga (e.g., M. aerilata, M. aerophila, M. flocculans, M. guangxiensis, M. lotononidis, M. lupini, M. subterranea, M. vignae, M. zambiensis), Monacrosporium (e.g., M. cionopagum), Mucor, Muscodor (e.g., M. albus such NRRL 30547, QST 20799 and SA-13, M. roseus strains such as NRRL 30548), Mycoderma, Myiophagus, Myriangium, Myrothecium (e.g., M. verrucaria), Nectria, Nematoctonus (e.g., N. geogenius, N. leiosporus), Neozygites, Nomuraea (e.g., N. rileyi strains such as SA86101, GU87401, SR86151, CG128 and VA9101), Nostoc (e.g., N. azollae, N. caeruleum, N. carneum, N. comminutum, N. commune, N. ellipsosporum, N. flagelliforme, N. linckia, N. longs taffi, N. microscopicum, N. muscorum, N. paludosum, N. pruniforme, N. punctifrome, N. sphaericum, N. sphaeroides, N. spongiaeforme, N. verrucosum), Ochrobactrum (e.g., O. anthropi, O. cicero, O. cytisi, O. daejeonense, O. gallinifaecis, O. grigonense, O. guangzhouense, O. haematophilum, O. intermedium, O. lupini, O. oryzae, O. pectoris, O. pituitosum, O. pseudointermedium, O. pseudogrignonense, O. rhizosphaerae, O. thiophenivorans, O. tritici), Oidiodendron, Paecilomyces (e.g., P. fumosoroseus strains such as FE991 and FE 9901, P. lilacinus strains such as 251, DSM 15169 and BCP2), Paenibacillus (e.g., P. alvei strains such as NAS6G6, P. azotofixans, P. polymyxa strains such as ABP166 (deposited as NRRL B-50211)), Pandora, Pantoea (e.g., P. agglomerans strains such as NRRL B-21856, P. vagans strains such as C9-1), Paraglomus (e.g., P. brazilianum), Paraisaria, Pasteuria, Pasteuria (e.g., P. nishizawae strains such as Pnl, P. penetrans, P. ramose, P. sp. strains such as ATCC PTA-9643 and ATCC SD-5832, P. thornea, P. usage), Penicillium (e.g., P. albidum, P. aurantiogriseum, P. bilaiae (formerly known as P. bilaii and P. bilaji) strains such as ATCC 18309, ATCC 20851, ATCC 22348, NRRL 50162, NRRL 50169, NRRL 50776, NRRL 50777, NRRL 50778, NRRL 50777, NRRL 50778, NRRL 50779, NRRL 50780, NRRL 50781, NRRL 50782, NRRL 50783, NRRL 50784, NRRL 50785, NRRL 50786, NRRL 50787, NRRL 50788 and RS7B-SD1, P. brevicompactum strains such as AgRF18, P. canescens strains such as ATCC 10419, P. chyrsogenum, P. citreonigrum, P. citrinum, P. digitatum, P. expansum strains such as ATCC 24692 and YT02, P. fellatanum strains such as ATCC 48694, P. frequentas, P. fuscum, P. fussiporus, P. gaestrivorus strains such as NRRL 50170, P. glabrum strains such as DAOM 239074 and CBS 229.28, P. glaucum, P. griseofulvum, P. implicatum, P. janthinellum strains such as ATCC 10455, P. lanosocoeruleum strains such as ATCC 48919, P. lilacinum, P. minioluteum, P. montanense, P. nigricans, P. oxalicum, P. pinetorum, P. pinophilum, P. purpurogenum, P. radicum strains such as ATCC 201836, FRR 4717, FRR 4719 and N93/47267, P. raistrickii strains such as ATCC 10490, P. rugulosum, P. simplicissimum, P. solitum, P. variabile, P. velutinum, P. viridicatum), Phingobacterium, Phlebiopsis (e.g., P. gigantea), Photorhabdus, Phyllobacterium (e.g., P. bourgognense, P. brassicacearum, P. catacumbae, P. endophyticum, P. ifriqiyense, P. leguminum, P. loti, P. myrsinacearum, P. sophorae, P. trifolii), Pichia (e.g., P. anomala strains such as WRL-076), Pisolithus (e.g., P. tinctorius), Planktothricoides, Plectonema, Pleurodesmospora, Pochonia (e.g., P. chlamydopora), Podonectria, Polycephalomyces, Prochlorocoous (e.g., P. marinus), Prochloron (e.g., P. didemni), Prochlorothrix, Pseudogibellula, Pseudomonas (e.g., P. agarici, P. antartica, P. aurantiaca, P. aureofaciens, P. azotiflgens, P. azotoformans, P. balearica, P. blatchfordae, P. brassicacearum, P. brenneri, P. cannabina, P. cedrina, P. cepacia, P. chlororaphis strains such as MA 342, P. congelans, P. corrugata, P. costantinii, P. denitri leans, P. entomophila, P. fluorescem strains such as ATCC 27663, CL 145 A and A506, P. fragii, P. fuscovaginae, P. fulva, P. gessardii, P. jessenii strains such as PS06, P. kilonensis, P. koreensis, P. libanensis, P. lili, P. lundensis, P. lutea, P. luteola, P. mandelii, P. marginalis, P. meditrranea, P. meridana, P. migulae, P. moraviensis, P. mucidolens, P. orientalis, P. oryzihabitans, P. palleroniana, P. panacis, P. parafulva, P. peli, P. pertucinogena, P. plecoglossicida, P. protogens, P. proteolytica, P. putida, P. pyrocina strains such as ATCC 15958, P. rhodesiae, P. sp. strains such as DSM 13134, P. striata, P. stutzeri, P. syringae, P. synxantha, P. taetrolens, P. thisvervalensis, P. tolaasii, P. veronii), Pseudozyma (e.g., P. flocculosa strains such as PF-A22 UL), Pythium (e.g., P. oligandrum strains such as DV 74), Rhizobium (e.g., R. aggregatum, R. alamii, R. alkalisoli, P. alvei, P. azibense, P. borbori, R. calliandrae, Rcauense, R. cellulosilyticum, R. daejeonense, R. endolithicum, R. endophyticum, R. etli, R. fabae, R. flavum, R. fredii, R. freirei, R. galegae, R. gallicum, R. giardinii, R. grahamii, R. hainanense, R. halophytocola, R. halotolerans, R. helanshanense, R. herbae, R. huautlense, R. indigoferae, R. jaguaris, R kunmingense, R. laguerreae, R. larrymoorei, R. leguminosarum strains such as S012A-2 (IDAC 080305-01), R. lemnae, R. leucaenae, R. loessense, R. lupini, R. lusitanum, R. mayense, R. mesoamericanum, R. mesosinicum, R. miluonense, R. mongolense, R. multihospitium, R. naphthalenivorans, R. nepotum, R. oryzae, R. pakistanensis, R. paknamense, R. paranaense, R. petrolearium, R. phaseoli, R. phenanthrenilyticum, R. pisi, R. pongamiae, R. populi, R. pseudoryzae, R. pusense, R. qilianshanese, R. radiobacter, R. rhizogenes, R rhizoryzae, R rozettif ormans, R. rubi, R. selenitireeducens, R. skierneiwicense, R. smilacinae, R. soli, R. sophorae, R sophoriradicis, R. sphaerophysae, R. straminoryzae, R. subbaraonis, R. sullae, R. taibaishanense, R. tarimense, R. tibeticum, R. trifolii strains such as RP113-7, R. tropici strains such as SEMIA 4080, R. tubonense, R. undicola, R. vallis, R. viciae strains such as P1NP3Cst, SU303 and WSM 1455, R. vignae, R. vitis, R. yanglingense, R. yantingense), Rhizoctonia, Rhizopogon (e.g., R. amylopogon, R. fulvigleba, R. luteolus, R. villosuli), Rhodococcus, Saccharopolyspora (e.g., S. spinosd), Scleroderma (e.g., S. cepa S. citrinum), Septobasidium, Serratia, Shinella (e.g., S. kummerowiae), Sinorhizoium (e.g., S. abri, S. adhaerens, S. americanum, S. arboris, S. chiapanecum, S. fredii strains such as CCBAU114 and USDA 205, S. garamanticus, S. indiaense, S. kostiense, S. kummerowiae, S. medicae, S. meliloti strains such as MSDJ0848, S. mexicanus, S. numidicus, S. psoraleae, S. saheli, S. sesbaniae, S. sojae, S. terangae, S. xinjiangense), Sorosporella, Sphaerodes (e.g., S. mycoparasitica strains such as IDAC 301008-01), Spodoptera (e.g., S. littoralis), Sporodiniella, Steinernema (e.g., S. carpocapsae, S. feltiae, S. kraussei strains such as L137), Stenotrophomonas, Streptomyces (e.g., S. NRRL B-30145, S. M1064, S. WYE 53 (deposited as ATCC 55750), S. cacaoi strains such as ATCC 19093, S. galbus strains such as NRRL 30232, S. griseoviridis strains such as K61, S. lydicus strains such as WYEC 108 (deposited as ATCC 55445), S. violaceusniger strains such as YCED-9 (deposited as ATCC 55660)), Streptosporangium, Stillbella, Swaminathania, Talaromyces (e.g., T. aculeatus, T. flavus strains such as V117b), Tetranacrium, Thiobacillus, Tilachlidium, Tolypocladium, Tolypothrix, Torrubiella, Torulospora, Trenomyces, Trichoderma (e.g., T. asperellum strains such as SKT-1, T. atroviride strains such as LC52 and CNCM 1-1237, T. fertile strains such as JM41R T. gamsii strains such as ICC 080, T. hamatum strains such as ATCC 52198, T. harzianum strains such as ATCC 52445, KRL-AG2, T-22, TH-35, T-39 and ICC012, T. polysporum, T. reesi strains such as ATCC 28217 T. stromaticum, T. virens strains such as ATCC 58678, GL-3, GL-21 and G-41, T. viridae strains such as ATCC 52440, ICC080 and TV1), Typhula, Ulocladium (e.g., U oudemansii strains such as HRU3), Uredinella, Variovorax, Verticillium (e.g., V. chlamydosporum, V. lecanii strains such as ATCC 46578), Vibrio, Xanthobacter, Xanthomonas. Xenorhadbus, Yersinia (e.g., Y. entomophaga strains such as 082KB8), and Zoophthora.

Some examples of nematode-antagonistic biocontrol agents include ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Nematode-antagonistic biocontrol agents may include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pasteuria usage, Brevibacillus laterosporus strain G4, Pseudomonas fluorescens, and Rhizobacteria.

Microbes useful in the methods and compositions disclosed herein may be obtained from any source. In some cases, microbes may be bacteria, archaea, protozoa, or fungi. The microbes of this disclosure may be phosphate solubilizing microbes, for example phosphate solubilizing bacteria, phosphate solubilizing archaea, phosphate solubilizing fungi, phosphate solubilizing yeast, or phosphate solubilizing protozoa. Microbes useful in the methods and compositions disclosed herein may be spore forming microbes, for example spore forming bacteria. In some cases, bacteria useful in the methods and compositions disclosed herein may be Gram positive bacteria or Gram negative bacteria. In some cases, the bacteria may be an endospore forming bacteria of the Firmicute phylum. In some cases, the bacteria may be a diazotroph. In some cases, the bacteria may not be a diazotroph.

The methods and compositions of this disclosure may be used with an archaea, such as, for example, Methanothermobacter thermoautotrophicus.

In some cases, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus), Bacillus amyloliquefaciens, Bacillus aneurinolyticus, Bacillus atrophaeus, Bacillus azotoformans, Bacillus badius, Bacillus cereus (synonyms: Bacillus endorhythmos and Bacillus medusa), Bacillus chitinosporus, Bacillus circulans, Bacillus coagulans, Bacillus endoparasiticus, Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis, Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some cases, the bacterium may be Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum, or Rhizobium etli.

In some cases, the bacterium may be a species of Clostridium, for example, Clostridium pasteurianum, Clostridium beijerinckii, Clostridium perfringens, Clostridium tetani, or Clostridium acetobutylicum.

In some cases, bacteria used with the methods and compositions of the present disclosure may be cyanobacteria. Examples of cyanobacterial genera include Anabaena (for example, Anagaena sp. PCC7120), Nostoc (for example, Nostoc punctiforme), or Synechocystis (for example, Synechocystis sp. PCC6803).

In some cases, bacteria used with the methods and compositions of the present disclosure may belong to the phylum Chlorobi, for example, Chlorobium tepidum.

The microbes, and methods of producing microbes described herein, may apply to microbes able to self-propagate efficiently on the leaf surface, root surface, or inside plant tissues without inducing a damaging plant defense reaction, or microbes that are resistant to plant defense responses. The microbes described herein may be isolated by culturing a plant tissue, plant tissue extract, root surface wash, or leaf surface wash in a medium with no added soluble phosphorous. The microbe described herein may be an endophyte or an epiphyte or a microbe inhabiting the plant rhizosphere (rhizospheric microbe). Endophytes are organisms that enter the interior of plants without causing disease symptoms or eliciting the formation of symbiotic structures and are of agronomic interest because they can enhance plant growth and improve the nutrition of plants (e.g., through phosphate solubilization). The microbe can be a seed-borne endophyte. Seed-borne endophytes include microbes associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne endophyte is capable of replicating within the plant tissue, for example, the interior of the seed. Also, in some cases, the seed-borne endophyte is capable of surviving desiccation.

The microbes according to methods of the disclosure, or used in methods or compositions of the disclosure, can comprise a plurality of different microbial taxa in combination. By way of example, the microbes may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium, and Halomonas), Firmicutes (such as Bacillus, Paenibacillus, Lactobacillus, Mycoplasma, and Acetabacterium), and Actinobacteria (such as Streptomyces, Rhodacoccus, Microbacterium, and Curtobacterium).

Bacteria that can be modified by the methods disclosed herein include Azotobacter sp., Bradyrhizobium sp., Klebsiella sp., and Sinorhizobium sp. In some cases, the bacteria may be selected from the group consisting of: Azotobacter vinelandii, Bradyrhizobium japonicum, Klebsiella pneumoniae, and Sinorhizobium meliloti. In some cases, the bacteria may be of the genus Enterobacter or Rahnella. In some cases, the bacteria may be of the genus Frankia or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some cases, the bacteria may be of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae sub sp. Larvae, Paenibacillus larvae sub sp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria according to methods of the disclosure can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some embodiments, a microbe of the present disclosure may be an Acinetobacter, Actinomycetes, Aegerita, Agrobacterium (e.g., A. radiobacter strains such as K1026 and K84), Akanthomyces, Alcaligenes, Alternaria, Aminobacter (e.g., A. aganoensis, A. aminovorans, A. anthyllidis, A. cicerones, A. lissarensis, A. niigataensis), Ampelomyces (e.g., A. quisqualis strains such as M-10), Anabaena (e.g., A. aequalis, A. affinis, A. angstumalis, A. angstumalis marchita, A. aphanizomendoides, A. azollae, A. bornetiana, A. catenula, A. cedrorum, A. circinalis, A. confervoides, A. constricta, A. cyanobacterium, A. cycadeae, A. cylindrica, A. echinispora, A. felisii, A. flos-aquae, A. flos-aquae minor, A. flos-aquae treleasei, A. helicoidea, A. inaequalis, A. lapponica, A. laxa, A. lemmermannii, A. levanderi, A. limnetica, A. macrospora, A. macrospora robusta, A. monticulosa, A. nostoc, A. ascillarioides, A. planctonica, A. raciborski, A. scheremetievi, A. sphaerica, A. spiroides crassa, A. spiroides sprroides, A. subcylindrica, A. torulosa, A. unispora, A. variabilis, A. verrucosa, A. viguieri, A. wisconsinense, A. zierlingii), Arthrobacter, Arthrobotrys (e.g., A. aggregata, A. alaskana, A. ameropora, A. anomala, A. apscheronica, A. arthrobotryoides, A. azerbaijanica, A. bakunika, A. botryospora, A. brochopaga, A. chazarica, A. chilensis, A. cladodes, A. calvispora, A. compacta, A. conoides, A. constringens, A. cylindrospora, A. dactyloides, A. deflectans, A. dendroides, A. doliiformis, A. drechsleri, A. elegans, A. ellipsospora, A. entomopaga, A. ferox, A. foliicola, A. fruticulosa, A. globospora, A. hatospora, A. hertziana, A. indica, A. irregularis, A. javanica, A. kirghizica, A. longa, A. longiphora, A. longiramulifera, A. longispora, A. mangrovispora, A. megaspora, A. microscaphoides, A. macrospora, A. multisecundaria, A. musiformis, A. nematopaga, A. nonseptata, A. oligospora, A. oudemansii, A, oviformis, A. perpasta, A. polycephala, A. pseudoclavata, A. pyriformis, A. recta, A. robusta, A. rosea, A. scaphoides, A. sclerohypha, A. shahriari, A. shizishanna, A. sinensis, A. soprunovii, A. stilbacea, A. straminicola, A. superba, A. tabrizica, A. venusta, A. vermicola, A. yunnanensis), Aschersonia, Ascophaera, Aspergillus (e.g., A. flavus strains such as NRRL 21882, A. parasiticus), Aulosira (e.g., A. aenigmatica, A. africana, A. bohemensis, A. bombayensis, A. confluens, A. fertilissima, A. fertilissma var. tenius, A. fits chii, A. godoyana, A. implexa, A. laxa, A. plantonica, A. proliftca, A. pseuodoramosa, A. schauinslandii, A. striata, A. terrestris, A. thermalis), Aureobacterium, Aureobasidium (e.g., A. pullulans strains such as DSM 14940 and DSM 14941), Azobacter, Azorhizobium (e.g., A. caulinodans, A. doebereinerae, A. oxalatiphilum), Azospirillum (e.g., A. amazonense strains such as BR 11140 (SpY2T), A. brasilense strains such as INTA Az-39, AZ39, XOH, BR 11002, BR 11005, Ab-V5 and Ab-V6. A. canadense, A. doebereinerae, A. formosense, A. halopraeferans, A. irakense, A. largimobile, A. lipoferum strains such as BR 11646, A. melinis, A. oryzae, A. picis, A. rugosum, A. thiophilum, A. zeae), Azotobacter (e.g., A. agilis, A. armeniacus, A. sp. AR, A. beijerinckii, A. chroococcum, A. DCU26, A. FA8, A. nigricans, A. paspali, A. salinestris, A. tropicalis, A. vinelandii), Bacillus (e.g., B. amyloliquefaciens strains such as D747, NRRL B-50349, TJ1000 (also known as 1BE, isolate ATCC BAA-390), FZB24, FZB42, IN937a, IT-45, TJ1000, MBI600, BS27 (deposited as NRRL B-5015), BS2084 (deposited as NRRL B-50013), 15AP4 (deposited as ATCC PTA-6507), 3AP4 (deposited as ATCC PTA-6506), LSSA01 (deposited as NRRL B-50104), ABP278 (deposited as NRRL B-50634), 1013 (deposited as NRRL B-50509), 918 (deposited as NRRL B-50508), 22CP1 (deposited as ATCC PTA-6508) and BS18 (deposited as NRRL B-50633), B. cereus strains such as 1-1562, B. flrmus strains such as 1-1582, B. laevolacticus, B. lichenformis strains such as BA842 (deposited as NRRL B-50516) and BL21 (deposited as NRRL B-50134), B. macerns, B. flrmus, B. mycoides strains such as NRRL B-21664, B. pasteurii, B. pumilus strains such as NRRL B-21662, NRRL B-30087, ATCC 55608, ATCC 55609, GB34, KFP9F and QST 2808, B. sphaericus, B. subtilis strains such as ATCC 55078, ATCC 55079, MBI 600, NRRL B-21661, NRRL B-21665, CX-9060, GB03, GB07, QST 713, FZB24, D747 and 3BP5 (deposited as NRRL B-50510), B. thuringiensis strains such as ATCC 13367, GC-91, NRRL B-21619, ABTS-1857, SAN 401 I, ABG-6305, ABG-6346, AM65-52, SA-12, SB4, ABTS-351, HD-1, EG 2348, EG 7826, EG 7841, DSM 2803, NB-125 and NB-176), Beijerinckia, Beauveria (e.g., B. bassiana strains such as ATCC 26851, ATCC 48023, ATCC 48585, ATCC 74040, ATCC-74250, DSM 12256 and PPM 5339), Beijerinckia, Blastodendrion, Bosea (e.g., B. eneae, B. lathyri, B. lupini, B. massiliensis, B. minatitlanensis, B. robiniae, B. thiooxidans, B. vestrisii), Bradyrhizobium (e.g., B. arachidis, B. bete, B. canariense, B. cytisi, B. daqingense, B. denitriflicans, B. diazoeffliciens, B. elkanii strains such as SEMIA 501, SEMIA 587 and SEMIA 5019, B. ganzhouense, B. huanghuauhaiense, B. icense, B. ingae, B. iriomotense, B. japonicum strains such as NRRL B-50586 (also deposited as NRRL B-59565), NRRL B-50587 (also deposited as NRRL B-59566), NRRL B-50588 (also deposited as NRRL B-59567), NRRL B-50589 (also deposited as NRRL B-59568), NRRL B-50590 (also deposited as NRRL B-59569), NRRL B-50591 (also deposited as NRRL B-59570), NRRL B-50592 (also deposited as NRRL B-59571), NRRL B-50593 (also deposited as NRRL B-59572), NRRL B-50594 (also deposited as NRRL B-50493), NRRL B-50608, NRRL B-50609, NRRL B-50610, NRRL B-50611, NRRL B-50612, NRRL B-50726, NRRL B-50727, NRRL B-50728, NRRL B-50729, NRRL B-50730, SEMIA 566, SEMIA 5079, SEMIA 5080, USDA 6, USDA 110, USDA 122, USDA 123, USDA 127, USDA 129 and USDA 532C, B. jicamae, B. lablabi, B. liaoningense, B. manausense, B. neotropicale, B. oligotrophicum, B. ottawaense, B. pachyrhizi, B. paxllaeri, B. retamae, B. rifense, B. valentinum, B. yuanmingense), Burkholderia (e.g., B. acidipaludis, B. ambifaria, B. andropogonis, B. anthina, B. arboris, B. bannensis, B. bryophila, B. caledonica, B. caribensis, B. caryophylli, B. cenocepacua, B. choica, B. cocovenenans, B. contaminans, B. denitriflcans, B. diazotrophica, B. diffusa, B. dilworthii, B. dolosa, B. eburnea, B. endofungorum, B. ferrariae, B. fungorum, B. ginsengisoli, B. gladioli, B. glathei, B. glumae, B. graminis, B. grimmiae, B. heleia, B. hospital, B. humi, B. kururiensis, B. lata, B. latens, B. mallei, B. megapolitana, B. metallica, B. mimosarum, B. multivorans, B. nodosa, B. norimbergensis, B. oklahomensis, B. phenazinium, B. phenoliruptrix, B. phymatum, B. phytofirmans, B. pickettii, B. plantarii, B. pseudomallei, B. pseudomultivorans, B. pyrrocinia, B. rhizoxinica, B. rhynchosiae, B. sabiae, B. sacchari, B. sartisoli, B. sediminicola, B. seminalis, B. silvatlantica, B. singaporensis, B. soli, B. sordidcola, B. sp. strains such as A396, B. sprentiae, B. stabilis, B. symbiotica, B. telluris, B. terrae, B. terrestris, B. terricola, B. thailandensis, B. tropica, B. tuberum, B. ubonensis, B. udeis, B. unamae, B. vandii, B. vietnamiensis, B. xenovorans, B. zhejiangensis), Brevibacillus, Burkholderia (e.g., B. sp. A396 nov. rinojensis NRRL B-50319), Calonectria, Candida (e.g., C. oleophila such 1-182, C. saitoand), Candidatus (e.g., C. Burkholderia calva, C. Burkholderia crenata, C. Burkholderia hispidae, C. Burkholderia kirkii, C. Burkholderia mamillata, C. Burkholderia nigropunctata, C. Burkholderia rigidae, C. Burkholderia schumannianae, C. Burkholderia verschuerenii, C. Burkholderia virens, C. Phytoplasma allocasuarinae, C. Phytoplasma americanum, C. Phytoplasma asteris, C. Phytoplasma aurantifolia, C. Phytoplasma australiense, C. Phytoplasma balanitae, C. Phytoplasma brasiliense, C. Phytoplasma caricae, C. Phytoplasma castaneae, C. Phytoplasma cocosnigeriae, C. Phytoplasma cocostanzaniae, C. Phytoplasma convolvuli, C. Phytoplasma costaricanum, C. Phytoplasma cynodontis, C. Phytoplasma fragariae, C. Phytoplasma fraxini, C. Phytoplasma graminis, C. Phytoplasma japonicum, C. Phytoplasma luffae, C. Phytoplasma lycopersici, C. Phytoplasma malasianum, C. Phytoplasma mali, C. Phytoplasma omanense, C. Phytoplasma oryzae, C. Phytoplasma palmae, C. Phytoplasma palmicola, C. Phytoplasma phoenicium, C. Phytoplasma pini, C. Phytoplasma pruni, C. Phytoplasma prunorum, C. Phytoplasma pyri, C. Phytoplasma rhamni, C. Phytoplasma rubi, C. Phytoplasma solani, C. Phytoplasma spartii, C. Phytoplasma sudamericanum, C. Phytoplasma tamaricis, C. Phytoplasma trifolii, C. Phytoplasma ulmi, C. Phytoplasma vitis, C. Phytoplasma ziziphi), Chromobacterium (e.g., C. subtsugae NRRL B-30655 and PRAA4-1, C. vaccinia strains such as NRRL B-50880, C. violaceum), Chryseomonas, Clavibacter, Clonostachys (e.g., C. rosea f. catenulata (also referred to as Gliocladium catenulatum) strains such as J1446), Clostridium, Coelemomyces, Coelomycidium, Colletotrichum (e.g., C. gloeosporioides strains such as ATCC 52634), Comomonas, Conidiobolus, Coniothyrium (e.g., C. minitans strains such as CON/M/91-08), Cordyceps, Corynebacterium, Couchia, Cryphonectria (e.g., C. parasitica), Cryptococcus (e.g., C. albidus), Cryptophlebia (e.g., C. leucotreta), Culicinomyces, Cupriavidus (e.g., C. alkaliphilus, C. basilensis, C. campinensis, C. gilardii, C. laharis, C. metallidurans, C. numazuensis, C. oxalaticus, C. pampae, C. pauculus, C. pinatubonensis, C. respiraculi, C. taiwanensis), Curtobacterium, Cydia (e.g., C. pomonella strains such as V03 and V22), Dactylaria (e.g., D. Candida), Delftia (e.g., D. acidovorans strains such as RAY209), Desulforibtio, Desulfovibrio, Devosia (e.g., D. neptuniae), Dilophosphora (e.g., D. alopecuri), Engyodontium, Enter obacter, Entomophaga, Entomophthora, Erynia, Escherichia (e.g., E. intermedia), Eupenicillium, Exiguobacaterium, Filariomyces, Filobasidiella, Flavobacterium (e.g., F. H492 NRRL B-50584), Frankia (e.g., F. alni), Fusarium (e.g., F. laterium, F. oxysporum, F. solani), Gibellula, Gigaspora (e.g. G. margarita), Gliocladium (e.g., G. virens strains such as ATCC 52045 and GL-21), Glomus (e.g., G. aggregatum, G. brasilianum G. clarumfi. deserticola JG. etunicatum JG. fasciculatum, G. intraradices strains such as RTI-801, G. monosporum, G. mosseae), Gluconobacter, Halospirulina, Harposporium (e.g., H. anguillulae), Hesperomyces, Hirsutella (e.g., H. minnesotensis, H. rhossiliensis, H. thorns onii strains such as ATCC 24874), Hydrogenophage, Hymenoscyphous (e.g., H. ericae), Hymenostilbe, Hypocrella, Isaria (e.g., I. fumosorosea strains such as Apopka-97 (deposited as ATCC 20874)), Klebsiella (e.g., K. pneumoniae, K. oxytoca), Kluyvera, Laccaria (e.g., L. bicolor, L. laccata), Lactobacillus, Lagenidium, Lecanicillium (e.g., L. lecanii strains such as KV01, L. longisporum strains such as KV42 and KV71), Leptolegnia, Lysobacter (e.g., L. antibioticus strains such as 13-1 and HS124, L. enzymogenes strains such as 3.1T8), Massospora, Meristacrum (e.g., M. asterospermum), Mesorhizobium (e.g., M. abyssinicae, M. albiziae, M. alhagi, M. amorphae, M. australicum, M. camelthorni, M. caraganae, M. chacoense, M. ciceri, M. gobiense, M. hawassense, M. huakuii, M. loti, M. mediterraneum, M. metallidurans, M. muleiense, M. opportunistum, M. plurifarium, M. qingshengii, M. robiniae, M. sangaii, M. septentrionale, M. shangrilense, M. shonense, M. silamurunense, M. tamadayense, M. tarimense, M. temperatum, M. thiogangeticum, M. tianshanense), Metarhizium (e.g., M. anisopliae (also referred to as M. brunneum, Metarrhizium anisopliae, and green muscadine) strains such as IMI 330189, FI-985, FI-1045, F52 (deposited as DSM 3884, DSM 3885, ATCC 90448, SD 170, and ARSEF 7711) and ICIPE 69), M. flavoviride strains such as ATCC 32969), Methylobacterium (e.g., M. adhaesivum, M. aerolatum, M. aminovorans, M. aquaticum, M. brachiatum, M. brachythecii, M. bullatum, M. cerastii, M. chloromethanicum, M. dankookense, M. dichloromethanicum, M. extorquens, M. fujisawaense, M. gnaphalii, M. goesingense, M. gossipiicola, M. gregans, M. haplocladii, M. hispanicum, M. iners, M. isbiliense, M. jeotgali, M. komagatae, M. longum, M. lusitanum, M. marchantiae, M. mesophilicum, M. nodulans, M. organophilum, M. oryzae, M. oxalidis, M. persicinum, M. phyllosphaerae, M. platani, M. podarium, M. populi, M. radiotolerans, M. rhodesianum, M. rhodinum, M. salsuginis, M. soli, M. suomiense, M. tardum, M. tarhaniae, M. thiocyanatum, M. thurigiense, M. trifolii, M. variabile, M. zatmanii), Metschnikowia (e.g., M. fructicola), Microbacterium (e.g., M. laevaniformans), Microdochium (e.g., M. dimerum), Microsphaeropsis (e.g., M. ochracea P130A), Microvirga (e.g., M. aerilata, M. aerophila, M. flocculans, M. guangxiensis, M. lotononidis, M. lupini, M. subterranea, M. vignae, M. zambiensis), Monacrosporium (e.g., M. cionopagum), Mucor, Muscodor (e.g., M. albus such NRRL 30547, QST 20799 and SA-13, M. roseus strains such as NRRL 30548), Mycoderma, Myiophagus, Myriangium, Myrothecium (e.g., M. verrucaria), Nectria, Nematoctonus (e.g., N. geogenius, N. leiosporus), Neozygites, Nomuraea (e.g., N. rileyi strains such as SA86101, GU87401, SR86151, CG128 and VA9101), Nostoc (e.g., N. azollae, N. caeruleum, N. carneum, N. comminutum, N. commune, N. ellipsosporum, N. flagelliforme, N. linckia, N. longstaffl, N. microscopicum, N. muscorum, N. paludosum, N. pruniforme, N. punctifrome, N. sphaericum, N. sphaeroides, N. spongiaeforme, N. verrucosum), Ochrobactrum (e.g., O. anthropi, O. cicero, O. cytisi, O. daejeonense, O. gallinifaecis, O. grigonense, O. guangzhouense, O. haematophilum, O. intermedium, O. lupini, O. oryzae, O. pectoris, O. pituitosum, O. pseudointermedium, O. pseudogrignonense, O. rhizosphaerae, O. thiophenivorans, O. tritici), Oidiodendron, Paecilomyces (e.g., P. fumosoroseus strains such as FE991 and FE 9901, P. lilacinus strains such as 251, DSM 15169 and BCP2), Paenibacillus (e.g., P. alvei strains such as NAS6G6, P. azotofixans, P. polymyxa strains such as ABP166 (deposited as NRRL B-50211)), Pandora, Pantoea (e.g., P. agglomerans strains such as NRRL B-21856, P. vagans strains such as C9-1), Paraglomus (e.g., P. brazilianum), Paraisaria, Pasteuria, Pasteuria (e.g., P. nishizawae strains such as Pnl, P. penetrans, P. ramose, P. sp. strains such as ATCC PTA-9643 and ATCC SD-5832, P. thornea, P. usage), Penicillium (e.g., P. albidum, P. aurantiogriseum, P. bilaiae (formerly known as P. bilaii and P. bilaji) strains such as ATCC 18309, ATCC 20851, ATCC 22348, NRRL 50162, NRRL 50169, NRRL 50776, NRRL 50777, NRRL 50778, NRRL 50777, NRRL 50778, NRRL 50779, NRRL 50780, NRRL 50781, NRRL 50782, NRRL 50783, NRRL 50784, NRRL 50785, NRRL 50786, NRRL 50787, NRRL 50788 and RS7B-SD1, P. brevicompactum strains such as AgRF18, P. canescens strains such as ATCC 10419, P. chyrsogenum, P. citreonigrum, P. citrinum, P. digitatum, P. expansum strains such as ATCC 24692 and YT02, P. fellatanum strains such as ATCC 48694, P. frequentas, P. fuscum, P. fussiporus, P. gaestrivorus strains such as NRRL 50170, P. glabrum strains such as DAOM 239074 and CBS 229.28, P. glaucum, P. griseofulvum, P. implicatum, P. janthinellum strains such as ATCC 10455, P. lanosocoeruleum strains such as ATCC 48919, P. lilacinum, P. minioluteum, P. montanense, P. nigricans, P. oxalicum, P. pinetorum, P. pinophilum, P. purpurogenum, P. radicum strains such as ATCC 201836, FRR 4717, FRR 4719 and N93/47267, P. raistrickii strains such as ATCC 10490, P. rugulosum, P. simplicissimum, P. solitum, P. variabile, P. velutinum, P. viridicatum), Phingobacterium, Phlebiopsis (e.g., P. gigantea), Photorhabdus, Phyllobacterium (e.g., P. bourgognense, P. brassicacearum, P. catacumbae, P. endophyticum, P. ifriqiyense, P. leguminum, P. loti, P. myrsinacearum, P. sophorae, P. trifolii), Pichia (e.g., P. anomala strains such as WRL-076), Pisolithus (e.g., P. tinctorius), Planktothricoides, Plectonema, Pleurodesmospora, Pochonia (e.g., P. chlamydopora), Podonectria, Polycephalomyces, Prochlorocoous (e.g., P. marinus), Prochloron (e.g., P. didemni), Prochlorothrix, Pseudogibellula, Pseudomonas (e.g., P. agarici, P. antartica, P. aurantiaca, P. aureofaciens, P. azotiflgens, P. azotoformans, P. balearica, P. blatchfordae, P. brassicacearum, P. brenneri, P. cannabina, P. cedrina, P. cepacia, P. chlororaphis strains such as MA 342, P. congelans, P. corrugata, P. costantinii, P. denitriflcans, P. entomophila, P. fluorescens strains such as ATCC 27663, CL 145 A and A506, P. fragii, P. fuscovaginae, P. fulva, P. gessardii, P. jessenii strains such as PS06, P. kilonensis, P. koreensis, P. libanensis, P. lili, P. lundensis, P. lutea, P. luteola, P. mandelii, P. marginalis, P. meditrranea, P. meridana, P. migulae, P. moraviensis, P. mucidolens, P. orientalis, P. oryzihabitans, P. palleroniana, P. panacis, P. parafulva, P. peli, P. pertucinogena, P. plecoglossicida, P. protogens, P. proteolytica, P. putida, P. pyrocina strains such as ATCC 15958, P. rhodesiae, P. sp. strains such as DSM 13134, P. striata, P. stutzeri, P. syringae, P. synxantha, P. taetrolens, P. thisvervalensis, P. tolaasii, P. veronii), Pseudozyma (e.g., P. flocculosa strains such as PF-A22 UL), Pythium (e.g., P. oligandrum strains such as DV 74), Rhizobium (e.g., R. aggregatum, R alamii, R. alkalisoli, P. alvei, P. azibense, P. borbori, R. calliandrae, Rcauense, R. cellulosilyticum, R. daejeonense, R. endolithicum, R. endophyticum, R. etli, R. fabae, R. flavum, R. fredii, R. freirei, R. galegae, R. gallicum, R. giardinii, R. grahamii, R. hainanense, R. halophytocola, R. halotolerans, R. helanshanense, R. herbae, R. huautlense, R. indigoferae, R. jaguaris, R kunmingense, R. laguerreae, R. larrymoorei, R. leguminosarum strains such as S012A-2 (IDAC 080305-01), R lemnae, R leucaenae, R. loessense, R. lupini, R. lusitanum, R. mayense, R mesoamericanum, R. mesosinicum, R. miluonense, R. mongolense, R. multihospitium, R. naphthalenivorans, R. nepotum, R. oryzae, R. pakistanensis, R. paknamense, R. paranaense, R. petrolearium, R. phaseoli, R. phenanthrenilyticum, R. pisi, R. pongamiae, R. populi, R. pseudoryzae, R. pusense, R. qilianshanese, R. radiobacter, R. rhizogenes, R. rhizoryzae, R. rozettiformans, R. rubi, R. selenitireeducens, R. skierneiwicense, R. smilacinae, R. soli, R. sophorae, R sophoriradicis, R. sphaerophysae, R. straminoryzae, R. subbaraonis, R. sullae, R. taibaishanense, R. tarimense, R. tibeticum, R. trifolii strains such as RP113-7, R. tropici strains such as SEMIA 4080, R. tubonense, R. undicola, R. vallis, R. viciae strains such as PlNP3Cst, SU303 and WSM 1455, R. vignae, R. vitis, R. yanglingense, R. yantingense), Rhizoctonia, Rhizopogon (e.g., R amylopogon, R. fulvigleba, R. luteolus, R. villosuli), Rhodococcus, Saccharopolyspora (e.g., S. spinosa), Scleroderma (e.g., S. cepa S. citrinum), Septobasidium, Serratia, Shinella (e.g., S. kummerowiae), Sinorhizoium (e.g., S. abri, S. adhaerens, S. americanum, S. arboris, S. chiapanecum, S. fredii strains such as CCBAU114 and USDA 205, S. garamanticus, S. indiaense, S. kostiense, S. kummerowiae, S. medicae, S. meliloti strains such as MSDJ0848, S. mexicanus, S. numidicus, S. psoraleae, S. saheli, S. sesbaniae, S. sojae, S. terangae, S. xinjiangense), Sorosporella, Sphaerodes (e.g., S. mycoparasitica strains such as IDAC 301008-01), Spodoptera (e.g., S. littoralis), Sporodiniella, Steinernema (e.g., S. carpocapsae, S. feltiae, S. kraussei strains such as L137), Stenotrophomonas, Streptomyces (e.g., S. NRRL B-30145, S. M1064, S. WYE 53 (deposited as ATCC 55750), S. cacaoi strains such as ATCC 19093, S. galbus strains such as NRRL 30232, S. griseoviridis strains such as K61, S. lydicus strains such as WYEC 108 (deposited as ATCC 55445), S. violaceusniger strains such as YCED-9 (deposited as ATCC 55660)), Streptosporangium, Stillbella, Swaminathania, Talaromyces (e.g., T. aculeatus, T. flavus strains such as V117b), Tetranacrium, Thiobacillus, Tilachlidium, Tolypocladium, Tolypothrix, Torrubiella, Torulospora, Trenomyces, Trichoderma (e.g., T. asperellum strains such as SKT-1, T. atroviride strains such as LC52 and CNCM 1-1237, T. fertile strains such as JM41R, T. gamsii strains such as ICC 080, T. hamatum strains such as ATCC 52198, T. harzianum strains such as ATCC 52445, KRL-AG2, T-22, TH-35, T-39 and ICC012, T. polysporum, T. reesi strains such as ATCC 28217 T. stromaticum, T. virens strains such as ATCC 58678, GL-3, GL-21 and G-41, T. viridae strains such as ATCC 52440, ICC080 and TV1), Typhula, Ulocladium (e.g., U oudemansii strains such as HRU3), Uredinella, Variovorax, Verticillium (e.g., V. chlamydosporum, V. lecanii strains such as ATCC 46578), Vibrio, Xanthobacter, Xanthomonas. Xenorhadbus, Yersinia (e.g., Y. entomophaga strains such as 082KB8), and Zoophthora.

The bacteria may be obtained from any general terrestrial environment, including its soils, plants, fungi, animals (including invertebrates), and other biota, including the sediments, water, and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand, and clays); the cryosphere and its meltwater; the atmosphere (for example, filtered aerial dusts, cloud, and rain droplets); urban, industrial, and other man-made environments (for example, accumulated organic and mineral matter on concrete, roadside gutters, roof surfaces, and road surfaces).

Microbes useful in methods and compositions disclosed herein can be obtained by extracting microbes from surfaces or tissues of plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizospheric bacteria, epiphytes, or endophytes. The isolates can be cultured in media containing calcium phosphate as the sole source of phosphate to enrich for bacteria that are able to solubilization calcium phosphate. Alternatively, microbes can be obtained from global strain banks.

In planta analytics are performed to assess microbial traits. In some embodiments, the plant tissue can be processed for screening by high-throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling include using methods of quantitative polymerase chain reaction (qPCR), molecular barcodes for transcript detection, Next Generation Sequencing, and microbe tagging with fluorescent markers.

Automated greenhouses can be used for in planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer may be used to screen for microbes which express phosphate solubilizing genes. A microbial population can also be enriched via single-cell, culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

Enriching for Microbes with Phosphate Solubilizing Capabilities Using Bioinformatics

Bioinformatics tools can be used to identify and isolate plant growth promoting microbes (PGPMs), which are selected based on their ability to perform phosphate solubilization. Microbes with high phosphate solubilization ability can promote favorable traits in plants. Bioinformatic modes of analysis for the identification of PGPMs include, but are not limited to, genomics, metagenomics, targeted isolation, gene sequencing, transcriptome sequencing, and modeling. Genomic analysis can be used to identify PGPMs and confirm the presence of mutations with methods of Next Generation Sequencing, as described herein, and microbe version control.

Metagenomics can be used to identify and isolate PGPMs using a prediction algorithm for colonization. Metadata can also be used to identify the presence of an engineered strain in environmental and greenhouse samples.

Transcriptomic sequencing can be used to predict genotypes leading to PGPM phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counter-selectable suicide vector can be incorporated into the genome of a microbe using recombineering methods. A counter-selectable suicide vector may consist of a deletion of the gene of interest, a selectable marker, and the counter-selectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Non-Transgenic Engineering of Microbes

A microbial population with favorable traits can be obtained via directed evolution. Directed evolution is an approach wherein the process of natural selection is mimicked to evolve proteins or nucleic acids towards a user-defined goal. An example of directed evolution is when random mutations are introduced into a microbial population, the microbes with the most favorable traits are selected, and the growth of the selected microbes is continued. The most favorable traits in plant growth promoting microbes (PGPMs) may be in phosphate solubilization. The method of directed evolution may be iterative and adaptive based on the selection process after each iteration.

Plant growth promoting microbes (PGPMs) with high capability of phosphate solubilization can be generated. The evolution of PGPMs can be carried out via the introduction of genetic variation. Genetic variation can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved PGPM properties, improved colonization of cereals, and increased phosphate solubilization. Intrageneric gene modifications can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis. An example of an intrageneric gene modification may be using a promoter from a highly expressed gene within an organism to control expression of a gene associated with phosphate solubilization in the organism.

The intra-generic gene modifications can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Modifications

In one aspect, the present disclosure provides a non-intergenic bacterium comprising one or more genetic variations introduced into one or more genes or non-coding polynucleotides associated with phosphorous solubilization, such that the bacterium is capable of solubilizing organic and/or inorganic phosphorous.

This disclosure further provides a method for improving phosphate solubilization by phosphate-solubilizing microbes. In some embodiments, the method comprises selecting a gene known to be involved in phosphate solubilization, or a gene homologous to a gene known to be involved in phosphate solubilization. In some cases, microbes may solubilize phosphate through the release of low molecular weight organic acids, which chelate the cations bound to phosphate, thereby converting it into soluble forms. Genes which may be associated with solubilization of organic phosphorous include non-specific acid phosphatases (NSAPs) such as phoC, napA, napD, napE, acpA, and appA, and phytases such as phy and appA. Genes which may be associated with solubilization of inorganic phosphorous include gluconic acid biosynthetic genes such as pqq biosynthetic genes, pqqA, pqqB, pqqC, pqqD, pqqE, gcd, and gabY. Genes which may be associated with phosphate solubilization also include alkaline phosphatases such as phoA, phoC, and phoD. Another example of a gene which may be associated with phosphate solubilization is glucose dehydrogenase (e.g., gcd). Genes which may be negatively associated with phosphorous solubilization include gntT (gluconic acid transporter) and gad (gluconate dehydrogenase). Examples of gad genes include gad1 and gad2. Examples of gluconic acid transporter genes include gntT and gntU. The expression level of genes involved in phosphate solubilization may be increased by codon randomizing the coding sequence to remove regulatory sequences, and reintroducing the codon randomized coding sequence in a plasmid. The plasmid may also comprise a promoter, an RBS, an origin of replication, a selectable marker, and other elements to control the expression level of the coding sequence or the copy number of the plasmid. The plasmid may be a high copy number plasmid, a moderate copy number plasmid, or a low copy number plasmid.

Genetic variations introduced into microbes may be classified as transgenic, cisgenic, intragenomic, intrageneric, intergeneric, synthetic, evolved, rearranged, or SNPs.

Genetic variation may be introduced into numerous metabolic pathways within microbes to elicit improvements in phosphate solubilization. Representative pathways include phosphate solubilization, organic acid transport, and organic acid production.

CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently linked to form a single molecule (also called a single guide RNA (“sgRNA”)). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing, deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity).

The microbes of the present disclosure may be identified by one or more genetic modifications or alterations, which have been introduced into the microbe. One method by which the genetic modification or alteration can be identified is via reference to a SEQ ID NO that contains a portion of the microbe's genomic sequence that is sufficient to identify the genetic modification or alteration.

In certain aspects, the disclosure provides for a sequence, which shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any sequence selected from the group consisting of SEQ ID NOs:1-93. In certain aspects, the disclosure provides for a sequence, which shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any sequence selected from the group consisting of SEQ ID NOs:3, 4, 15-21, 29-48, and 52-54.

In certain aspects, the disclosure provides for a microbe that comprises a sequence, which shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any sequence selected from the group consisting of SEQ ID NOs:1-71.

In certain aspects, the disclosure provides for a microbe that comprises a sequence, which shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any sequence selected from the group consisting of SEQ ID NOs:3, 4, 15-21, 29-48, and 52-54.

In some aspects, the disclosure provides for a microbe that comprises an amino acid sequence, which shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any sequence selected from the group consisting of SEQ ID NOs:72-93.

### Serial Passage

Production of bacteria to improve plant traits (e.g., phosphate solubilization) can be achieved through serial passage. The production of these bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic variation into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as 10 to 20 plants, or 20 or more, 50 or more, 100 or more, 300 or more, 500 or more, or 1000 or more plants.

In addition to obtaining a plant with an improved trait, a bacterial population comprising bacteria comprising one or more genetic variations introduced into one or more genes (e.g., genes regulating phosphate solubilization) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis may occur of isolated bacteria in step (a). Phenotypic and/or genotypic information may be obtained using techniques including: high through-put screening of chemical components of plant origin, sequencing techniques including high-throughput sequencing of genetic material, differential display techniques (including DDRT-PCR and DD-PCR), nucleic acid microarray techniques, RNA-sequencing (Whole Transcriptome Shotgun Sequencing), and qRT-PCR (quantitative real time PCR). Information gained can be used to obtain community profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, and nifD gene. Example processes of taxonomic profiling to determine taxa present in a population are described in U.S. Patent Application Publication No. 2014/0155283. Bacterial identification may comprise characterizing activity of one or more genes or one or more signaling pathways, such as genes associated with phosphate solubilization. Synergistic interactions (where two components, by virtue of their combination, increase a desired effect by more than an additive amount) between different bacterial species may also be present in the bacterial populations.

### Guided Microbial Remodeling—An Overview

Guided microbial remodeling is a method to systematically identify and improve the role of species within the crop microbiome. In some aspects, and according to a particular methodology of grouping/categorization, the method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters within a microbe's genome, and 3) screening and selection of new microbial genotypes that produce desired crop phenotypes.

To systematically assess the improvement of strains, a model is created that links colonization dynamics of the microbial community to genetic activity by key species. The model is used to predict genetic targets for non-intergeneric genetic remodeling (i.e., engineering the genetic architecture of the microbe in a non-transgenic fashion). Rational improvement of the crop microbiome may be used to increase soil biodiversity, tune impact of keystone species, and/or alter timing and expression of important metabolic pathways.

The metabolism of the species of interest can be mapped and linked to genetics. For example, genes associated with phosphate solubilization can be characterized. The pathway that is being characterized can be examined under a range of environmental conditions. For example, the microbe's ability to fix solubilize phosphate in the presence of various levels, and types, of soluble and insoluble phosphate in its environment can be examined. Examples of genes associated with phosphate solubilization are listed above.

Afterwards, a targeted non-intergeneric genomic alteration can be introduced to the microbe's genome, using methods including, but not limited to: conjugation and recombination, chemical mutagenesis, adaptive evolution, and gene editing. The targeted non-intergeneric genomic alteration can include an insertion, disruption, deletion, alteration, perturbation, modification, etc. of the genome.

Derivative remodeled microbes, which comprise the desired phenotype resulting from the remodeled underlying genotype, are then used to inoculate crops.

The present disclosure provides, in certain embodiments, non-intergeneric remodeled microbes that are able to solubilize phosphates. In aspects, these non-intergeneric remodeled microbes are able to solubilize phosphate at a greater level than the un-remodeled microbes. In some embodiments, the present disclosure finds microbial species that have desired colonization characteristics, and then utilizes those species in the subsequent remodeling process.

In some embodiments, the GMR platform comprises the following steps:
A. Isolation—Obtain microbes from the soil, rhizosphere, surface, etc. of a crop plant of interest.
B. Characterization—Involves characterizing the isolated microbes for genotype/phenotypes of interest (e.g., genome sequence, colonization ability, phosphate solubilization ability, etc.).
C. Domestication—Development of a molecular protocol for non-intergeneric genetic modification of the microbe.
D. Non-Intergeneric Engineering Campaign and Optimization—Generation of derivative non-intergeneric microbial strains with genetic modifications in key pathways (e.g., phosphate solubilization genes).
E. Analytics—Evaluation of derived non-intergeneric strains for phenotypes of interest both in vitro (e.g., phosphate solubilization) and in planta (e.g., colonization assays).
F. Iterate Engineering Campaign/Analytics—Iteration of steps D and E for further improvement of microbial strain.

### Isolation of Microbes

Microbes may be isolated from soil and/or roots of a plant. In one example, plants may be grown in a laboratory or a greenhouse in small pots. Soil samples may be obtained from various agricultural areas. For example, soils with diverse texture characteristics can be collected, including loam (e.g., peaty clay loam or sandy loam), clay soil (e.g., heavy clay or silty clay), sandy soil, silty soil, peaty soil, chalky soil, and the like.

Seeds of a bait plant (a plant of interest, e.g., corn, wheat, rice, sorghum, millet, soybean, vegetables, fruits, etc.) may be planted into each soil type. In one example, different varieties of a bait plant may be planted in various soil types. For example, if the plant of interest is corn, seeds of different varieties of corn such as field corn, sweet corn, heritage corn, etc. may be planted in various soil types as described above.

Plants may be harvested by uprooting them after a few weeks (e.g., 2-4 weeks) of growth. Alternative to growing plants in a laboratory/greenhouse, soil and/or roots of the plant of interest can be collected directly from the fields with different soil types.

To isolate rhizosphere microbes and epiphytes, plants may be removed gently by saturating the soil with distilled water or gently loosening the soil by hand to avoid damage to the roots. If larger soil particles are present, these particles may be removed by submerging the roots in a still pool of distilled water and/or by gently shaking the roots. The root may be cut and a slurry of the soil sticking to the root may be prepared by placing the root in a plate or tube with a small amount of distilled water and gently shaking the plate/tube on a shaker or centrifuging the tube at low speed. This slurry may be processed as described below.

To isolate endophytes, excess soil on root surfaces may be removed with deionized water. Following soil removal, plants may be surface sterilized and rinsed vigorously in sterile water. A cleaned, 1 cm section of root may be excised from the plant and placed in a phosphate buffered saline solution containing 3 mm steel beads. A slurry may be generated by vigorous shaking of the solution with a Qiagen TissueLyser II.

The soil and/or root slurry can be processed in various ways depending on the desired plant-beneficial trait of microbes to be isolated. For example, the soil and root slurry can be diluted and inoculated onto various types of screening media to isolate rhizospheric, endophytic, epiphytic, and other plant-associated microbes. For example, if the desired plant-beneficial trait is phosphate solubilization, then the soil/root slurry may be plated on media containing calcium phosphate as the sole source of phosphorus. Phosphate solubilizing bacteria (PSB) can solubilize calcium phosphate and assimilate and release phosphates. This reaction is manifested as a halo or a clear zone on the plate and can be used as an initial step for isolating PSB.

Populations of microbes obtained in the previous step may be streaked to obtain single colonies (pure cultures). A part of the pure culture may be resuspended in a suitable medium (e.g., a mixture of R2A and glycerol) and subjected to PCR analysis to screen for the presence of one or more genes of interest. For example, to identify bacteria that may be able to solubilize phosphates, purified cultures of isolated microbes can be subjected to a PCR analysis to detect the presence of genes associated with phosphate solubilization. Purified cultures of isolated strains may be stored, for example at −80° C., for future reference and analysis.

### Characterization of Isolated Microbes

Isolated microbes may be analyzed for phylogenetic characterization (assignment of genus and species) and the whole genome of the microbes may be sequenced. For phylogenetic characterization, 16S rDNA of the isolated microbe may be sequenced using degenerate 16S rDNA primers to generate phylogenetic identity. The 16S rDNA sequence reads may be mapped to a database to initially assign the genus, species, and strain name for isolated microbes. Whole genome sequencing may be used as the final step to assign phylogenetic genus/species to the microbes.

The whole genome of the isolated microbes may be sequenced to identify key pathways. For the whole genome sequencing, the genomic DNA may be isolated using a genomic DNA isolation kit (e.g., QIAmp DNA mini kit from QIAGEN) and a total DNA library may be prepared using the methods known in the art. The whole genome may be sequenced using high-throughput sequencing (also called Next Generation Sequencing) methods known in the art. For example, Illumina, Inc., Roche, and Pacific Biosciences provide whole genome sequencing tools that can be used to prepare total DNA libraries and perform whole genome sequencing.

The whole genome sequence for each isolated strain may be assembled; genes of interest may be identified; annotated; and noted as potential targets for remodeling. The whole genome sequences may be stored in a database.

Isolated microbes may be characterized for the colonization of host plants in a greenhouse. For this, seeds of the desired host plant (e.g., corn, wheat, rice, sorghum, and soybean) may be inoculated with cultures of isolated microbes individually or in combination and planted into soil. Alternatively, cultures of isolated microbes, individually or in combination, can be applied to the roots of the host plant by inoculating the soil directly over the roots. The colonization potential of the microbes may be assayed, for example, using a quantitative PCR (qPCR) method described in a greater detail below.

Isolated microbes may be assessed for colonization of the desired host plant in small-scale field trials. Additionally, RNA may be isolated from colonized root samples to obtain transcriptome data for the strain in a field environment. These small-scale field trials are referred to herein as CAT (Colonization and Transcript) trials, as these trials provide Colonization and Transcript data for the strain in a field environment.

For these trials, seeds of the host plant (e.g., corn, wheat, rice, sorghum, and soybean) may be inoculated using cultures of isolated microbes individually or in combination and planted into soil. Alternatively, cultures of isolated microbes, individually or in combination, can be applied to the roots of the host plant by inoculating the soil directly over the roots. The CAT trials can be conducted in a variety of soils and/or under various temperature and/or moisture conditions to assess the colonization potential and obtain transcriptome profile of the microbe in various soil types and environmental conditions.

Colonization of roots of the host plant by the inoculated microbe(s) may be assessed, for example, using a qPCR method.

In one protocol, the colonization potential of isolated microbes may be assessed as follows. One day after planting of corn seeds, 1 ml of microbial overnight culture (SOB media) may be drenched right at the spot where the seed is located. 1 mL of this overnight culture may contain approximately 10\hat{ }9 cfu, varying within 3-fold of each other, depending on which strain is being used. Each seedling may be fertilized 3× weekly with 50 mL modified Hoagland's solution supplemented with either 2.5 mM or 0.25 mM ammonium nitrate. At four weeks after planting, root samples may be collected for DNA extraction. Soil debris may be washed away using pressurized water spray. These tissue samples may then be homogenized using QIAGEN Tissuelyzer and the DNA extracted using QIAmp DNA Mini Kit (QIAGEN) according to the recommended protocol. qPCR assay may be performed, for example using Stratagene Mx3005P RT-PCR on the DNA extracts using primers designed (using, for example, NCBI's Primer BLAST) to be specific to a loci in each of the microbe's genome.

The number of copies of each microbial genome may be quantified, which may reflect the colonization potential of the microbe. Identity of the microbial species may be confirmed by sequencing the PCR amplification products.

Additionally, RNA may be isolated from colonized root and/or soil samples and sequenced. Unlike the DNA profile, an RNA profile varies depending on the environmental conditions. Therefore, sequencing of RNA isolated from colonized roots and/or soil may reflect the transcriptional activity of genes in planta in the rhizosphere. RNA can be isolated from colonized root and/or soil samples at different time points to analyze the changes in the RNA profile of the colonized microbe at these time points. For example, RNA can be isolated from colonized root and/or soil samples right after fertilization of the field and a few weeks after fertilization of the field and sequenced to generate corresponding transcriptional profile. Similarly, RNA sequencing can be carried out under high phosphate and low phosphate conditions to understand which genes are transcriptionally active or repressed under these conditions.

Methods for transcriptomic/RNA sequencing are known in the art. Briefly, total RNA may be isolated from the purified culture of the isolated microbe; cDNA may be prepared using reverse transcriptase; and the cDNA may be sequenced using high-throughput sequencing tools described above. Sequencing reads from the transcriptome analysis can be mapped to the genomic sequence and transcriptional promoters for the genes of interest can be identified.

The plant-beneficial activity of isolated microbes may be assessed. For example, phosphate solubilizing microbes may be assayed for phosphate solubilization. In some cases, phosphate solubilizing microbes may be assayed by measuring the pH of the media when the microbes are cultured in a medium lacking soluble phosphate. A drop in pH may indicate secretion of phosphate solubilizing acids. Any parameter of interest can be utilized, and an appropriate assay developed for such. For instance, assays could include growth curves for colonization metrics. This step may confirm the phenotype of interest and eliminate any false positives.

The data generated in the above steps may be used to select microbes for further development. For example, microbes showing a desired combination of colonization potential, phosphate solubilization, and/or relevant DNA and RNA profile may be selected for domestication and remodeling.

The selected microbes may be domesticated; wherein, the microbes may be converted to a form that is genetically tractable and identifiable. One way to domesticate the microbes is to engineer them with antibiotic resistance. For this, the wild type microbial strain may be tested for sensitivity to various antibiotics. If the strain is sensitive to the antibiotic, then the antibiotic can be a good candidate for use in genetic tools/vectors for remodeling the strain.

Vectors that are conditional for their replication (e.g., a suicide plasmid) may be constructed to domesticate the selected microbes (host microbes). For example, a suicide plasmid containing an appropriate antibiotic resistance marker, a counter selectable marker, an origin of replication for maintenance in a donor microbe (e.g., E. coli), a gene encoding a fluorescent protein (GFP, RFP, YFP, CFP, and the like) to screen for insertion through fluorescence, an origin of transfer for conjugation into the host microbe, and a polynucleotide sequence comprising homology arms to the host genome with a desired genetic variation may be constructed. The vector may comprise a SceI site and other additional elements.

Exemplary antibiotic resistance markers include ampicillin resistance marker, kanamycin resistance marker, tetracycline resistance marker, chloramphenicol resistance marker, erythromycin resistance marker, streptomycin resistance marker, spectinomycin resistance marker, etc. Exemplary counter selectable markers include sacB, rpsL, tetAR, pheS, thyA, lacY, gata-1, ccdB, etc.

In one protocol, a suicide plasmid containing an appropriate antibiotic resistance marker, a counter selectable marker, the λpir origin of replication for maintenance in E. coli ST18 containing the pir replication initiator gene, a gene encoding green fluorescent protein (GFP) to screen for insertion through fluorescence, an origin of transfer for conjugation into the host microbe, and a polynucleotide sequence comprising homology arms to the host genome with a desired genetic variation (e.g., a promoter from within the microbe's own genome for insertion into a heterologous location such as proximal to a phosphate solubilization gene) may be transformed into E. coli ST18 (an auxotroph for aminolevulinic acid, ALA) to generate donor microbes.

Donor microbes may be mixed with host microbes (selected candidate microbes from step B5) to allow conjugative integration of the plasmid into the host genome. The mixture of donor and host microbes may be plated on a medium containing the antibiotic and not containing ALA. The suicide plasmid is able to replicate in donor microbes (E. coli ST18), but not in the host. Therefore, when the mixture containing donor and host microbes is plated on a medium containing the antibiotic and not containing ALA, host cells that integrated the plasmid into its genome may be selected for.

A proper integration of the suicide plasmid containing the fluorescent protein marker, the antibiotic resistance marker, the counter selectable marker, etc. at the intended locus of the host microbe may be confirmed through fluorescence of colonies on the plate and using colony PCR.

A second round of homologous recombination in the host microbes may loop out (remove) the plasmid backbone leaving the desired genetic variation (e.g., a promoter from within the microbe's own genome for insertion into a heterologous location) integrated into the host genome of a certain percentage of host microbes, while reverting a certain percentage back to wild type.

Colonies of host microbes that have looped out the plasmid backbone (and therefore, looped out the counter selectable marker) may be recovered by growing them on an appropriate medium.

For example, if sacB is used as a counter selectable marker, loss of this marker due to the loss of the plasmid backbone may be tested by growing the colonies on a medium containing sucrose (sacB confers sensitivity to sucrose). Colonies that grow on this medium would have lost the sacB marker and the plasmid backbone and would either contain the desired genetic variation or be reverted to wild type. The colonies that grew better on the sucrose-containing medium (or other appropriate media depending on the counter selectable marked used) may be picked and the presence of the genetic variation at the intended locus may be confirmed by screening the colonies using colony PCR.

In some isolates, the sacB or other counterselectable markers may not confer full sensitivity to sucrose or other counterselection mechanisms, which necessitates screening large numbers of colonies to isolate a successful loop-out. In those cases, loop-out may be aided by use of a “helper plasmid” that replicates independently in the host cell and expresses a restriction endonuclease, e.g., SceI, which recognizes a site in the integrated suicide plasmid backbone. The strain with the integrated suicide plasmid may be transformed with the helper plasmid containing an antibiotic resistance marker, an origin of replication compatible with the host strain, and a gene encoding a restriction endonuclease controlled by a constitutive or inducible promoter. The double-strand break induced in the integrated plasmid backbone by the restriction endonuclease may promote homologous recombination to loop-out the suicide plasmid. This may increase the number of looped-out colonies on the counterselection plate and decrease the number of colonies that need to be screened to find a colony containing the desired mutation. The helper plasmid may be removed from the strain by culture and serial passaging in the absence of antibiotic selection for the plasmid. The passaged cultures can be streaked for single colonies, colonies picked and screened for sensitivity to the antibiotic used for selection of the helper plasmid, as well as absence of the plasmid confirmed by colony PCR. Finally, the genome may be sequenced, and the absence of helper plasmid DNA can be confirmed.

Although this example describes one protocol for domesticating the microbe and introducing genetic variation into the microbe, one of ordinary skill in the art would understand that the genetic variation can be introduced into the selected microbes using a variety of other techniques known in the art such as: polymerase chain reaction mutagenesis, oligonucleotide-directed mutagenesis, saturation mutagenesis, fragment shuffling mutagenesis, homologous recombination, ZFN, TALENS, CRISPR systems (Cas9, Cpf1, etc.), chemical mutagenesis, and combinations thereof.

### Non-Intergeneric Engineering Campaign and Optimization

Selected microbes may be engineered/remodeled to improve performance of the plant-beneficial activity. For this, gene targets for improving the plant-beneficial activity may be identified.

Gene targets may be identified in various ways. For example, genes of interest can be identified while annotating the genes from the whole genome sequencing of isolated microbes.

A desired genetic variation for improving the plant-beneficial activity can comprise promoter swapping, in which the native promoter for a target gene is replaced with a stronger or weaker promoter (when compared to the native promoter) from within the microbe's genome, or a differently regulated promoter. If the expression of a target gene increases the plant-beneficial activity (e.g., phoC, the expression of which may enhance phosphate solubilization microbes), the desired promoter for promoter swapping is a stronger promoter (compared to the native promoter of the target gene) that would further increase the expression level of the target gene compared to the native promoter. If the expression of a target gene may decrease the plant-beneficial activity (e.g., gluconate dehydrogenase which may decrease phosphate solubilization), the desired promoter for promoter swapping is a weak promoter (compared to the native promoter of the target gene) that would substantially decrease the expression level of the target gene compared to the native promoter. Promoters can be inserted into genes to “knock-out” a gene's expression, while at the same time upregulating the expression of a downstream gene, or to “knock-out” a first coding sequence in an operon and increase expression of a second coding sequence in the operon.

Promoters for promoter swapping can be selected based on RNA sequencing data. For example, RNA sequencing data can be used to identify strong and weak promoters, or constitutively active vs. inducible promoters.

For example, to identify strong and weak promoters, or constitutively active vs. inducible promoters, for use with phosphate solubilization associated genes, selected microbes may be cultured in vitro under phosphate-depleted and phosphate-replete conditions, RNA of the microbes may be isolated from these cultures, and sequenced.

In an example protocol, the RNA profile of a microbe under phosphate-depleted and phosphate-replete conditions may be compared and active promoters with a desired transcription level may be identified.

Promoters may also be selected using RNA sequencing data that reflects the RNA profile of the microbe in planta in the host plant rhizosphere. RNA sequencing under various conditions allows for selection of promoters that: a) are active in the rhizosphere during the host plant growth cycle in fertilized field conditions, and b) are also active in relevant in vitro conditions so they can be rapidly screened.

In an exemplary protocol, in planta RNA sequencing data from colonization assays may be used to measure the expression levels of genes in isolated microbes. In one embodiment, the level of gene expression may be calculated as reads per kilobase per million mapped reads (RPKM). The expression level of various genes may be compared to the expression level of a target gene and at least the top 10, 20, 30, 40, 50, 60, or 70 promoters, associated with the various genes that show the highest or lowest level of expression compared to the target gene are selected as possible candidates for promoter swapping.

For example, if the target gene is upregulation of phoC, the first 10, 20, 30, 40, 50, or 60 promoters for genes that show the highest level of expression compared to phoC are selected as possible candidates for promoter swapping.

These candidates can be further short-listed based on in vitro RNA sequencing data. For example, for phoC as the target gene, possible promoter candidates selected based on the in planta RNA sequencing data are further selected by choosing promoters with similar or increased gene expression levels compared to phoA under in vitro phosphate-deplete vs. phosphate-replete conditions.

The set of promoters selected in this step are used to swap the native promoter of the target gene (e.g., phoA). Remodeled strains with swapped promoters are tested in in vitro assays, strains with lower than expected activity are eliminated, and strains with expected or higher than expected activity are tested in field. The cycle of promoter selection may be repeated on remodeled strains to further improve their plant-beneficial activity.

Based on the previous steps identifying gene targets and identifying promoters for promoter swaps, non-intergeneric genetic variations may be designed.

The term non-intergeneric indicates that the genetic variation to be introduced into the host does not contain a nucleic acid sequence from outside the host genus (i.e., no transgenic DNA). Although vectors and/or other genetic tools may be used to introduce the genetic variation into the host microbe, the methods of the present disclosure include steps to loop-out (remove) the backbone vector sequences or other genetic tools introduced into the host microbe leaving only the desired genetic variation into the host genome. Thus, the resulting microbe is non-transgenic.

Exemplary non-intergeneric genetic variations include a mutation in the gene of interest that may improve the function of the protein encoded by the gene; a constitutionally active promoter that can replace the endogenous promoter of the gene of interest to increase the expression of the gene; a mutation that may inactivate the gene of interest; the insertion of a promoter from within the host's genome into a heterologous location, e.g., insertion of the promoter into a gene that results in inactivation of the gene and upregulation of a downstream gene; and the like. The mutations can be point mutations, insertions, and/or deletions (full or partial deletion of the gene). For example, in one protocol, to improve the phosphate solubilization activity of the host microbe, a desired genetic variation may comprise an inactivating mutation of a gene which negatively impacts phosphate solubilization and replacing the endogenous promoter of a gene which positively impacts phosphate solubilization with a more highly expressed promoter.

After designing the non-intergeneric genetic variations, the remodeled strains may be generated as described above.

A purified culture of the remodeled microbe may be preserved in a bank, so that gDNA can be extracted for whole genome sequencing.

The genomic DNA of the remodeled microbe may be extracted and the whole genome sequencing may be performed on the genomic DNA using methods described previously. The resulting reads may be mapped to the reads previously stored in a laboratory information management system (LIMS) to confirm: a) presence of the desired genetic variation and b) complete absence of reads mapping to vector sequences (e.g., plasmid backbone or helper plasmid sequence) that were used to generate the remodeled microbe.

This step may allow sensitive detection of non-host genus DNA (transgenic DNA) that may remain in the strain after looping out of the vector backbone (e.g., suicide plasmid) method and could provide a control for accidental off-target insertion of the genetic variation, etc.

### Analytics Upon Remodeled Microbes

The plant-beneficial activity and growth kinetics of the remodeled microbes may be assessed in vitro.

For example, strains remodeled for improving phosphate solubilization function may be assessed for phosphate solubilization activity and fitness through in vitro assays and colonization assays.

This step allows rapid, medium, to high-throughput screening of remodeled strains for the phenotypes of interest.

Remodeled strains may be assessed for colonization of the host plant either in the greenhouse or in the field using the steps described previously. Additionally, RNA may be isolated from colonized root and/or soil samples and sequenced to analyze the transcriptional activity of target genes. Target genes comprise the genes containing the genetic variation introduced and may also comprise other genes that play a role in the phosphate solubilizing activity of the microbe.

This step allows determination of the fitness of top in vitro performing strains in the rhizosphere and allows measurement of the transcriptional activity of altered genes in planta.

### Iterate Engineering Campaign/Analytics

The data from in vitro and in planta analytics may be used to iteratively stack beneficial mutations.

Furthermore, steps described above may be repeated to fine-tune the phosphate solubilizing activity of the microbes. For example, plants may be inoculated using microbial strains remodeled in the first round; harvested after a few weeks of growth; and microbes from the soil and/or roots of the plants may be isolated. The functional activity (phosphate solubilizing activity and colonization potential) and the DNA and RNA profile of isolated microbes may be characterized in order to select microbes with improved phosphate solubilizing activity and colonization potential. The selected microbes may be remodeled to further improve the phosphate solubilizing activity. Remodeled microbes may be screened for the functional activity, for example phosphate solubilizing activity and colonization potential, and the top performing strains may be selected. If desired, the entire process, or parts of the process, can be repeated to further improve the plant-beneficial activity of the remodeled microbes from the second round. The process, or parts of the process, can be repeated for 1, 2, 3, 4, 5, 6, 7, 8, 9, 10, or more rounds.

### Compositions

Compositions comprising microbes or microbial populations produced according to methods described herein and/or having characteristics as described herein can be in the form of a liquid, a foam, or a dry product. In some examples, a composition comprising microbial populations may be in the form of a dry powder, a slurry of powder and water, or a flowable seed treatment.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple microbes (e.g., bacteria) or microbial (e.g., bacterial) populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Acidovorax, Agrobacterium, Bacillus, Burkholderia, Chryseobacterium, Curtobacterium, Enterobacter, Escherichia, Methylobacterium, Paenibacillus, Pantoea, Pseudomonas, Ralstonia, Saccharibacillus, Sphingomonas, and Stenotrophomonas.

In some examples, at least two, at least three, at least four, at least five, at least six, at least seven, at least eight, at least nine, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, and Pleosporaceae.

In some examples, at least two, at least three, at least four, at least five, at least six, at least seven, at least eight, at least night, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, and Pleosporaceae.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The microbial (e.g., bacterial) species may be present in compositions at a concentration of between 10⁸ to 10¹⁰ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier.

The compositions comprising the microbial (e.g., bacterial) populations described herein may be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth or populate the leaves of the plant with viable bacterial growth. In general, an effective amount is an amount sufficient to result in plants with improved traits (e.g., a desired level of phosphorous solubilization).

Microbial (e.g., bacterial) compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a desiccant, a bactericide, a nutrient, or any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, microbes (e.g., bacteria) are mixed with an agriculturally acceptable carrier. The carrier can be a solid carrier or liquid carrier, and in various forms including microspheres, powders, emulsions, and the like. The carrier may be any one or more of a number of carriers that confer a variety of properties, such as increased stability, wettability, or dispersibility. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like; microencapsulated particles, and the like; liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, organic phosphorous, inorganic phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions can help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, gum Arabic, xanthan gum, mineral oil, polyethylene glycol (PEG), Polyvinyl pyrrolidone (PVP), arabino-galactan, methyl cellulose, PEG 400, chitosan, polyacrylamide, polyacrylate, polyacrylonitrile, glycerol, triethylene glycol, vinyl acetate, gellan gum, polystyrene, polyvinyl, carboxymethyl cellulose, gum ghatti, and polyoxyethylene-polyoxybutylene block copolymers.

In some embodiments, the adhesives can be, e.g., a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, an oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticides) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N (US), Inhance (Brandt), P-28 (Wilfarm), and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis), and Century (Precision). In some embodiments, the surfactant is present at a concentration from 0.01% v/v to 10% v/v. In some other embodiments, the surfactant is present at a concentration from 0.1% v/v to 1% v/v.

In certain cases, the formulation may include a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used and should promote the ability of the microbial population to survive application on the seeds and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, from about 10% to about 40%, from about 15% to about 35%, or from about 20% to about 30%. In some cases, it is advantageous for the formulation to contain agents such as a fungicide, an antibacterial agent, an herbicide, a nematicide, an insecticide, a plant growth regulator, a rodenticide, a bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to, the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi or secrete toxins or other substances which can kill or otherwise prevent the growth of fungi. Any type of fungicide, particularly ones that are commonly used on plants, can be used as a control agent in a seed composition.

In some examples, the seed coating composition comprises a control agent which has antibacterial properties. In one embodiment, the control agent with antibacterial properties is selected from the compounds described elsewhere herein. In another embodiment, the compound is streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, a growth regulator is selected from the group consisting of: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl, and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Nematode-antagonistic biocontrol agents may include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pasteuria usage, Brevibacillus laterosporus strain G4, Pseudomonas fluorescens, and Rhizobacteria.

Some examples of nutrients can be selected from the group consisting of a nitrogen fertilizer including, but not limited to urea, ammonium nitrate, ammonium sulfate, non-pressure nitrogen solutions, aqua ammonia, anhydrous ammonia, ammonium thiosulfate, sulfur-coated urea, urea-formaldehydes, IBDU, polymer-coated urea, calcium nitrate, ureaform, and methylene urea, phosphorous fertilizers such as diammonium phosphate, monoammonium phosphate, ammonium polyphosphate, concentrated superphosphate and triple superphosphate, and potassium fertilizers such as potassium chloride, potassium sulfate, potassium-magnesium sulfate, potassium nitrate. Such compositions can exist as free salts or ions within the seed coat composition. Alternatively, nutrients/fertilizers can be complexed or chelated to provide sustained release over time.

Some examples of rodenticides may include substances selected from the group consisting of 2-isovalerylindan-1,3-dione, 4-(quinoxalin-2-ylamino) benzenesulfonamide, alpha-chlorohydrin, aluminum phosphide, ANTU, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin, and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, Fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

### Application of Microbial (e.g., Bacterial) Populations on Crops

The composition of the microbes (e.g., bacteria) or microbial (e.g., bacterial) population described herein can be applied in furrow, in talc, or as seed treatment. The composition can be applied to a seed package in bulk, in mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, sorghum, potato, rice, vegetables, cereals, pseudocereals, and oilseeds. Examples of cereals may include barley, fonio, oats, palmer's grass, rye, pearl millet, sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic, or conventional.

Additives such as micro-fertilizer, PGR, herbicide, insecticide, and fungicide can be used additionally to treat the crops. Examples of additives include crop protectants such as insecticides, nematicides, fungicides, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition may improve plant traits, such as promoting plant growth, maintaining high chlorophyll content in leaves, increasing fruit or seed numbers, and increasing fruit or seed unit weight. Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low phosphorous stress, phosphorous use efficiency, resistance to nematode stress, resistance to a fungal pathogen, resistance to a bacterial pathogen, resistance to a viral pathogen, level of a metabolite, modulation in level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved phosphate utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without the seed treatment formulation.

In some cases, plants are inoculated with bacteria or bacterial populations that are isolated from the same species of plant as the plant element of the inoculated plant. For example, bacteria or bacterial populations that are normally found in one variety of Zea mays (corn) are associated with a plant element of a plant of another variety of Zea mays that in its natural state lacks the bacteria and bacterial populations. In some embodiments, the bacteria and bacterial populations are derived from a plant of a related species of plant as the plant element of the inoculated plant. For example, bacteria and bacterial populations that are normally found in Zea diploperennis can be applied to a Zea mays, or vice versa. In some cases, plants are inoculated with bacteria and bacterial populations that are heterologous to the plant element of the inoculated plant. In one embodiment, the bacteria and bacterial populations are derived from a plant of another species. For example, bacteria and bacterial populations that are normally found in dicots are applied to a monocot plant (e.g., inoculating corn with a soybean-derived bacteria and bacterial populations), or vice versa. In other cases, the bacteria and bacterial populations to be inoculated onto a plant are derived from a related species of the plant that is being inoculated. In one embodiment, the bacteria and bacterial populations are derived from a related taxon, for example, from a related species. The plant of another species can be an agricultural plant. In another embodiment, the bacteria and bacterial populations are part of a designed composition inoculated into any host plant element.

In some examples, the bacteria or bacterial population is exogenous, wherein the bacteria or bacterial population is isolated from a different plant than the inoculated plant. For example, in certain embodiments, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present disclosure's detection and isolation of bacteria and bacterial populations within the mature tissues of plants after coating on the exterior of a seed may demonstrate their ability to move from seed exterior into the vegetative tissues of a maturing plant. Therefore, in various embodiments, the bacteria and bacterial populations are capable of moving from the seed exterior into the vegetative tissues of a plant. In some embodiments, the bacteria and bacterial populations that are coated onto the seed of a plant is capable, upon germination of the seed into a vegetative state, of localizing to a different tissue of the plant. For example, bacteria and bacterial populations can be capable of localizing to any one of the tissues in the plant, including the: root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In certain embodiments, the bacteria and bacterial populations are capable of localizing to the root and/or the root hair of the plant. In some embodiments, the bacteria and bacterial populations are capable of localizing to the photosynthetic tissues, for example, leaves and shoots of the plant. In other cases, the bacteria and bacterial populations are localized to the vascular tissues of the plant, for example, in the xylem and phloem. In still another embodiment, the bacteria and bacterial populations are capable of localizing to the reproductive tissues (flower, pollen, pistil, ovaries, stamen, or fruit) of the plant. In certain embodiments, the bacteria and bacterial populations are capable of localizing to the root, shoots, leaves, and reproductive tissues of the plant. In various embodiments, the bacteria and bacterial populations colonize a fruit or seed tissue of the plant. In certain embodiments, the bacteria and bacterial populations are able to colonize the plant such that it they present on the surface of the plant (i.e., its presence is detectably present on the plant exterior or the episphere of the plant). In some embodiments, the bacteria and bacterial populations are capable of localizing to substantially all, or all, tissues of the plant. In certain embodiments, the bacteria and bacterial populations are not localized to the root of a plant. In other cases, the bacteria and bacterial populations are not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In some examples, bacteria may localize to any one of the tissues in the plant, including the: root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In certain embodiments, the bacteria or bacterial population is capable of localizing to the photosynthetic tissues, for example, leaves and shoots of the plant. In other cases, the bacteria and bacterial populations are localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations are capable of localizing to the root, shoots, leaves, and reproductive tissues of the plant. In another embodiment, the bacteria or bacterial population colonizes a fruit or seed tissue of the plant. In still another embodiment, the bacteria or bacterial population is able of colonizing the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations are not localized to the photosynthetic tissues of the plant.

The effectiveness of the microbial (e.g., bacterial) compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production, and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, and asparagus.

In some examples, plants that may be obtained or improved using the methods and compositions disclosed herein may include plants that are important or interesting for agriculture, horticulture, biomass for the production of biofuel molecules and other chemicals, and/or forestry. Some examples of these plants may include pineapple, banana, coconut, lily, grass peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, and brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot and sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Naj adales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to a plant of another type (e.g., a crop plant) to confirm conferral of the improved trait.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1—Refactoring Expression of phoC1 and phoC2

To improve solubilization of organic phosphate by CI019, multiple plasmids containing refactored expression cassettes for the CI019 phoC genes were designed and constructed. First, the open reading frames encoding phoC1 and phoC2 were codon-randomized to remove any internal regulatory sequences, (SEQ ID NOs:3 and 4). Next, three heterologous promoter-RBS parts were designed: 1) a part containing an engineered constitutive lac promoter and an optimized synthetic ribosome binding site (RBS aggagg) as previously published (Vick et al. 2011, SEQ ID NO:5), 2) a part containing promoter BBa_J23110 and RBS BBa_B0032 from the Registry of Standard Biological Parts (parts.igem.org/Catalog, SEQ ID NOs:6 and 7), and 3) a part containing the promoter and RBS of the constitutively and highly-expressed lpp gene of an Enterobacter sacchari strain (CI006, SEQ ID NO:8). Finally, constructs were assembled containing each promoter-RBS part and phoC codon-randomized gene combination into a plasmid backbone with a KanR resistance marker and CloDF13 origin of replication, (SEQ ID NOs:9 and 10). CI019 was transformed with each of the resulting plasmids, and the transformants were stocked and used for downstream greenhouse experiments.

### Example 2—Analysis of Refactored phoC1 and phoC2 Expression in Planta

To assess strain transcriptional activity in planta, 600 g of autoclaved sand was measured out into 656 mL pots, which were saturated with sterile DI H₂O and allowed to drain 24 hours, at which time corn seeds (DKC 66-40) were planted 1 cm deep. The plants were maintained under fluorescent lamps for four weeks with 16-hour day length at room temperatures averaging 22° C. (night) to 26° C. (day). Five days after planting, seedlings were inoculated with a 1 ml suspension of cells drenched directly over the emerging coleoptile. Inoculum was prepared from 5 ml overnight cultures in SOB, which were spun down and resuspended in PBS to final dilution to OD₅₉₀ of 1.0 (approximately 10⁹ CFU/ml). Control plants were treated with sterile PBS, and each treatment was applied to five replicate plants. Plants were watered as needed and fertilized Monday-Wednesday-Friday with 25 mL of a modified Hoagland solution. After 17 days, roots were harvested and shaken free of sand, then immersed in an RNA stabilization solution for 30 minutes and stored at −80° C. for subsequent root and microbial RNA and DNA extraction.

After thawing, the roots were then briefly rinsed with sterile deionized water. Samples were homogenized using bead beating with ½-inch stainless steel ball bearings in a tissue lyser (TissueLyser II, Qiagen P/N 85300) in 2 ml of lysis buffer (Qiagen P/N 79216). Genomic DNA extraction was performed with ZR-96 Quick-gDNA kit (Zymo Research P/N D3010), and RNA extraction using the RNeasy kit (Qiagen P/N 74104).

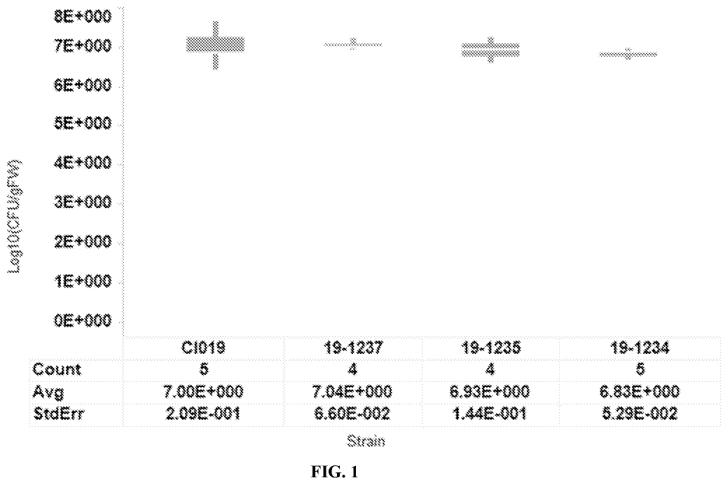

Root colonization was measured by qPCR with primers designed to amplify unique regions of the wild type parent strain. qPCR reaction efficiency was measured using a standard curve generated from a known quantity of gDNA from the target genome. Data was normalized to genome copies per g fresh weight using the tissue weight and extraction volume. The data plotted in FIG. 1 show that the phoC1 and phoC2 expressing strains colonize corn roots at similar levels to the wild-type parent (CI019).

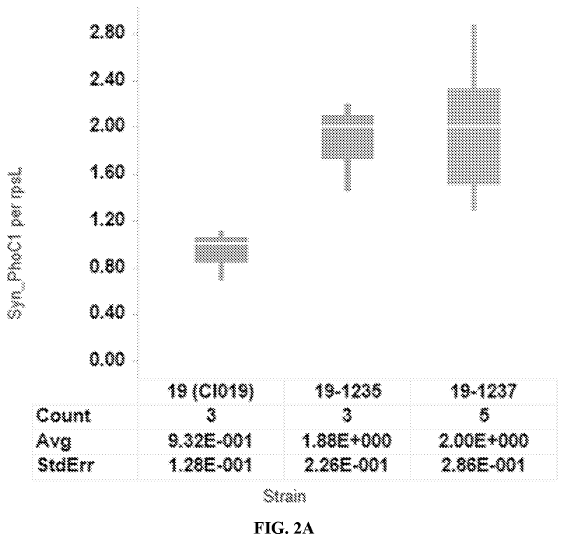

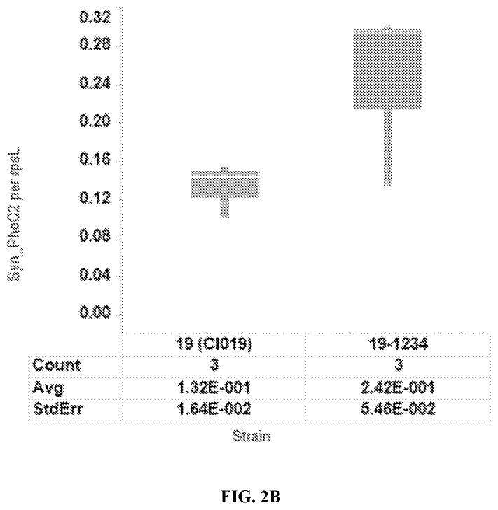

Transcript levels for target genes were measured by Nanostring analysis. Purified RNA was processed on an nCounter Sprint (Core Diagnostics, Hayward, Calif.). The copy number was normalized to the housekeeping gene rpsL to account for differences in total RNA yield and microbial colonization from the root tissue samples. The data plotted in FIG. 2A (expression mutants 19-1235 and 19-1237 (phoC1)) and FIG. 2B (expression mutant 19-1234 (phoC2)) show increased phoC transcript levels in planta compared to their wild-type parent (CI019).

### Example 3: Phosphate Solubilization by Klebsiella variicola Strains

One wild type (WT) and 7 mutant strains of Klebsiella variicola 137 were subjected to a phosphate solubilization screen. Briefly, this assay comprised culturing each strain in the presence of an insoluble form of phosphate, collecting the supernatant of the cultured microbes, and performing a colorimetric assay on the supernatant to determine the soluble phosphate content.

A list and brief description of each strain assayed is provided in Table 1. Sequences for each strain are provided in Table 9.

TABLE 1
Klebsiella variicola strain descriptions

| Strain ID | Lineage | Mutagenic DNA Description | Genotype |
|---|---|---|---|
| 137 | N/A | N/A | WT |
| 137-1855 | Mutant of | Insertion of a 300 bp fragment of the | pqq_operon::prm15.2 |
| | 137 | region upstream of the rplN gene | |
| | | (Prm15.2) inserted 126 bp upstream of | |
| | | pqqB. | |
| 137-1856 | Mutant of | Deletion of the entire gad gene | Δgad |
| | 137 | | |
| 137-1914 | Mutant of | Insertion of a 299 bp fragment of the | pqq_operon::prm8.2 |
| | 137 | region 80 bp from the start of the nlpI | |
| | | gene (Prm8.2) inserted 126 bp upstream | |
| | | of pqqB. | |
| 137-1916 | Mutant of | Insertion of a 71 bp fragment of the | pqq_operon::prm16.1 |
| | 137 | region upstream of an hypothetical | |
| | | protein CDS (PROKKA-00884) | |
| | | (Prm16.1) inserted 126 bp upstream of | |
| | | pqqB. | |
| 137-2142 | Mutant of | Deletion of the entire gntT gene | ΔgntT |
| | 137 | | |
| 137-2234 | Mutant of | Insertion of a 71 bp fragment of the | gcd::prm16.1 |
| | 137 | region upstream of an hypothetical | |
| | | protein CDS (PROKKA-00884) | |
| | | (Prm16.1) inserted upstream of gcd. | |
| | | 54 bp of the original upstream sequence | |
| | | of gcd was deleted and replaced with | |
| | | prm16.1 | |
| 137-2311 | Mutant of | Insertion of a 299 bp fragment of the | gcd::prm8.2 |
| | 137 | region 80 bp from the start of the nlpI | |
| | | gene (Prm8.2) inserted upstream of gcd. | |
| | | 54 bp of the original upstream sequence | |
| | | of gcd was deleted and replaced with | |
| | | prm8.2 | |

All strains were streaked out from frozen stocks on super optimal broth (SOB) plates. Plates were incubated at 30° C. overnight. For each strain, 3 individual colonies from the overnight plates were inoculated into 5 mL of liquid SOB. These cultures were grown at 30° C. with shaking overnight. The optical density at 590 nm (OD590) of each of the overnight SOB cultures was measured. In order to normalize the starting optical densities across strains, sufficient volume of each culture was added 25 mL of National Botanical Research Institute's Phosphate-Bromo Phenol Blue (NBRIP-BPB) growth media in a 125 mL conical flask, resulting in a starting OD590 of 0.03 for each culture, (modified from Dash, N., Pahari, A., Dangar, T., 2017. Functionalities of Phosphate-Solubilizing Bacteria of Rice Rhizosphere: Techniques and Perspectives. Recent Advances in Applied Microbiology, 151-163.). NBRIP-BPB contains 5 g/L Ca₃(PO₄)₂, an insoluble form of inorganic phosphate. Flasks were transferred to a shaker/incubator set to a temperature of 30° C. and shaking at 200 rpm. Along with culture flasks, two flasks containing 25 mL of uninoculated NBRIP-BPB growth media were incubated as a negative control.

On the day of inoculation (D0), for each culture 1.5 mL of uninoculated NBRIP-BPB growth media was transferred to each of two 2 mL centrifuge tubes. These were spun down in a benchtop centrifuge at 13.2 krpm for 5 minutes. The supernatant was removed and tested for soluble phosphate concentration. At D0, background soluble phosphate was measured at about 3 μg/mL.

At subsequent timepoints day 1 after inoculation (D1), day 4 after inoculation (D4), day 7 after inoculation (D7), and day 10 after inoculation (D10), the flasks were removed from the shaker/incubator, samples were collected as described above, and the supernatant was collected by centrifugation as described above. The flasks were each returned to continue the incubation after supernatant collection.

Soluble phosphate content of supernatant samples was quantified using a modified version of the ascorbic acid method, (Murphy, J., Riley, J. P., 1962. A modified single solution method for determination of phosphate in natural waters. Analytica Chimica Acta 27, 31-36). A standard curve was prepared in the range 0.5 μg/mL P to 10 μg/mL P by dissolving a known concentration of potassium phosphate in molecular water. Samples were diluted using molecular water until their soluble phosphate concentration fell within the range of the standard curve. 200 μL of each standard and sample was added to the wells of a clear microplate, and 40 μL of active reagent was added to each well and mixed by pipetting. The active reagent was prepared as 25 mL 5 N sulfuric acid, 7.5 mL of 40 g/L ammonium molybdate, 15 mL of 0.1 M ascorbic acid, and 2.5 mL potassium antimonyl tartrate, (1 mg Sb/mL) fresh on each day the colorimetric assays were carried out. The plate was incubated at RT, and the absorbance of each well at 882 nm measured. Sample concentrations were calculated by extrapolation from standard concentrations fitted to a rectangular hyperbola.

Table 2 shows the average soluble phosphate of three replicate cultures of Klebsiella variicola 137 and remodeled strains. A >10% increase in the average soluble phosphate is observed in all remodeled strains at Day 7, and a >20% increase in the average soluble phosphate is observed for all remodeled strains at Day 10.

TABLE 2
Klebsiella variicola average soluble phosphate produced

Average Soluble Phosphate (μg/mL)

| Strain | Day 1 | Day 4 | Day 7 | Day 10 |
|---|---|---|---|---|
| 137 | 248.4 | 283.8 | 235.5 | 152.5 |
| 137-1855 | 258.8 | 307.7 | 315.7 | 250.5 |
| 137-1856 | 247.6 | 296.1 | 274.8 | 212.3 |
| 137-1914 | 246.9 | 295.5 | 276.2 | 283.3 |
| 137-1916 | 144.6 | 288.2 | 321.0 | 293.3 |
| 137-2142 | 232.7 | 297.3 | 263.7 | 258.8 |
| 137-2234 | 232.3 | 284.1 | 302.1 | 184.5 |
| 137-2311 | 227.2 | 272.4 | 368.4 | 274.2 |

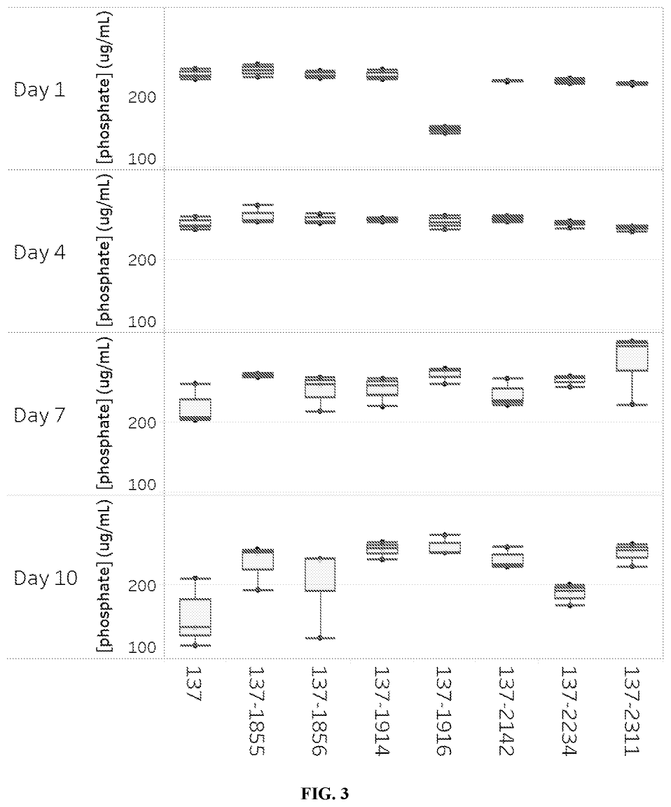

Soluble phosphate in three replicate cultures of Klebsiella variicola (137) and remodeled strains are shown in FIG. 3. All strains solubilized phosphate above the D0 background level of about 3 μg/mL. At Day 10, a statistically significant increase in soluble phosphate in cultures of 137-1914 (p=0.04), 137-1916 (p=0.02), and 137-2311 (p=0.04) was observed compared to that of wild-type 137. P values were determined using a two-tailed two-sample unequal variance T test.

### Example 4: Phosphate Solubilization by Rahnella aquatilis Strains

One wild type (WT) and 3 mutant strains of Rahnella aquatilis C1019 were subjected to a phosphate solubilization screen. Briefly, this assay comprised culturing each strain in the presence of an insoluble form of phosphate, collecting the supernatant of the cultured microbes, and performing a colorimetric assay on the supernatant to determine the soluble phosphate content.

A list and brief description of each strain assayed is provided in Table 3. Sequences for each strain are provided in Table 9.

TABLE 3
Rahnella aquatilis strain descriptions

| Strain ID | Lineage | Mutagenic DNA Description | Genotype |
|---|---|---|---|
| CI019 | N/A | N/A | WT |
| 19-1276 | Mutant of | Deletion and replacement of the gad1 gene | Δgad1::specR- |
| | CI019 | with an insertion of a 1638 bp specR-sacB | sacB |
| | | cassette 54 bp upstream of the gad1 stop | |
| | | codon | |
| 19-1277 | Mutant of | Complete deletion of the gad2 gene | Δgad2::specR- |
| | CI019 | replaced with a 1638 bp specR-sacB | sacB |
| | | cassette. | |
| 19-1280 | Mutant of | Complete deletion of the gntT gene replaced | ΔgntT::specR- |
| | CI019 | with a 1638 bp specR-sacB cassette. | sacB |

All strains were streaked out from frozen stocks on super optimal broth (SOB) plates. Plates were incubated at 30° C. overnight. For each strain, 3 individual colonies from the overnight plates were inoculated into 5 mL of liquid SOB. These cultures were grown at 30° C. with shaking overnight. The optical density at 590 nm (OD590) of each of the overnight SOB cultures was measured. In order to normalize the starting optical densities across strains, sufficient volume of each culture was added 25 mL of National Botanical Research Institute's Phosphate-Bromo Phenol Blue (NBRIP-BPB) (modified from Dash et al., 2017) growth media in a 125 mL conical flask, resulting in a starting OD590 of 0.03 for each culture. NBRIP-BPB contains 5 g/L Ca₃(PO₄)₂, an insoluble form of inorganic phosphate. Flasks were transferred to a shaker/incubator set to a temperature of 30° C. and shaking at 200 rpm. Along with culture flasks, two flasks containing 25 mL of uninoculated NBRIP-BPB growth media were incubated as a negative control.

On the day of inoculation (D0), for each culture 1.5 mL of uninoculated NBRIP-BPB growth media was transferred to each of two 2 mL centrifuge tubes. These were spun down in a benchtop centrifuge at 13.2 krpm for 5 minutes. The supernatant was removed and tested for soluble phosphate concentration. At D0, background soluble phosphate was measured at about 3 μg/mL.

At subsequent timepoints day 1 after inoculation (D1), day 4 after inoculation (D4), day 7 after inoculation (D7), and day 10 after inoculation (D10), the flasks were removed from the shaker/incubator, samples were collected as described above, and the supernatant was collected by centrifugation as described above. The flasks were each returned to continue the incubation after supernatant collection.

Soluble phosphate content of supernatant samples was quantified using a modified version of the ascorbic acid method (Murphy and Riley, 1962). A standard curve was prepared in the range 0.5 μg/mL P to 10 μg/mL P by dissolving a known concentration of potassium phosphate in molecular water. Samples were diluted using molecular water until their soluble phosphate concentration fell within the range of the standard curve. 200 μL of each standard and sample was added to the wells of a clear microplate, and 40 μL of active reagent was added to each well and mixed by pipetting. The active reagent was prepared as 25 mL 5N sulfuric acid, 7.5 mL of 40 g/L ammonium molybdate, 15 mL of 0.1 M ascorbic acid, and 2.5 mL potassium antimonyl tartrate, (1 mg Sb/mL) fresh on each day the colorimetric assays were carried out. The plate was incubated at RT, and the absorbance of each well at 882 nm measured. Sample concentrations were calculated by extrapolation from standard concentrations fitted to a rectangular hyperbola.

Table 4 shows the average soluble phosphate of three replicate cultures of Rahnella aquatilis CI019 and remodeled strains. At days 1 and 7, all remodeled strains show at least a 10% increase in soluble phosphate compared to WT CI019

TABLE 4
Rahnella aquatilis strain CI019 and derivatives
average soluble phosphate produced

Average Soluble Phosphate (μg/mL)

| Strain | Day 1 | Day 4 | Day 7 | Day 10 |
|---|---|---|---|---|
| CI019 | 257.3 | 229.8 | 128.2 | 88.5 |
| 19-1276 | 289.2 | 275.2 | 162.0 | 91.5 |
| 19-1277 | 300.3 | 244.1 | 158.8 | 110.0 |
| 19-1280 | 298.4 | 245.9 | 149.1 | 113.2 |

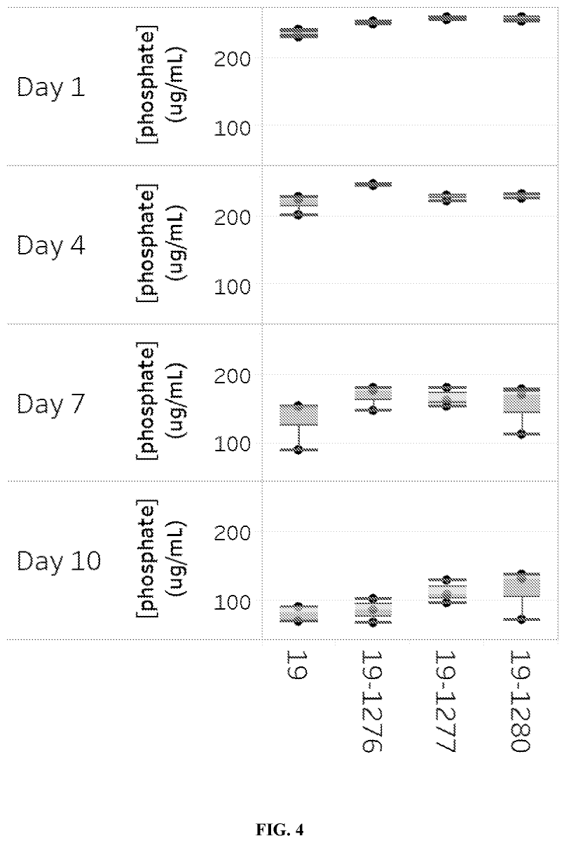

Soluble phosphate in three replicate cultures of Rahnella aquatilis CI019 and remodeled strains are shown in FIG. 4. All strains solubilized phosphate above the D0 background level of about 3 μg/mL. At Day 1, all three strains showed significant increases in soluble phosphate compared to that of WT CI019 (p<0.03). P values were determined using a two-tailed two-sample unequal variance T test.

### Example 5: Phosphate Solubilization by Further Rahnella aquatilis Strains

One wild type (WT) and 8 mutant strains of Rahnella aquatilis 63 were subjected to a phosphate solubilization screen. Briefly, this assay comprised culturing each strain in the presence of an insoluble form of phosphate, collecting the supernatant of the cultured microbes, and performing a colorimetric assay on the supernatant to determine the soluble phosphate content.

A list and brief description of each strain assayed is provided in Table 5. Sequences for each strain are provided in Table 9. Some further strains which were generated but have not been screened for phosphate solubilization are listed in Table 7.

TABLE 5
Rahnella aquatilis descriptions

| Strain ID | Lineage | Mutagenic DNA Description | Genotype |
|---|---|---|---|
| 63 | N/A | N/A | WT |
| 63-1461 | Mutant of | Deletion of the entire gntT gene | ΔgntT |
| | 63 | | |
| 63-1463 | Mutant of | Insertion of a 500 bp fragment of the region | gcd1::Prm1.2 |
| | 63 | 299 bp upstream of the thrS stop codon to | |
| | | 126 bp downstream of the infC start codon | |
| | | (Prm1.2) inserted upstream of gcd1. 215 bp | |
| | | of the original upstream sequence of gcd1 | |
| | | was deleted and replaced with prm1.2 | |
| 63-1467 | Mutant of | Insertion of a 500 bp fragment of the region | gcd2::Prm1.2 |
| | 63 | 299 bp upstream of the thrS stop codon to | |
| | | 126 bp downstream of the infC start codon | |
| | | (Prm1.2) inserted upstream of gcd2. 166 bp | |
| | | of the original upstream sequence of gcd2 | |
| | | was deleted and replaced with prm1.2 | |
| 63-1477 | Mutant of | Insertion of a 500 bp fragment of the region | pqqA::Prm1.2 |
| | 63 | 299 bp upstream of the thrS stop codon to | |
| | | 126 bp downstream of the infC start codon | |
| | | (Prm1.2) inserted 129 bp upstream of pqqB. | |
| | | 170 bp of the original sequence upstream | |
| | | 129 bp of pqqB was deleted and replaced | |
| | | with prm1.2 | |
| 63-1479 | Mutant of | Insertion of a 170 bp fragment of the region | pqqA::Prm3.1 |
| | 63 | upstream of the rplN (Prm3.1) inserted | |
| | | 129 bp upstream of pqqB. 170 bp of the | |
| | | original sequence upstream 129 bp of pqqB | |
| | | was deleted and replaced with prm3.1. | |
| 63-1570 | Mutant of | Deletion of the entire gad1 gene | Δgad1 |
| | 63 | | |
| 63-1644 | Mutant of | Insertion of a 142 bp fragment of the region | pqqA::Prm6.1 |
| | 63 | upstream of an hypothetical protein CDS | |
| | | (PROKKA-01887) (Prm6.1) inserted 129 bp | |
| | | upstream of pqqB. 170 bp of the original | |
| | | sequence upstream 129 bp of pqqB was | |
| | | deleted and replaced with prm6.1. | |
| 63-1647 | Mutant of | Insertion of a 170 bp fragment of the region | gcd1::Prm3.1 |
| | 63 | upstream of the rplN (Prm3.1) inserted | |
| | | upstream of gcd1. 215 bp of the original | |
| | | upstream sequence of gcd1 was deleted and | |
| | | replaced with prm3.1 | |

All strains were streaked out from frozen stocks on super optimal broth (SOB) plates. Plates were incubated at 30° C. overnight. For each strain, 3 individual colonies from the overnight plates were inoculated into 5 mL of liquid SOB. These cultures were grown at 30° C. with shaking overnight. The optical density at 590 nm (OD590) of each of the overnight SOB cultures was measured. In order to normalize the starting optical densities across strains, sufficient volume of each culture was added 25 mL of National Botanical Research Institute's Phosphate-Bromo Phenol Blue (NBRIP-BPB) (modified from Dash et al., 2017) growth media in a 125 mL conical flask, resulting in a starting OD590 of 0.03 for each culture. NBRIP—BPB contains 5 g/L Ca₃(PO₄)₂, an insoluble form of inorganic phosphate. Flasks were transferred to a shaker/incubator set to a temperature of 30° C. and shaking at 200 rpm. Along with culture flasks, two flasks containing 25 mL of uninoculated NBRIP-BPB growth media were incubated as a negative control.

On the day of inoculation (D0), for each culture 1.5 mL of uninoculated NBRIP-BPB growth media was transferred to each of two 2 mL centrifuge tubes. These were spun down in a benchtop centrifuge at 13.2 krpm for 5 minutes. The supernatant was removed and tested for soluble phosphate concentration. At D0, background soluble phosphate was measured at about 3 μg/mL.

At subsequent timepoints day 1 after inoculation (D1), day 4 after inoculation (D4), day 7 after inoculation (D7), and day 10 after inoculation (D10), the flasks were removed from the shaker/incubator, samples were collected as described above, and the supernatant was collected by centrifugation as described above. The flasks were each returned to continue the incubation after supernatant collection.

Soluble phosphate content of supernatant samples was quantified using a modified version of the ascorbic acid method (Murphy and Riley, 1962). A standard curve was prepared in the range 0.5 μg/mL P to 10 μg/mL P by dissolving a known concentration of potassium phosphate in molecular water. Samples were diluted using molecular water until their soluble phosphate concentration fell within the range of the standard curve. 200 μL of each standard and sample was added to the wells of a clear microplate, and 40 μL of active reagent was added to each well and mixed by pipetting. The active reagent was prepared as 25 mL 5 N sulfuric acid, 7.5 mL of 40 g/L ammonium molybdate, 15 mL of 0.1 M ascorbic acid, and 2.5 mL potassium antimonyl tartrate, (1 mg Sb/mL) fresh on each day the colorimetric assays were carried out. The plate was incubated at RT, and the absorbance of each well at 882 nm was measured. Sample concentrations were calculated by extrapolation from standard concentrations fitted to a rectangular hyperbola.

Table 6 shows the average soluble phosphate of three replicate cultures of Rahnella aquatilis 63 and remodeled strains. At Days 7 and 10, all strains except 63-1570 show an increase in average solubilized phosphate.

TABLE 6
Rahnella aquatilis strain 63 and
derivatives average soluble phosphate

Average Soluble Phosphate (μg/mL)

| Strain | Day 1 | Day 4 | Day 7 | Day 10 |
|---|---|---|---|---|
| 63 | 280.1 | 214.9 | 139.7 | 101.8 |
| 63-1461 | 279.3 | 221.0 | 168.7 | 115.7 |
| 63-1463 | 280.8 | 221.1 | 149.4 | 116.2 |
| 63-1467 | 274.0 | 222.3 | 161.8 | 118.5 |
| 63-1477 | 273.6 | 209.7 | 171.2 | 107.9 |
| 63-1479 | 287.8 | 208.4 | 172.0 | 137.6 |
| 63-1570 | 287.6 | 188.9 | 89.9 | 56.5 |
| 63-1644 | 228.8 | 203.2 | 164.0 | 113.8 |
| 63-1647 | 292.8 | 225.4 | 169.9 | 122.6 |

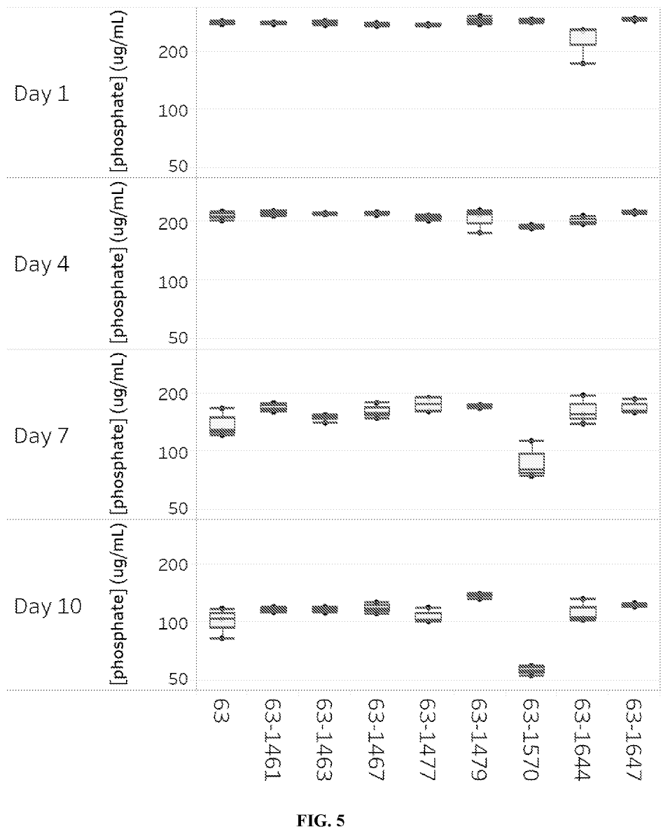

Soluble phosphate in three replicate cultures of Rahnella aquatilis 63 and remodeled strains are shown in FIG. 5. All strains solubilized phosphate above the D0 background level of about 3 μg/mL. At Day 1, 63-1647 showed a statistically significant increase in solubilized phosphate compared to that of WT 63 (p=0.04). At day 10, 63-1479 showed a nearly significant increase in phosphate solubilized compared to that of WT 63 (p=0.06). P values were determined using a two-tailed two-sample unequal variance T test.

TABLE 7
Further Rahnella aquatilis strains

| Strain ID | Lineage | Mutagenic DNA Description | Genotype |
|---|---|---|---|
| 63-1465 | Mutant of | Insertion of a 142 bp fragment of the | gcd1::Prm6.1 |
| | 63 | region upstream of an hypothetical | |
| | | protein CDS (PROKKA-01887) | |
| | | (Prm6.1) inserted upstream of gcd1. | |
| | | 215 bp of the original upstream | |
| | | sequence of gcd1 was deleted and | |
| | | replaced with prm6.1 | |
| 63-1469 | Mutant of | Insertion of a 500 bp fragment of the | phoC2::Prm1.2 |
| | 63 | region 299 bp upstream of the thrS stop | |
| | | codon to 126 bp downstream of the infC | |
| | | start codon (Prm1.2) inserted upstream | |
| | | of phoC2. 70 bp of the original | |
| | | upstream sequence of phoC2 was | |
| | | deleted and replaced with prm1.2 | |
| 63-1471 | Mutant of | Insertion of a 170 bp fragment of the | phoC2::Prm3.1 |
| | 63 | region upstream of the rplN (Prm3.1) | |
| | | inserted upstream of phoC2. 70 bp of | |
| | | the original upstream sequence of | |
| | | phoC2 was deleted and replaced with | |
| | | prm3.1 | |
| 63-1473 | Mutant of | Insertion of a 142 bp fragment of the | phoC2::Prm6.1 |
| | 63 | region upstream of an hypothetical | |
| | | protein CDS (PROKKA-01887) | |
| | | (Prm6.1) inserted upstream of phoC2. | |
| | | 70 bp of the original upstream sequence | |
| | | of phoC2 was deleted and replaced with | |
| | | prm6.1 | |
| 63-1475 | Mutant of | Insertion of a 293 bp fragment of the | phoC2::Prm7.1 |
| | 63 | region upstream of the lpp (Prm7.1) | |
| | | inserted upstream of phoC2. 70 bp of | |
| | | the original upstream sequence of | |
| | | phoC2 was deleted and replaced with | |
| | | prm3.1 | |
| 63-1481 | Mutant of | Insertion of a 293 bp fragment of the | pqqA::Prm7.1 |
| | 63 | region upstream of the rplN (Prm7.1) | |
| | | inserted 129 bp upstream of pqqB. | |
| | | 170 bp of the original sequence | |
| | | upstream 129 bp of pqqB was deleted | |
| | | and replaced with prm7.1. | |
| 63-1511 | Mutant of | Insertion of a 293 bp fragment of the | phoC1::Prm7.1 |
| | 63 | region upstream of the lpp (Prm7.1) | |
| | | inserted upstream of phoC1. 145 bp of | |
| | | the original upstream sequence of | |
| | | phoC1 was deleted and replaced with | |
| | | prm7.1 | |
| 63-1575 | Mutant of | Insertion of a 170 bp fragment of the | phoC1::Prm3.1 |
| | 63 | region upstream of the rplN (Prm3.1) | |
| | | inserted upstream of phoC1. 145 bp of | |
| | | the original upstream sequence of | |
| | | phoC1 was deleted and replaced with | |
| | | prm3.1 | |
| 63-1638 | Mutant of | Insertion of a 170 bp fragment of the | gcd2::Prm3.1 |
| | 63 | region upstream of the rplN (Prm3.1) | |
| | | inserted upstream of gcd2. 166 bp of the | |
| | | original upstream sequence of gcd2 was | |
| | | deleted and replaced with prm3.2 | |
| 63-1640 | Mutant of | Insertion of a 142 bp fragment of the | gcd2::Prm6.1 |
| | 63 | region upstream of an hypothetical | |
| | | protein CDS (PROKKA-01887) | |
| | | (Prm6.1) inserted upstream of gcd2. | |
| | | 166 bp of the original upstream | |
| | | sequence of gcd2 was deleted and | |
| | | replaced with prm6.1 | |
| 63-1642 | Mutant of | Insertion of a 142 bp fragment of the | phoC1::Prm6.1 |
| | 63 | region upstream of an hypothetical | |
| | | protein CDS (PROKKA-01887) | |
| | | (Prm6.1) inserted upstream of phoC1. | |
| | | 145 bp of the original upstream | |
| | | sequence of phoC1 was deleted and | |
| | | replaced with prm6.1 | |
| 63-1649 | Mutant of | Insertion of a 293 bp fragment of the | gcd1::Prm7.1 |
| | 63 | region upstream of the lpp (Prm7.1) | |
| | | inserted upstream of gcd1. 215 bp of the | |
| | | original upstream sequence of gcd1 was | |
| | | deleted and replaced with prm7.1 | |

TABLE 8
Sequences referenced in this disclosure

| SEQ ID NO: | Description |
|---|---|
| 1 | PhoC1 native sequence |
| 2 | PhoC2 native sequence |
| 3 | PhoC1 codon randomized |
| 4 | PhoC2 codon randomized |
| 5 | Plac′ + RBS |
| 6 | Bba_J23110 |
| 7 | Bba_B0032 |
| 8 | Plpp + RBS |
| 9 | CloDF13 |
| 10 | KanR |

TABLE 9
Further Sequences

| SEQ ID NO: | Strain ID | Gene/mutation | Sequence Type |
|---|---|---|---|
| 11 | 137 | pqq operon | nucleotide |
| 12 | 137 | pqqA | nucleotide |
| 13 | 137 | pqqB | nucleotide |
| 14 | 137 | pqqC | nucleotide |
| 15 | 137 | pqqD | nucleotide |
| 16 | 137 | pqqE | nucleotide |
| 17 | 137 | gad | nucleotide |
| 18 | 137 | gntT | nucleotide |
| 19 | 137 | gcd | nucleotide |
| 20 | 137 | prm8.2 | nucleotide |
| 21 | 137 | prm15.2 | nucleotide |
| 22 | 137 | prm16.1 | nucleotide |
| 23 | 137-1855 | pqq_operon::prm15.2 | nucleotide |
| 24 | 137-1856 | Δgad | nucleotide |
| 25 | 137-1914 | pqq_operon::prm8.2 | nucleotide |
| 26 | 137-1916 | pqq_operon::prm16.1 | nucleotide |
| 27 | 137-2142 | ΔgntT | nucleotide |
| 28 | 137-2234 | gcd::prm16.1 | nucleotide |
| 29 | 137-2311 | gcd::prm8.2 | nucleotide |
| 30 | 63 | gntT | nucleotide |
| 31 | 63 | gcd1 | nucleotide |
| 32 | 63 | gcd2 | nucleotide |
| 33 | 63 | phoC1 | nucleotide |
| 34 | 63 | phoC2 | nucleotide |
| 35 | 63 | pqq operon | nucleotide |
| 36 | 63 | pqqA | nucleotide |
| 37 | 63 | pqqB | nucleotide |
| 38 | 63 | pqqC | nucleotide |
| 39 | 63 | pqqD | nucleotide |
| 40 | 63 | pqqE | nucleotide |
| 41 | 63 | gad1 | nucleotide |
| 42 | 63 | Prm1.2 | nucleotide |
| 43 | 63 | Prm3.1 | nucleotide |
| 44 | 63 | Prm6.1 | nucleotide |
| 45 | 63 | Prm7.1 | nucleotide |
| 46 | 63-1461 | ΔgntT | nucleotide |
| 47 | 63-1463 | gcd1::Prm1.2 | nucleotide |
| 48 | 63-1465 | gcd1::Prm6.1 | nucleotide |
| 49 | 63-1467 | gcd2::Prm1.2 | nucleotide |
| 50 | 63-1469 | phoC2::Prm1.2 | nucleotide |
| 51 | 63-1471 | phoC2::Prm3.1 | nucleotide |
| 52 | 63-1473 | phoC2::Prm6.1 | nucleotide |
| 53 | 63-1475 | phoC2::Prm7.1 | nucleotide |
| 54 | 63-1477 | pqqA::Prm1.2 | nucleotide |
| 55 | 63-1479 | pqqA::Prm3.1 | nucleotide |
| 56 | 63-1481 | pqqA::Prm7.1 | nucleotide |
| 57 | 63-1511 | phoC1::Prm7.1 | nucleotide |
| 58 | 63-1570 | Δgad1 | nucleotide |
| 59 | 63-1575 | phoC1::Prm3.1 | nucleotide |
| 60 | 63-1638 | gcd2::Prm3.1 | nucleotide |
| 61 | 63-1640 | gcd2::Prm6.1 | nucleotide |
| 62 | 63-1642 | phoC1::Prm6.1 | nucleotide |
| 63 | 63-1644 | pqqA::Prm6.1 | nucleotide |
| 64 | 63-1647 | gcd1::Prm3.1 | nucleotide |
| 65 | 63-1649 | gcd1::Prm7.1 | nucleotide |
| 66 | CI019 | gad1 | nucleotide |
| 67 | CI019 | gad2 | nucleotide |
| 68 | CI019 | gntT | nucleotide |
| 69 | 19-1276 | Δgad1::specR-sacB | nucleotide |
| 70 | 19-1277 | Δgad2::specR-sacB | nucleotide |
| 71 | 19-1280 | ΔgntT::specR-sacB | nucleotide |
| 72 | 137 | pqqA | amino acid |
| 73 | 137 | pqqB | amino acid |
| 74 | 137 | pqqC | amino acid |
| 75 | 137 | pqqD | amino acid |
| 76 | 137 | pqqE | amino acid |
| 77 | 137 | gad | amino acid |
| 78 | 137 | gntT | amino acid |
| 79 | 137 | gcd | amino acid |
| 80 | 63 | gntT | amino acid |
| 81 | 63 | gcd1 | amino acid |
| 82 | 63 | gcd2 | amino acid |
| 83 | 63 | phoC1 | amino acid |
| 84 | 63 | phoC2 | amino acid |
| 85 | 63 | pqqA | amino acid |
| 86 | 63 | pqqB | amino acid |
| 87 | 63 | pqqC | amino acid |
| 88 | 63 | pqqD | amino acid |
| 89 | 63 | pqqE | amino acid |
| 90 | 63 | gad1 | amino acid |
| 91 | CI019 | gad1 | amino acid |
| 92 | CI019 | gad2 | amino acid |
| 93 | CI019 | gntT | amino acid |

1. An engineered microbe comprising an alteration in a gene associated with phosphate solubilization, wherein said gene associated with phosphate solubilization is native to said microbe, whereby said engineered microbe solubilizes phosphates at a greater capacity or rate as compared to a non-engineered microbe of the same species.

2. An engineered microbe comprising an alteration in a gene associated with phosphate solubilization, wherein said engineered microbe is a non-intergeneric engineered microbe, and wherein said engineered microbe solubilizes phosphates at a greater capacity or rate as compared to a non-engineered microbe of the same species.

3. The engineered microbe of claim 1, wherein said gene associated with phosphate solubilization is a non-specific acid phosphatase gene, a phytase gene, a gluconic acid biosynthetic gene, a gluconic acid transporter, a gluconate dehydrogenase, a glucose dehydrogenase, or a functional variant thereof, or any combination thereof.

4.-9. (canceled)

10. The engineered microbe of claim 1 wherein said engineered microbe comprises altered expression of said gene associated with phosphate solubilization as compared to a microbe of the same species which lacks the alteration in the gene.

11. The engineered microbe of claim 1, wherein said alteration in a gene associated with phosphate solubilization comprises an insertion of a regulatory element.

12.-18. (canceled)

19. The engineered microbe of claim 1, wherein said alteration in a gene associated with phosphate comprises codon optimization or codon randomization.

20. (canceled)

21. The engineered microbe of claim 1, wherein said alteration in a gene associated with phosphate comprises a reduction in gene function, a loss of function mutation, or a gene deletion.

22.-23. (canceled)

24. The engineered microbe of claim 1, wherein said engineered microbe is an engineered bacterium, an engineered fungus, or a biocontrol microbe.

25.-31. (canceled)

32. The engineered microbe of claim 1, wherein said engineered microbe fixes nitrogen.

33.-49. (canceled)

50. A method of increasing an amount of soluble phosphate in soil, the method comprising contacting soil comprising insoluble phosphate with the engineered microbe of claim 1.

51.-52. (canceled)

53. A method of producing an engineered microbe with improved phosphate solubilization activity, the method comprising:
a) altering a codon usage of a native coding sequence associated with phosphate solubilization to yield a codon altered coding sequence associated with phosphate solubilization;
b) functionally linking the codon altered coding sequence associated with phosphate solubilization to a promoter; and
c) introducing the promoter and the codon altered coding sequence associated with phosphate solubilization into a microbe to produce the improved microbe.

54. The method of claim 52, wherein said native coding sequence is identified from a microbe of the same species as said improved microbe.

55.-137. (canceled)


SEQUENCE LISTING (93 sequences)

SEQ ID NO: 1 - DNA, 699 bp, Rahnella aquatilis
gtgcgcaagaccctcgttgctctgctgcttttcaccctcggttccacgtttgccgtccaggccgctgacgaagccaaaccctttatcaccagtcaggaactggatctgaccaaatatctgcccgcgccaccggcggatgattcggcgcagaccaaagcggaactgaaagaattgctggaaattcaggccacccgtacaccggagcaggaaaaagcggcgattgccgatgctgaggaaaacgtctggcgttttgccgacgtgatggggccggactttgatgccgcaaaactgccgaaaaccgcggcactgtttgatcgtattgttgcgaccgaagatgtggtggacgatcacgccaagaaagcctttaaccgcccgcgtccttacatgctggatgaacagatccatccgctgctgaaaaaatccaaatccggctcatggccttccggtcattcgactatcggttacctgatggcgaccgtgctcggtgaaatggtgccggaaaaacgtaatgcgctgtttacccgtgcagccggttatgccgaaaaccgtctggtggctggtttccattaccgttctgataccgtgatgagccgcaccggtgctgcgctgattgcccagaaaatggaagagcaaccggatttcaaaaccgaattcgacgcggcgaaagcggaagttcgtgcacagcttggtctgaaataa

SEQ ID NO: 2 - DNA, 738 bp, Rahnella aquatilis
atgaaaattattactactttttgcctcgccagctttttttctgtcagctcgtttgccctgaccggcaatgatgcaaccaccaaaccagacctttactacttaaaaaatgatcaggcgattaacagcctggcgctgctgccgccaccacctgcggtgggcagtatcgctttcctaaacgatcaggccatgtatgaacaggggcgtctgctgcgctcaactgaacgtggcaaactggctgccgaagatgccaacctgagtgccggtggcgtcgcgaacgctttctcgggcgcattcggttcgccgatcaccgctaaagacacaccggaattgcacaaactgctgaccaatatgattgaagatgcaggtgaccttgcgacacgttctgctaaagaaaagtacatgcgcatccgcccgtttgccttctatggtgtgccgacctgtaacaccactgagcaggataagctgtccaaaaacggttcgtatccttccgggcacacttctattggctgggctacggcgctggtactcacagaaattaacccgcagcgccaggatcaaatcctgcaacgtggtttcgatttaggacaaagccgtgtgatttgtggctaccactggcaaagtgatgtcgatgcggcccgtatcgtgggctccgctgtggtcgctaccttgcatacgaattcggctttccagcaacagttgcaaaaagccaaagaagagtttgcgaaacagcatccgtaa

SEQ ID NO: 3 - DNA, 699 bp, Artificial Sequence
  source: /note=“Description of Artificial Sequence Synthetic polynucleotide”
atgcgcaaaaccctggtggcgctgctgctgtttaccctgggcagcacctttgcggtgcaggcggcggatgaagcgaaaccgtttattaccagccaggaactggatctgaccaaatatctgccggcgccgccggcggatgatagcgcgcagaccaaagcggaactgaaagaactgctggaaattcaggcgacccgcaccccggaacaggaaaaagcggcgattgcggatgcggaagaaaacgtgtggcgctttgcggatgtgatgggcccggattttgatgcggcgaaactgccgaaaaccgcggcgctgtttgatcgcattgtggcgaccgaagatgtggtggatgatcatgcgaaaaaagcgtttaaccgcccgcgcccgtatatgctggatgaacagattcatccgctgctgaaaaaaagcaaaagcggcagctggccgagcggccatagcaccattggctatctgatggcgaccgtgctgggcgaaatggtgccggaaaaacgcaacgcgctgtttacccgcgcggcgggctatgcggaaaaccgcctggtggcgggctttcattatcgcagcgataccgtgatgagccgcaccggcgcggcgctgattgcgcagaaaatggaagaacagccggattttaaaaccgaatttgatgcggcgaaagcggaagtgcgcgcgcagctgggcctgaaatga

SEQ ID NO: 4 - DNA, 738 bp, Artificial Sequence
  source: /note=“Description of Artificial Sequence Synthetic polynucleotide”
atgaaaattattaccaccttttgcctggcgagcttttttagcgtgagcagctttgcgctgaccggcaacgatgcgaccaccaaaccggatctgtattatctgaaaaacgatcaggcgattaacagcctggcgctgctgccgccgccgccggcggtgggcagcattgcgtttctgaacgatcaggcgatgtatgaacagggccgcctgctgcgcagcaccgaacgcggcaaactggcggcggaagatgcgaacctgagcgcgggcggcgtggcgaacgcgtttagcggcgcgtttggcagcccgattaccgcgaaagataccccggaactgcataaactgctgaccaacatgattgaagatgcgggcgatctggcgacccgcagcgcgaaagaaaaatatatgcgcattcgcccgtttgcgttttatggcgtgccgacctgcaacaccaccgaacaggataaactgagcaaaaacggcagctatccgagcggccataccagcattggctgggcgaccgcgctggtgctgaccgaaattaacccgcagcgccaggatcagattctgcagcgcggctttgatctgggccagagccgcgtgatttgcggctatcattggcagagcgatgtggatgcggcgcgcattgtgggcagcgcggtggtggcgaccctgcataccaacagcgcgtttcagcagcagctgcagaaagcgaaagaagaatttgcgaaacagcatccgtga

SEQ ID NO: 5 - DNA, 104 bp, Artificial Sequence
  source: /note=“Description of Artificial Sequence Synthetic polynucleotide”
acaggctcactcattaggcaccccaggctttacactttatgcttccggctcgtatgttgtgtggaattgtgagcgtctagtagaaggaggagatctggatccat

SEQ ID NO: 6 - DNA, 35 bp, Artificial Sequence
  source: /note=“Description of Artificial Sequence Synthetic oligonucleotide”
tttacggctagctcagtcctaggtacaatgctagc

SEQ ID NO: 7 - DNA, 13 bp, Artificial Sequence
  source: /note=“Description of Artificial Sequence Synthetic oligonucleotide”
tcacacaggaaag

SEQ ID NO: 8 - DNA, 313 bp, Enterobacter sacchari
acagtaataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaata

SEQ ID NO: 9 - DNA, 739 bp, Artificial Sequence
  source: /note=“Description of Artificial Sequence Synthetic vector sequence”
gatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgc

SEQ ID NO: 10 - DNA, 852 bp, Artificial Sequence
  source: /note=“Description of Artificial Sequence Synthetic vector sequence”
ttagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgagacggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacag

SEQ ID NO: 11 - DNA, 5215 bp, Klebsiella variicola
tggcaattgttgcgggcatgtgaatttatgcttgccccaggagataaatccgttaccgtggcaggcatttttcaccacttaagaacgcaggaacccgctatggcgatttttgatggtcacaatgacctgctgttgaatctctggcttcaccatcgtgaggatccggtaagcgccttctttgtcgggatagaaaacggccatctcgactatccgcgtatccgccagggcgggctggccggcggcctgttcgccctctttgtgccgccgcaggagtatatcgcccgcgtggcgccgcagtacgccagcgaggcgtgggatccgctggcgatcctctggcagcagttggctatcctcaaggcgatttgcgcccatgacgccagccgcgcgcggctgtgcctgagcgcggcggatatcgaacgctgtcgccaggataacgcgctggcgatggtggcgcacatcgaaggtgctggcgggttcgacgcgcaaggcgaagatctgcaggccttttatcgcgccggggtgcgcagcattggtccgttctggaacatcgccaatcgcttcggctgcggggtcaccggcgcctttcccggcagcccggacagcgggccgggtctcactcgcgagggcattgcgctcatcgctcaggccaacgccctgaagatgcagatcgatgtttcgcacatgaatgaacaggccttttgggataccgcccaccactccaccgccccactggtggccacccactctaatgcccacgccctgtgcccacagccgcgcaatctgaccgaccggcaactgcgggcgatccgcgacagcggcggcgtagtgggcgtcaatttcggcaatgctttcctgcgcgccgacggacggcgcgacagcgacacgcccttaaccaccattgtccggcatatcgactatcttattaacataatgggtgaggatcatgtggctctcggctcggattttgacggcatcaccctgcccgacgagctgggcgatgtggccggcttaccgcggcttatcaatgccttgcgggacaatggctatgatcaattggtgctggataagctgctgtggaacaactggctgcgggtattaaaaaaggtttggcaacaataggttatattgtaaacataaagttaattccttacagattccgggttgatctctctcccggtgaagcgcaatatgcggcggccgtcacagatccagcattatccgtgaggcctgtcgtcatttttaagaggaacaaaacatgtggaagaaacctgcttttatcgatttacgtctcggtctggaagtgacgctgtacatttctaaccgttaatcgccccgcccgccgttcgcgcgggcaccttcattcattacccggtccgtcttcatgttcattaaagtcctcggctccgccgccggcggcggtttcccgcaatggaactgcaactgcgccaactgtcagggtctgcgcaacggcaccattcaggccagtgcccgcacccagtcgtcgatcatcgtcagcgataacggcaaagagtgggtgctgtgcaatgcctcgccggatatcagccagcagattgcccatacccccgagttaaataaacccggcgtactgcgcgggacgtctatcggcggcattattctcaccgacagccagatcgaccacaccaccgggttgctgagcctgcgcgaaggctgcccgcaccaggtgtggtgcacgccggaggttcatcaggatctctccaccggcttcccggtgtttaccatgctgcgacactggaacggcggcctggtgcatcatcccatcgcgccgcagcagccttttaccgttgacgcctgccctgatttgcagtttaccgccgtgcctatcgccagcaacgcgccgccctattcgccgtatcgcgaccggccgctgccgggccataacgtggcgctgtttatcgaaaaccgccgcaacgggcagacgctgttctatgccccggggctgggtgagccggatgaagcccttctgccgtggctgcaaaaagcggactgtctgctgatcgatggcaccgtctggcaggatgacgagctgcaggccgccggcgtcgggcgcaataccggtcgcgatatgggacacctggcgctcagcgatgagcacgggatgatggccttgctggcctccctgccggcaaaacgcaaaattctcattcatattaataacaccaacccgatccttaacgaacagtctccccagcgccaggcgctaacgcaacaggggattgaagtgagctgggacgggatggcaatcacccttcaggataccgcatgctgatcaccgacacgctgtcgccgcaggcctttgcagaggctctgcgggctaaaggcgccttctaccatattcaccacccttaccacatcgccatgcataacggcgaagcgacccgcgagcaaattcagggttgggtggcgaaccggttttattaccagaccaccattccgctgaaagacgcggcgattatggctaactgcccggatgcgcagacccggcgcaaatgggtgcagcggatcctcgaccacgacggtagccacggcgaagatggcgggattgaagcctggctgcggctgggggaagcggtcggtttgagccgcgacgacctgctcagcgagcgtcacgtgctgcccggcgtgcgcttcgcggtggatgcctatcttaatttcgctcgtcgcgcctgctggcaggaggcggcctgcagctccctgaccgagctgttcgccccacagatccatcagtcgcgcctcgacagctggccgcagcactatccgtggatcaaagaggaaggctatttttacttccgcagtcgtctgagccaggctaaccgcgacgttgagcatggtctggcgctggcgaagacctactgtgacagcgctgaaaaacagaaccggatgctggagatcctgcagtttaagctcgacatcctgtggtcgatgctcgatgccatgaccatggcctacgctctgcagcgcccgccctatcacacggtcaccgacaaggcggcctggcacacgacccgactggtgtaatcatgcaaaaaacgtccatcgttgcctttcgtcgcggctaccgactgcagtgggaagccgcccaggagagccatgtgatcctctatccggagggaatggctaaactcaatgagaccgccgcggcgatcctcgagctggtcgatggccggcgcgacgtcgcggcgattatcgccatgcttaacgaacgtttcccggaagccggcggcgtcgatgacgacgtcgtcgagttcctgcagatcgcctgtcaacagaagtggatcacctgccgtgagccagaataaacccgccgtcaatccgccgctgtggctgctggcggagctgacctaccgctgcccgctgcagtgtccctactgttccaatccgctggacttcgcccggcaggaaaaggagctgaccaccgaacaatggatcgaggtctttcgccaggcgcgagcgatgggcagcgtacagctgggcttttccggcggcgagccgctgacccgtaaagatctgccggagctgatccgcgccgcgcgcgacctcgggttctataccaacctgatcacctcgggaattgggctaaccgagagcaaactcgacgccttcagcgaggccggactggaccatatccagattagcttccaggccagcgatgaggtgctcaacgccgctcttgccggcaataaaaaagccttccagcagaagctggcgatggccagagcggtgaaagcgcgcgactacccgatggtgctgaacttcgtcctccaccggcataacatcgaccagctcgataaaattatcgagctgtgcattgagctggaagccgatgacgtcgagctcgccacctgccagttttacggctgggcgtttcttaatcgcgaggggttactgccgacccgggaacagatcgcccgcgccgagcaggtggtcgccgattaccggcagaaaatggccgccagcggtaacctcaccaacctgctattcgtcaccccggactattacgaggaacgcccgaaaggctgtatgggcggctggggatcgattttcctcagcgtcactccggaaggcactgcgttgccgtgccacagcgcgcgccagctgccggtggcgttcccgtcggtgctggagcagagtctggaatcgatctggtatgactcgttcggcttcaaccgttatcgcgggtatgactggatgccggagccgtgccgctcctgtgatgaaaaagagaaagacttcggcggctgccgctgtcaggcctttatgctgaccggcagcgccgataacgccgacccggtgtgcagcaaatccccacatcatcacaaaatccttgaggcccggcgcgaagcggcctgcagcgacatcaaagtcagccagctgcagttccgcaaccgtacccgctcgcagcttatctacaaaacccgggaactgtaatgacgctggcgacccgcactgtcactctgccgggcggcctgcaggctaccctggttcatcagccgcaggccgatcgcgcggcggccctggtgcgggttgccgccggcagccaccatgaaccgtcgtgcttccccggtctggcgcacctgctggaacacctgctgttttacggcggtgagcgctaccgcaatgatgaacggctgatgagctgggtgcagcgccaggcagggaatgtgaatgccaccaccctgtcccgccacagcgctttctttttcgaggtcgccgccgaggatctggctgacggcgtcgcgcgcctgcaggagatgctgcaggcgccgctgctgctcagggacgatattcaacgcgaagtcgcggttatcgacgccgaaaaccgcctgatccaacagcatgagttgtcgcgacgggaagccgccgtgcgtcacgccgccatcgcgcccgcggcgtttcgccgctttcaggtcggcgacgccgggtcgctgggggaggatttcctcgcgctacaggcggccttacgtgactttcaccgcagccactacgtcgcccgccggatgcaactctggctgcaggggccgcagtcgctggaggtgctcggcgaactggcgacccgtttcgccaccgggcttgccccgggcgaggcaccgccgccagcgccgccgctcactctgggcgagccccctcaactgcagctggccgtctccagccagcccgcgctgtggcgctgcccgct

SEQ ID NO: 12 - DNA, 1020 bp, Klebsiella variicola
atggcgatttttgatggtcacaatgacctgctgttgaatctctggcttcaccatcgtgaggatccggtaagcgccttctttgtcgggatagaaaacggccatctcgactatccgcgtatccgccagggcgggctggccggcggcctgttcgccctctttgtgccgccgcaggagtatatcgcccgcgtggcgccgcagtacgccagcgaggcgtgggatccgctggcgatcctctggcagcagttggctatcctcaaggcgatttgcgcccatgacgccagccgcgcgcggctgtgcctgagcgcggcggatatcgaacgctgtcgccaggataacgcgctggcgatggtggcgcacatcgaaggtgctggcgggttcgacgcgcaaggcgaagatctgcaggccttttatcgcgccggggtgcgcagcattggtccgttctggaacatcgccaatcgcttcggctgcggggtcaccggcgcctttcccggcagcccggacagcgggccgggtctcactcgcgagggcattgcgctcatcgctcaggccaacgccctgaagatgcagatcgatgtttcgcacatgaatgaacaggccttttgggataccgcccaccactccaccgccccactggtggccacccactctaatgcccacgccctgtgcccacagccgcgcaatctgaccgaccggcaactgcgggcgatccgcgacagcggcggcgtagtgggcgtcaatttcggcaatgctttcctgcgcgccgacggacggcgcgacagcgacacgcccttaaccaccattgtccggcatatcgactatcttattaacataatgggtgaggatcatgtggctctcggctcggattttgacggcatcaccctgcccgacgagctgggcgatgtggccggcttaccgcggcttatcaatgccttgcgggacaatggctatgatcaattggtgctggataagctgctgtggaacaactggctgcgggtattaaaaaaggtttggcaacaatag

SEQ ID NO: 13 - DNA, 927 bp, Klebsiella variicola
atgttcattaaagtcctcggctccgccgccggcggcggtttcccgcaatggaactgcaactgcgccaactgtcagggtctgcgcaacggcaccattcaggccagtgcccgcacccagtcgtcgatcatcgtcagcgataacggcaaagagtgggtgctgtgcaatgcctcgccggatatcagccagcagattgcccatacccccgagttaaataaacccggcgtactgcgcgggacgtctatcggcggcattattctcaccgacagccagatcgaccacaccaccgggttgctgagcctgcgcgaaggctgcccgcaccaggtgtggtgcacgccggaggttcatcaggatctctccaccggcttcccggtgtttaccatgctgcgacactggaacggcggcctggtgcatcatcccatcgcgccgcagcagccttttaccgttgacgcctgccctgatttgcagtttaccgccgtgcctatcgccagcaacgcgccgccctattcgccgtatcgcgaccggccgctgccgggccataacgtggcgctgtttatcgaaaaccgccgcaacgggcagacgctgttctatgccccggggctgggtgagccggatgaagcccttctgccgtggctgcaaaaagcggactgtctgctgatcgatggcaccgtctggcaggatgacgagctgcaggccgccggcgtcgggcgcaataccggtcgcgatatgggacacctggcgctcagcgatgagcacgggatgatggccttgctggcctccctgccggcaaaacgcaaaattctcattcatattaataacaccaacccgatccttaacgaacagtctccccagcgccaggcgctaacgcaacaggggattgaagtgagctgggacgggatggcaatcacccttcaggataccgcatgctga

SEQ ID NO: 14 - DNA, 756 bp, Klebsiella variicola
atgctgatcaccgacacgctgtcgccgcaggcctttgcagaggctctgcgggctaaaggcgccttctaccatattcaccacccttaccacatcgccatgcataacggcgaagcgacccgcgagcaaattcagggttgggtggcgaaccggttttattaccagaccaccattccgctgaaagacgcggcgattatggctaactgcccggatgcgcagacccggcgcaaatgggtgcagcggatcctcgaccacgacggtagccacggcgaagatggcgggattgaagcctggctgcggctgggggaagcggtcggtttgagccgcgacgacctgctcagcgagcgtcacgtgctgcccggcgtgcgcttcgcggtggatgcctatcttaatttcgctcgtcgcgcctgctggcaggaggcggcctgcagctccctgaccgagctgttcgccccacagatccatcagtcgcgcctcgacagctggccgcagcactatccgtggatcaaagaggaaggctatttttacttccgcagtcgtctgagccaggctaaccgcgacgttgagcatggtctggcgctggcgaagacctactgtgacagcgctgaaaaacagaaccggatgctggagatcctgcagtttaagctcgacatcctgtggtcgatgctcgatgccatgaccatggcctacgctctgcagcgcccgccctatcacacggtcaccgacaaggcggcctggcacacgacccgactggtgtaa

SEQ ID NO: 15 - DNA, 279 bp, Klebsiella variicola
atgcaaaaaacgtccatcgttgcctttcgtcgcggctaccgactgcagtgggaagccgcccaggagagccatgtgatcctctatccggagggaatggctaaactcaatgagaccgccgcggcgatcctcgagctggtcgatggccggcgcgacgtcgcggcgattatcgccatgcttaacgaacgtttcccggaagccggcggcgtcgatgacgacgtcgtcgagttcctgcagatcgcctgtcaacagaagtggatcacctgccgtgagccagaataa

SEQ ID NO: 16 - DNA, 1143 bp, Klebsiella variicola
gtgagccagaataaacccgccgtcaatccgccgctgtggctgctggcggagctgacctaccgctgcccgctgcagtgtccctactgttccaatccgctggacttcgcccggcaggaaaaggagctgaccaccgaacaatggatcgaggtctttcgccaggcgcgagcgatgggcagcgtacagctgggcttttccggcggcgagccgctgacccgtaaagatctgccggagctgatccgcgccgcgcgcgacctcgggttctataccaacctgatcacctcgggaattgggctaaccgagagcaaactcgacgccttcagcgaggccggactggaccatatccagattagcttccaggccagcgatgaggtgctcaacgccgctcttgccggcaataaaaaagccttccagcagaagctggcgatggccagagcggtgaaagcgcgcgactacccgatggtgctgaacttcgtcctccaccggcataacatcgaccagctcgataaaattatcgagctgtgcattgagctggaagccgatgacgtcgagctcgccacctgccagttttacggctgggcgtttcttaatcgcgaggggttactgccgacccgggaacagatcgcccgcgccgagcaggtggtcgccgattaccggcagaaaatggccgccagcggtaacctcaccaacctgctattcgtcaccccggactattacgaggaacgcccgaaaggctgtatgggcggctggggatcgattttcctcagcgtcactccggaaggcactgcgttgccgtgccacagcgcgcgccagctgccggtggcgttcccgtcggtgctggagcagagtctggaatcgatctggtatgactcgttcggcttcaaccgttatcgcgggtatgactggatgccggagccgtgccgctcctgtgatgaaaaagagaaagacttcggcggctgccgctgtcaggcctttatgctgaccggcagcgccgataacgccgacccggtgtgcagcaaatccccacatcatcacaaaatccttgaggcccggcgcgaagcggcctgcagcgacatcaaagtcagccagctgcagttccgcaaccgtacccgctcgcagcttatctacaaaacccgggaactgtaa

SEQ ID NO: 17 - DNA, 5676 bp, Klebsiella variicola
aagagcgtaacctgggcctggtcttccagtcctatgccctgtggccgcacaaaacggtgttcgacaacgtggcctatccgctgaagctgcgtaaagtcgccgccggggagataaaagagcgggtgcagcgcgtgctggatcaactggggctggggcacctcggcaaccgccatccgcaccagctctccggtggccagcagcagcgggtggccatcggccgggcgctggtctacaacccgccggtgatcctgctggatgaaccgctctccaaccttgacgccaaactgcgcgaagaggcgcgggtgtttctgcgcgagctgatcatcaagctgggcctgtcggcgctgatggtcacccacgatcagaatgaagcaatggccatctccgatcgtattctgctgctgaacaatggcgtcattgagcagcaggggacaccgcaggagatgtacggcagcccggcgacgctgttcgccgccgagtttatgggcagcaataaccgtctgcacggtaaggtgacggcgctggaaaacggtcgtgcgagggttgaaggcgccagctggacgctgtggggtcgagcgggcgagggggtcagcgtcggccagccggcgacggcggtgatccgcgtcgagcgcctgcgtctcgacggcgcagcgcaggataacagcctccagctaccgctgctgaccagcatgtacctcggcgaccgctgggagtacctgttccgtaccgaaggcgacgactttccgctgcgcgcctacggaacggcgctgcgcgatgccgaacactgccatctgacgctcccggcggaggatgtgtggatttttccgcagcggtaaccgcccgttaagacgccagatggcacacggcttcaatattgtgtccgtctggcgcaatcacaaaggcggcgtagtaactggcgtggtagtttggccgtaaccccggcgcgccgttgtcctttcctccggcggccagcgccgcctgccagaaggcatcgacctgctcgcggctgtcggcgcgaaaggcaatgtgcagcggcgccggtttttccgcactttgatagaggcatagagagacatcgccgctgccggccagctctgcgccgtaggtcggctcgccttcgccaaccagcgttacgccaagcggggccagggcctgtaaaaagaacgctttgctggcggcatagtcgctagcgccgaacttcacatgatcgaacatatctactcttctcctgtggatggggatcgccagctgaacgcgttagccgggaaatagatgcaaaatagttaacaacggttattaacttttttaacagctcaaaatgtttaattattcctaatacgagtatttacagacgtttaatctgccatgtaccataaacaaaaaataacatttgtgatacaagtcactcttttatggcgcatgggtaagctcagcgcggtgtgaatgccgctggtttaccgcctcaaggttatggcagcacccttctcacgatgacattccggtgaatttctctccgatttcccgcgtttcgttgagaatcgtctactatccaaacgataaccctataaatgatatcgcctctggcgatgttttatccgacttgccgggccgggccagacgctaagcggtttgaaagcgcaatggtatcggttccagcacgacattcactgggtttgtctgcgacaaaaacagtaattttcaatgacaggaatgtgatgatgtcgagcgagaaaaccaataattccaggcgtgatttcctggtgaaatcgatggcgctgatcccgacggtggtgatcggcggcgcgggagcaggggccattggcgtggccaccagcgcgaccgcgcaggcggcccccgcgtctgagcctgcttccgggaacacggcggcggccagcgactggaagccgcagttcttcaacgatcgtgagtgggcgtttatcaacgccgccgtcgctcgcttaatcccggcggatgaactcggtcccggcgccaaagaggccggcgtcccggagtttatcgaccgccagctcaataccccctacgccaccggctccatctggtatatgcaggggcccttcaaccccgacgtgccgaaagagatgggttatcagctgccgctggtgcctaaacagatctataacctcgggatcgctgatgccgaggcgtggtgccaggacaaatatcataaaaccttcgctgaactgagcaacgagcagcaggacgaggcgctcggcctgtgggaatccggcaaagccgagttcaaacagctgccggcctcgctgttcttcacctatctgctacagaacacccgcgaagggttcttcagcgacccgatccatggcggcaataaaggcatggtcggctggacgctgattaattttcccggcgcgcgcgccgactttatggactgggttgaacggggcgaacgctaccccttcccgccggtatcaattaatggggagagggcgtaatcatggccaccgtattgaaaaaaaccgatgtcgcgatcgtcggcttcggctgggttggggcgatcatggccaaagagctgaccgaagccgggctcaacgtcgtcgcgctggagcgcggcccgatgcgcgacacctggccggatggcgcctatccgcaggtgattgacgagctgacctacaacatccgccgcaagctgttccaggatctgtcgaaaagcactgtcaccatccggcataacaccagccagcaggcggtgccgtatcgccagctggcggccttcctgccgggtaccggcgtgggcggggccgggctgcactggtccggcgtccatttccgcgtcgatcccatcgaactgcggatgcgcagccactatgaagaacgctacggcaaaaacttcattccccaggatatgatcatccaggattttggcgtcacctacgacgagctggaacccttcttcgataaagcggaaaaagtgttcggcacctccgggaccgcctggtcgatcaaaggcaaggtcgtcggcaaaggccgcggcggcaacgccttcgccccggaccgctcagacgacttcccgctgccggcgcagaaaaacacctggtcggcgcagctgtttgaaaaagcggcgctggaagtggggtatcacccctataacctgccgtcggccaacacttccgactcctataccaacccctacggcgcgcagatgggcccgtgcaacttctgcggtttctgcagcggctacgcctgctacatgtactccaaagcctcgccgaacgtgaacatcctgccggcgctgcgccaggaaaaacgctttgagctgcggaccaacgccaacgtgctgaaggtcaacctgaccgacgacaaatcccgcgccaccggcgtgacctacgtcgacggccaggggcgcgaaatggaacagccggcggacctggtgattatcggcgccttccagttccacaacgtgcacctgatgctgctctccgggatcggcaaaccgtacaatccggagaccggcgaaggggtggtggggcgtaacttcgcctaccagaacatgaccaccatcaaggccattttcgacaaagacacctataccaacccgtttatcggcgcgggcggcaacggcgtcggggtcgacgacttcaacgccgacaacttcgaccacggcgcggcgggctttgtgggcggttcgccattctgggtcaaccaggccgggaccaagcccatctccggtttcccggtaccgccgggcaccccggcgtggggcagcaagtggaaagcggcggttgccgatacctacacccatcacctgtcgatggatgcccacggcgcgcaccagtcctatcggcagaactacctcgatcttgatccgaactacaaaaacgtcttcggccagccgctgctgcgcatgaccttcgactggcaggaaaacgacatcaagatggcgcagtttatgttcgataagatggcgccgatcgccaaagcgatgaagccgaaatatatcctcggcagcccgaaaaacgccaacagccactttgataccaccacctaccagaccacccatatgaacggcggggcggtgatgggggaagatccgaaaaccagcgccgttaaccgttatctgcaaagctgggacgtgcataatgtcttcgtcatcggcgcctccgcgttcccgcaggggctgggctacaacccaaccggcacggtggccgcgctggcctactggtcagcgaaggcgatccgcgagcagtatctgaaaaatccgggacccctggtgcaggcataaaggaaggcagatgatgaaaatgcaatggttatcggccctggtgcttggggcattgagctgcgcggcttttgccgaagaggcgcctgcggacagcaatctgattaagcagggggagtatctggcgcgggcgggggactgtgtcgcctgccacaccaacggcaaaacggggaaacctttcgccggcggtctgccgatggagacgccgatcggcaccatctactccaccaatatcacgccggataaagaacacggcatcggcgggtacaccttcgaagagttcgacgacgcggtgcgcaagggcgtgcggaaagacggttccacgctctatccggcgatgccgtatccctcgttcgcgcggatcagtgaagcggacatgcgcgccatgtacgcctactttatgcatggcgtggaaccggtaaatgccgccaacaaggacaccgacatcccgtggccgctgtcgatgcgctggccgctggcgttctggcgcggcatcttcgccccgacgccgagcgactttgtcgccaacccgcaggttgacccggtgctggagcgcggccgctatctggtggaaggcctgggccactgcggcgcctgccataccccgcgcagtctgacgatgcaggaaaaagcgctcagcgaaagcgaaggcgatgattacctggcgggcagcaatgcgccgattgacggctgggtcgcctccagcctgcgcggcgaaaaccgcgacggtctggggacctggagcgaggctgagctggccgagttcctgaaaaccggacgcaacgataaatcggtggtcttcggcggcatgagcgatgtggtggagcacagtctgcagtatctttctgatgacgacatcaccgccatcgcccgctatctgaagtcgctcccgccgcgcggcggcaaacagaccccagccccggtggaagacagcgtggcgaaagatctgtggaagggtaacgacagtaaaaccggcgccgcgctgtacgttgataactgcgccgcctgccaccgcaccgacggcgcgggctataaacgcgccttcccgtcgctgaagggcaacccggtggtacagaccgaagatgccacttcgcttatccatatcgttctgaccgggagcaccacgccggcggtgaaagatgcggtctccaacctgaccatgccgtcgttcggctggcgtctggacgaccagcaggtggcggatgtggtcaacttcatccgcaccagctggggcaacaatgcgccggcggtcagcgccagcgatgtggcgaaggtgcgtaaggagaccgcggcgcacgatgagaaggcgttaggcaacgccgatatctcgaagctgccgggggccggacagtaatcgtcagggttgcgtcgcccgcgcggcgcaacccatacttagccacgacgtaaaatactgtctgaat

SEQ ID NO: 18 - DNA, 4957 bp, Klebsiella variicola
cccaccgctgagcggtctggtccagcagttgaagtttcaccatcgtccggagctggggcccgccctggcgcgtctgctgcagctgcgccttaagcagcggcacgatttgccgccggtggacggtatggtcggcgtaccgctgtggcatcgccgccgctggcgccggggttataatcagtgtgacgaactgtgccgaccgttagcccgctggcgaggttgcgtctggcatcgggaaggcctgactcgccagcgagccggcgccgtccaacattcgctcaatgcccgccagcgcaggcagaacctgaaaaatgcctttcagcttgaatttgccgtggagggactccatatcgctatcgtggatgatgtcgtcacgaccggcagcaccgtcgccgagatatcccgcctgcttttgcgaaacggcgccgcgacggttcaggtatggtgtttgtgccgtaccttgtagacccccgctgatgggcgtattataacccagtaaaatagtcaactattaggccaacgctatgatccgtatttccgatgctgcacaagcgcactttgccaaactgctggcaaatcaggaagaagggacgcaaattcgcgtatttgtgattaatcccggcactccaaacgctgagtgcggcgtatcttactgcccaccggatgcggtggaagacaccgacacggcgctgaaatttgaacagctgaccgcctatgtcgatgagctcagcgcgccctatctggaagacgctgaaattgatttcgtcaccgaccagcttggctcgcagctgacgctgaaagcgccgaacgccaagatgcgcaaagtctctgacgatgccccgctgatggagcgcgtcgagtacctgctgcagtcgcagattaacccgcagctggcgggccacggcggccgcgtgtcgctgatggagatcaccgacgacggtctggctattctgcagttcggcggcggatgcaacggctgttcgatggttgacgtgactctgaaagaaggcattgaaaagcagctgctgaacgagtttccggagctgaaaggggttcgcgatctgaccgagcatcagcgaggcgagcactcctactactaagccttcctccggcggcaccctgccgccggtcctccctggccctttctgtgttaccgcgcaacatctcccgctaaatgaccggcaaaaaccggggaaagagtatgacttaagtctcatatttaacattttcccccgtcaggtgattctcaaccctcacatgttacccgtatcatccccttcaggcaccaccggcaaaaaaatagccagctatcctttctctgggtaaggataatgcaagtatttacggtgccagactattttgctcccacatggggcagatgtcttacaggttgaatctttgacgttacccataacaaattaaggccaggtaaatcatgccattaatcatcgttgctatcggggttgccctgttattgctgttaatgatccgtttcaaaatgaacggcttcattgcgctggttctggtggcgctcgccgtcggattaatgcagggtatgccgctggataaagtgatcgtctctatcaagaacggcgttggcggcacccttggcagcctcgcgctgatcatgggcttcggggctatgctcggtaaactgctggcagactgcggcggcgcccagcgtatcgccaccacccttatcaataagttcggtaagaagcatattcagtgggcggttgtgctgaccggcttcaccgtcggcttcgcgctgttctatgaagtgggcttcgtgctgatgctgccgctggtgtttaccatcgccgcctcggcccgcatcccgctgctgtacgtcggtgtaccgatggccgccgctctctctgtcacccacggcttcctgccgccgcatccgggtccgaccgcgatcgccaccatcttccatgccgatatgggcaaaacgctgctctacggcaccattctggctatcccgacggttatcctggcagggccggtgtttgcgcgcttcctgaaaggcattgataagccgatcccggaaggcctgcataacccgaaagtgttcacagaagaagagatgcctggctttggcgtcagcgtctggacctcgttggttccggtgatcctgatggcgatgcgcgccgtggccgagatgatcctgccgaaggggcacgccttcctgccgattgctgagttcttcggcgacccggtgatggccaccctgattgcggtactgatcgccctgtttaccttcggtttgaaccgcggccgctccatggagcagatcaacgataccctgacttcttctattaaaatcatcgccatgatgctgttgatcatcggcggcggcggcgccttcaagcaggtgctggtggatagcggcatggataaatacatcgcctctatcatgcacgaatcgaacatgtctccgctgtttatggcgtggtctatcgccgcagtactgcgtatcgcgctgggttcagcgaccgttgccgccatcaccgcaggcgggattgccgcgccgctgatcgccaccaccggcgtcagcccggaactgatggttatcgccgttggctccggcagcgttatcttctctcacgtcaacgatccgggtttctggctgttcaaagagtactttaacctgaccatcggcgagaccatcaggtcctggtcggtgctggaaaccattatctcagtatgcggcctggtaggctgtctgctgctggggatggtggtgtaaggagactcccgccacagaaaaaggccggtccgcattatgcgggccggcctttttttcagacatgctcttcgccgcgcagcgaacgacgcggcgggaagtcatctggcggcggctttcttgctcgccgccttgcgccgtttatccagatccttgatcagcttattgacctgttcatcggcaaacatctgctccagggtgacggacagcttccggcgccagttcgggtactcagtgctggtgcccggaatgttaaccggcgacgccatctccagccagtcttccggctgcaggcccagtagcgcactgttactgtcggcaatgtaacgctgcatcccccggttgaggatcccggtcatgctcatcagcgaggccttatgcccggcacgtttgggcagacagccgtacttatgcaacgcatccagcagcccttgcttcgccagctcgcggtcctggtacagcccgcgcagcaccacctcatcaggatagagccccagcgctttacctagcgtcagatcgccgctttcccagtagccgcggagggtgggcaggtcgtgcgtcgtcgccacagccattgattgctccgggtacagcgccggcgcgcggaacgttttctctgcatcactctcaaaatagagcaccttataagaatagacgccgctgttgcgtaacttgctgacaatctccaccggtaccgtgcccaggtcttcgccgatcaccatgcagcgatgacgctggctttccagcgccagaagcgacagcagatcgtcgacgggatactgaacgtaagcgccatggtcggcggtttcaccataggggatccaccacaaacgcagcaccgacatcacgtgatcgatgcgcagcgcgccgcagttctgcatattggcgcgcagcagatcaataaacggctcataggcgcgagcggcaataatatgcggatccatcggcggcaggccccagttctggcccagcgggcccagaatatccggcggcgcgcccacggacgctttcaggcagtacagctcacggtcgcaccaggtctccgacccgccttcggcgacgcccaccgccagatcgcggtagaggccaatcggcatcccgtcgcgctggctggtttcccagcaggcggcgaactggctccaggccagccactgcagccagagatagaaactgacgtcgtcctcatgctcaatgcagaaggctttcacctccgggctgtcgatatcctgaaaggccttcggccaggccggccagccccagcgcagcgggtcctgttgtacctgccaggcgtgaagcgcatcgaaagcggcctgccagtagaggctttccccttcgcgcagaacaaactcgcggaacgctgtcatctgctcatcttcacgacgagagaatcgtttccacgccatacgcagcgcggtcattttcagcgtggtgaccgcggtatagtcgacatcatccgtttgccgcgccgcctgcaaggcctgctgggtcgctgcggactgccaccacgcctgcgcctcttcgctacgctggaaatcttcaaccgcattaacgtcgatgtagatgacgttcagccagcgacgtgaagacgggctatacggactggcgctctccgggttcgccggatagagagcatgtatcgggttaaggccaataaacgacccgccgcggcgggcgatttccggcaacatggcccgcagatcgccaaaatcgccgatcccccagtttttctccgagcgcagggtataaagctgcacgcaggtgccccacagctttttcccctctttcagcggctgcggttcgtagcagcgcgccggcgcgacaatggtccggcagtgccaacgctctccctcttgggtgagcgtcagggagtggtacccctccggcagtttagccggcagcggcagcgtctcgccgccgcgggttttcccttgatactgcttaccgtcctcagtggtcaggatccactgatactcaccgcgtcctgccaccggcagcgacatttttttgccgtgggtgaaaattttcacgttcggcagcgggttaaccgccactttcgtggcggcggtagagcgatgcatcgcatccagcaggcgctgtttggtgacagccgcgatagactgcggcttaccgtgcgcgttgatatagctggggctgatgcccgccgccagcgcggcattatcgaggcgtttactctccataggccttccttta

SEQ ID NO: 19 - DNA, 4865 bp, Klebsiella variicola
cgaacgggttgaagttaaattcctgcggcaccaggccaagctgacgcttggcattgacgacgtctttttccagatcgtagccaaagacattgacctgaccggaggttttattcaccagcgaactgatgatgccgatagtcgtggattttccggccccgttcggccccagcagggcatagaaatcccccgcctcgacctgcaggtcgattccccgcagcgcctgaaccccgccggggtaggtttttttgagctgttttaattccagagcaatggtcattaaatatctcttctcgtgtcgtgacacgtcattaacccgcctgactcacgtttgcgttctactggcgctggcgccgccaacgcatcggtagcctgacgtatcacgggttaagatggttttaaaaaaggtgagcttaccctatattagcgcaacgcacttacttggttacaggccgataacgtccatgaatgatatagatacactcatcagcaataatgcactatggtcaaaaatgctggtggaagaggaccccggcttttttgagaaactgtcgcagacgcagaaaccgcgcttcctttggattggctgctctgacagccgcgttcccgctgagcgacttaccggcctggaacctggcgaactgtttgtccatcgtaacgtcgctaacctggtgatccacaccgacctgaactgcctgtcggtggtgcagtatgccgtcgatgtcctcgaagtcgagcacattattatttgtggccactacggctgcggcggcgtgcaggcggcggtggaaaacccggagctggggcttatcgacaactggctcctgcatatccgcgatatctggttcaagcacagctcactgctaggtgaaatgcctgaggagcgccgtctggataccctttgcgaactcaacgtgatggagcaggtgtacaacctgggacattcgacgattatgcaatcggcgtggaagcgcggacagaaggtcaccatccacggctgggcatatggcattcacgacgggctgctgcgcgatctggatgtcaccgccgtgagccgggaaaccctcgaacagcgctatcgccacggcatctccaacctcaagatcaagcacatcaaccacaaatagccttcaggcttagcccccctggagagcgcagcgtgcgctgtcctggggggatatctcttactcgtccagcagggtaactttgccaatatacggcagatgacggtagcgctgggcgtagtcgataccgtagcccaccacgaactcgtccggaatggcgaaaccgacgaactcaaccgggacattcacttcacgacggcttggtttatccagcagcgtacaaatggccagcgatttcggctcgcgcaggctgaggatctcacgcactttagacagggtattgccggaatcgatgatgtcttccacgatcagtacatctttaccacggatgtcttcatccagatctttgaggattttcacatcacgggtggtggacatgccgctgccgtagctggaggcggtcataaaatcgacttcatgcggcacctgcacttcacggcacaggtcggccatgaacataaatgagccgcgaagcagccccaccagcaccatttcgctgccgctgtcctgataatgttcgttgatttgacgacccagttccgcgatgcgcgctttgatctcggattccgggatcatcacttctacagtatgtttcataagactaaccatttgattctaaatataaatcatcaaagccgctgctttcgccccgacgatgaccggcaagccatccagtataccagcaaaaagaaatcttcggtgcagggattaataaagccatatttgtgattccgatcacacttgttaaaacctataattaattgcagttaaaaaattaacaaacacatgagtaactttctatggctgaaacaaagtctcaacaatcaaggctcctggtgacgctgactgcgctatttgcagcgttctgcggcctctatcttttaatcggcggagcatggctggtcgtgcttggcggctcctggtactaccctatcgccggtctggtgatgctgggcgtgaccgtgatgctgctgcgcggcaaacgcgctgcgctgtggctgtacgctgcgctgctgctggcgacgatgatctggggcgtctgggaagtcggcttcgacttctgggcgctgacgccgcgtagcgacatcctcgtcttctttggtatctggctgatcctgccctttgtctggcgtcgtctgtccgtgccttccgctggcgccgtaggcgcgctggtcgtcgccctgctgatcagcggcggcatgctgacctgggctggttttaacgacccgcaggaagtcaacggcaccctgagcgccgacgccaccccggccgcgccgatctccaacgttgccgacggcgactggccggcctatggccgcaaccaggaaggccagcgcttctctccgctgaagcagattaacgccgacaacgtgaagaacctgaaggaagcctgggtattccgcaccggcgacctgaagcaaccgaacgacccgggtgaaatcaccaacgaagtgacgccgattaaagtcggcgacacgctcttcctgtgtaccgcccaccagcgtctgttcgcgctggacgcggccaccggtaaagagaagtggcattttgacccgcagctgaacgccgatccgtcgttccagcacgtcacctgccgcggcgtctcttatcacgaagccaaagcggataacgctcctgccgacgtcgtcgccgactgtccgcgccgtattatcctgccggtgaacgatggccgcctgttcgcggtgaacgccgacaacggtaagctgtgcgaaacctttgccaataaaggtattctcaacctgcagaccaacatgccggtcaccacgccgggtatgtatgaacccacttctccgccgattatcaccgataaaaccatcgtgatagccggcgcggtcaccgataacttctcaacccgcgagccgtctggcgtcatccgcggctttgatgtgaataccggtaaactcttgtgggccttcgatccgggtgcgaaagatccgaacgccatcccgagcgatgaacatcacttcacgctcaactcaccgaactcctgggcgcctgccgcctacgacgctaagctggatctggtctatctgccgatgggcgtgaccaccccggatatctggggcggcaaccgcacgccggagcaagagcgttacgccagctcgattgtggcgctgaacgccaccaccggtaagctggcctggagctaccagaccgtccaccacgatctgtgggatatggatatgccctcccagccgacgctggctgacattgaggttaatggtaaaaccgtaccggtcgtctatgccccggcgaaaaccggcaacatcttcgtactggatcgccgtaatggcgagctggtggtcccggccccggaaaaaccggttccgcagggcgctgccaaaggggattacgtcgccaaaacccagccgttctccgatctgagcttccgtccgaagaaagatctgaccggggcggacatgtggggcgccactatgttcgaccagttggtgtgtcgcgtgatcttccaccagatgcgctatgaaggtatcttcaccccgccatctgagcagggcactctcgtcttcccgggtaacctggggatgtttgaatggggcggtatctccgtcgatccgaaccgtcaggtggctattgccaacccgatggcgctgccgttcgtctctaaactgatcccacgcggcccgggcaacccgatggagccgccgaaagatgcgaaaggctctggcaccgagtccggcgtgcagccacagtacggtgtcccgtacggcgtcactctgaatccgttcctgtcgccgttcggtctgccgtgcaaacaaccggcctggggctatatctccgcgctggatctgaaaaccaacgaagtggtatggaaaaaacgtatcggtacgccgcaggacagcctgccgttcccgatgccggttaagctgccgttcaccatggggatgccgatgcttggcggcccgatctctacggccggtaacgtcctgttcatcggcgcgaccgcagataactacctgcgcgcgtacaatatgagcaacggcgagaagctgtgggaagcccgtctgccagccggcggtcaggccaccccaatgacctacgaagtgaatggcaaacagtatgttgtcatctctgccggcggtcacggctcgttcggtaccaaaatgggcgattacatcgtcgcctatgcgctcccggatgacgccaaataaaacgcaaaacggcaacgaaagttgccgttttttttgtgtttatcccttctccccatggaagatggcagcaggccgccgagccctaaaccgtgaatcccaacatcatcccggtgtcttcatgctccagcaggtggcagtgggccatataggcaaactccttcggcgccggatgatcaaacttcaccaacacttcactgacgtcaccttccacgcggacagtatctttccagccgctgcggtgcgcggctggcggcttgccgttctcgctgaggatacggaactgggtgccgtggatatggaacggatgcagcatcatgtcgcccttacccgatatcacccagcgctcatactggcctctggccgcggcaaacatcggcacattcatgtcgaaggctttgccgttgatgcggttggcgttatggaaatcaaaccccttgcctgacgacatgccatgatcccgaccagcctcgtggttcatattgctcatattgctcatgtcgccatggtgcatattgctcatgtcgcccatgccgccatgccccatcatgccgtg

SEQ ID NO: 20 - DNA, 299 bp, Klebsiella variicola
cgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatc

SEQ ID NO: 21 - DNA, 300 bp, Klebsiella variicola
acgaccaaactgcacgtacatgacgagaacaacgaatgcggtatcggtgacgtggttgaaatccgcgaatgccgtccgctgtccaagactaagtcctggacgctggttcgcgttgtagagaaagcggttctgtaatagagtacgcattctcgatacggataaacggctcagcgatgagccgtttattttttctacccatatctggtttgtggtgttataatgccgcgccctcgatatggggctttttaacgaccctaattttcgggactcagtagtagttgacattagcggagcactaaa

SEQ ID NO: 22 - DNA, 71 bp, Klebsiella variicola
gaatttacttacattaaggcggcgaggggcgcctatacttgatagttctgataccagaagaaggaagaact

SEQ ID NO: 23 - DNA, 6770 bp, Artificial Sequence
  source: /note=“Description of Artificial Sequence Synthetic polynucleotide”
aacgcgctggcgatggtggcgcacatcgaaggtgctggcgggttcgacgcgcaaggcgaagatctgcaggccttttatcgcgccggggtgcgcagcattggtccgttctggaacatcgccaatcgcttcggctgcggggtcaccggcgcctttcccggcagcccggacagcgggccgggtctcactcgcgagggcattgcgctcatcgctcaggccaacgccctgaagatgcagatcgatgtttcgcacatgaatgaacaggccttttgggataccgcccaccactccaccgccccactggtggccacccactctaatgcccacgccctgtgcccacagccgcgcaatctgaccgaccggcaactgcgggcgatccgcgacagcggcggcgtagtgggcgtcaatttcggcaatgctttcctgcgcgccgacggacggcgcgacagcgacacgcccttaaccaccattgtccggcatatcgactatcttattaacataatgggtgaggatcatgtggctctcggctcggattttgacggcatcaccctgcccgacgagctgggcgatgtggccggcttaccgcggcttatcaatgccttgcgggacaatggctatgatcaattggtgctggataagctgctgtggaacaactggctgcgggtattaaaaaaggtttggcaacaatagacgaccaaactgcacgtacatgacgagaacaacgaatgcggtatcggtgacgtggttgaaatccgcgaatgccgtccgctgtccaagactaagtcctggacgctggttcgcgttgtagagaaagcggttctgtaatagagtacgcattctcgatacggataaacggctcagcgatgagccgtttattttttctacccatatctggtttgtggtgttataatgccgcgccctcgatatggggctttttaacgaccctaattttcgggactcagtagtagttgacattagcggagcactaaaatgtggaagaaacctgcttttatcgatttacgtctcggtctggaagtgacgctgtacatttctaaccgttaatcgccccgcccgccgttcgcgcgggcaccttcattcattacccggtccgtcttcatgttcattaaagtcctcggctccgccgccggcggcggtttcccgcaatggaactgcaactgcgccaactgtcagggtctgcgcaacggcaccattcaggccagtgcccgcacccagtcgtcgatcatcgtcagcgataacggcaaagagtgggtgctgtgcaatgcctcgccggatatcagccagcagattgcccatacccccgagttaaataaacccggcgtactgcgcgggacgtctatcggcggcattattctcaccgacagccagatcgaccacaccaccgggttgctgagcctgcgcgaaggctgcccgcaccaggtgtggtgcacgccggaggttcatcaggatctctccaccggcttcccggtgtttaccatgctgcgacactggaacggcggcctggtgcatcatcccatcgcgccgcagcagccttttaccgttgacgcctgccctgatttgcagtttaccgccgtgcctatcgccagcaacgcgccgccctattcgccgtatcgcgaccggccgctgccgggccataacgtggcgctgtttatcgaaaaccgccgcaacgggcagacgctgttctatgccccggggctgggtgagccggatgaagcccttctgccgtggctgcaaaaagcggactgtctgctgatcgatggcaccgtctggcaggatgacgagctgcaggccgccggcgtcgggcgcaataccggtcgcgatatgggacacctggcgctcagcgatgagcacgggatgatggccttgctggcctccctgccggcaaaacgcaaaattctcattcatattaataacaccaacccgatccttaacgaacagtctccccagcgccaggcgctaacgcaacaggggattgaagtgagctgggacgggatggcaatcacccttcaggataccgcatgctgatcaccgacacgctgtcgccgcaggcctttgcagaggctctgcgggctaaaggcgccttctaccatattcaccacccttaccacatcgccatgcataacggcgaagcgacccgcgagcaaattcagggttgggtggcgaaccggttttattaccagaccaccattccgctgaaagacgcggcgattatggctaactgcccggatgcgcagacccggcgcaaatgggtgcagcggatcctcgaccacgacggtagccacggcgaagatggcgggattgaagcctggctgcggctgggggaagcggtcggtttgagccgcgacgacctgctcagcgagcgtcacgtgctgcccggcgtgcgcttcgcggtggatgcctatcttaatttcgctcgtcgcgcctgctggcaggaggcggcctgcagctccctgaccgagctgttcgccccacagatccatcagtcgcgcctcgacagctggccgcagcactatccgtggatcaaagaggaaggctatttttacttccgcagtcgtctgagccaggctaaccgcgacgttgagcatggtctggcgctggcgaagacctactgtgacagcgctgaaaaacagaaccggatgctggagatcctgcagtttaagctcgacatcctgtggtcgatgctcgatgccatgaccatggcctacgctctgcagcgcccgccctatcacacggtcaccgacaaggcggcctggcacacgacccgactggtgtaatcatgcaaaaaacgtccatcgttgcctttcgtcgcggctaccgactgcagtgggaagccgcccaggagagccatgtgatcctctatccggagggaatggctaaactcaatgagaccgccgcggcgatcctcgagctggtcgatggccggcgcgacgtcgcggcgattatcgccatgcttaacgaacgtttcccggaagccggcggcgtcgatgacgacgtcgtcgagttcctgcagatcgcctgtcaacagaagtggatcacctgccgtgagccagaataaacccgccgtcaatccgccgctgtggctgctggcggagctgacctaccgctgcccgctgcagtgtccctactgttccaatccgctggacttcgcccggcaggaaaaggagctgaccaccgaacaatggatcgaggtctttcgccaggcgcgagcgatgggcagcgtacagctgggcttttccggcggcgagccgctgacccgtaaagatctgccggagctgatccgcgccgcgcgcgacctcgggttctataccaacctgatcacctcgggaattgggctaaccgagagcaaactcgacgccttcagcgaggccggactggaccatatccagattagcttccaggccagcgatgaggtgctcaacgccgctcttgccggcaataaaaaagccttccagcagaagctggcgatggccagagcggtgaaagcgcgcgactacccgatggtgctgaacttcgtcctccaccggcataacatcgaccagctcgataaaattatcgagctgtgcattgagctggaagccgatgacgtcgagctcgccacctgccagttttacggctgggcgtttcttaatcgcgaggggttactgccgacccgggaacagatcgcccgcgccgagcaggtggtcgccgattaccggcagaaaatggccgccagcggtaacctcaccaacctgctattcgtcaccccggactattacgaggaacgcccgaaaggctgtatgggcggctggggatcgattttcctcagcgtcactccggaaggcactgcgttgccgtgccacagcgcgcgccagctgccggtggcgttcccgtcggtgctggagcagagtctggaatcgatctggtatgactcgttcggcttcaaccgttatcgcgggtatgactggatgccggagccgtgccgctcctgtgatgaaaaagagaaagacttcggcggctgccgctgtcaggcctttatgctgaccggcagcgccgataacgccgacccggtgtgcagcaaatccccacatcatcacaaaatccttgaggcccggcgcgaagcggcctgcagcgacatcaaagtcagccagctgcagttccgcaaccgtacccgctcgcagcttatctacaaaacccgggaactgtaatgacgctggcgacccgcactgtcactctgccgggcggcctgcaggctaccctggttcatcagccgcaggccgatcgcgcggcggccctggtgcgggttgccgccggcagccaccatgaaccgtcgtgcttccccggtctggcgcacctgctggaacacctgctgttttacggcggtgagcgctaccgcaatgatgaacggctgatgagctgggtgcagcgccaggcagggaatgtgaatgccaccaccctgtcccgccacagcgctttctttttcgaggtcgccgccgaggatctggctgacggcgtcgcgcgcctgcaggagatgctgcaggcgccgctgctgctcagggacgatattcaacgcgaagtcgcggttatcgacgccgaaaaccgcctgatccaacagcatgagttgtcgcgacgggaagccgccgtgcgtcacgccgccatcgcgcccgcggcgtttcgccgctttcaggtcggcgacgccgggtcgctgggggaggatttcctcgcgctacaggcggccttacgtgactttcaccgcagccactacgtcgcccgccggatgcaactctggctgcaggggccgcagtcgctggaggtgctcggcgaactggcgacccgtttcgccaccgggcttgccccgggcgaggcaccgccgccagcgccgccgctcactctgggcgagccccctcaactgcagctggccgtctccagccagcccgcgctgtggcgctgcccgctgatcgccttaagtgacaatgtcacgttactgcgcgagtttttgctggatgaagcccccggtagcctgatggccggcctgcgccagcgcgggatggccgaggacgtggcgctgaactggctgtatcaggatcagcacttcggctggctggcgctgattttcgccagcgaccggccggaacaggtcgaccggcagataacccactggttgcaggcgctacagcagacgacgcctgagcagcagcaacactactatcagctgtcccggcgccgttttcaggcgctgtcgcccctcgatcagctgcgccagcgggcattcggctttgcccccggggcgccgcccaccgggttcgccgatttttgcgccgccctgctggccgcccccacggtcagcctggcctgccagacgcagccccccggagcaacggtagccacccagggctttagcctgccgctcagccgctggtcgccacgtccggtctctgacccggcgctggcattcgctttttatccgcaggccgctggcgagctcgtggccgaaagcccggcggaagccgcgccactgcgtcacctcccgtcaccgggagagccgccgacgctcctgctgcgaccgcccttctactgctcgcccacgccggccggggggctggcgcgcggggaacagctgcgtccattacttgccgccctgcgccatgccgggggacacggcgagtggcatctgttcgacggcagctggcagctgatcctgcagttgcctgcgtccggccaacggccggaggcgattctgcaggccatcgtgcggcagctcgcgctcccggtcgccccgctgcccccaccgccggagagtattgcgatccgtcatctcatggcccagctccccgaacggctgggtacgtcagcgcaccaggaaggttggctggcggccctgattggcggcagcgcggaagatgcgcagtgggtagcgcgtcagctgagccggcttaccgtcccggttaatccgccgatgcccgctccggccccctgccgcggcggcgtcgagcggctggcttatccccggggcgacacggcgctactggtctttcttccgctgccggaaggcgcttcattggcggccctgcgggtgctggcgcagttctgcgagccgccgtttttccagcgcctgcgggtggagcagcagataggctatgtggtgagctgccgctatcagcgcgttgccgatcgcgacggactgctgatggcgctccagtccccggatcgccgtcccggagaactcctccgctgctgtaaaacctttctgcgccagctggaccccatggatgaggcgaccttcaggttgttacagcagcggctggccgctcaggcccgctcccgggtaccgccgaaggtgcgcgccctggacgcgctgcgccaggagtataacttgccgggggtgacgccgcaggcggctgacgcgctgcgcgttgaagaggtggtcgccctgtggcgtgagatgacccgccggcgtcgtcgctggcgagtgctgttcacgacagggagttaatggccgggggcatccggggtcagaaagtcgctgacgaagcgggtgaggatcgccgcggcctgcgggctctcctggacctcgtcgtgtaactgttgtgggtcgatgccttccgcctgcagcaccgcctcccggtagcggatcagcgagcgggcaatcggcgcggtgaattccgggtgaaactgcgtggagaccgcctgcggcccgtagcgcacaatctggtggggatcgtgggaggatgcggccagcacggtagccccctccggcaaacgggtcacggtttgcaaat

SEQ ID NO: 24 - DNA, 4012 bp, Artificial Sequence
  source: /note=“Description of Artificial Sequence Synthetic polynucleotide”
ccgttaagacgccagatggcacacggcttcaatattgtgtccgtctggcgcaatcacaaaggcggcgtagtaactggcgtggtagtttggccgtaaccccggcgcgccgttgtcctttcctccggcggccagcgccgcctgccagaaggcatcgacctgctcgcggctgtcggcgcgaaaggcaatgtgcagcggcgccggtttttccgcactttgatagaggcatagagagacatcgccgctgccggccagctctgcgccgtaggtcggctcgccttcgccaaccagcgttacgccaagcggggccagggcctgtaaaaagaacgctttgctggcggcatagtcgctagcgccgaacttcacatgatcgaacatatctactcttctcctgtggatggggatcgccagctgaacgcgttagccgggaaatagatgcaaaatagttaacaacggttattaacttttttaacagctcaaaatgtttaattattcctaatacgagtatttacagacgtttaatctgccatgtaccataaacaaaaaataacatttgtgatacaagtcactcttttatggcgcatgggtaagctcagcgcggtgtgaatgccgctggtttaccgcctcaaggttatggcagcacccttctcacgatgacattccggtgaatttctctccgatttcccgcgtttcgttgagaatcgtctactatccaaacgataaccctataaatgatatcgcctctggcgatgttttatccgacttgccgggccgggccagacgctaagcggtttgaaagcgcaatggtatcggttccagcacgacattcactgggtttgtctgcgacaaaaacagtaattttcaatgacaggaatgtgtcatggccaccgtattgaaaaaaaccgatgtcgcgatcgtcggcttcggctgggttggggcgatcatggccaaagagctgaccgaagccgggctcaacgtcgtcgcgctggagcgcggcccgatgcgcgacacctggccggatggcgcctatccgcaggtgattgacgagctgacctacaacatccgccgcaagctgttccaggatctgtcgaaaagcactgtcaccatccggcataacaccagccagcaggcggtgccgtatcgccagctggcggccttcctgccgggtaccggcgtgggcggggccgggctgcactggtccggcgtccatttccgcgtcgatcccatcgaactgcggatgcgcagccactatgaagaacgctacggcaaaaacttcattccccaggatatgatcatccaggattttggcgtcacctacgacgagctggaacccttcttcgataaagcggaaaaagtgttcggcacctccgggaccgcctggtcgatcaaaggcaaggtcgtcggcaaaggccgcggcggcaacgccttcgccccggaccgctcagacgacttcccgctgccggcgcagaaaaacacctggtcggcgcagctgtttgaaaaagcggcgctggaagtggggtatcacccctataacctgccgtcggccaacacttccgactcctataccaacccctacggcgcgcagatgggcccgtgcaacttctgcggtttctgcagcggctacgcctgctacatgtactccaaagcctcgccgaacgtgaacatcctgccggcgctgcgccaggaaaaacgctttgagctgcggaccaacgccaacgtgctgaaggtcaacctgaccgacgacaaatcccgcgccaccggcgtgacctacgtcgacggccaggggcgcgaaatggaacagccggcggacctggtgattatcggcgccttccagttccacaacgtgcacctgatgctgctctccgggatcggcaaaccgtacaatccggagaccggcgaaggggtggtggggcgtaacttcgcctaccagaacatgaccaccatcaaggccattttcgacaaagacacctataccaacccgtttatcggcgcgggcggcaacggcgtcggggtcgacgacttcaacgccgacaacttcgaccacggcgcggcgggctttgtgggcggttcgccattctgggtcaaccaggccgggaccaagcccatctccggtttcccggtaccgccgggcaccccggcgtggggcagcaagtggaaagcggcggttgccgatacctacacccatcacctgtcgatggatgcccacggcgcgcaccagtcctatcggcagaactacctcgatcttgatccgaactacaaaaacgtcttcggccagccgctgctgcgcatgaccttcgactggcaggaaaacgacatcaagatggcgcagtttatgttcgataagatggcgccgatcgccaaagcgatgaagccgaaatatatcctcggcagcccgaaaaacgccaacagccactttgataccaccacctaccagaccacccatatgaacggcggggcggtgatgggggaagatccgaaaaccagcgccgttaaccgttatctgcaaagctgggacgtgcataatgtcttcgtcatcggcgcctccgcgttcccgcaggggctgggctacaacccaaccggcacggtggccgcgctggcctactggtcagcgaaggcgatccgcgagcagtatctgaaaaatccgggacccctggtgcaggcataaaggaaggcagatgatgaaaatgcaatggttatcggccctggtgcttggggcattgagctgcgcggcttttgccgaagaggcgcctgcggacagcaatctgattaagcagggggagtatctggcgcgggcgggggactgtgtcgcctgccacaccaacggcaaaacggggaaacctttcgccggcggtctgccgatggagacgccgatcggcaccatctactccaccaatatcacgccggataaagaacacggcatcggcgggtacaccttcgaagagttcgacgacgcggtgcgcaagggcgtgcggaaagacggttccacgctctatccggcgatgccgtatccctcgttcgcgcggatcagtgaagcggacatgcgcgccatgtacgcctactttatgcatggcgtggaaccggtaaatgccgccaacaaggacaccgacatcccgtggccgctgtcgatgcgctggccgctggcgttctggcgcggcatcttcgccccgacgccgagcgactttgtcgccaacccgcaggttgacccggtgctggagcgcggccgctatctggtggaaggcctgggccactgcggcgcctgccataccccgcgcagtctgacgatgcaggaaaaagcgctcagcgaaagcgaaggcgatgattacctggcgggcagcaatgcgccgattgacggctgggtcgcctccagcctgcgcggcgaaaaccgcgacggtctggggacctggagcgaggctgagctggccgagttcctgaaaaccggacgcaacgataaatcggtggtcttcggcggcatgagcgatgtggtggagcacagtctgcagtatctttctgatgacgacatcaccgccatcgcccgctatctgaagtcgctcccgccgcgcggcggcaaacagaccccagccccggtggaagacagcgtggcgaaagatctgtggaagggtaacgacagtaaaaccggcgccgcgctgtacgttgataactgcgccgcctgccaccgcaccgacggcgcgggctataaacgcgccttcccgtcgctgaagggcaacccggtggtacagaccgaagatgccacttcgcttatccatatcgttctgaccgggagcaccacgccggcggtgaaagatgcggtctccaacctgaccatgccgtcgttcggctggcgtctggacgaccagcaggtggcggatgtggtcaacttcatccgcaccagctggggcaacaatgcgccggcggtcagcgccagcgatgtggcgaaggtgcgtaaggagaccgcggcgcacgatgagaaggcgttaggcaacgccgatatctcgaagctgccgggggccggacagta

SEQ ID NO: 25 - DNA, 3385 bp, Artificial Sequence
  source: /note=“Description of Artificial Sequence Synthetic polynucleotide”
aacgcgctggcgatggtggcgcacatcgaaggtgctggcgggttcgacgcgcaaggcgaagatctgcaggccttttatcgcgccggggtgcgcagcattggtccgttctggaacatcgccaatcgcttcggctgcggggtcaccggcgcctttcccggcagcccggacagcgggccgggtctcactcgcgagggcattgcgctcatcgctcaggccaacgccctgaagatgcagatcgatgtttcgcacatgaatgaacaggccttttgggataccgcccaccactccaccgccccactggtggccacccactctaatgcccacgccctgtgcccacagccgcgcaatctgaccgaccggcaactgcgggcgatccgcgacagcggcggcgtagtgggcgtcaatttcggcaatgctttcctgcgcgccgacggacggcgcgacagcgacacgcccttaaccaccattgtccggcatatcgactatcttattaacataatgggtgaggatcatgtggctctcggctcggattttgacggcatcaccctgcccgacgagctgggcgatgtggccggcttaccgcggcttatcaatgccttgcgggacaatggctatgatcaattggtgctggataagctgctgtggaacaactggctgcgggtattaaaaaaggtttggcaacaatagcgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcatgtggaagaaacctgcttttatcgatttacgtctcggtctggaagtgacgctgtacatttctaaccgttaatcgccccgcccgccgttcgcgcgggcaccttcattcattacccggtccgtcttcatgttcattaaagtcctcggctccgccgccggcggcggtttcccgcaatggaactgcaactgcgccaactgtcagggtctgcgcaacggcaccattcaggccagtgcccgcacccagtcgtcgatcatcgtcagcgataacggcaaagagtgggtgctgtgcaatgcctcgccggatatcagccagcagattgcccatacccccgagttaaataaacccggcgtactgcgcgggacgtctatcggcggcattattctcaccgacagccagatcgaccacaccaccgggttgctgagcctgcgcgaaggctgcccgcaccaggtgtggtgcacgccggaggttcatcaggatctctccaccggcttcccggtgtttaccatgctgcgacactggaacggcggcctggtgcatcatcccatcgcgccgcagcagccttttaccgttgacgcctgccctgatttgcagtttaccgccgtgcctatcgccagcaacgcgccgccctattcgccgtatcgcgaccggccgctgccgggccataacgtggcgctgtttatcgaaaaccgccgcaacgggcagacgctgttctatgccccggggctgggtgagccggatgaagcccttctgccgtggctgcaaaaagcggactgtctgctgatcgatggcaccgtctggcaggatgacgagctgcaggccgccggcgtcgggcgcaataccggtcgcgatatgggacacctggcgctcagcgatgagcacgggatgatggccttgctggcctccctgccggcaaaacgcaaaattctcattcatattaataacaccaacccgatccttaacgaacagtctccccagcgccaggcgctaacgcaacaggggattgaagtgagctgggacgggatggcaatcacccttcaggataccgcatgctgatcaccgacacgctgtcgccgcaggcctttgcagaggctctgcgggctaaaggcgccttctaccatattcaccacccttaccacatcgccatgcataacggcgaagcgacccgcgagcaaattcagggttgggtggcgaaccggttttattaccagaccaccattccgctgaaagacgcggcgattatggctaactgcccggatgcgcagacccggcgcaaatgggtgcagcggatcctcgaccacgacggtagccacggcgaagatggcgggattgaagcctggctgcggctgggggaagcggtcggtttgagccgcgacgacctgctcagcgagcgtcacgtgctgcccggcgtgcgcttcgcggtggatgcctatcttaatttcgctcgtcgcgcctgctggcaggaggcggcctgcagctccctgaccgagctgttcgccccacagatccatcagtcgcgcctcgacagctggccgcagcactatccgtggatcaaagaggaaggctatttttacttccgcagtcgtctgagccaggctaaccgcgacgttgagcatggtctggcgctggcgaagacctactgtgacagcgctgaaaaacagaaccggatgctggagatcctgcagtttaagctcgacatcctgtggtcgatgctcgatgccatgaccatggcctacgctctgcagcgcccgccctatcacacggtcaccgacaaggcggcctggcacacgacccgactggtgtaatcatgcaaaaaacgtccatcgttgcctttcgtcgcggctaccgactgcagtgggaagccgcccaggagagccatgtgatcctctatccggagggaatggctaaactcaatgagaccgccgcggcgatcctcgagctggtcgatggccggcgcgacgtcgcggcgattatcgccatgcttaacgaacgtttcccggaagccggcggcgtcgatgacgacgtcgtcgagttcctgcagatcgcctgtcaacagaagtggatcacctgccgtgagccagaataaacccgccgtcaatccgccgctgtggctgctggcggagctgacctaccgctgcccgctgcagtgtccctactgttccaatccgctggacttcgcccggcaggaaaaggagctgaccaccgaacaatggatcgaggtctttcgccaggcgcgagcgatgggcagcgtacagctgggcttttccggcggcgagccgctgacccgtaaagatctgccggagctgatccgcgccgcgcgcgacctcgggttctataccaacctgatcacctcgggaattgggctaaccgagagcaaactcgacgccttcagcgaggccgga

SEQ ID NO: 26 - DNA, 6384 bp, Artificial Sequence
  source: /note=“Description of Artificial Sequence Synthetic polynucleotide”
aacgcgctggcgatggtggcgcacatcgaaggtgctggcgggttcgacgcgcaaggcgaagatctgcaggccttttatcgcgccggggtgcgcagcattggtccgttctggaacatcgccaatcgcttcggctgcggggtcaccggcgcctttcccggcagcccggacagcgggccgggtctcactcgcgagggcattgcgctcatcgctcaggccaacgccctgaagatgcagatcgatgtttcgcacatgaatgaacaggccttttgggataccgcccaccactccaccgccccactggtggccacccactctaatgcccacgccctgtgcccacagccgcgcaatctgaccgaccggcaactgcgggcgatccgcgacagcggcggcgtagtgggcgtcaatttcggcaatgctttcctgcgcgccgacggacggcgcgacagcgacacgcccttaaccaccattgtccggcatatcgactatcttattaacataatgggtgaggatcatgtggctctcggctcggattttgacggcatcaccctgcccgacgagctgggcgatgtggccggcttaccgcggcttatcaatgccttgcgggacaatggctatgatcaattggtgctggataagctgctgtggaacaactggctgcgggtattaaaaaaggtttggcaacaataggaatttacttacattaaggcggcgaggggcgcctatacttgatagttctgataccagaagaaggaagaactatgtggaagaaacctgcttttatcgatttacgtctcggtctggaagtgacgctgtacatttctaaccgttaatcgccccgcccgccgttcgcgcgggcaccttcattcattacccggtccgtcttcatgttcattaaagtcctcggctccgccgccggcggcggtttcccgcaatggaactgcaactgcgccaactgtcagggtctgcgcaacggcaccattcaggccagtgcccgcacccagtcgtcgatcatcgtcagcgataacggcaaagagtgggtgctgtgcaatgcctcgccggatatcagccagcagattgcccatacccccgagttaaataaacccggcgtactgcgcgggacgtctatcggcggcattattctcaccgacagccagatcgaccacaccaccgggttgctgagcctgcgcgaaggctgcccgcaccaggtgtggtgcacgccggaggttcatcaggatctctccaccggcttcccggtgtttaccatgctgcgacactggaacggcggcctggtgcatcatcccatcgcgccgcagcagccttttaccgttgacgcctgccctgatttgcagtttaccgccgtgcctatcgccagcaacgcgccgccctattcgccgtatcgcgaccggccgctgccgggccataacgtggcgctgtttatcgaaaaccgccgcaacgggcagacgctgttctatgccccggggctgggtgagccggatgaagcccttctgccgtggctgcaaaaagcggactgtctgctgatcgatggcaccgtctggcaggatgacgagctgcaggccgccggcgtcgggcgcaataccggtcgcgatatgggacacctggcgctcagcgatgagcacgggatgatggccttgctggcctccctgccggcaaaacgcaaaattctcattcatattaataacaccaacccgatccttaacgaacagtctccccagcgccaggcgctaacgcaacaggggattgaagtgagctgggacgggatggcaatcacccttcaggataccgcatgctgatcaccgacacgctgtcgccgcaggcctttgcagaggctctgcgggctaaaggcgccttctaccatattcaccacccttaccacatcgccatgcataacggcgaagcgacccgcgagcaaattcagggttgggtggcgaaccggttttattaccagaccaccattccgctgaaagacgcggcgattatggctaactgcccggatgcgcagacccggcgcaaatgggtgcagcggatcctcgaccacgacggtagccacggcgaagatggcgggattgaagcctggctgcggctgggggaagcggtcggtttgagccgcgacgacctgctcagcgagcgtcacgtgctgcccggcgtgcgcttcgcggtggatgcctatcttaatttcgctcgtcgcgcctgctggcaggaggcggcctgcagctccctgaccgagctgttcgccccacagatccatcagtcgcgcctcgacagctggccgcagcactatccgtggatcaaagaggaaggctatttttacttccgcagtcgtctgagccaggctaaccgcgacgttgagcatggtctggcgctggcgaagacctactgtgacagcgctgaaaaacagaaccggatgctggagatcctgcagtttaagctcgacatcctgtggtcgatgctcgatgccatgaccatggcctacgctctgcagcgcccgccctatcacacggtcaccgacaaggcggcctggcacacgacccgactggtgtaatcatgcaaaaaacgtccatcgttgcctttcgtcgcggctaccgactgcagtgggaagccgcccaggagagccatgtgatcctctatccggagggaatggctaaactcaatgagaccgccgcggcgatcctcgagctggtcgatggccggcgcgacgtcgcggcgattatcgccatgcttaacgaacgtttcccggaagccggcggcgtcgatgacgacgtcgtcgagttcctgcagatcgcctgtcaacagaagtggatcacctgccgtgagccagaataaacccgccgtcaatccgccgctgtggctgctggcggagctgacctaccgctgcccgctgcagtgtccctactgttccaatccgctggacttcgcccggcaggaaaaggagctgaccaccgaacaatggatcgaggtctttcgccaggcgcgagcgatgggcagcgtacagctgggcttttccggcggcgagccgctgacccgtaaagatctgccggagctgatccgcgccgcgcgcgacctcgggttctataccaacctgatcacctcgggaattgggctaaccgagagcaaactcgacgccttcagcgaggccggactggaccatatccagattagcttccaggccagcgatgaggtgctcaacgccgctcttgccggcaataaaaaagccttccagcagaagctggcgatggccagagcggtgaaagcgcgcgactacccgatggtgctgaacttcgtcctccaccggcataacatcgaccagctcgataaaattatcgagctgtgcattgagctggaagccgatgacgtcgagctcgccacctgccagttttacggctgggcgtttcttaatcgcgaggggttactgccgacccgggaacagatcgcccgcgccgagcaggtggtcgccgattaccggcagaaaatggccgccagcggtaacctcaccaacctgctattcgtcaccccggactattacgaggaacgcccgaaaggctgtatgggcggctggggatcgattttcctcagcgtcactccggaaggcactgcgttgccgtgccacagcgcgcgccagctgccggtggcgttcccgtcggtgctggagcagagtctggaatcgatctggtatgactcgttcggcttcaaccgttatcgcgggtatgactggatgccggagccgtgccgctcctgtgatgaaaaagagaaagacttcggcggctgccgctgtcaggcctttatgctgaccggcagcgccgataacgccgacccggtgtgcagcaaatccccacatcatcacaaaatccttgaggcccggcgcgaagcggcctgcagcgacatcaaagtcagccagctgcagttccgcaaccgtacccgctcgcagcttatctacaaaacccgggaactgtaatgacgctggcgacccgcactgtcactctgccgggcggcctgcaggctaccctggttcatcagccgcaggccgatcgcgcggcggccctggtgcgggttgccgccggcagccaccatgaaccgtcgtgcttccccggtctggcgcacctgctggaacacctgctgttttacggcggtgagcgctaccgcaatgatgaacggctgatgagctgggtgcagcgccaggcagggaatgtgaatgccaccaccctgtcccgccacagcgctttctttttcgaggtcgccgccgaggatctggctgacggcgtcgcgcgcctgcaggagatgctgcaggcgccgctgctgctcagggacgatattcaacgcgaagtcgcggttatcgacgccgaaaaccgcctgatccaacagcatgagttgtcgcgacgggaagccgccgtgcgtcacgccgccatcgcgcccgcggcgtttcgccgctttcaggtcggcgacgccgggtcgctgggggaggatttcctcgcgctacaggcggccttacgtgactttcaccgcagccactacgtcgcccgccggatgcaactctggctgcaggggccgcagtcgctggaggtgctcggcgaactggcgacccgtttcgccaccgggcttgccccgggcgaggcaccgccgccagcgccgccgctcactctgggcgagccccctcaactgcagctggccgtctccagccagcccgcgctgtggcgctgcccgctgatcgccttaagtgacaatgtcacgttactgcgcgagtttttgctggatgaagcccccggtagcctgatggccggcctgcgccagcgcgggatggccgaggacgtggcgctgaactggctgtatcaggatcagcacttcggctggctggcgctgattttcgccagcgaccggccggaacaggtcgaccggcagataacccactggttgcaggcgctacagcagacgacgcctgagcagcagcaacactactatcagctgtcccggcgccgttttcaggcgctgtcgcccctcgatcagctgcgccagcgggcattcggctttgcccccggggcgccgcccaccgggttcgccgatttttgcgccgccctgctggccgcccccacggtcagcctggcctgccagacgcagccccccggagcaacggtagccacccagggctttagcctgccgctcagccgctggtcgccacgtccggtctctgacccggcgctggcattcgctttttatccgcaggccgctggcgagctcgtggccgaaagcccggcggaagccgcgccactgcgtcacctcccgtcaccgggagagccgccgacgctcctgctgcgaccgcccttctactgctcgcccacgccggccggggggctggcgcgcggggaacagctgcgtccattacttgccgccctgcgccatgccgggggacacggcgagtggcatctgttcgacggcagctggcagctgatcctgcagttgcctgcgtccggccaacggccggaggcgattctgcaggccatcgtgcggcagctcgcgctcccggtcgccccgctgcccccaccgccggagagtattgcgatccgtcatctcatggcccagctccccgaacggctgggtacgtcagcgcaccaggaaggttggctggcggccctgattggcggcagcgcggaagatgcgcagtgggtagcgcgtcagctgagccggcttaccgtcccggttaatccgccgatgcccgctccggccccctgccgcggcggcgtcgagcggctggcttatccccggggcgacacggcgctactggtctttcttccgctgccggaaggcgcttcattggcggccctgcgggtgctggcgcagttctgcgagccgccgtttttccagcgcctgcgggtggagcagcagataggctatgtggtgagctgccgctatcagcgcgttgccgatcgcgacggactgctgatggcgctccagtccccggatcgccgtcccggagaactcctccgctgctgtaaaacctttctgcgccagctggaccccatggatgaggcgaccttcaggttgttacagcagcggctggccgctcaggcccgctcccgggtaccgccgaaggtgcgcgccctggacgcgctgcgccaggagtataacttgccgggggtgacgccgcaggcggctgacgcgctgcgcgttgaagaggtggtcgccctgtggcgtgagatgacccgccggcgtcgtcgctggcgagtgctgttcacgacagggagttaatggccgggggcatccggggtcagaaagtcgctgacgaagcgggtgaggatcgccgcggcctgcgggctctcctggacctcgtcgtgtaactgttgtgggtcgatgccttccgcctgcagcaccgcctcc

SEQ ID NO: 27 - DNA, 3858 bp, Artificial Sequence
  source: /note=“Description of Artificial Sequence Synthetic polynucleotide”
caaatatgctaacagcacacagcttatgctggctatgccaaatgccgctggcgctcgcccactgggggttctgctctcgttgcagccgcgccctgctcgcctgccccccgctctgcccgcagtgtggcctgccggcggcgacatcccatcacccctgcggccgctgcctgcaaaagccgcctccctggcatcggctggtggcggtcaacgactatcgcccaccgctgagcggtctggtccagcagttgaagtttcaccatcgtccggagctggggcccgccctggcgcgtctgctgcagctgcgccttaagcagcggcacgatttgccgccggtggacggtatggtcggcgtaccgctgtggcatcgccgccgctggcgccggggttataatcagtgtgacgaactgtgccgaccgttagcccgctggcgaggttgcgtctggcatcgggaaggcctgactcgccagcgagccggcgccgtccaacattcgctcaatgcccgccagcgcaggcagaacctgaaaaatgcctttcagcttgaatttgccgtggagggactccatatcgctatcgtggatgatgtcgtcacgaccggcagcaccgtcgccgagatatcccgcctgcttttgcgaaacggcgccgcgacggttcaggtatggtgtttgtgccgtaccttgtagacccccgctgatgggcgtattataacccagtaaaatagtcaactattaggccaacgctatgatccgtatttccgatgctgcacaagcgcactttgccaaactgctggcaaatcaggaagaagggacgcaaattcgcgtatttgtgattaatcccggcactccaaacgctgagtgcggcgtatcttactgcccaccggatgcggtggaagacaccgacacggcgctgaaatttgaacagctgaccgcctatgtcgatgagctcagcgcgccctatctggaagacgctgaaattgatttcgtcaccgaccagcttggctcgcagctgacgctgaaagcgccgaacgccaagatgcgcaaagtctctgacgatgccccgctgatggagcgcgtcgagtacctgctgcagtcgcagattaacccgcagctggcgggccacggcggccgcgtgtcgctgatggagatcaccgacgacggtctggctattctgcagttcggcggcggatgcaacggctgttcgatggttgacgtgactctgaaagaaggcattgaaaagcagctgctgaacgagtttccggagctgaaaggggttcgcgatctgaccgagcatcagcgaggcgagcactcctactactaagccttcctccggcggcaccctgccgccggtcctccctggccctttctgtgttaccgcgcaacatctcccgctaaatgaccggcaaaaaccggggaaagagtatgacttaagtctcatatttaacattttcccccgtcaggtgattctcaaccctcacatgttacccgtatcatccccttcaggcaccaccggcaaaaaaatagccagctatcctttctctgggtaaggataatgcaagtatttacggtgccagactattttgctcccacatggggcagatgtcttacaggttgaatctttgacgttacccataacaaattaaggccaggtaaatcggagactcccgccacagaaaaaggccggtccgcattatgcgggccggcctttttttcagacatgctcttcgccgcgcagcgaacgacgcggcgggaagtcatctggcggcggctttcttgctcgccgccttgcgccgtttatccagatccttgatcagcttattgacctgttcatcggcaaacatctgctccagggtgacggacagcttccggcgccagttcgggtactcagtgctggtgcccggaatgttaaccggcgacgccatctccagccagtcttccggctgcaggcccagtagcgcactgttactgtcggcaatgtaacgctgcatcccccggttgaggatcccggtcatgctcatcagcgaggccttatgcccggcacgtttgggcagacagccgtacttatgcaacgcatccagcagcccttgcttcgccagctcgcggtcctggtacagcccgcgcagcaccacctcatcaggatagagccccagcgctttacctagcgtcagatcgccgctttcccagtagccgcggagggtgggcaggtcgtgcgtcgtcgccacagccattgattgctccgggtacagcgccggcgcgcggaacgttttctctgcatcactctcaaaatagagcaccttataagaatagacgccgctgttgcgtaacttgctgacaatctccaccggtaccgtgcccaggtcttcgccgatcaccatgcagcgatgacgctggctttccagcgccagaagcgacagcagatcgtcgacgggatactgaacgtaagcgccatggtcggcggtttcaccataggggatccaccacaaacgcagcaccgacatcacgtgatcgatgcgcagcgcgccgcagttctgcatattggcgcgcagcagatcaataaacggctcataggcgcgagcggcaataatatgcggatccatcggcggcaggccccagttctggcccagcgggcccagaatatccggcggcgcgcccacggacgctttcaggcagtacagctcacggtcgcaccaggtctccgacccgccttcggcgacgcccaccgccagatcgcggtagaggccaatcggcatcccgtcgcgctggctggtttcccagcaggcggcgaactggctccaggccagccactgcagccagagatagaaactgacgtcgtcctcatgctcaatgcagaaggctttcacctccgggctgtcgatatcctgaaaggccttcggccaggccggccagccccagcgcagcgggtcctgttgtacctgccaggcgtgaagcgcatcgaaagcggcctgccagtagaggctttccccttcgcgcagaacaaactcgcggaacgctgtcatctgctcatcttcacgacgagagaatcgtttccacgccatacgcagcgcggtcattttcagcgtggtgaccgcggtatagtcgacatcatccgtttgccgcgccgcctgcaaggcctgctgggtcgctgcggactgccaccacgcctgcgcctcttcgctacgctggaaatcttcaaccgcattaacgtcgatgtagatgacgttcagccagcgacgtgaagacgggctatacggactggcgctctccgggttcgccggatagagagcatgtatcgggttaaggccaataaacgacccgccgcggcgggcgatttccggcaacatggcccgcagatcgccaaaatcgccgatcccccagtttttctccgagcgcagggtataaagctgcacgcaggtgccccacagctttttcccctctttcagcggctgcggttcgtagcagcgcgccggcgcgacaatggtccggcagtgccaacgctctccctcttgggtgagcgtcagggagtggtacccctccggcagtttagccggcagcggcagcgtctcgccgccgcgggttttcccttgatactgcttaccgtcctcagtggtcaggatccactgatactcaccgcgtcctgccaccggcagcgacatttttttgccgtgggtgaaaattttcacgttcggcagcgggttaaccgccactttcgtggcggcggtagagcgatgcatcgcatccagcaggcgctgtttggtgacagccgcgatagactgcggcttaccgtgcgcgttgatatagctggggctgatgcccgccgccagcgcggcattatcgaggcgtttactctccataggccttcctttag

SEQ ID NO: 28 - DNA, 5454 bp, Artificial Sequence
  source: /note=“Description of Artificial Sequence Synthetic polynucleotide”
catgaatgatatagatacactcatcagcaataatgcactatggtcaaaaatgctggtggaagaggaccccggcttttttgagaaactgtcgcagacgcagaaaccgcgcttcctttggattggctgctctgacagccgcgttcccgctgagcgacttaccggcctggaacctggcgaactgtttgtccatcgtaacgtcgctaacctggtgatccacaccgacctgaactgcctgtcggtggtgcagtatgccgtcgatgtcctcgaagtcgagcacattattatttgtggccactacggctgcggcggcgtgcaggcggcggtggaaaacccggagctggggcttatcgacaactggctcctgcatatccgcgatatctggttcaagcacagctcactgctaggtgaaatgcctgaggagcgccgtctggataccctttgcgaactcaacgtgatggagcaggtgtacaacctgggacattcgacgattatgcaatcggcgtggaagcgcggacagaaggtcaccatccacggctgggcatatggcattcacgacgggctgctgcgcgatctggatgtcaccgccgtgagccgggaaaccctcgaacagcgctatcgccacggcatctccaacctcaagatcaagcacatcaaccacaaatagccttcaggcttagcccccctggagagcgcagcgtgcgctgtcctggggggatatctcttactcgtccagcagggtaactttgccaatatacggcagatgacggtagcgctgggcgtagtcgataccgtagcccaccacgaactcgtccggaatggcgaaaccgacgaactcaaccgggacattcacttcacgacggcttggtttatccagcagcgtacaaatggccagcgatttcggctcgcgcaggctgaggatctcacgcactttagacagggtattgccggaatcgatgatgtcttccacgatcagtacatctttaccacggatgtcttcatccagatctttgaggattttcacatcacgggtggtggacatgccgctgccgtagctggaggcggtcataaaatcgacttcatgcggcacctgcacttcacggcacaggtcggccatgaacataaatgagccgcgaagcagccccaccagcaccatttcgctgccgctgtcctgataatgttcgttgatttgacgacccagttccgcgatgcgcgctttgatctcggattccgggatcatcacttctacagtatgtttcataagactaaccatttgattctaaatataaatcatcaaagccgctgctttcgccccgacgatgaccggcaagccatccagtataccagcaaaaagaaatcttcggtgcagggattaataaagccatatttgtgattccgatcacacttgttagaatttacttacattaaggcggcgaggggcgcctatacttgatagttctgataccagaagaaggaagaactatggctgaaacaaagtctcaacaatcaaggctcctggtgacgctgactgcgctatttgcagcgttctgcggcctctatcttttaatcggcggagcatggctggtcgtgcttggcggctcctggtactaccctatcgccggtctggtgatgctgggcgtgaccgtgatgctgctgcgcggcaaacgcgctgcgctgtggctgtacgctgcgctgctgctggcgacgatgatctggggcgtctgggaagtcggcttcgacttctgggcgctgacgccgcgtagcgacatcctcgtcttctttggtatctggctgatcctgccctttgtctggcgtcgtctgtccgtgccttccgctggcgccgtaggcgcgctggtcgtcgccctgctgatcagcggcggcatgctgacctgggctggttttaacgacccgcaggaagtcaacggcaccctgagcgccgacgccaccccggccgcgccgatctccaacgttgccgacggcgactggccggcctatggccgcaaccaggaaggccagcgcttctctccgctgaagcagattaacgccgacaacgtgaagaacctgaaggaagcctgggtattccgcaccggcgacctgaagcaaccgaacgacccgggtgaaatcaccaacgaagtgacgccgattaaagtcggcgacacgctcttcctgtgtaccgcccaccagcgtctgttcgcgctggacgcggccaccggtaaagagaagtggcattttgacccgcagctgaacgccgatccgtcgttccagcacgtcacctgccgcggcgtctcttatcacgaagccaaagcggataacgctcctgccgacgtcgtcgccgactgtccgcgccgtattatcctgccggtgaacgatggccgcctgttcgcggtgaacgccgacaacggtaagctgtgcgaaacctttgccaataaaggtattctcaacctgcagaccaacatgccggtcaccacgccgggtatgtatgaacccacttctccgccgattatcaccgataaaaccatcgtgatagccggcgcggtcaccgataacttctcaacccgcgagccgtctggcgtcatccgcggctttgatgtgaataccggtaaactcttgtgggccttcgatccgggtgcgaaagatccgaacgccatcccgagcgatgaacatcacttcacgctcaactcaccgaactcctgggcgcctgccgcctacgacgctaagctggatctggtctatctgccgatgggcgtgaccaccccggatatctggggcggcaaccgcacgccggagcaagagcgttacgccagctcgattgtggcgctgaacgccaccaccggtaagctggcctggagctaccagaccgtccaccacgatctgtgggatatggatatgccctcccagccgacgctggctgacattgaggttaatggtaaaaccgtaccggtcgtctatgccccggcgaaaaccggcaacatcttcgtactggatcgccgtaatggcgagctggtggtcccggccccggaaaaaccggttccgcagggcgctgccaaaggggattacgtcgccaaaacccagccgttctccgatctgagcttccgtccgaagaaagatctgaccggggcggacatgtggggcgccactatgttcgaccagttggtgtgtcgcgtgatcttccaccagatgcgctatgaaggtatcttcaccccgccatctgagcagggcactctcgtcttcccgggtaacctggggatgtttgaatggggcggtatctccgtcgatccgaaccgtcaggtggctattgccaacccgatggcgctgccgttcgtctctaaactgatcccacgcggcccgggcaacccgatggagccgccgaaagatgcgaaaggctctggcaccgagtccggcgtgcagccacagtacggtgtcccgtacggcgtcactctgaatccgttcctgtcgccgttcggtctgccgtgcaaacaaccggcctggggctatatctccgcgctggatctgaaaaccaacgaagtggtatggaaaaaacgtatcggtacgccgcaggacagcctgccgttcccgatgccggttaagctgccgttcaccatggggatgccgatgcttggcggcccgatctctacggccggtaacgtcctgttcatcggcgcgaccgcagataactacctgcgcgcgtacaatatgagcaacggcgagaagctgtgggaagcccgtctgccagccggcggtcaggccaccccaatgacctacgaagtgaatggcaaacagtatgttgtcatctctgccggcggtcacggctcgttcggtaccaaaatgggcgattacatcgtcgcctatgcgctcccggatgacgccaaataaaacgcaaaacggcaacgaaagttgccgttttttttgtgtttatcccttctccccatggaagatggcagcaggccgccgagccctaaaccgtgaatcccaacatcatcccggtgtcttcatgctccagcaggtggcagtgggccatataggcaaactccttcggcgccggatgatcaaacttcaccaacacttcactgacgtcaccttccacgcggacagtatctttccagccgctgcggtgcgcggctggcggcttgccgttctcgctgaggatacggaactgggtgccgtggatatggaacggatgcagcatcatgtcgcccttacccgatatcacccagcgctcatactggcctctggccgcggcaaacatcggcacattcatgtcgaaggctttgccgttgatgcggttggcgttatggaaatcaaaccccttgcctgacgacatgccatgatcccgaccagcctcgtggttcatattgctcatattgctcatgtcgccatggtgcatattgctcatgtcgcccatgccgccatgccccatcatgccgtggtccattcccgccatcgcctggtcgccatacttctccatcagcgcctgcatgcccatcctgtcgagcatcggatccatcgacagctggagctgacgctgcgtcagccccgtcagggacggtaacgccggcagcgtggccagcgtatccagcagtttgccggaggcgggaatgaccagcggctgaacgcgcagcaccggctgcggtttatcaaaaggcgcaatggccatgcccatctggctcaccggcagggtcaccagatcgaaaggtttaccgtcgctggtgtccaccagcacctcaaagcgttcccccatcaacaccggcaattcatcgaccttcaccggctcggccaacagaccgccgtcactggccaccacgtacagcggacgcttatcgctggtggcaaaattgagggaacgggcattacagccgtttagcaagcgcagacgtagccagccccgcggggcggcatgttcgggatagagagcgccattggtcagcagcgtatcgccaaaccagccgacagcggcgctcatcacatccagctggtagtcgatctcgccggcggcggtgaacttcttgtcctggacaatcaccggcacatcatcgattccccattgcttaggcagcagcaggcgtccgctctcttcatcctcgatcagcaccaggccggccagccccatggccacctgccggccggtggtcccatgttgatgcggatgaaaccagcaggtggccgcccgctgcgtcggcgtgaagctcaccgtacgggtggcgcccggggcgataaccccctgcggtccgccgtcgacctcgcccgggacctccaggccatgccagtgcagcgtggtctcttccgccagctggttggtaatgtcaaccgtaatggtcttcccctgcgtcaactgtagcgcaggacccagcagtgaaccgttatagccccaggtggtggcgtgcagagcgccaaactgtgttttgcccgcctggatcgtcagggcgatacggttgcgcgcatcggccgttaacagcgtcg

SEQ ID NO: 29 - DNA, 5807 bp, Artificial Sequence
  source: /note=“Description of Artificial Sequence Synthetic polynucleotide”
catgaatgatatagatacactcatcagcaataatgcactatggtcaaaaatgctggtggaagaggaccccggcttttttgagaaactgtcgcagacgcagaaaccgcgcttcctttggattggctgctctgacagccgcgttcccgctgagcgacttaccggcctggaacctggcgaactgtttgtccatcgtaacgtcgctaacctggtgatccacaccgacctgaactgcctgtcggtggtgcagtatgccgtcgatgtcctcgaagtcgagcacattattatttgtggccactacggctgcggcggcgtgcaggcggcggtggaaaacccggagctggggcttatcgacaactggctcctgcatatccgcgatatctggttcaagcacagctcactgctaggtgaaatgcctgaggagcgccgtctggataccctttgcgaactcaacgtgatggagcaggtgtacaacctgggacattcgacgattatgcaatcggcgtggaagcgcggacagaaggtcaccatccacggctgggcatatggcattcacgacgggctgctgcgcgatctggatgtcaccgccgtgagccgggaaaccctcgaacagcgctatcgccacggcatctccaacctcaagatcaagcacatcaaccacaaatagccttcaggcttagcccccctggagagcgcagcgtgcgctgtcctggggggatatctcttactcgtccagcagggtaactttgccaatatacggcagatgacggtagcgctgggcgtagtcgataccgtagcccaccacgaactcgtccggaatggcgaaaccgacgaactcaaccgggacattcacttcacgacggcttggtttatccagcagcgtacaaatggccagcgatttcggctcgcgcaggctgaggatctcacgcactttagacagggtattgccggaatcgatgatgtcttccacgatcagtacatctttaccacggatgtcttcatccagatctttgaggattttcacatcacgggtggtggacatgccgctgccgtagctggaggcggtcataaaatcgacttcatgcggcacctgcacttcacggcacaggtcggccatgaacataaatgagccgcgaagcagccccaccagcaccatttcgctgccgctgtcctgataatgttcgttgatttgacgacccagttccgcgatgcgcgctttgatctcggattccgggatcatcacttctacagtatgtttcataagactaaccatttgattctaaatataaatcatcaaagccgctgctttcgccccgacgatgaccggcaagccatccagtataccagcaaaaagaaatcttcggtgcagggattaataaagccatatttgtgattccgatcacacttgttacgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcatggctgaaacaaagtctcaacaatcaaggctcctggtgacgctgactgcgctatttgcagcgttctgcggcctctatcttttaatcggcggagcatggctggtcgtgcttggcggctcctggtactaccctatcgccggtctggtgatgctgggcgtgaccgtgatgctgctgcgcggcaaacgcgctgcgctgtggctgtacgctgcgctgctgctggcgacgatgatctggggcgtctgggaagtcggcttcgacttctgggcgctgacgccgcgtagcgacatcctcgtcttctttggtatctggctgatcctgccctttgtctggcgtcgtctgtccgtgccttccgctggcgccgtaggcgcgctggtcgtcgccctgctgatcagcggcggcatgctgacctgggctggttttaacgacccgcaggaagtcaacggcaccctgagcgccgacgccaccccggccgcgccgatctccaacgttgccgacggcgactggccggcctatggccgcaaccaggaaggccagcgcttctctccgctgaagcagattaacgccgacaacgtgaagaacctgaaggaagcctgggtattccgcaccggcgacctgaagcaaccgaacgacccgggtgaaatcaccaacgaagtgacgccgattaaagtcggcgacacgctcttcctgtgtaccgcccaccagcgtctgttcgcgctggacgcggccaccggtaaagagaagtggcattttgacccgcagctgaacgccgatccgtcgttccagcacgtcacctgccgcggcgtctcttatcacgaagccaaagcggataacgctcctgccgacgtcgtcgccgactgtccgcgccgtattatcctgccggtgaacgatggccgcctgttcgcggtgaacgccgacaacggtaagctgtgcgaaacctttgccaataaaggtattctcaacctgcagaccaacatgccggtcaccacgccgggtatgtatgaacccacttctccgccgattatcaccgataaaaccatcgtgatagccggcgcggtcaccgataacttctcaacccgcgagccgtctggcgtcatccgcggctttgatgtgaataccggtaaactcttgtgggccttcgatccgggtgcgaaagatccgaacgccatcccgagcgatgaacatcacttcacgctcaactcaccgaactcctgggcgcctgccgcctacgacgctaagctggatctggtctatctgccgatgggcgtgaccaccccggatatctggggcggcaaccgcacgccggagcaagagcgttacgccagctcgattgtggcgctgaacgccaccaccggtaagctggcctggagctaccagaccgtccaccacgatctgtgggatatggatatgccctcccagccgacgctggctgacattgaggttaatggtaaaaccgtaccggtcgtctatgccccggcgaaaaccggcaacatcttcgtactggatcgccgtaatggcgagctggtggtcccggccccggaaaaaccggttccgcagggcgctgccaaaggggattacgtcgccaaaacccagccgttctccgatctgagcttccgtccgaagaaagatctgaccggggcggacatgtggggcgccactatgttcgaccagttggtgtgtcgcgtgatcttccaccagatgcgctatgaaggtatcttcaccccgccatctgagcagggcactctcgtcttcccgggtaacctggggatgtttgaatggggcggtatctccgtcgatccgaaccgtcaggtggctattgccaacccgatggcgctgccgttcgtctctaaactgatcccacgcggcccgggcaacccgatggagccgccgaaagatgcgaaaggctctggcaccgagtccggcgtgcagccacagtacggtgtcccgtacggcgtcactctgaatccgttcctgtcgccgttcggtctgccgtgcaaacaaccggcctggggctatatctccgcgctggatctgaaaaccaacgaagtggtatggaaaaaacgtatcggtacgccgcaggacagcctgccgttcccgatgccggttaagctgccgttcaccatggggatgccgatgcttggcggcccgatctctacggccggtaacgtcctgttcatcggcgcgaccgcagataactacctgcgcgcgtacaatatgagcaacggcgagaagctgtgggaagcccgtctgccagccggcggtcaggccaccccaatgacctacgaagtgaatggcaaacagtatgttgtcatctctgccggcggtcacggctcgttcggtaccaaaatgggcgattacatcgtcgcctatgcgctcccggatgacgccaaataaaacgcaaaacggcaacgaaagttgccgttttttttgtgtttatcccttctccccatggaagatggcagcaggccgccgagccctaaaccgtgaatcccaacatcatcccggtgtcttcatgctccagcaggtggcagtgggccatataggcaaactccttcggcgccggatgatcaaacttcaccaacacttcactgacgtcaccttccacgcggacagtatctttccagccgctgcggtgcgcggctggcggcttgccgttctcgctgaggatacggaactgggtgccgtggatatggaacggatgcagcatcatgtcgcccttacccgatatcacccagcgctcatactggcctctggccgcggcaaacatcggcacattcatgtcgaaggctttgccgttgatgcggttggcgttatggaaatcaaaccccttgcctgacgacatgccatgatcccgaccagcctcgtggttcatattgctcatattgctcatgtcgccatggtgcatattgctcatgtcgcccatgccgccatgccccatcatgccgtggtccattcccgccatcgcctggtcgccatacttctccatcagcgcctgcatgcccatcctgtcgagcatcggatccatcgacagctggagctgacgctgcgtcagccccgtcagggacggtaacgccggcagcgtggccagcgtatccagcagtttgccggaggcgggaatgaccagcggctgaacgcgcagcaccggctgcggtttatcaaaaggcgcaatggccatgcccatctggctcaccggcagggtcaccagatcgaaaggtttaccgtcgctggtgtccaccagcacctcaaagcgttcccccatcaacaccggcaattcatcgaccttcaccggctcggccaacagaccgccgtcactggccaccacgtacagcggacgcttatcgctggtggcaaaattgagggaacgggcattacagccgtttagcaagcgcagacgtagccagccccgcggggcggcatgttcgggatagagagcgccattggtcagcagcgtatcgccaaaccagccgacagcggcgctcatcacatccagctggtagtcgatctcgccggcggcggtgaacttcttgtcctggacaatcaccggcacatcatcgattccccattgcttaggcagcagcaggcgtccgctctcttcatcctcgatcagcaccaggccggccagccccatggccacctgccggccggtggtcccatgttgatgcggatgaaaccagcaggtggccgcccgctgcgtcggcgtgaagctcaccgtacgggtggcgcccggggcgataaccccctgcggtccgccgtcgacctcgcccgggacctccaggccatgccagtgcagcgtggtctcttccgccagctggttggtaatgtcaaccgtaatggtcttcccctgcgtcaactgtagcgcaggacccagcagtgaaccgttatagccccaggtggtggcgtgcagagcgccaaactgtgttttgcccgcctggatcgtcagggcgatacggttgcgcgcatcggccgttaacagcgtcggtatcggcagagccgggcgactggccgcaaaaaccgcacggctccatagcggtaatgcgctggccacgctaaccgcggcggtcagtttgaggaagtctcgacgttgcatattcacatccttttta

SEQ ID NO: 30 - DNA, 3260 bp, Rahnella aquatilis
aagttaaagaaagttgcgctaacagcatgcatccggaaactggtgacaatcctgaatgcgatggtgcgagatggccgtgaatgggcagcaaattaatcgctgccggattgacattcgacacagctgctcccccttcgcagggggaggagaaagtcttacttctgtaccgtcttcaaagccgcaatacaacgatctaccacaccttcaaacttatggtcgatattcacccgcaacacatccggttcgtcttcgcccggttcttcgagcgcatcgaactggctgcgcagtaaatcggtcggcatgaaatgtccggcacgcgctttcaggcgctcggcgatcacttcaaaactgcctttcatgtagatgaataccatgccttcgttgtctttacgcagggcgtcacggtagcgacgtttcagtgcggaacagacaatgatgccggtttcatttttatggcgcaggctgtaagccgcgtcgttgaggcgcaataaccacggcgcgcggtcatcgtcgttcagtggcgtgccgctggccatcttttgaatattggcgcggggatgaagatcatcgccgtcgataaatttcgcgttaatttcacgggccagtgccgcgccgacggtggatttaccgctgcctgacacgcccatcagaatgatgctttgtcctgccataagtatgatctctatcccaaaatgaacattttgactgcgtagtgagtcctaatattaccatgttaccggtatcatgataccggtaacaaatggagatgtgactaatatcacaaaggaatgcactgctgttttagcggtcgcggtctgtacccgtagcactgttaattcctacaacatcttgaaaattaaaacaatgcttcacccggtttttagccatacccgggtgacagcgtgaggggaagaatatgccattagttattgtagcggtgggtgtcgcactgctgttgttacttatgatccgatttaagcttaatggcttcattgcactgattcttgtcgcgctggcggtggggattatgcagggaatgccggtcgataaagtcatcacctcgatcaaaaacggggtgggtgggacgcttggcagtcttgcgctgatcatgggctttggtgccatgctcggtaaaatgctggcggattgcggtggcgcacaacgtatcgcgaccacgcttatcgaaaaatttggtcgcgaacacattcagtgggcgattgtgctgaccggtttcatcgtcgggtttgccctgttctatgaagtcggtttcgtgctgatgttgccactggtcttcactgtggccgcggcggcgcgtttgccattgctgtatgtcggcgttccgatggcggccgcgttatccgtcacacacggattcctgcctccgcaccctggcccgacggcgattgcgacgattttccatgcggatatgggtaaaacactgctgtttggttcgctgctggcggtgccgaccgttattctggccggtcctgtgtatgcgcgcttcctgaaaggtatcgacaaaccggtgccggaaggtctgtttaacccgaaaaccttcacggaagaagagatgccgggctttggcgtcagcgttgcgacgtcactggtaccggtcattctgatggcattccgcgcgctttgcgaaatgatcctgccgaaaggccacccggtgctggcgtatgctgaattcttcggcgacccggtgatggcaacgctgattgcagtgctgattgctatcttcaccttcggtctgaaccgtggccgtaagatggaagatgtaatggcgaccgtgaccgactccatcaaaattatcgcgatgatgttgttaatcatcggcggtggcggggctttcaaacaggtgctggtggacagcggtattgaaaaatacatcgcggccctgatgcacggcagtactctgtcgccaatcctgctggcctggtccattgcggcagttctgcgtatcgcgttggggtctgctactgtggcagcgattactgcgggcggtatcgccgcaccactgatcgccaccaccggtgtcagccctgagttgatggtgatcgccgtcgggtccggcagcgtgattttctcgcacgtcaacgatccgggcttctggttgttcaaggaatatttcaacctgagcattgtcgaaaccttcaaatcctggtcggtgctggaaaccatcatttccctgtgcggtctggcgggatgtctgctgttgtcgatggtcgtctgacgacaaattgttagcataaaaaggcgagccagacggctcgcctttttcgtttcttctgcctgctaaatcttcagatacgccctgatcccatcgaggaacatctgcgttgagagcatgaccagaatcagacccatcaggcgttcgagggcgctgacgcctttgtcgccgagcaggcgcagaaacaggtttgagagcagaaggatcgccatcgacatgccccaggcgataaacagcgccagcgtgagatgactcatctgattcgggtactggtgcgaaagcagcatcagcgtcgcgagaatcgacggaccagcaaccagcgggatggccatcggtacgaggaagggttcttcaccggcaggcaggccactgctgcttttctcttccgacgggaaaatcattttgatggcaatcaggaacaggataataccgccggaaattgacacggtttcggtacgtaaattcaggaatgacaggattttttccccggcgaacaggaaaatcagcatcaggatcagtgcgatcagcatttcgcggattaacacgacccggcgacgtttcgggtcgagatgttttaaaaccgacataaaaatcggtaagttgcccagcggatccataattaaaaacagcaaaatggttgctgaaatcatttcggtcatgactacctcagaaaaaatgcctgttataagttgcgctgctattaaaagctgcactattgattaaattcacttgccacttctactacattttgtaaggtggtagaccagcgcgttaacggggcacagtgcatcaaaacaggcactaacttagtgcaaatgcgcgttcaagccgttttaagcagttatcaccctcagggcaggacagatattatgaaaaatgtaggtttcgttggctggcgcgggatggtcggttctgtactcatgcaacgcatgattgaagaacgcgatttcgacgctatccgtccggtattcttctccacctctcagcacggtcaggccgcgccttctttcacgggtcagtccggaacctt

SEQ ID NO: 31 - DNA, 3292 bp, Rahnella aquatilis
aataaaaaaagcctgaatttcttcaggcccgatctttgccatcctgatgacagattactggaagtaaatatcgccgttttcggctttgaggatcttgccgtcggtgtcagtgatcagtgcgtagctgccgccgatataagtccagtggctacccgcatccggtgccggcaggtgacgttcctgccagccgacgatctcatacggcttggtacggtacagcgcagggacctgatcgccgatggaatacaatttggaatcatcgtagaacgatttgatctcgtgtgagtttgacgcggcaaacgccgtgttggccaggggtaacaacgatgaaacaaccagggctgataacataacaattttagaatgacgcatagattatcccgttaaaaccatcaacttaatgcctgcctcggtgatatgccgccagtccgttgtatgagcagtaaagccgaagcctgtgctttttctgtaggacaggataataacgagaaagtgagtgcaggctgggagatttttttgtgtcgaaatgcaaaccgtgcgtcgttttatctgaacacgccgattttgtactgaacaatgtctcggttctgtaaacttttgagacacattagcgctgtcacttttcccttgaaaataaaaggattaacggtttcctgctgttttatccccgcctggtgtcgccataactgtttaatgctacatatcctttaataaccttttcctgcaaaagaaatcactatgcgcaatgaacggttttttagctggtcgttcatgaccgggcttgtcacggctgtcatcgggctggcttatctggtgctgggcgtctggctggcggccctcggcggctcaccctggtatgtgctttttggcagcggatatctgctcagcggcatttttatcgcgcgccgccatgccagcgggatctggctttatctgctgacatttctgctttgctgcgtctggtcggtatgggaagtcggactggatggctggcaactgatgccgcgtttgtttgtgatggcgttattaggcgtctggtgcagcctgccgttgatcacccgtcaggtgatggcaacgcggggcaatcaccgcaccggtacgttcgccgggctggtgtatgtggtggcgatcgtgggtattttttacagcggctggcaggtgacagggtcacgttttgtccatcgtcagcctgttccggcgcaatcgggtgatatacaggccacgtcgccggaaagcaacgactggcgctattacggcaggacagaagccggacaacgttattcgccgctgacacagattacacccgctaatgtcagtcagcttaaacccgcctgggaatttcacaccggcgatgtgatgagaaagggtgaggataaagacggacgggaatttaatttcgaagtcacaccggtcaaagtcgggaactcgctgtttatctgtacgccgcaccgcgaagtgattgcgctaaacgccaccaccggcgcgcagcgatggaaattcgatcctaaatctgacacctcggccaatgagtatctggcatgtcgcggcgtggcatacagccagtcagccggtgataaagtgtgtccggaaaaaatcatcgccaccaccagtgaggcgcggatggtggcgctgaatgcacagaccggcgaaccctgttcatcctttggtcagaacgggtttgtcagcctgaccgaccatatgggcgacgtgccgccgggcttccacttcattacctcgcagccgatggtgatggatggccgcatcgtgctgggcggctggatttatgacaaccagtctacgggtgaaccttccggcgtcgtccgggctttcgatgtgaacaccggccagcttgcatgggcgtgggatatgggccgggatccgcaaaatgcgccgctcaaaccgggtgaagtgtatacccggggcacgccaaacggctggggaacctataccggtgatccgaaactggggctggtttatattccgctcggcaatgcgacgccggattattacggtgccggacgccgtccgtttgacgaaaaatattcgagttcgctcgtcgcgctggatattcacaccggcgaggaacgctggcacttccagactgtgcatcatgatgtctgggactttgacttgccgatcggaccgacactggtggatttgccgtcaccggaggggataaccgtgcctgcgctggtgcagaccaccaaaatgggacagcttttcctgctcgaccggcgtaccggcaaaccgctggcgcaggtaaacgaaaaaccggtgaatacctctccctctttgccgggcgaacatctgtcgccaacgcagccggattctgtcggcatgcccagtctctctccgccagatctgaaagaaaccgacgcgtggggcgcgacaccgattgatcagttgtattgccgcatccagttcaaaagtgcccgctatcaggggcagttcaccccgccagcggaaggtaaatccattgcttatccggcctttgacggcgtgatggactggtacggcgcttcggtggatccgatccgccatgtgctgattgccaataccagttacatcccgttcacgatggaagtgaaaaagtcagccgatgcgatcaaagaagggctgatgcacaaatgggccggatggggcagcaaccagccttatccaaaacccaaagagttttcggttggcccgcaatatggcacgccgtgggcggcgatcgtcaaaccgtggctgagttttctgcaggcaccctgtaatgcgccgccgtggggaaaactggttgcggttgatctgaccacccgaaaaatcgcctgggaaagaccggcaggcacgacccgggatatgaacatttttggcacgcataccaacgtgccattgccgaccgggatttttatgatgggcggtaacatcattacccaaagtggcctgattttcaccggcgcaacggcagacaactatttccgcgcattcgacgaaacgacgggtaacgaactctggcgagcgcgacttccggcgggcgggcaggcgacgccgatgacatataccggcgatgatggccgccagtttgtggtgattgccgccggcggacacggcgggctggggacgacgtccggtgatgcgctggtggcgtatgcattaccggccagatagggttggcactcaaacgcaggatattcagaaccgtctgatgatgcccacaggcggcgcagcatggtctatttttaagatcccttcttttttaaataaggagagtttatgaacaacaaaacaatcccgacatccctgaatgaacctc

SEQ ID NO: 32 - DNA, 5150 bp, Rahnella aquatilis
gccaactgaaagtattctctggtaaatacgtgtcaggtgtcttttttgtttttgataaatatgtttattatcaatgaattgtatattgtgtaatcgttaccaccgggacgcagataaataccaggcaagggtgggatgaattttcgaatgaaagcgaatttttatttcaggtggggataaaaacagtaacagacgagtaaacccccatattttgcacggagatggaatttgaaaatgaggtcagatgtacactgcgtgcggtattttgttaaaaagttatatttgtaaagttgcgtatgtactaacaggaagaaaaaatctatagtgaggcccttcacggctctcaatcagattggtcgatttatgtctacttcttatgtccaaaattgagagatgcgtcgtcttttttgactcaaccttttaacgtaaaacaaaagaactcataaagtgaacgccaaaacctctgcttttatcatcacgtttctgtcatttttgaacccggtatcagtcagttacgcccagtcagccaccgcgacgccgaaaggtgagacggatgacgcatccgatgcttcgacgatgatggtcatcaaacgggcaagcaccggtgagttgtctgaactcgacaccccggcggcagtcagcgtgattgatggcgaagatttcagaaacagcaaagcgcaggtcaatctgtctgaaagcctgggcagcgtccccggtttacaggttcagaaccgccagaactatgcacaggatctgcaactttccgttcgcggattcggcagccgttctatgtatggcgtgcgcggcgtgcgtatttacgttgatggtatcccggccaccatgccggacggccaggggcagacctccaatatcgatctcaactcggtagagcgtgttgaagtgttgcgcgggccgttctcggcgctgtacggcaacgcctccggcggtgtggtaaacgtcgatacccaaaccggttcacagcctgccagcctggaagccggcacttattttggcagctacggttcgtttcgtaacagcgtcaaagccagcggcgcaaccggcgacggcacccatgccggtgatgtgaattacctgatttccggttcccgtttcaccactgatggttaccgtgaccacagcggtacgcagaaaaatctcggcaacggcaaactgggcgtgcgcattgatgatgccagcaccctgacactgatgtttaacagcgtgtccgtggatgccaacgatccgggcggcctgacagaatctgaatggcacgatgacccgaagcaggccagacgcgccgatcagtacaacgcacgtaaaagtcttgatcagacgcaggtcggcctgcgttatcagcgtcagatgggcgaaaacgatgagttcagcctgatgacttatcacggcgagcgtcacaccacgcaatatcagactatcccggctacgtcgcagaaaaatccgctgaacgcgggcggggtgattgttctggaacgtaaatactccggcgtagacacccgctggaaacatgacgatcagataggttccgtgccgttctcggttaccggcggtctggattatgaaaccatgaccgaacgtcgtttcggctatgagaacttcaacagtgaaggcgatctgggtgtgaaaggcgacgaacgccgtaatgaaaaaaacgtgatgtggaacctcgatccctatctgcaaaccacgtggaacctgacatcgcgctggacactcgatgccggtgcgcactacagcaccgtgagtttcgactcgcaggattattacattaccggcagcaacccggatgacagcggttcgcgccgttatcataaactgctgccgatggcgtccctgaactatgccgtgacgcctgcactgaacacctatatttctgccggtcgtggttttgaaacgccgaccattaatgaactgtcttaccgcaccgacggtaaatcgggcctgaatctggggctggaaccttccaccagtaccaccgtggagctgggcagcaaatggcgcgtgggcaacggtttggtgaccgccgccgtcttccagactgatactgatgacgagctgattgtggcacaaagtaccggcggtcggtcgagctacaccaacgccggtaagacacgccgtcgggggctggaactgtcattagatcagcagattgaagaaaactggcgcgtaaaaatggcctggacgctgctcgatgcgaccttccgcaacgagacctgtggcgcgggtgattgcacacccgcgggcaaccgtctgccgggtattgcgcgcaacatgggctacgcttctctggagtgggcgccggttgagggatggcacgcgggtgccgatatccgctacatgagcgacattgaagtcaacgacgaaaacagtgaacaggcaccggcatataccgtcgccagcgtgaatgcgggttaccgcttcaactggaacaacgtgacgctcgacttgtttacccgtgtcgacaatttgttcgacagaaattatgtcgggtctgtgattgtcaatgaaggcaacggccgttatttcgaaccggcacctggcagaaactacggtggcggagctacgctttcttatagtttcgaataattttcagtcagtaaccacaacgaaccacgcggtgctgcccgttacctgcactttttgaagcagggcagcacatgcaaggaatgacacccggtgaaaaccggatgttcttgaagattcaggctatccctgaatcgacgattataaaaaatcagtaaggtcatcaactatggaaacgaaagcctcgttatcacgcattgtcgtcataattaccgctttgttcgccgcgttaagcgggatttaccttcttgccggtggtatctggctggcaaaattaggaggttctctttattacatcattgccggtgttatttcgctggttactgcgtggctgctttaccgtcgccgctcttcggcattattgctctatgccatcttcctgttcggcaccactgtctgggctgtatgggaagtgggcacagacttctgggcactgacgccgcgtctggacgtcaccttcttcctgggcctgtggatcctgctgcccgtggtttataaccagatgctggcgaaaaacgcctttgcacgcggcgcgctggcagtttctctgctgttcaccgtgatcgtgctgggctacgccatctttaacgacccgcaggtgattaacggcaccatcaaagcggcggattctgctccggcaaaatctgagtccggcatccctgatggcgactggccggcgtatggtcgtactcagggcggtacccgttactcgccactgaaccagatcaacgataaaaacgtcagcaagctcgacgtggcctggactttccgtaccggtgacctgaagaccccgaacgatccgggcgaaatcactgacgaagtcacgccaatcaaaatcggcgacatgctttatctgtgtacgccgcatcagaagctgtttgctctggatgccgcaaccggtaaagagaagtggaagttcgatcctgagctgaaacccaacccaaccttccagcacgtgacctgtcgtggtgtgtcttatcatgagaccacaccagccgcagaaggcaacgccaccaacggcgcggcgcctgctgtctgttcccgtcgtatcattctgcctgtcaacgatggccgtctgtttgcgctggacgctgaaaccggcgcacgttgcccggcatttggcaacaacggtgagctgaacctgcaaggcaacatgccttatgcaaccccaggccactacgagccaacttcaccacctatcatcaccaaatctgtgatcatcgtggcgggtgcggttaccgataactactcaaaccgcgagccttccggcgtgatccgtggttttgatgttgagaccggtaaactgctgtgggccttcgatccgggtgcggctgagccgaacaaaatccctgaggatggtcagcatttcacgccgaactccccgaactcatgggcacctgcggcttatgatgacaaactggatctggtttacctgccaatcggcgtggcaacccctgatatctggggcggcaaccgtactccggaaatggaacgtttcgcgagcggcctgctggcgctgaatgcgaccaccggtaaactggcctggttctatcagactgtgcatcacgacctgtgggatatggacgtgccagcgcagccaacgctggctgatatcactgacaagagcggcaacaaagttccggcgatttatgtaccgaccaaaaccggtaacatcttcgtgctggatcgccgtgacggtaagctgattgttgatgcgcctgagaaacctgttccgcaaggcgcggcgaaaggcgaccatgtgtctccgacccagccattctccaaactgactttccgtcctgaagccaaactgaccggtaaagacatgtggggcgctacgatctacgaccaactgatgtgtcgtgtgatcttccacaaactgcgttatgaaggcaccttcacgccgccatccgagcagggcactctggtgttcccgggcaacctcggtatgttcgagtggggcggtatttctgtggatacagaccgtcaggtggctatcgcgaacccgattgcgctgccattcgtgtctaaactgatcccacgcggtccgggcaaccctatcgagccagatgcgaacgataaaggcggttccggtactgagactggcattcagccgcagtacggcgtgccatttggcgtgacgctgaatccgttcctgtctccgctgggcttcccatgtaaacagcctgcatggggttacatttccggtgttgacctgaaaaccaacgatatcgtgtggaaaaaacgtatcggtaccgtgcgcgacagctcaccattaccgctgccgttcaaaatgggtatgccaatgctgggcgcaccggtttctaccgccggtaacgtgttcttcatcgcggcaaccgcagataactacctgcgcgcgttcaacatgagcaacggtgacaaactgtgggaagcacgtctgccagcaggcggccaggcaaccccgatgacttactccgtcaatggcaaacagtacgttgttatcgcggcaggcggtcacggttcgtttggcaccaaactgggcgattacatcatcgcttacgcactgccag

SEQ ID NO: 33 - DNA, 2571 bp, Rahnella aquatilis
aaaaggccgttccctggcccttgatgcagcgggtgatttacgggtcggtctgaggtcaggcgttcagtaaagacgctgtgctctcatgcaattcatcaagcaaggtataacgcttgcggtattcagaacgtttcttgctggcaatctcttccatcggtttgcgcggcagttccagtggaacgtgatataaggctgaagatgcgggcgtggcactgatggaatcccagaattcgttataactggcgacaaacacgttcttcttgcggtgacggtaacgcaggctgcggaatacgtgaccggtatcgctgacggcaagaatgcgctgcacgccggttccggcagcgacacgttgcaggctttccagcagcagacgtttcgggaacaagccgtaacaggatttagtcgcctgtttaatcacttcatgtgccgttgaacggtgcgcgccctggaatccgccgataatcagtgtcctcccttccgggcgctgcacaatgctaaacgtcagtgccgcaagcagcgtgtcctccatataaaggaacatgttggcttcgccttcgcgctcagatttgccgatggaaccgagctccacccggaaggcttcaccgtctttgccggtgaaccgggtgatggtattgccggtcgcgctcagaaaggtattgcgcaggcgggcgttttccaggcttttgacgaaggcgtagtgatcaaccagtgcgtcagctcgtccggctgaatgcagaccgagatacagatacggcttgtgaattttactcggtagtttggtctgcacctggaaagcttcctgccagagggtttccgccgccatcttattcagcactttcagggtatcgagcggatgcagtaaggtgcgaacggcatatttgacccggaacatccggtcgcgccacagattgtccggtacgcacgctccgctcagaagatcgacaaacaggctgaatccgctggttttttttgtggtttgttcaacgggatatgcttcgctgagggactcggtttcattgagagggatatgttccatgatctgtttccgctgccagaaataatcacggtatcacctgatttttaaacctctttacaacgttgttttaaagagaagcgaggttatctgaataagtgcaggctgtttagctgccgtttattctggctgactgaaagcgtctcactgtctgatatctattgatatgcgtgatgagtttctcgttttagtacatcatacgaacgtaattacgcagaatttggttaacttagaaatgttgggaaatgttacgaaaatgaattcgtgatgatcattcagcagttgtaaggaaactttgatgaaaattattactactttttgcctcgccagccttttttctgtcaacgcgtttgccctgaccggtaacgatgcgaccaccaaacccgatctttactacctgaaaaacgatcaggcgattaacagcctggcgctgcttccgcccccgcctgcggtgggcagtatcgcttttctaaacgatcaggccatgtatgaacagggtcgtctgctgcgctcaacagaacgtggaaaactggcggcagaagatgccaacctgagtgccggtggcgtggcgaacgccttctcgggtgccttcggttcgccaatcaccgccaaagacagcccggaactgcataaactgctgaccaatatgattgaagatgcgggtgatctggcgacacgctccgccaaagaaaagtatatgcgcattcgtccgtttgccttctacggtgtgccgacctgtaacaccaccgagcaggataagctgtcgaaaaacggttcgtatccctccggtcacacctcaattggctgggccaccgcgctggtgctcaccgaaattaacccgcagcgccaggaccaaatcctgcaacgcggtttcgatttaggccagagccgggtaatttgcggctaccactggcaaagtgatgtcgatgcagcgcgcatcgtcggttccgccgtagtggctaccctgcacactaaccctgctttccagcaacaactgcaaaaagccaaagaagaatttgctaaacagcatccgtaaatattgggcctccccgacctggtcttcgtaaaagccggggagtctgtaatgccacatctctcattgatactaaatttctgaaaattgcttttttgcaacggtcactaattgttaactccccttcacaggcataacgtcattcctcattttaaaaaggagtttcattatggctagccccgcatatctctggctatacgatgccaatggcgcactactttatggcggttctgaggttttaagccgtgaaggcgcgatcgagatccaaagcttcacgcatggtctttccgtacccttcgatggtaataccggtcggctgacatctacccgtgttcatcaaaccatgggactggtaaaagagttcgataaatctactccctacctctaccgtgccgtcgccaccagcgaaaagctgcaaaaggcggtgattaaatggtatcgcattaatgcggctggtatggaggaagagtttttgaatatgacaatggaaggggtgcgcatactt

SEQ ID NO: 34 - DNA, 1922 bp, Rahnella aquatilis
ttccagattgagaaaaagtgacaggttaagctctgatttcaactgaaccggagcctgtcatcatgaataccaccgcaacaattctgccctcgtcccctgaacgctgcgtgattgtcatcaatcagcaacttgccgcaggacatgccgccaatgccgccgcggtcctggcgctgacgctcggacaacgccatccggcgctggtgggcgcaccgctgattgatgccgataaccgcgaacatccggggctgatcccgattggtatcagcgtgttagtcgctgatgctcaacaactcacacaacttcatcagcatctgctaaatgacgatgaaatggacggcattattttcccggtcgaagggcagcaaaccaccgattacgccgcctttcgtgaggcggtatcggttgtgccgaccaacaacctgcaactgctcggcatcgcgctggcggggaataaaaaagtggtgcgtaagctgaccggcaaactgggattactgggctgatgcctgtcatttaagcgtcacatttcttctctattctcctgatattcaaactaatatcaggagtttcaccgtgcgtaagtccctcgtcgctctgctgcttttcactctgggttccacctttgctgttcaggctactgaagaagccaaaccctttatcaccagtcaggaactggatctgacccaatatctgccagcgccaccggcggatgattcggcgcagacccaagcggagctgaaagaattgctccaaattcaggccacccgcacgccggagcaggaaaaagcggcgattgctgatgcgcaagaaaacgtctggcgttttgccgatgtgatggggccgggctttgatgccgagaaactgccgaaaaccgccgcgctgtttgagcgtattgtggcgacagaagacgtggtggacgatcacgccaagaaagcgtttaaccgtccgcgtccttatatgctggatgaacaaattcatccgctgctgaaaaagtctaaatccggttcatggccttccggtcattccaccatcggttatctgatggcgacggtgctgggcgaaatggtgccggaaaaacgcaatgcgctgtttgcccgtgcatccggttatgccgaaaaccgtctggtggctggtttccattaccgttctgataccgtcatgagccgcaccggtgccgcgctgattgctcagaaaatggaagaacagccagatttcaaaaccgaattcgacgcggcgaaagcggaactgcgtacgcagctgggcctgaaataattttcgtcagtttctgcctttccggtaaagtatggaacgtaaaacttgcagaggaagggactgatggaatacagattcaagcggatggcatcaccggtcgggttactcacactggcggcgaaaggcgacaagctgaccgccattttgtgggaatgtgaaatcgacgggcgcgtgcctttgggtgaaatgctggaagacccggcgtttccgattttgctgaaaaccgaacaacagctgaacgagtatttcgcgggtaaacgcacgtgctttgagctggaccttgatttcaccggcaccgcattccagaaggaagtctgggcggcgttgctggaaattccgtttggtgagacgcgcagctacggcgacatcgcccgccgcatcggccgcccgaaagcggttcgcgccgtgggcgctgccaatggtcgtaatccgatttctatcgtagcaccctgtcaccgggtgatcggttcttccggcaaattaaccggctttgccggcggactggagaacaagttgttattactgaggctggaaggccgtaaatcgtaaggttttttatgggggaataaattgcttgatgtccggttgtcagtcgatgcatagtcaaaaaataagcagtaaataaaaaagacaacatcacttacagacggagt

SEQ ID NO: 35 - DNA, 2643 bp, Rahnella aquatilis
tggcggcgataggccgccccccaagtcggtgtgggccgacgcccacggtcttgaccttgacctggacattgattttaaaatcgccccctttgcaccgatccgtgcaaaaaaaccaaaacttacataattccccatgtttcaacatgcttttataaagtaccattaacatcatgaacaccatccctcatccccctgtctttgatggtcataacgacctgttactccgcctatggctgcatgatgcagcagatcctgctgccttgtttcttgatggctcactggaaggacatctcgatttcaggcgctgtcgtctgggtggtttcgcaggtgggttatttgccattttcgtaccacctgcttcatatatgccacaactgaagcccgattcacctgcggaacctcatgacgcttttgccattactcgggcacagatttcactgctggaacgccttgaaacgcagtccgccgggcgggcaaaaatctgccggacggtcggtgaaattgaagcgtgtattacacagaatgtgctggcgatggtgatgcatatcgaaggggcggaagcactcggcgatgatttctcgcggctggagcgctggtatgaaaaggggctgcgcagcatcggcccgttatggaacttacccaaccagtttggtaccggcgttaagggcgatttcccgggatcaccggataccggagatggcctgacgcccgccgggcttggattgctgcatgaatgtaaccggaaaaggattctgttcgatgtctcgcacatgaacgaaaaagccttctggcagacggcaaaattcagcgatgcgccgctggttgccacccattcaaatgtgcacgcgttatgtccgcaaccgcgtaatctgaccgataaacaactggctgccattgccgaaagcaacggcttcgttggcgttaatttcggcacggcttttctgcgggcggatggaaaacgcaacggcgacacccccatcaccgaaattgttaaacatcttgataaccttgttggtaaactgggggaagaaaatgtcggttttggttcggatttcgacggtatcaatgtgccggatacgctcggtgatgtcgccggattaccgttgctgcttcaggctatgtctgatgcgggatacggcgatgcattgatcgaaaaaatcgcgtaccgcaactggctgaaagtattaaagcaaacctggggtgaatagtttttcatccgatattttgcgctaatccctgtggcttacagggcaatatcggtttgagctcatcgcaacaattttttagccaaaccagcgatatttgacaataagccgatcacctgccgcaataaaaggggcagtgaacttaacactgtaatcatcaagaggacgttattatgtggactaaaccttcattcgaagacctgcgtttaggcttagaagtgacactgtacatttctaaccgctaagcctttatgcccacggttaactgtgggcatcttccttccctttcccctggttctcacatgcagattatcgtacttggttccgcggcaggcggcggcttcccgcagtggaactgcaattgcagcaactgtcagggtgtgcgtaatggcaccatgaaaacgtccccccgcacgcaatcttcgattgccgtcagcgacaatggcaccgactgggtgctgtgtaacgcctcgccggatatttgccaccagattgccgccacgccggaactgataaaacaagacgttttacgcggcaccgccattggttccattatcctgactgacagccagattgatcactgcaccggcctgctgaatttgcgtgaaggctgtccgcatcaggtgtggtgtacgccggaagtccacgaagacctcaccaccggtttcccgattttcaccatgctttctcactggaatggcggcctgcaacaccacgctatcaggccggagaaccgcttctccgttgccgtctgcccgaatcttacattcactgccattccgctgctgagcaacgcgccaccgtattcgaaataccgcggcaaaccgctccccggccacaatatcgcgctctttattgaagacacaaaaaccggcacctcgctgctgtacgcaccgggtctgggcgaaccggacgatgaactgctgaaatggctgcataaagccgattgcctgctgattgacggcacgctgtggcaggacaacgagctggcgaccaccggcgtcggccgcaataccggcaaagacatgggccatctggcgcttgccgaagaacaagggctgatcgccctgctgtcgtcacttccggcaaaacgcaaaattctcatccatattaataataccaacccgatcctcaatgaatcctctgccgagcggcaggcgctgacgcaacaaaacatcgaagtcagccgggacgggatgcgcatcgaactgtagggcaaaacgaccatgagcatatcgacaacacagacgtcaccgatgacgccgcaagaatttgaacaggcgctgcgtgccaaaggcgcgttttatcacatccatcatccctaccatattgcgatgcataacggtcaggcgacccgcgagcaaattcagggctgggtggcgaaccgtttctattatcagaccagcattccgctgaaagacgcggcgattatggccaa

SEQ ID NO: 36 - DNA, 1038 bp, Rahnella aquatilis
atgaacaccatccctcatccccctgtctttgatggtcataacgacctgttactccgcctatggctgcatgatgcagcagatcctgctgccttgtttcttgatggctcactggaaggacatctcgatttcaggcgctgtcgtctgggtggtttcgcaggtgggttatttgccattttcgtaccacctgcttcatatatgccacaactgaagcccgattcacctgcggaacctcatgacgcttttgccattactcgggcacagatttcactgctggaacgccttgaaacgcagtccgccgggcgggcaaaaatctgccggacggtcggtgaaattgaagcgtgtattacacagaatgtgctggcgatggtgatgcatatcgaaggggcggaagcactcggcgatgatttctcgcggctggagcgctggtatgaaaaggggctgcgcagcatcggcccgttatggaacttacccaaccagtttggtaccggcgttaagggcgatttcccgggatcaccggataccggagatggcctgacgcccgccgggcttggattgctgcatgaatgtaaccggaaaaggattctgttcgatgtctcgcacatgaacgaaaaagccttctggcagacggcaaaattcagcgatgcgccgctggttgccacccattcaaatgtgcacgcgttatgtccgcaaccgcgtaatctgaccgataaacaactggctgccattgccgaaagcaacggcttcgttggcgttaatttcggcacggcttttctgcgggcggatggaaaacgcaacggcgacacccccatcaccgaaattgttaaacatcttgataaccttgttggtaaactgggggaagaaaatgtcggttttggttcggatttcgacggtatcaatgtgccggatacgctcggtgatgtcgccggattaccgttgctgcttcaggctatgtctgatgcgggatacggcgatgcattgatcgaaaaaatcgcgtaccgcaactggctgaaagtattaaagcaaacctggggtgaatag

SEQ ID NO: 37 - DNA, 912 bp, Rahnella aquatilis
atgcagattatcgtacttggttccgcggcaggcggcggcttcccgcagtggaactgcaattgcagcaactgtcagggtgtgcgtaatggcaccatgaaaacgtccccccgcacgcaatcttcgattgccgtcagcgacaatggcaccgactgggtgctgtgtaacgcctcgccggatatttgccaccagattgccgccacgccggaactgataaaacaagacgttttacgcggcaccgccattggttccattatcctgactgacagccagattgatcactgcaccggcctgctgaatttgcgtgaaggctgtccgcatcaggtgtggtgtacgccggaagtccacgaagacctcaccaccggtttcccgattttcaccatgctttctcactggaatggcggcctgcaacaccacgctatcaggccggagaaccgcttctccgttgccgtctgcccgaatcttacattcactgccattccgctgctgagcaacgcgccaccgtattcgaaataccgcggcaaaccgctccccggccacaatatcgcgctctttattgaagacacaaaaaccggcacctcgctgctgtacgcaccgggtctgggcgaaccggacgatgaactgctgaaatggctgcataaagccgattgcctgctgattgacggcacgctgtggcaggacaacgagctggcgaccaccggcgtcggccgcaataccggcaaagacatgggccatctggcgcttgccgaagaacaagggctgatcgccctgctgtcgtcacttccggcaaaacgcaaaattctcatccatattaataataccaacccgatcctcaatgaatcctctgccgagcggcaggcgctgacgcaacaaaacatcgaagtcagccgggacgggatgcgcatcgaactgtag

SEQ ID NO: 38 - DNA, 768 bp, Rahnella aquatilis
atgagcatatcgacaacacagacgtcaccgatgacgccgcaagaatttgaacaggcgctgcgtgccaaaggcgcgttttatcacatccatcatccctaccatattgcgatgcataacggtcaggcgacccgcgagcaaattcagggctgggtggcgaaccgtttctattatcagaccagcattccgctgaaagacgcggcgattatggccaactgcccggatgcgcaaacccgccgtaaatgggtgcagcgtattctcgatcacgacggacatggcggcagtgaaggcggtatcgaagcctggctgcgtctgggcgaagcggtggggttagaccgcgatgtgctgctttcagaagaaagggtgttaccgggggtgcgttttgcggtcgatgcctacgtcaattttgcccgccgcgccgtctggcaggaagccgcgtgcagctcgctcaccgaactgttcgccccgcaaatccatcaggcgcgtctcgacacctggccacagcattacacatggattgaggaagaaggttacggttatttccgcagccgcctgagccaggctaaccgcgacgtcgaacacggcctgcaactggccctggagtattgcgataccgtcgaaaaacaacagcgcatgctggaaatcctgcaattcaaactcgatattttgtggagcatgctcgattccatgagcatggcctacgaactgaaccgcccgccgtaccacagcgtgacgcagcaggcggtctggcataaaggaagactcctgtga

SEQ ID NO: 39 - DNA, 288 bp, Rahnella aquatilis
gtgatcaccattaccgaacactacacgccgatgtttcgtcgcggctaccgcatgcagtttgagaaaacgcaggactgccatgtgattttgtatccggaagggatggcgaaactcaacgacagtgcgaccttcattttacaactggtggatggcgggcggacaattgccaatattattgatgaactgaatgcccgctttccgcaggccggtggcgtgaatgacgacgtcaaagacttctttgctcaggcccatgcccaaaagtggattatcttccgtgaacctgcttaa

SEQ ID NO: 40 - DNA, 1134 bp, Rahnella aquatilis
gtgaacctgcttaaacctgcggtcaaaccgccgctgtggttgctggccgaactgacctaccgctgtccgctgcaatgcccgtattgctcgaacccgctcgatttcgctaagcaggaaaaagagctgaccaccgcgcagtggatcaaagtattcgaagaagcgcgggaaatgggcgcggtgcaaatcggtttctccggcggcgaaccgctggtgcgtaaagatttgccggagcttatccgtgccgcgcgggatctcggtttttacaccaacctgatcacctccggcatcgggctgaccgaaaagaaaattgatgcttttgctgaggccggactggaccatatccagatcagttttcaggccagcgatgaaacgctgaacgccgcgctggccggtaacgccaaagcgttccggcaaaaactggtgatggccaaagcggtaaaggcccacggttatccgatggtgctgaactttgtgctgcaccgtcataacatcgatcagatcgacaagattatcgacctcagtatcgaactggaagccgatgacgtcgagctggcgacctgtcagttttacggctgggcacagctcaaccgcgaaggtttgctgccgacccgtgagcagatcgcccgtgccgaacaggtggtgcatcagtaccgcgaaaaaatggccggaaccggtaatctggctaacctgttgtttgtgacgccggattattacgaagagcgtccgaaaggttgcatgggcggctggggggcgattttcctcagtgtgacgccggaaggcatggcgctgccgtgccacagcgcgcgccagttgccggtggaattcccgtcggtgctggaaaatacgttgcaggaaatctggtacgactccttcggctttaacaaataccgcggcttcgactggatgccggaaccctgccgctcctgttctgaaaaagagaaagactttggcggctgccgctgtcaggcatttatgcttaccggcaatgccgacaacgccgatccggtatgctccaaatctgagcatcacggcaaaatcctcgccgcgcgcgaacaggccaactgcaccaacattcagatcaaccagttgcagttccgcaaccgcgtgaactctcagctgattttcaaaggctga

SEQ ID NO: 41 - DNA, 5334 bp, Rahnella aquatilis
  modified_base (4721)..(4721): a, c, t, g, unknown or other
atcatcttctacaaaaacgattttattcattggcttgctgctttaagaaatccggtgattagcatagcgtgacccgcaggacgacgctatcgtgctagcgtaatagaacatgtcagtttccgttacgcaaagcaccggaagcgactggcaaagtgtcagtgaattatgccattatatagctgaatatcttcggcgttatacattgattaaataattgattgaccctgaccaggaagaggaagaggggatgaatgaaaaccgcctggtacccgatgcgcgcagtcaacaagcctcctttgactacatggaatacttgtccgcatcctgcaaaaagaaatggagttttgttgacgctatttatggcgtgatgccgttctttggcatggttctgaaatcccgttcgataaaagaaaaatcccggcaggaagcgttgcgcgcactggccttacaggtagtttccacccaggtcagcgacgagaccaatattgtccggctgattgaactggcagagcagcaaaatatgtacaacattgatatcaacctgccgtactcgctgaccgaagaacagctcactgcaataaaagtggaatgcaaacatctggtgcaattaagccagaataatgatcatctgttagtacagattatgcagatgccttcccggcactgatttctcggtgatgcattcaaaataaaggccgctttcgcggcctttatcatttcagtcgtttgccgtttatttcttcagttcggcgcgtaagtctttcacgtctgaggcagaaaccggcgcggcggcatttccccagctgttacggatatacgtcagcacgttagccacatcggcatcgctcatattggcatcaaacgatggcatcgacggcgcggtaggtttgctgtcggtagccacagggcggctgcccgtcagcaccacgcggataagcgacgtggcgttggtctggttgatgagcggtgaatctgccagccgtgggaacagaccttcctgacccataccgcctggcgtatgacatgcggaacaccggtcagcgtagatattcttcccggcaatcattgccttatcgtcagctttcactggcgcaggggcgttattgccgctgtcatgaccgctgtctttcagatagaccgccacggcttcaaggtcggcatccgtccagtgccgggatgagttcgtcacttcctccgccatcggaccggaagcaatatcgaaacggttagaaccggttttcagatactgcatcagatcttcctgcgtccagttaccgacaccagtgtgcttgttgctggtgatgtccggtgcgacccagttttctatcaccgccccttgcaggaactcgctgtccttatcgccgccgagcatatttttcggtgtatgacaggtaccgcagtgtcccagaccttcaacgatgtaggcgccgcggttccattgcgccgatttatccatctgcggtttgaattcgcctttgctgaagttcagccagttccagcccatcagactggtacgcacgttgaacgggaacggcaactggttagtttcgatttcgttgtgcaccggttgcagggtctgcaaatacgcccagatcgccgcgttatcttcgcgggtcactttggtgtacgccgtgaacggcatcgcaccgtaaagacgtttgccgccgtggccgatgccttcggacatcgcgcgctggaagtcatcaaaactccatttaccgataccggtttgcacatccggcgtaatgtttgcaccgactaaacggccgaacggggtttcaattggcacaccgccggcaaaaggtttggcgtccggcgtggaggcagtatgacaggcggcgcagtcgcccactgtcgccagataacggccacgctcaacctgttcgaaagagccgtcgcccgccgcgtgggccagcccgctgatgctcatggccagcaaaccgagtgagaatgaggttaactttttcatgcgatcatctctcccggttttttcagatagtaatgacggatcgcatgcgcggctttgaatgccagcgcacccacggtaccggtcgggttgtaacccggattttgcgggaaggcagacgcgccgaccacgaacagattaggcacgtcccagacttgcagatgtttgttcactgagctggtggacggatccgcgcccatcacaaaaccgccgacgacgtgggatgactggtacggcgtaccgttccagtgacctttcatcgggctggcgaccagctgacgcggattcatcgctttggcgatttcggccactttaccggtgcaatacgcggccattttcaggtcattttccgggaagtcgaacgtcacgcgcaacagcgggcgacccagacggtctttgtagttcggatccagcgacagatagttggttttggtggtgtagctgctggcctcacaaccgatcgacatcgagctgaggtagttggcgaccgtcgcttttttccactcgctgccccatttcggcgttcccggcggaacagggcggtagccgatgggggcgccgccgatgggggtcacgcggatactgccgccgccaaagaagcccagaccgctgtggtcaaagttgtcattgttgaaatcgtcaatgcccatcccgaccgcacctgcgccgatgaacggattaaagttcttgtcatcaaagaacaactgcacaccgttagcggtctgataggcataattacggcccgtggtgccgcttaacgtgaccggatcgtaaggtttgccgatgccggaaagcagcatcagacggacgttttcgaaggtaaacgcactcacgatcaccagatcagccggttgttcccattcatcgccggaagaatcgatatagaccacgcctgtcgcgcgtttgccggtggaatcggtcagcacctgcatcacttcacagttggtacgggcttcgaaattctccttgcggatcagtgccggtaatacggtagtgatagcgctggccttggaatagttcgcacagccgaagttggtgcagaacccgcaaaatgtacatggccccattttcacgccgagcggattggtgtaaggctcggaaatcagggaagagggtacagggaacgctttataacccatattgcgcgcggcttgcgcaaacaaggtcgggccataaggctgatccagcgggcgggtcggatattcaatggagcgcatcccttcatacggattgccgccttcatgatgttcgcctttgacgttgctggctttaccggaaacgccggccagacgctcgaaagaggcgtaatgcggttccatttcttcccagtcggtgccccagtcttgcagcaccaactcttccgggatggcattagcgccgtaacgttcggtcagatggcttttcaggcggaattcatccggctgaaaacggaaagtaatccccgcccagtggttgcccgcaccgccggtaccgtttcccgggtggaaagaaccccattcgcgcatcggtaatgccgtctgcgccgggttgtttcggatagtgatggtgttctggcgggtgcgtaacatcagttcctggcgggagctgtagcgtaactcatccgtcaccgtcgacacgttgaaatcacgggcggtatcacgccacggaccacgttcaatcgccaccacattcagaccttcgtcacacaattcattggcgatgatcgaacctgcccagccaagcccgatcaccaccacgtctgttttgggtaatttttttgccataatgttgttctgcccttagcttttcattttccatgcgtcgccgccggaaatactcagcggttcgagatcgagtttctggttatgtttgccgatatattcgcggtagtcgtagcgcgcgccggggaagcccagcattttccacgacaccatattgcggttaccgccataaatcgggtcagcaaagaaaccttccattgtgttcttcagtgcgatggcgaagaacaatttagaatcgatcccttcaagggcaatttttccgctctcaagacctgacaggatttgatcctgctggtcgccgggcagatctttgaataatttctgatgctgtgaccgggcatatttatccaatgcggccaggccaagacggtagcgttgttgcggcaccagcggcgactgatcgccttgctccggagtgccctcctggaaagggccttgcatataaaggcgggaataggtgccgtagaatccggccagctgacggtcgataaacaccgcgcatccggcgtctttaccgcccacgctcagatcatcggcagggaccagacggtcaacaatggcttccatggcagcggcttcggcttcggtgaaaaacacccagccatcggtctcaatctgcggcggcgggctggcgtcaaaggggctccacacgggcgtgcctttgagcgtcaccgcgttggcggctttggcggcacccagtactgcggaggcggccgtgaacgagagaatctgtctgcgggtcacgccgggcaaggtttttttgctcatgtatgttccctgtttgtatacatcactttaaggtccggacgcgctctgcaaaggcagtcgggtggcgtcgggataatcgttaacctgctaaggataaatgaaaacagactgtaaagataaatggttcgtttgttaaaaatgttattaaatgtgtgcaattgatatctttgatgtaattttaggtgatatttttgtgatgtggtgacatgttatccatgtattcatcntggaaatgaattttcatgatttcacctaatacaacgatgtatgtggccgttcgtggcatgcaggttgaattatgctgtctgtacgttaagatcaaaccttactcataaaatgtgcaaggtatgactgaagtcacagattttttttctgatttattgaagcgatttgcaggaaatccctgcgcctctttgcttattgccgggggagctgccttctatgctgtcagcgccagtgcgctgacggcagaacccaccgaatatggcgtttccggttatcacctcagcggttttctgcctgccagtgacgaaagttccgtttttaataacggagccccggcccaaccttcccggatgttacccagcctcggcagccagtatatacaaaccaataccggaccgacggccacggaaaaacagctcgcaaccacgctgaaagagctgggcaccaccaccacagacagcgatgatcccttacgtgtgcaggcggagacgctggcactcagtcaggcggcgaaactggtgcaaaaagaaaccagcagtttgctttcgccgctgggtaccgtcagcacctcgctggatatgagcggccgtaatcttgacggcagctccgggcggc

SEQ ID NO: 42 - DNA, 500 bp, Rahnella aquatilis
tgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 43 - DNA, 170 bp, Rahnella aquatilis
tacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaac

SEQ ID NO: 44 - DNA, 142 bp, Rahnella aquatilis
aattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccct

SEQ ID NO: 45 - DNA, 293 bp, Rahnella aquatilis
ttaaaaacgtgaccacgagcattaatgaacgctgcgaaatgtggcgtttatttattcaaaaagtatcttctttcataaaaagtgctaaatgcagtagccgcaaaattgggataagtcccatggaatacggctgttttcgctgcaatttttaactttttcgtaaaaaaagatgcttctttgagcgaacgatcaaaatatagcgcttaccgacaaaaaattattctcattagaaaatagtttgtgtaatacttgtaacgctacatggagattaacttaatctagagggttttata

SEQ ID NO: 46 - DNA, 1786 bp, Artificial Sequence
  source: /note=“Description of Artificial Sequence Synthetic polynucleotide”
agaggttttacgacggtctcatcgctcggggaaagttaaagaaagttgcgctaacagcatgcatccggaaactggtgacaatcctgaatgcgatggtgcgagatggccgtgaatgggcagcaaattaatcgctgccggattgacattcgacacagctgctcccccttcgcagggggaggagaaagtcttacttctgtaccgtcttcaaagccgcaatacaacgatctaccacaccttcaaacttatggtcgatattcacccgcaacacatccggttcgtcttcgcccggttcttcgagcgcatcgaactggctgcgcagtaaatcggtcggcatgaaatgtccggcacgcgctttcaggcgctcggcgatcacttcaaaactgcctttcatgtagatgaataccatgccttcgttgtctttacgcagggcgtcacggtagcgacgtttcagtgcggaacagacaatgatgccggtttcatttttatggcgcaggctgtaagccgcgtcgttgaggcgcaataaccacggcgcgcggtcatcgtcgttcagtggcgtgccgctggccatcttttgaatattggcgcggggatgaagatcatcgccgtcgataaatttcgcgttaatttcacgggccagtgccgcgccgacggtggatttaccgctgcctgacacgcccatcagaatgatgctttgtcctgccataagtatgatctctatcccaaaatgaacattttgactgcgtagtgagtcctaatattaccatgttaccggtatcatgataccggtaacaaatggagatgtgactaatatcacaaaggaatgcactgctgttttagcggtcgcggtctgtacccgtagcactgttaattcctacaacatcttgaaaattaaaacaatgcttcacccggtttttagccatacccgggtgacagcgtgaggggaagaatcgacaaattgttagcataaaaaggcgagccagacggctcgcctttttcgtttcttctgcctgctaaatcttcagatacgccctgatcccatcgaggaacatctgcgttgagagcatgaccagaatcagacccatcaggcgttcgagggcgctgacgcctttgtcgccgagcaggcgcagaaacaggtttgagagcagaaggatcgccatcgacatgccccaggcgataaacagcgccagcgtgagatgactcatctgattcgggtactggtgcgaaagcagcatcagcgtcgcgagaatcgacggaccagcaaccagcgggatggccatcggtacgaggaagggttcttcaccggcaggcaggccactgctgcttttctcttccgacgggaaaatcattttgatggcaatcaggaacaggataataccgccggaaattgacacggtttcggtacgtaaattcaggaatgacaggattttttccccggcgaacaggaaaatcagcatcaggatcagtgcgatcagcatttcgcggattaacacgacccggcgacgtttcgggtcgagatgttttaaaaccgacataaaaatcggtaagttgcccagcggatccataattaaaaacagcaaaatggttgctgaaatcatttcggtcatgactacctcagaaaaaatgcctgttataagttgcgctgctattaaaagctgcactattgattaaattcacttgccacttctactacattttgtaaggtggtagaccagcgcgttaacggggcacagtgcatcaaaacaggcactaacttagtgcaaatgcgcgttcaagccgttttaagcag

SEQ ID NO: 47 - DNA, 4740 bp, Artificial Sequence
  source: /note=“Description of Artificial Sequence Synthetic polynucleotide”
tgggaagccccggagagaaagagccaaatgctcattgatgatttttgaaagttcgcgttgttcgtccggcaatatggcgttgaacactgcgtgaacctaccctgttcacgcataatgatatgtttatttacttgatatttccgctctttattactttcgaaagtgatatttccggcgcgggaatcccaataaaaaaagcctgaatttcttcaggcccgatctttgccatcctgatgacagattactggaagtaaatatcgccgttttcggctttgaggatcttgccgtcggtgtcagtgatcagtgcgtagctgccgccgatataagtccagtggctacccgcatccggtgccggcaggtgacgttcctgccagccgacgatctcatacggcttggtacggtacagcgcagggacctgatcgccgatggaatacaatttggaatcatcgtagaacgatttgatctcgtgtgagtttgacgcggcaaacgccgtgttggccaggggtaacaacgatgaaacaaccagggctgataacataacaattttagaatgacgcatagattatcccgttaaaaccatcaacttaatgcctgcctcggtgatatgccgccagtccgttgtatgagcagtaaagccgaagcctgtgctttttctgtaggacaggataataacgagaaagtgagtgcaggctgggagatttttttgtgttgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatcatgcgcaatgaacggttttttagctggtcgttcatgaccgggcttgtcacggctgtcatcgggctggcttatctggtgctgggcgtctggctggcggccctcggcggctcaccctggtatgtgctttttggcagcggatatctgctcagcggcatttttatcgcgcgccgccatgccagcgggatctggctttatctgctgacatttctgctttgctgcgtctggtcggtatgggaagtcggactggatggctggcaactgatgccgcgtttgtttgtgatggcgttattaggcgtctggtgcagcctgccgttgatcacccgtcaggtgatggcaacgcggggcaatcaccgcaccggtacgttcgccgggctggtgtatgtggtggcgatcgtgggtattttttacagcggctggcaggtgacagggtcacgttttgtccatcgtcagcctgttccggcgcaatcgggtgatatacaggccacgtcgccggaaagcaacgactggcgctattacggcaggacagaagccggacaacgttattcgccgctgacacagattacacccgctaatgtcagtcagcttaaacccgcctgggaatttcacaccggcgatgtgatgagaaagggtgaggataaagacggacgggaatttaatttcgaagtcacaccggtcaaagtcgggaactcgctgtttatctgtacgccgcaccgcgaagtgattgcgctaaacgccaccaccggcgcgcagcgatggaaattcgatcctaaatctgacacctcggccaatgagtatctggcatgtcgcggcgtggcatacagccagtcagccggtgataaagtgtgtccggaaaaaatcatcgccaccaccagtgaggcgcggatggtggcgctgaatgcacagaccggcgaaccctgttcatcctttggtcagaacgggtttgtcagcctgaccgaccatatgggcgacgtgccgccgggcttccacttcattacctcgcagccgatggtgatggatggccgcatcgtgctgggcggctggatttatgacaaccagtctacgggtgaaccttccggcgtcgtccgggctttcgatgtgaacaccggccagcttgcatgggcgtgggatatgggccgggatccgcaaaatgcgccgctcaaaccgggtgaagtgtatacccggggcacgccaaacggctggggaacctataccggtgatccgaaactggggctggtttatattccgctcggcaatgcgacgccggattattacggtgccggacgccgtccgtttgacgaaaaatattcgagttcgctcgtcgcgctggatattcacaccggcgaggaacgctggcacttccagactgtgcatcatgatgtctgggactttgacttgccgatcggaccgacactggtggatttgccgtcaccggaggggataaccgtgcctgcgctggtgcagaccaccaaaatgggacagcttttcctgctcgaccggcgtaccggcaaaccgctggcgcaggtaaacgaaaaaccggtgaatacctctccctctttgccgggcgaacatctgtcgccaacgcagccggattctgtcggcatgcccagtctctctccgccagatctgaaagaaaccgacgcgtggggcgcgacaccgattgatcagttgtattgccgcatccagttcaaaagtgcccgctatcaggggcagttcaccccgccagcggaaggtaaatccattgcttatccggcctttgacggcgtgatggactggtacggcgcttcggtggatccgatccgccatgtgctgattgccaataccagttacatcccgttcacgatggaagtgaaaaagtcagccgatgcgatcaaagaagggctgatgcacaaatgggccggatggggcagcaaccagccttatccaaaacccaaagagttttcggttggcccgcaatatggcacgccgtgggcggcgatcgtcaaaccgtggctgagttttctgcaggcaccctgtaatgcgccgccgtggggaaaactggttgcggttgatctgaccacccgaaaaatcgcctgggaaagaccggcaggcacgacccgggatatgaacatttttggcacgcataccaacgtgccattgccgaccgggatttttatgatgggcggtaacatcattacccaaagtggcctgattttcaccggcgcaacggcagacaactatttccgcgcattcgacgaaacgacgggtaacgaactctggcgagcgcgacttccggcgggcgggcaggcgacgccgatgacatataccggcgatgatggccgccagtttgtggtgattgccgccggcggacacggcgggctggggacgacgtccggtgatgcgctggtggcgtatgcattaccggccagatagggttggcactcaaacgcaggatattcagaaccgtctgatgatgcccacaggcggcgcagcatggtctatttttaagatcccttcttttttaaataaggagagtttatgaacaacaaaacaatcccgacatccctgaatgaacctcatgttgatgcgtatccgaccccgccgtttgagcatcagaaacagccattcccggggctggccagcaaaatgaacccggtacccgatcacggcgagaaaacctacaaaggcagtgcccggctggaaggccgtaaggcgctgattaccggcggtgactccggtattggccgtgcggtggcgattgccttcgcccgtgaaggtgcacaggtcgccatcaattatctgccggacgaagaggccgatgccaaagaagtgatcgacctgctgtgggccgaagggcggaaagtgattgccattccgggggatattcgcgacgagaaattctgtcagcaactggtgaaggaagcggaagagaaactcggcgggctggatctgctcgtcaataacgccggtcgtcagcagttctgtgattcgattaaggatctgaccaccgaagccttcgacgccacgttcaaaactaacgtgtacgccatgttctggataaccaaagcggcgctggaattcattccgcgaggcggtgcgatcattaatacgacttctgttcaggcgttttcaccgagtgataatttgctcgattactcctccaccaaggcctcgatcatggccttcaccaaagggctggccaaacagctggcgggggacggaattcgggtgaacggtgttgcgccggggccgtactggacgccgttgcaaatttccggcggacagcctcaggaaaaaatcgaatcctttggtcagcaggcaccgctgaaacgtccgggacagccagcggaaatcgccccgctgtacgtgacgctggcctcaaatgaaaacagttatgcctcagggcaggtctggtgttctgatggcggtaccggcacggtctgattaccggtcggaaacgccaaagcccgtcatattgacgggcttttttcatgcaggcagatatccggcgttttttgccgacatcggcacgtcataaataattacgttgctaat

SEQ ID NO: 48 - DNA, 4382 bp, Artificial Sequence
  source: /note=“Description of Artificial Sequence Synthetic polynucleotide”
tgggaagccccggagagaaagagccaaatgctcattgatgatttttgaaagttcgcgttgttcgtccggcaatatggcgttgaacactgcgtgaacctaccctgttcacgcataatgatatgtttatttacttgatatttccgctctttattactttcgaaagtgatatttccggcgcgggaatcccaataaaaaaagcctgaatttcttcaggcccgatctttgccatcctgatgacagattactggaagtaaatatcgccgttttcggctttgaggatcttgccgtcggtgtcagtgatcagtgcgtagctgccgccgatataagtccagtggctacccgcatccggtgccggcaggtgacgttcctgccagccgacgatctcatacggcttggtacggtacagcgcagggacctgatcgccgatggaatacaatttggaatcatcgtagaacgatttgatctcgtgtgagtttgacgcggcaaacgccgtgttggccaggggtaacaacgatgaaacaaccagggctgataacataacaattttagaatgacgcatagattatcccgttaaaaccatcaacttaatgcctgcctcggtgatatgccgccagtccgttgtatgagcagtaaagccgaagcctgtgctttttctgtaggacaggataataacgagaaagtgagtgcaggctgggagatttttttgtgtaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctatgcgcaatgaacggttttttagctggtcgttcatgaccgggcttgtcacggctgtcatcgggctggcttatctggtgctgggcgtctggctggcggccctcggcggctcaccctggtatgtgctttttggcagcggatatctgctcagcggcatttttatcgcgcgccgccatgccagcgggatctggctttatctgctgacatttctgctttgctgcgtctggtcggtatgggaagtcggactggatggctggcaactgatgccgcgtttgtttgtgatggcgttattaggcgtctggtgcagcctgccgttgatcacccgtcaggtgatggcaacgcggggcaatcaccgcaccggtacgttcgccgggctggtgtatgtggtggcgatcgtgggtattttttacagcggctggcaggtgacagggtcacgttttgtccatcgtcagcctgttccggcgcaatcgggtgatatacaggccacgtcgccggaaagcaacgactggcgctattacggcaggacagaagccggacaacgttattcgccgctgacacagattacacccgctaatgtcagtcagcttaaacccgcctgggaatttcacaccggcgatgtgatgagaaagggtgaggataaagacggacgggaatttaatttcgaagtcacaccggtcaaagtcgggaactcgctgtttatctgtacgccgcaccgcgaagtgattgcgctaaacgccaccaccggcgcgcagcgatggaaattcgatcctaaatctgacacctcggccaatgagtatctggcatgtcgcggcgtggcatacagccagtcagccggtgataaagtgtgtccggaaaaaatcatcgccaccaccagtgaggcgcggatggtggcgctgaatgcacagaccggcgaaccctgttcatcctttggtcagaacgggtttgtcagcctgaccgaccatatgggcgacgtgccgccgggcttccacttcattacctcgcagccgatggtgatggatggccgcatcgtgctgggcggctggatttatgacaaccagtctacgggtgaaccttccggcgtcgtccgggctttcgatgtgaacaccggccagcttgcatgggcgtgggatatgggccgggatccgcaaaatgcgccgctcaaaccgggtgaagtgtatacccggggcacgccaaacggctggggaacctataccggtgatccgaaactggggctggtttatattccgctcggcaatgcgacgccggattattacggtgccggacgccgtccgtttgacgaaaaatattcgagttcgctcgtcgcgctggatattcacaccggcgaggaacgctggcacttccagactgtgcatcatgatgtctgggactttgacttgccgatcggaccgacactggtggatttgccgtcaccggaggggataaccgtgcctgcgctggtgcagaccaccaaaatgggacagcttttcctgctcgaccggcgtaccggcaaaccgctggcgcaggtaaacgaaaaaccggtgaatacctctccctctttgccgggcgaacatctgtcgccaacgcagccggattctgtcggcatgcccagtctctctccgccagatctgaaagaaaccgacgcgtggggcgcgacaccgattgatcagttgtattgccgcatccagttcaaaagtgcccgctatcaggggcagttcaccccgccagcggaaggtaaatccattgcttatccggcctttgacggcgtgatggactggtacggcgcttcggtggatccgatccgccatgtgctgattgccaataccagttacatcccgttcacgatggaagtgaaaaagtcagccgatgcgatcaaagaagggctgatgcacaaatgggccggatggggcagcaaccagccttatccaaaacccaaagagttttcggttggcccgcaatatggcacgccgtgggcggcgatcgtcaaaccgtggctgagttttctgcaggcaccctgtaatgcgccgccgtggggaaaactggttgcggttgatctgaccacccgaaaaatcgcctgggaaagaccggcaggcacgacccgggatatgaacatttttggcacgcataccaacgtgccattgccgaccgggatttttatgatgggcggtaacatcattacccaaagtggcctgattttcaccggcgcaacggcagacaactatttccgcgcattcgacgaaacgacgggtaacgaactctggcgagcgcgacttccggcgggcgggcaggcgacgccgatgacatataccggcgatgatggccgccagtttgtggtgattgccgccggcggacacggcgggctggggacgacgtccggtgatgcgctggtggcgtatgcattaccggccagatagggttggcactcaaacgcaggatattcagaaccgtctgatgatgcccacaggcggcgcagcatggtctatttttaagatcccttcttttttaaataaggagagtttatgaacaacaaaacaatcccgacatccctgaatgaacctcatgttgatgcgtatccgaccccgccgtttgagcatcagaaacagccattcccggggctggccagcaaaatgaacccggtacccgatcacggcgagaaaacctacaaaggcagtgcccggctggaaggccgtaaggcgctgattaccggcggtgactccggtattggccgtgcggtggcgattgccttcgcccgtgaaggtgcacaggtcgccatcaattatctgccggacgaagaggccgatgccaaagaagtgatcgacctgctgtgggccgaagggcggaaagtgattgccattccgggggatattcgcgacgagaaattctgtcagcaactggtgaaggaagcggaagagaaactcggcgggctggatctgctcgtcaataacgccggtcgtcagcagttctgtgattcgattaaggatctgaccaccgaagccttcgacgccacgttcaaaactaacgtgtacgccatgttctggataaccaaagcggcgctggaattcattccgcgaggcggtgcgatcattaatacgacttctgttcaggcgttttcaccgagtgataatttgctcgattactcctccaccaaggcctcgatcatggccttcaccaaagggctggccaaacagctggcgggggacggaattcgggtgaacggtgttgcgccggggccgtactggacgccgttgcaaatttccggcggacagcctcaggaaaaaatcgaatcctttggtcagcaggcaccgctgaaacgtccgggacagccagcggaaatcgccccgctgtacgtgacgctggcctcaaatgaaaacagttatgcctcagggcaggtctggtgttctgatggcggtaccggcacggtctgattaccggtcggaaacgccaaagcccgtcatattgacgggcttttttcatgcaggcagatatccggcgttttttgccgacatcggcacgtcataaataattacgttgctaat

SEQ ID NO: 49 - DNA, 4334 bp, Artificial Sequence
  source: /note=“Description of Artificial Sequence Synthetic polynucleotide”
gattgttctggaacgtaaatactccggcgtagacacccgctggaaacatgacgatcagataggttccgtgccgttctcggttaccggcggtctggattatgaaaccatgaccgaacgtcgtttcggctatgagaacttcaacagtgaaggcgatctgggtgtgaaaggcgacgaacgccgtaatgaaaaaaacgtgatgtggaacctcgatccctatctgcaaaccacgtggaacctgacatcgcgctggacactcgatgccggtgcgcactacagcaccgtgagtttcgactcgcaggattattacattaccggcagcaacccggatgacagcggttcgcgccgttatcataaactgctgccgatggcgtccctgaactatgccgtgacgcctgcactgaacacctatatttctgccggtcgtggttttgaaacgccgaccattaatgaactgtcttaccgcaccgacggtaaatcgggcctgaatctggggctggaaccttccaccagtaccaccgtggagctgggcagcaaatggcgcgtgggcaacggtttggtgaccgccgccgtcttccagactgatactgatgacgagctgattgtggcacaaagtaccggcggtcggtcgagctacaccaacgccggtaagacacgccgtcgggggctggaactgtcattagatcagcagattgaagaaaactggcgcgtaaaaatggcctggacgctgctcgatgcgaccttccgcaacgagacctgtggcgcgggtgattgcacacccgcgggcaaccgtctgccgggtattgcgcgcaacatgggctacgcttctctggagtgggcgccggttgagggatggcacgcgggtgccgatatccgctacatgagcgacattgaagtcaacgacgaaaacagtgaacaggcaccggcatataccgtcgccagcgtgaatgcgggttaccgcttcaactggaacaacgtgacgctcgacttgtttacccgtgtcgacaatttgttcgacagaaattatgtcgggtctgtgattgtcaatgaaggcaacggccgttatttcgaaccggcacctggcagaaactacggtggcggagctacgctttcttatagtttcgaataatgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatcatggaaacgaaagcctcgttatcacgcattgtcgtcataattaccgctttgttcgccgcgttaagcgggatttaccttcttgccggtggtatctggctggcaaaattaggaggttctctttattacatcattgccggtgttatttcgctggttactgcgtggctgctttaccgtcgccgctcttcggcattattgctctatgccatcttcctgttcggcaccactgtctgggctgtatgggaagtgggcacagacttctgggcactgacgccgcgtctggacgtcaccttcttcctgggcctgtggatcctgctgcccgtggtttataaccagatgctggcgaaaaacgcctttgcacgcggcgcgctggcagtttctctgctgttcaccgtgatcgtgctgggctacgccatctttaacgacccgcaggtgattaacggcaccatcaaagcggcggattctgctccggcaaaatctgagtccggcatccctgatggcgactggccggcgtatggtcgtactcagggcggtacccgttactcgccactgaaccagatcaacgataaaaacgtcagcaagctcgacgtggcctggactttccgtaccggtgacctgaagaccccgaacgatccgggcgaaatcactgacgaagtcacgccaatcaaaatcggcgacatgctttatctgtgtacgccgcatcagaagctgtttgctctggatgccgcaaccggtaaagagaagtggaagttcgatcctgagctgaaacccaacccaaccttccagcacgtgacctgtcgtggtgtgtcttatcatgagaccacaccagccgcagaaggcaacgccaccaacggcgcggcgcctgctgtctgttcccgtcgtatcattctgcctgtcaacgatggccgtctgtttgcgctggacgctgaaaccggcgcacgttgcccggcatttggcaacaacggtgagctgaacctgcaaggcaacatgccttatgcaaccccaggccactacgagccaacttcaccacctatcatcaccaaatctgtgatcatcgtggcgggtgcggttaccgataactactcaaaccgcgagccttccggcgtgatccgtggttttgatgttgagaccggtaaactgctgtgggccttcgatccgggtgcggctgagccgaacaaaatccctgaggatggtcagcatttcacgccgaactccccgaactcatgggcacctgcggcttatgatgacaaactggatctggtttacctgccaatcggcgtggcaacccctgatatctggggcggcaaccgtactccggaaatggaacgtttcgcgagcggcctgctggcgctgaatgcgaccaccggtaaactggcctggttctatcagactgtgcatcacgacctgtgggatatggacgtgccagcgcagccaacgctggctgatatcactgacaagagcggcaacaaagttccggcgatttatgtaccgaccaaaaccggtaacatcttcgtgctggatcgccgtgacggtaagctgattgttgatgcgcctgagaaacctgttccgcaaggcgcggcgaaaggcgaccatgtgtctccgacccagccattctccaaactgactttccgtcctgaagccaaactgaccggtaaagacatgtggggcgctacgatctacgaccaactgatgtgtcgtgtgatcttccacaaactgcgttatgaaggcaccttcacgccgccatccgagcagggcactctggtgttcccgggcaacctcggtatgttcgagtggggcggtatttctgtggatacagaccgtcaggtggctatcgcgaacccgattgcgctgccattcgtgtctaaactgatcccacgcggtccgggcaaccctatcgagccagatgcgaacgataaaggcggttccggtactgagactggcattcagccgcagtacggcgtgccatttggcgtgacgctgaatccgttcctgtctccgctgggcttcccatgtaaacagcctgcatggggttacatttccggtgttgacctgaaaaccaacgatatcgtgtggaaaaaacgtatcggtaccgtgcgcgacagctcaccattaccgctgccgttcaaaatgggtatgccaatgctgggcgcaccggtttctaccgccggtaacgtgttcttcatcgcggcaaccgcagataactacctgcgcgcgttcaacatgagcaacggtgacaaactgtgggaagcacgtctgccagcaggcggccaggcaaccccgatgacttactccgtcaatggcaaacagtacgttgttatcgcggcaggcggtcacggttcgtttggcaccaaactgggcgattacatcatcgcttacgcactgccagacgctgatgctaaataattagcggtgttataagttctgaaatctgaaaggccgcgaaagcggccttttttacaactgctgcgcgggtaacattatctgtcacttgctgatgttcattaggttttcctcaggcacccaacctgattctccagatttgttgcatgcccagaaccagccgttgagctttttctccagcgtgaggacttcccccggagcaaccgataattcatatgccgtataattctcagtgcaaattccccggtccggagcactaagggtaaatatttgctggggcgcccatcctgcattgcctgaactctgacgga

SEQ ID NO: 50 - DNA, 3431 bp, Artificial Sequence
  source: /note=“Description of Artificial Sequence Synthetic polynucleotide”
cgcgggcgtttcaggcgctgcatggcagggatctgcgcattgtgcgccagactgcgctcgacggcttgctcagcgcgctgggcgggcatctcacctggcaaccgccgcgtttgcagggcgaagcgccgtggtcacggttggcgcatcaggcgcgggaatatctgcatgcccatctgcatgaagatgtcggcctcgaacaactggcggcggcaaccggtgaaagccgttttgtggtcagccgtgcgtttaaagccgaattcggattgccgccgcacgcctatcttattcagctgaaactgacccgcgcccgcgaagggctggcgctcgggcgtacaccggcagccgtggccgccgatttaggctttgccgatcagagccatctcgggcgctggttccgccgtgcgtatcagctcacgccagcgcattaccggcgtctgtgcacaaatcttccagattgagaaaaagtgacaggttaagctctgatttcaactgaaccggagcctgtcatcatgaataccaccgcaacaattctgccctcgtcccctgaacgctgcgtgattgtcatcaatcagcaacttgccgcaggacatgccgccaatgccgccgcggtcctggcgctgacgctcggacaacgccatccggcgctggtgggcgcaccgctgattgatgccgataaccgcgaacatccggggctgatcccgattggtatcagcgtgttagtcgctgatgctcaacaactcacacaacttcatcagcatctgctaaatgacgatgaaatggacggcattattttcccggtcgaagggcagcaaaccaccgattacgccgcctttcgtgaggcggtatcggttgtgccgaccaacaacctgcaactgctcggcatcgcgctggcggggaataaaaaagtggtgcgtaagctgaccggcaaactgggattactgggctgatgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatcgtgcgtaagtccctcgtcgctctgctgcttttcactctgggttccacctttgctgttcaggctactgaagaagccaaaccctttatcaccagtcaggaactggatctgacccaatatctgccagcgccaccggcggatgattcggcgcagacccaagcggagctgaaagaattgctccaaattcaggccacccgcacgccggagcaggaaaaagcggcgattgctgatgcgcaagaaaacgtctggcgttttgccgatgtgatggggccgggctttgatgccgagaaactgccgaaaaccgccgcgctgtttgagcgtattgtggcgacagaagacgtggtggacgatcacgccaagaaagcgtttaaccgtccgcgtccttatatgctggatgaacaaattcatccgctgctgaaaaagtctaaatccggttcatggccttccggtcattccaccatcggttatctgatggcgacggtgctgggcgaaatggtgccggaaaaacgcaatgcgctgtttgcccgtgcatccggttatgccgaaaaccgtctggtggctggtttccattaccgttctgataccgtcatgagccgcaccggtgccgcgctgattgctcagaaaatggaagaacagccagatttcaaaaccgaattcgacgcggcgaaagcggaactgcgtacgcagctgggcctgaaataattttcgtcagtttctgcctttccggtaaagtatggaacgtaaaacttgcagaggaagggactgatggaatacagattcaagcggatggcatcaccggtcgggttactcacactggcggcgaaaggcgacaagctgaccgccattttgtgggaatgtgaaatcgacgggcgcgtgcctttgggtgaaatgctggaagacccggcgtttccgattttgctgaaaaccgaacaacagctgaacgagtatttcgcgggtaaacgcacgtgctttgagctggaccttgatttcaccggcaccgcattccagaaggaagtctgggcggcgttgctggaaattccgtttggtgagacgcgcagctacggcgacatcgcccgccgcatcggccgcccgaaagcggttcgcgccgtgggcgctgccaatggtcgtaatccgatttctatcgtagcaccctgtcaccgggtgatcggttcttccggcaaattaaccggctttgccggcggactggagaacaagttgttattactgaggctggaaggccgtaaatcgtaaggttttttatgggggaataaattgcttgatgtccggttgtcagtcgatgcatagtcaaaaaataagcagtaaataaaaaagacaacatcacttacagacggagttttaccttaatggatcagattcaatccctcgaacagttcttatcctcagttcagcaacgcgacccgcatcaggtcgaattctcccaggccgtacgtgaagtcatgaccacgctatggcctttcctcgaacaacatccgcaataccgccgttccgcattgctggagcgtctggttgagccggaacgtgtcattcagtttcgtatcgcgtgggtggatgacacaaatcaggtgcaggttaaccgtggctggcgagtacagttcagttcagccatcggcccctataaaggcgggatgcgcttccacccgtcggtcaatttatcgattctcaaattcctcggcttcgaacaaactttcaaaaatgcgctgaccacattgccgatgggcggcggcaagggcggcagcgatttcaacccgaaaggcaaaagcgaaggcgagatcatgcgtttctgtcaggcgctgatgctggagctttaccgtcatctcgggccggatactgatgtgcctgcgggtgatatcggcgttggcgggcgtgaagtcgggtatatggcggggatgatgaaaaagctctccaacaataccgccagtgtgttcaccggcaaagggttgtcgttcggcggcagtctgatccgtcc

SEQ ID NO: 51 - DNA, 3101 bp, Artificial Sequence
  source: /note=“Description of Artificial Sequence Synthetic polynucleotide”
cgcgggcgtttcaggcgctgcatggcagggatctgcgcattgtgcgccagactgcgctcgacggcttgctcagcgcgctgggcgggcatctcacctggcaaccgccgcgtttgcagggcgaagcgccgtggtcacggttggcgcatcaggcgcgggaatatctgcatgcccatctgcatgaagatgtcggcctcgaacaactggcggcggcaaccggtgaaagccgttttgtggtcagccgtgcgtttaaagccgaattcggattgccgccgcacgcctatcttattcagctgaaactgacccgcgcccgcgaagggctggcgctcgggcgtacaccggcagccgtggccgccgatttaggctttgccgatcagagccatctcgggcgctggttccgccgtgcgtatcagctcacgccagcgcattaccggcgtctgtgcacaaatcttccagattgagaaaaagtgacaggttaagctctgatttcaactgaaccggagcctgtcatcatgaataccaccgcaacaattctgccctcgtcccctgaacgctgcgtgattgtcatcaatcagcaacttgccgcaggacatgccgccaatgccgccgcggtcctggcgctgacgctcggacaacgccatccggcgctggtgggcgcaccgctgattgatgccgataaccgcgaacatccggggctgatcccgattggtatcagcgtgttagtcgctgatgctcaacaactcacacaacttcatcagcatctgctaaatgacgatgaaatggacggcattattttcccggtcgaagggcagcaaaccaccgattacgccgcctttcgtgaggcggtatcggttgtgccgaccaacaacctgcaactgctcggcatcgcgctggcggggaataaaaaagtggtgcgtaagctgaccggcaaactgggattactgggctgatacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaacgtgcgtaagtccctcgtcgctctgctgcttttcactctgggttccacctttgctgttcaggctactgaagaagccaaaccctttatcaccagtcaggaactggatctgacccaatatctgccagcgccaccggcggatgattcggcgcagacccaagcggagctgaaagaattgctccaaattcaggccacccgcacgccggagcaggaaaaagcggcgattgctgatgcgcaagaaaacgtctggcgttttgccgatgtgatggggccgggctttgatgccgagaaactgccgaaaaccgccgcgctgtttgagcgtattgtggcgacagaagacgtggtggacgatcacgccaagaaagcgtttaaccgtccgcgtccttatatgctggatgaacaaattcatccgctgctgaaaaagtctaaatccggttcatggccttccggtcattccaccatcggttatctgatggcgacggtgctgggcgaaatggtgccggaaaaacgcaatgcgctgtttgcccgtgcatccggttatgccgaaaaccgtctggtggctggtttccattaccgttctgataccgtcatgagccgcaccggtgccgcgctgattgctcagaaaatggaagaacagccagatttcaaaaccgaattcgacgcggcgaaagcggaactgcgtacgcagctgggcctgaaataattttcgtcagtttctgcctttccggtaaagtatggaacgtaaaacttgcagaggaagggactgatggaatacagattcaagcggatggcatcaccggtcgggttactcacactggcggcgaaaggcgacaagctgaccgccattttgtgggaatgtgaaatcgacgggcgcgtgcctttgggtgaaatgctggaagacccggcgtttccgattttgctgaaaaccgaacaacagctgaacgagtatttcgcgggtaaacgcacgtgctttgagctggaccttgatttcaccggcaccgcattccagaaggaagtctgggcggcgttgctggaaattccgtttggtgagacgcgcagctacggcgacatcgcccgccgcatcggccgcccgaaagcggttcgcgccgtgggcgctgccaatggtcgtaatccgatttctatcgtagcaccctgtcaccgggtgatcggttcttccggcaaattaaccggctttgccggcggactggagaacaagttgttattactgaggctggaaggccgtaaatcgtaaggttttttatgggggaataaattgcttgatgtccggttgtcagtcgatgcatagtcaaaaaataagcagtaaataaaaaagacaacatcacttacagacggagttttaccttaatggatcagattcaatccctcgaacagttcttatcctcagttcagcaacgcgacccgcatcaggtcgaattctcccaggccgtacgtgaagtcatgaccacgctatggcctttcctcgaacaacatccgcaataccgccgttccgcattgctggagcgtctggttgagccggaacgtgtcattcagtttcgtatcgcgtgggtggatgacacaaatcaggtgcaggttaaccgtggctggcgagtacagttcagttcagccatcggcccctataaaggcgggatgcgcttccacccgtcggtcaatttatcgattctcaaattcctcggcttcgaacaaactttcaaaaatgcgctgaccacattgccgatgggcggcggcaagggcggcagcgatttcaacccgaaaggcaaaagcgaaggcgagatcatgcgtttctgtcaggcgctgatgctggagctttaccgtcatctcgggccggatactgatgtgcctgcgggtgatatcggcgttggcgggcgtgaagtcgggtatatggcggggatgatgaaaaagctctccaacaataccgccagtgtgttcaccggcaaagggttgtcgttcggcggcagtctgatccgtcc

SEQ ID NO: 52 - DNA, 3073 bp, Artificial Sequence
  source: /note=“Description of Artificial Sequence Synthetic polynucleotide”
cgcgggcgtttcaggcgctgcatggcagggatctgcgcattgtgcgccagactgcgctcgacggcttgctcagcgcgctgggcgggcatctcacctggcaaccgccgcgtttgcagggcgaagcgccgtggtcacggttggcgcatcaggcgcgggaatatctgcatgcccatctgcatgaagatgtcggcctcgaacaactggcggcggcaaccggtgaaagccgttttgtggtcagccgtgcgtttaaagccgaattcggattgccgccgcacgcctatcttattcagctgaaactgacccgcgcccgcgaagggctggcgctcgggcgtacaccggcagccgtggccgccgatttaggctttgccgatcagagccatctcgggcgctggttccgccgtgcgtatcagctcacgccagcgcattaccggcgtctgtgcacaaatcttccagattgagaaaaagtgacaggttaagctctgatttcaactgaaccggagcctgtcatcatgaataccaccgcaacaattctgccctcgtcccctgaacgctgcgtgattgtcatcaatcagcaacttgccgcaggacatgccgccaatgccgccgcggtcctggcgctgacgctcggacaacgccatccggcgctggtgggcgcaccgctgattgatgccgataaccgcgaacatccggggctgatcccgattggtatcagcgtgttagtcgctgatgctcaacaactcacacaacttcatcagcatctgctaaatgacgatgaaatggacggcattattttcccggtcgaagggcagcaaaccaccgattacgccgcctttcgtgaggcggtatcggttgtgccgaccaacaacctgcaactgctcggcatcgcgctggcggggaataaaaaagtggtgcgtaagctgaccggcaaactgggattactgggctgaaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctgtgcgtaagtccctcgtcgctctgctgcttttcactctgggttccacctttgctgttcaggctactgaagaagccaaaccctttatcaccagtcaggaactggatctgacccaatatctgccagcgccaccggcggatgattcggcgcagacccaagcggagctgaaagaattgctccaaattcaggccacccgcacgccggagcaggaaaaagcggcgattgctgatgcgcaagaaaacgtctggcgttttgccgatgtgatggggccgggctttgatgccgagaaactgccgaaaaccgccgcgctgtttgagcgtattgtggcgacagaagacgtggtggacgatcacgccaagaaagcgtttaaccgtccgcgtccttatatgctggatgaacaaattcatccgctgctgaaaaagtctaaatccggttcatggccttccggtcattccaccatcggttatctgatggcgacggtgctgggcgaaatggtgccggaaaaacgcaatgcgctgtttgcccgtgcatccggttatgccgaaaaccgtctggtggctggtttccattaccgttctgataccgtcatgagccgcaccggtgccgcgctgattgctcagaaaatggaagaacagccagatttcaaaaccgaattcgacgcggcgaaagcggaactgcgtacgcagctgggcctgaaataattttcgtcagtttctgcctttccggtaaagtatggaacgtaaaacttgcagaggaagggactgatggaatacagattcaagcggatggcatcaccggtcgggttactcacactggcggcgaaaggcgacaagctgaccgccattttgtgggaatgtgaaatcgacgggcgcgtgcctttgggtgaaatgctggaagacccggcgtttccgattttgctgaaaaccgaacaacagctgaacgagtatttcgcgggtaaacgcacgtgctttgagctggaccttgatttcaccggcaccgcattccagaaggaagtctgggcggcgttgctggaaattccgtttggtgagacgcgcagctacggcgacatcgcccgccgcatcggccgcccgaaagcggttcgcgccgtgggcgctgccaatggtcgtaatccgatttctatcgtagcaccctgtcaccgggtgatcggttcttccggcaaattaaccggctttgccggcggactggagaacaagttgttattactgaggctggaaggccgtaaatcgtaaggttttttatgggggaataaattgcttgatgtccggttgtcagtcgatgcatagtcaaaaaataagcagtaaataaaaaagacaacatcacttacagacggagttttaccttaatggatcagattcaatccctcgaacagttcttatcctcagttcagcaacgcgacccgcatcaggtcgaattctcccaggccgtacgtgaagtcatgaccacgctatggcctttcctcgaacaacatccgcaataccgccgttccgcattgctggagcgtctggttgagccggaacgtgtcattcagtttcgtatcgcgtgggtggatgacacaaatcaggtgcaggttaaccgtggctggcgagtacagttcagttcagccatcggcccctataaaggcgggatgcgcttccacccgtcggtcaatttatcgattctcaaattcctcggcttcgaacaaactttcaaaaatgcgctgaccacattgccgatgggcggcggcaagggcggcagcgatttcaacccgaaaggcaaaagcgaaggcgagatcatgcgtttctgtcaggcgctgatgctggagctttaccgtcatctcgggccggatactgatgtgcctgcgggtgatatcggcgttggcgggcgtgaagtcgggtatatggcggggatgatgaaaaagctctccaacaataccgccagtgtgttcaccggcaaagggttgtcgttcggcggcagtctgatccgtcc

SEQ ID NO: 53 - DNA, 3224 bp, Artificial Sequence
  source: /note=“Description of Artificial Sequence Synthetic polynucleotide”
cgcgggcgtttcaggcgctgcatggcagggatctgcgcattgtgcgccagactgcgctcgacggcttgctcagcgcgctgggcgggcatctcacctggcaaccgccgcgtttgcagggcgaagcgccgtggtcacggttggcgcatcaggcgcgggaatatctgcatgcccatctgcatgaagatgtcggcctcgaacaactggcggcggcaaccggtgaaagccgttttgtggtcagccgtgcgtttaaagccgaattcggattgccgccgcacgcctatcttattcagctgaaactgacccgcgcccgcgaagggctggcgctcgggcgtacaccggcagccgtggccgccgatttaggctttgccgatcagagccatctcgggcgctggttccgccgtgcgtatcagctcacgccagcgcattaccggcgtctgtgcacaaatcttccagattgagaaaaagtgacaggttaagctctgatttcaactgaaccggagcctgtcatcatgaataccaccgcaacaattctgccctcgtcccctgaacgctgcgtgattgtcatcaatcagcaacttgccgcaggacatgccgccaatgccgccgcggtcctggcgctgacgctcggacaacgccatccggcgctggtgggcgcaccgctgattgatgccgataaccgcgaacatccggggctgatcccgattggtatcagcgtgttagtcgctgatgctcaacaactcacacaacttcatcagcatctgctaaatgacgatgaaatggacggcattattttcccggtcgaagggcagcaaaccaccgattacgccgcctttcgtgaggcggtatcggttgtgccgaccaacaacctgcaactgctcggcatcgcgctggcggggaataaaaaagtggtgcgtaagctgaccggcaaactgggattactgggctgattaaaaacgtgaccacgagcattaatgaacgctgcgaaatgtggcgtttatttattcaaaaagtatcttctttcataaaaagtgctaaatgcagtagccgcaaaattgggataagtcccatggaatacggctgttttcgctgcaatttttaactttttcgtaaaaaaagatgcttctttgagcgaacgatcaaaatatagcgcttaccgacaaaaaattattctcattagaaaatagtttgtgtaatacttgtaacgctacatggagattaacttaatctagagggttttatagtgcgtaagtccctcgtcgctctgctgcttttcactctgggttccacctttgctgttcaggctactgaagaagccaaaccctttatcaccagtcaggaactggatctgacccaatatctgccagcgccaccggcggatgattcggcgcagacccaagcggagctgaaagaattgctccaaattcaggccacccgcacgccggagcaggaaaaagcggcgattgctgatgcgcaagaaaacgtctggcgttttgccgatgtgatggggccgggctttgatgccgagaaactgccgaaaaccgccgcgctgtttgagcgtattgtggcgacagaagacgtggtggacgatcacgccaagaaagcgtttaaccgtccgcgtccttatatgctggatgaacaaattcatccgctgctgaaaaagtctaaatccggttcatggccttccggtcattccaccatcggttatctgatggcgacggtgctgggcgaaatggtgccggaaaaacgcaatgcgctgtttgcccgtgcatccggttatgccgaaaaccgtctggtggctggtttccattaccgttctgataccgtcatgagccgcaccggtgccgcgctgattgctcagaaaatggaagaacagccagatttcaaaaccgaattcgacgcggcgaaagcggaactgcgtacgcagctgggcctgaaataattttcgtcagtttctgcctttccggtaaagtatggaacgtaaaacttgcagaggaagggactgatggaatacagattcaagcggatggcatcaccggtcgggttactcacactggcggcgaaaggcgacaagctgaccgccattttgtgggaatgtgaaatcgacgggcgcgtgcctttgggtgaaatgctggaagacccggcgtttccgattttgctgaaaaccgaacaacagctgaacgagtatttcgcgggtaaacgcacgtgctttgagctggaccttgatttcaccggcaccgcattccagaaggaagtctgggcggcgttgctggaaattccgtttggtgagacgcgcagctacggcgacatcgcccgccgcatcggccgcccgaaagcggttcgcgccgtgggcgctgccaatggtcgtaatccgatttctatcgtagcaccctgtcaccgggtgatcggttcttccggcaaattaaccggctttgccggcggactggagaacaagttgttattactgaggctggaaggccgtaaatcgtaaggttttttatgggggaataaattgcttgatgtccggttgtcagtcgatgcatagtcaaaaaataagcagtaaataaaaaagacaacatcacttacagacggagttttaccttaatggatcagattcaatccctcgaacagttcttatcctcagttcagcaacgcgacccgcatcaggtcgaattctcccaggccgtacgtgaagtcatgaccacgctatggcctttcctcgaacaacatccgcaataccgccgttccgcattgctggagcgtctggttgagccggaacgtgtcattcagtttcgtatcgcgtgggtggatgacacaaatcaggtgcaggttaaccgtggctggcgagtacagttcagttcagccatcggcccctataaaggcgggatgcgcttccacccgtcggtcaatttatcgattctcaaattcctcggcttcgaacaaactttcaaaaatgcgctgaccacattgccgatgggcggcggcaagggcggcagcgatttcaacccgaaaggcaaaagcgaaggcgagatcatgcgtttctgtcaggcgctgatgctggagctttaccgtcatctcgggccggatactgatgtgcctgcgggtgatatcggcgttggcgggcgtgaagtcgggtatatggcggggatgatgaaaaagctctccaacaataccgccagtgtgttcaccggcaaagggttgtcgttcggcggcagtctgatccgtcc

SEQ ID NO: 54 - DNA, 3551 bp, Artificial Sequence
  source: /note=“Description of Artificial Sequence Synthetic polynucleotide”
cttgtttcttgatggctcactggaaggacatctcgatttcaggcgctgtcgtctgggtggtttcgcaggtgggttatttgccattttcgtaccacctgcttcatatatgccacaactgaagcccgattcacctgcggaacctcatgacgcttttgccattactcgggcacagatttcactgctggaacgccttgaaacgcagtccgccgggcgggcaaaaatctgccggacggtcggtgaaattgaagcgtgtattacacagaatgtgctggcgatggtgatgcatatcgaaggggcggaagcactcggcgatgatttctcgcggctggagcgctggtatgaaaaggggctgcgcagcatcggcccgttatggaacttacccaaccagtttggtaccggcgttaagggcgatttcccgggatcaccggataccggagatggcctgacgcccgccgggcttggattgctgcatgaatgtaaccggaaaaggattctgttcgatgtctcgcacatgaacgaaaaagccttctggcagacggcaaaattcagcgatgcgccgctggttgccacccattcaaatgtgcacgcgttatgtccgcaaccgcgtaatctgaccgataaacaactggctgccattgccgaaagcaacggcttcgttggcgttaatttcggcacggcttttctgcgggcggatggaaaacgcaacggcgacacccccatcaccgaaattgttaaacatcttgataaccttgttggtaaactgggggaagaaaatgtcggttttggttcggatttcgacggtatcaatgtgccggatacgctcggtgatgtcgccggattaccgttgctgcttcaggctatgtctgatgcgggatacggcgatgcattgatcgaaaaaatcgcgtaccgcaactggctgaaagtattaaagcaaacctggggtgaatagtgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatcatgtggactaaaccttcattcgaagacctgcgtttaggcttagaagtgacactgtacatttctaaccgctaagcctttatgcccacggttaactgtgggcatcttccttccctttcccctggttctcacatgcagattatcgtacttggttccgcggcaggcggcggcttcccgcagtggaactgcaattgcagcaactgtcagggtgtgcgtaatggcaccatgaaaacgtccccccgcacgcaatcttcgattgccgtcagcgacaatggcaccgactgggtgctgtgtaacgcctcgccggatatttgccaccagattgccgccacgccggaactgataaaacaagacgttttacgcggcaccgccattggttccattatcctgactgacagccagattgatcactgcaccggcctgctgaatttgcgtgaaggctgtccgcatcaggtgtggtgtacgccggaagtccacgaagacctcaccaccggtttcccgattttcaccatgctttctcactggaatggcggcctgcaacaccacgctatcaggccggagaaccgcttctccgttgccgtctgcccgaatcttacattcactgccattccgctgctgagcaacgcgccaccgtattcgaaataccgcggcaaaccgctccccggccacaatatcgcgctctttattgaagacacaaaaaccggcacctcgctgctgtacgcaccgggtctgggcgaaccggacgatgaactgctgaaatggctgcataaagccgattgcctgctgattgacggcacgctgtggcaggacaacgagctggcgaccaccggcgtcggccgcaataccggcaaagacatgggccatctggcgcttgccgaagaacaagggctgatcgccctgctgtcgtcacttccggcaaaacgcaaaattctcatccatattaataataccaacccgatcctcaatgaatcctctgccgagcggcaggcgctgacgcaacaaaacatcgaagtcagccgggacgggatgcgcatcgaactgtagggcaaaacgaccatgagcatatcgacaacacagacgtcaccgatgacgccgcaagaatttgaacaggcgctgcgtgccaaaggcgcgttttatcacatccatcatccctaccatattgcgatgcataacggtcaggcgacccgcgagcaaattcagggctgggtggcgaaccgtttctattatcagaccagcattccgctgaaagacgcggcgattatggccaactgcccggatgcgcaaacccgccgtaaatgggtgcagcgtattctcgatcacgacggacatggcggcagtgaaggcggtatcgaagcctggctgcgtctgggcgaagcggtggggttagaccgcgatgtgctgctttcagaagaaagggtgttaccgggggtgcgttttgcggtcgatgcctacgtcaattttgcccgccgcgccgtctggcaggaagccgcgtgcagctcgctcaccgaactgttcgccccgcaaatccatcaggcgcgtctcgacacctggccacagcattacacatggattgaggaagaaggttacggttatttccgcagccgcctgagccaggctaaccgcgacgtcgaacacggcctgcaactggccctggagtattgcgataccgtcgaaaaacaacagcgcatgctggaaatcctgcaattcaaactcgatattttgtggagcatgctcgattccatgagcatggcctacgaactgaaccgcccgccgtaccacagcgtgacgcagcaggcggtctggcataaaggaagactcctgtgatcaccattaccgaacactacacgccgatgtttcgtcgcggctaccgcatgcagtttgagaaaacgcaggactgccatgtgattttgtatccggaagggatggcgaaactcaacgacagtgcgaccttcattttacaactggtggatggcgggcggacaattgccaatattattgatgaactgaatgcccgctttccgcaggccggtggcgtgaatgacgacgtcaaagacttctttgctcaggcccatgcccaaaagtggattatcttccgtgaacctgct

SEQ ID NO: 55 - DNA, 3221 bp, Artificial Sequence
  source: /note=“Description of Artificial Sequence Synthetic polynucleotide”
cttgtttcttgatggctcactggaaggacatctcgatttcaggcgctgtcgtctgggtggtttcgcaggtgggttatttgccattttcgtaccacctgcttcatatatgccacaactgaagcccgattcacctgcggaacctcatgacgcttttgccattactcgggcacagatttcactgctggaacgccttgaaacgcagtccgccgggcgggcaaaaatctgccggacggtcggtgaaattgaagcgtgtattacacagaatgtgctggcgatggtgatgcatatcgaaggggcggaagcactcggcgatgatttctcgcggctggagcgctggtatgaaaaggggctgcgcagcatcggcccgttatggaacttacccaaccagtttggtaccggcgttaagggcgatttcccgggatcaccggataccggagatggcctgacgcccgccgggcttggattgctgcatgaatgtaaccggaaaaggattctgttcgatgtctcgcacatgaacgaaaaagccttctggcagacggcaaaattcagcgatgcgccgctggttgccacccattcaaatgtgcacgcgttatgtccgcaaccgcgtaatctgaccgataaacaactggctgccattgccgaaagcaacggcttcgttggcgttaatttcggcacggcttttctgcgggcggatggaaaacgcaacggcgacacccccatcaccgaaattgttaaacatcttgataaccttgttggtaaactgggggaagaaaatgtcggttttggttcggatttcgacggtatcaatgtgccggatacgctcggtgatgtcgccggattaccgttgctgcttcaggctatgtctgatgcgggatacggcgatgcattgatcgaaaaaatcgcgtaccgcaactggctgaaagtattaaagcaaacctggggtgaatagtacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaacatgtggactaaaccttcattcgaagacctgcgtttaggcttagaagtgacactgtacatttctaaccgctaagcctttatgcccacggttaactgtgggcatcttccttccctttcccctggttctcacatgcagattatcgtacttggttccgcggcaggcggcggcttcccgcagtggaactgcaattgcagcaactgtcagggtgtgcgtaatggcaccatgaaaacgtccccccgcacgcaatcttcgattgccgtcagcgacaatggcaccgactgggtgctgtgtaacgcctcgccggatatttgccaccagattgccgccacgccggaactgataaaacaagacgttttacgcggcaccgccattggttccattatcctgactgacagccagattgatcactgcaccggcctgctgaatttgcgtgaaggctgtccgcatcaggtgtggtgtacgccggaagtccacgaagacctcaccaccggtttcccgattttcaccatgctttctcactggaatggcggcctgcaacaccacgctatcaggccggagaaccgcttctccgttgccgtctgcccgaatcttacattcactgccattccgctgctgagcaacgcgccaccgtattcgaaataccgcggcaaaccgctccccggccacaatatcgcgctctttattgaagacacaaaaaccggcacctcgctgctgtacgcaccgggtctgggcgaaccggacgatgaactgctgaaatggctgcataaagccgattgcctgctgattgacggcacgctgtggcaggacaacgagctggcgaccaccggcgtcggccgcaataccggcaaagacatgggccatctggcgcttgccgaagaacaagggctgatcgccctgctgtcgtcacttccggcaaaacgcaaaattctcatccatattaataataccaacccgatcctcaatgaatcctctgccgagcggcaggcgctgacgcaacaaaacatcgaagtcagccgggacgggatgcgcatcgaactgtagggcaaaacgaccatgagcatatcgacaacacagacgtcaccgatgacgccgcaagaatttgaacaggcgctgcgtgccaaaggcgcgttttatcacatccatcatccctaccatattgcgatgcataacggtcaggcgacccgcgagcaaattcagggctgggtggcgaaccgtttctattatcagaccagcattccgctgaaagacgcggcgattatggccaactgcccggatgcgcaaacccgccgtaaatgggtgcagcgtattctcgatcacgacggacatggcggcagtgaaggcggtatcgaagcctggctgcgtctgggcgaagcggtggggttagaccgcgatgtgctgctttcagaagaaagggtgttaccgggggtgcgttttgcggtcgatgcctacgtcaattttgcccgccgcgccgtctggcaggaagccgcgtgcagctcgctcaccgaactgttcgccccgcaaatccatcaggcgcgtctcgacacctggccacagcattacacatggattgaggaagaaggttacggttatttccgcagccgcctgagccaggctaaccgcgacgtcgaacacggcctgcaactggccctggagtattgcgataccgtcgaaaaacaacagcgcatgctggaaatcctgcaattcaaactcgatattttgtggagcatgctcgattccatgagcatggcctacgaactgaaccgcccgccgtaccacagcgtgacgcagcaggcggtctggcataaaggaagactcctgtgatcaccattaccgaacactacacgccgatgtttcgtcgcggctaccgcatgcagtttgagaaaacgcaggactgccatgtgattttgtatccggaagggatggcgaaactcaacgacagtgcgaccttcattttacaactggtggatggcgggcggacaattgccaatattattgatgaactgaatgcccgctttccgcaggccggtggcgtgaatgacgacgtcaaagacttctttgctcaggcccatgcccaaaagtggattatcttccgtgaacctgct

SEQ ID NO: 56 - DNA, 3344 bp, Artificial Sequence
  source: /note=“Description of Artificial Sequence Synthetic polynucleotide”
cttgtttcttgatggctcactggaaggacatctcgatttcaggcgctgtcgtctgggtggtttcgcaggtgggttatttgccattttcgtaccacctgcttcatatatgccacaactgaagcccgattcacctgcggaacctcatgacgcttttgccattactcgggcacagatttcactgctggaacgccttgaaacgcagtccgccgggcgggcaaaaatctgccggacggtcggtgaaattgaagcgtgtattacacagaatgtgctggcgatggtgatgcatatcgaaggggcggaagcactcggcgatgatttctcgcggctggagcgctggtatgaaaaggggctgcgcagcatcggcccgttatggaacttacccaaccagtttggtaccggcgttaagggcgatttcccgggatcaccggataccggagatggcctgacgcccgccgggcttggattgctgcatgaatgtaaccggaaaaggattctgttcgatgtctcgcacatgaacgaaaaagccttctggcagacggcaaaattcagcgatgcgccgctggttgccacccattcaaatgtgcacgcgttatgtccgcaaccgcgtaatctgaccgataaacaactggctgccattgccgaaagcaacggcttcgttggcgttaatttcggcacggcttttctgcgggcggatggaaaacgcaacggcgacacccccatcaccgaaattgttaaacatcttgataaccttgttggtaaactgggggaagaaaatgtcggttttggttcggatttcgacggtatcaatgtgccggatacgctcggtgatgtcgccggattaccgttgctgcttcaggctatgtctgatgcgggatacggcgatgcattgatcgaaaaaatcgcgtaccgcaactggctgaaagtattaaagcaaacctggggtgaatagttaaaaacgtgaccacgagcattaatgaacgctgcgaaatgtggcgtttatttattcaaaaagtatcttctttcataaaaagtgctaaatgcagtagccgcaaaattgggataagtcccatggaatacggctgttttcgctgcaatttttaactttttcgtaaaaaaagatgcttctttgagcgaacgatcaaaatatagcgcttaccgacaaaaaattattctcattagaaaatagtttgtgtaatacttgtaacgctacatggagattaacttaatctagagggttttataatgtggactaaaccttcattcgaagacctgcgtttaggcttagaagtgacactgtacatttctaaccgctaagcctttatgcccacggttaactgtgggcatcttccttccctttcccctggttctcacatgcagattatcgtacttggttccgcggcaggcggcggcttcccgcagtggaactgcaattgcagcaactgtcagggtgtgcgtaatggcaccatgaaaacgtccccccgcacgcaatcttcgattgccgtcagcgacaatggcaccgactgggtgctgtgtaacgcctcgccggatatttgccaccagattgccgccacgccggaactgataaaacaagacgttttacgcggcaccgccattggttccattatcctgactgacagccagattgatcactgcaccggcctgctgaatttgcgtgaaggctgtccgcatcaggtgtggtgtacgccggaagtccacgaagacctcaccaccggtttcccgattttcaccatgctttctcactggaatggcggcctgcaacaccacgctatcaggccggagaaccgcttctccgttgccgtctgcccgaatcttacattcactgccattccgctgctgagcaacgcgccaccgtattcgaaataccgcggcaaaccgctccccggccacaatatcgcgctctttattgaagacacaaaaaccggcacctcgctgctgtacgcaccgggtctgggcgaaccggacgatgaactgctgaaatggctgcataaagccgattgcctgctgattgacggcacgctgtggcaggacaacgagctggcgaccaccggcgtcggccgcaataccggcaaagacatgggccatctggcgcttgccgaagaacaagggctgatcgccctgctgtcgtcacttccggcaaaacgcaaaattctcatccatattaataataccaacccgatcctcaatgaatcctctgccgagcggcaggcgctgacgcaacaaaacatcgaagtcagccgggacgggatgcgcatcgaactgtagggcaaaacgaccatgagcatatcgacaacacagacgtcaccgatgacgccgcaagaatttgaacaggcgctgcgtgccaaaggcgcgttttatcacatccatcatccctaccatattgcgatgcataacggtcaggcgacccgcgagcaaattcagggctgggtggcgaaccgtttctattatcagaccagcattccgctgaaagacgcggcgattatggccaactgcccggatgcgcaaacccgccgtaaatgggtgcagcgtattctcgatcacgacggacatggcggcagtgaaggcggtatcgaagcctggctgcgtctgggcgaagcggtggggttagaccgcgatgtgctgctttcagaagaaagggtgttaccgggggtgcgttttgcggtcgatgcctacgtcaattttgcccgccgcgccgtctggcaggaagccgcgtgcagctcgctcaccgaactgttcgccccgcaaatccatcaggcgcgtctcgacacctggccacagcattacacatggattgaggaagaaggttacggttatttccgcagccgcctgagccaggctaaccgcgacgtcgaacacggcctgcaactggccctggagtattgcgataccgtcgaaaaacaacagcgcatgctggaaatcctgcaattcaaactcgatattttgtggagcatgctcgattccatgagcatggcctacgaactgaaccgcccgccgtaccacagcgtgacgcagcaggcggtctggcataaaggaagactcctgtgatcaccattaccgaacactacacgccgatgtttcgtcgcggctaccgcatgcagtttgagaaaacgcaggactgccatgtgattttgtatccggaagggatggcgaaactcaacgacagtgcgaccttcattttacaactggtggatggcgggcggacaattgccaatattattgatgaactgaatgcccgctttccgcaggccggtggcgtgaatgacgacgtcaaagacttctttgctcaggcccatgcccaaaagtggattatcttccgtgaacctgct

SEQ ID NO: 57 - DNA, 3148 bp, Artificial Sequence
  source: /note=“Description of Artificial Sequence Synthetic polynucleotide”
gtcaggcgttcagtaaagacgctgtgctctcatgcaattcatcaagcaaggtataacgcttgcggtattcagaacgtttcttgctggcaatctcttccatcggtttgcgcggcagttccagtggaacgtgatataaggctgaagatgcgggcgtggcactgatggaatcccagaattcgttataactggcgacaaacacgttcttcttgcggtgacggtaacgcaggctgcggaatacgtgaccggtatcgctgacggcaagaatgcgctgcacgccggttccggcagcgacacgttgcaggctttccagcagcagacgtttcgggaacaagccgtaacaggatttagtcgcctgtttaatcacttcatgtgccgttgaacggtgcgcgccctggaatccgccgataatcagtgtcctcccttccgggcgctgcacaatgctaaacgtcagtgccgcaagcagcgtgtcctccatataaaggaacatgttggcttcgccttcgcgctcagatttgccgatggaaccgagctccacccggaaggcttcaccgtctttgccggtgaaccgggtgatggtattgccggtcgcgctcagaaaggtattgcgcaggcgggcgttttccaggcttttgacgaaggcgtagtgatcaaccagtgcgtcagctcgtccggctgaatgcagaccgagatacagatacggcttgtgaattttactcggtagtttggtctgcacctggaaagcttcctgccagagggtttccgccgccatcttattcagcactttcagggtatcgagcggatgcagtaaggtgcgaacggcatatttgacccggaacatccggtcgcgccacagattgtccggtacgcacgctccgctcagaagatcgacaaacaggctgaatccgctggttttttttgtggtttgttcaacgggatatgcttcgctgagggactcggtttcattgagagggatatgttccatgatctgtttccgctgccagaaataatcacggtatcacctgatttttaaacctctttacaacgttgttttaaagagaagcgaggttatctgaataagtgcaggctgtttagctgccgtttattctggctgactgaaagcgtctcactgtctttaaaaacgtgaccacgagcattaatgaacgctgcgaaatgtggcgtttatttattcaaaaagtatcttctttcataaaaagtgctaaatgcagtagccgcaaaattgggataagtcccatggaatacggctgttttcgctgcaatttttaactttttcgtaaaaaaagatgcttctttgagcgaacgatcaaaatatagcgcttaccgacaaaaaattattctcattagaaaatagtttgtgtaatacttgtaacgctacatggagattaacttaatctagagggttttataatgaaaattattactactttttgcctcgccagccttttttctgtcaacgcgtttgccctgaccggtaacgatgcgaccaccaaacccgatctttactacctgaaaaacgatcaggcgattaacagcctggcgctgcttccgcccccgcctgcggtgggcagtatcgcttttctaaacgatcaggccatgtatgaacagggtcgtctgctgcgctcaacagaacgtggaaaactggcggcagaagatgccaacctgagtgccggtggcgtggcgaacgccttctcgggtgccttcggttcgccaatcaccgccaaagacagcccggaactgcataaactgctgaccaatatgattgaagatgcgggtgatctggcgacacgctccgccaaagaaaagtatatgcgcattcgtccgtttgccttctacggtgtgccgacctgtaacaccaccgagcaggataagctgtcgaaaaacggttcgtatccctccggtcacacctcaattggctgggccaccgcgctggtgctcaccgaaattaacccgcagcgccaggaccaaatcctgcaacgcggtttcgatttaggccagagccgggtaatttgcggctaccactggcaaagtgatgtcgatgcagcgcgcatcgtcggttccgccgtagtggctaccctgcacactaaccctgctttccagcaacaactgcaaaaagccaaagaagaatttgctaaacagcatccgtaaatattgggcctccccgacctggtcttcgtaaaagccggggagtctgtaatgccacatctctcattgatactaaatttctgaaaattgcttttttgcaacggtcactaattgttaactccccttcacaggcataacgtcattcctcattttaaaaaggagtttcattatggctagccccgcatatctctggctatacgatgccaatggcgcactactttatggcggttctgaggttttaagccgtgaaggcgcgatcgagatccaaagcttcacgcatggtctttccgtacccttcgatggtaataccggtcggctgacatctacccgtgttcatcaaaccatgggactggtaaaagagttcgataaatctactccctacctctaccgtgccgtcgccaccagcgaaaagctgcaaaaggcggtgattaaatggtatcgcattaatgcggctggtatggaggaagagtttttgaatatgacaatggaaggggtgcgcatacttaacatcaatcctcatatgcacaatttcaaacacgctgacggacaggccagtatgcccacggaatccattggccttgggtatcagaaaatcacatggttgtacctcgacggacatatcagttttaccgatcagtggaactccagcatttacgcataaacggagacagggatgatttcatgcagagtggattttaatcgtctttatgagggcggaaaaattgcaaaactttattgtgctggtgtggggatcttccctgtattttcaggacttggttcactgctcaacaggcagggatgttcagatttaaaaaatggtgccattccgccaggaaagtactggattgttgatcgccctgccggcggtttgaattcgtggattgtacaaatgagaaaggagtggaaaacgggtaacgattacgattcatggtttgcgctttatagacaggattctcttatcgatgattggacaaatataggcgccagg

SEQ ID NO: 58 - DNA, 2123 bp, Artificial Sequence
  source: /note=“Description of Artificial Sequence Synthetic polynucleotide”
cgctttcgcggcctttatcatttcagtcgtttgccgtttatttcttcagttcggcgcgtaagtctttcacgtctgaggcagaaaccggcgcggcggcatttccccagctgttacggatatacgtcagcacgttagccacatcggcatcgctcatattggcatcaaacgatggcatcgacggcgcggtaggtttgctgtcggtagccacagggcggctgcccgtcagcaccacgcggataagcgacgtggcgttggtctggttgatgagcggtgaatctgccagccgtgggaacagaccttcctgacccataccgcctggcgtatgacatgcggaacaccggtcagcgtagatattcttcccggcaatcattgccttatcgtcagctttcactggcgcaggggcgttattgccgctgtcatgaccgctgtctttcagatagaccgccacggcttcaaggtcggcatccgtccagtgccgggatgagttcgtcacttcctccgccatcggaccggaagcaatatcgaaacggttagaaccggttttcagatactgcatcagatcttcctgcgtccagttaccgacaccagtgtgcttgttgctggtgatgtccggtgcgacccagttttctatcaccgccccttgcaggaactcgctgtccttatcgccgccgagcatatttttcggtgtatgacaggtaccgcagtgtcccagaccttcaacgatgtaggcgccgcggttccattgcgccgatttatccatctgcggtttgaattcgcctttgctgaagttcagccagttccagcccatcagactggtacgcacgttgaacgggaacggcaactggttagtttcgatttcgttgtgcaccggttgcagggtctgcaaatacgcccagatcgccgcgttatcttcgcgggtcactttggtgtacgccgtgaacggcatcgcaccgtaaagacgtttgccgccgtggccgatgccttcggacatcgcgcgctggaagtcatcaaaactccatttaccgataccggtttgcacatccggcgtaatgtttgcaccgactaaacggccgaacggggtttcaattggcacaccgccggcaaaaggtttggcgtccggcgtggaggcagtatgacaggcggcgcagtcgcccactgtcgccagataacggccacgctcaacctgttcgaaagagccgtcgcccgccgcgtgggccagcccgctgatgctcatggccagcaaaccgagtgagaatgaggttaacttttaatgttgttctgcccttagcttttcattttccatgcgtcgccgccggaaatactcagcggttcgagatcgagtttctggttatgtttgccgatatattcgcggtagtcgtagcgcgcgccggggaagcccagcattttccacgacaccatattgcggttaccgccataaatcgggtcagcaaagaaaccttccattgtgttcttcagtgcgatggcgaagaacaatttagaatcgatcccttcaagggcaatttttccgctctcaagacctgacaggatttgatcctgctggtcgccgggcagatctttgaataatttctgatgctgtgaccgggcatatttatccaatgcggccaggccaagacggtagcgttgttgcggcaccagcggcgactgatcgccttgctccggagtgccctcctggaaagggccttgcatataaaggcgggaataggtgccgtagaatccggccagctgacggtcgataaacaccgcgcatccggcgtctttaccgcccacgctcagatcatcggcagggaccagacggtcaacaatggcttccatggcagcggcttcggcttcggtgaaaaacacccagccatcggtctcaatctgcggcggcgggctggcgtcaaaggggctccacacgggcgtgcctttgagcgtcaccgcgttggcggctttggcggcacccagtactgcggaggcggccgtgaacgagagaatctgtctgcgggtcacgccgggcaaggtttttttgctcatgtatgttccctgtttgtatacatcactttaaggtccggacgcgctctgcaaaggcagtcgggtggcgtcgggataatcgttaacctgct

SEQ ID NO: 59 - DNA, 3025 bp, Artificial Sequence
  source: /note=“Description of Artificial Sequence Synthetic polynucleotide”
gtcaggcgttcagtaaagacgctgtgctctcatgcaattcatcaagcaaggtataacgcttgcggtattcagaacgtttcttgctggcaatctcttccatcggtttgcgcggcagttccagtggaacgtgatataaggctgaagatgcgggcgtggcactgatggaatcccagaattcgttataactggcgacaaacacgttcttcttgcggtgacggtaacgcaggctgcggaatacgtgaccggtatcgctgacggcaagaatgcgctgcacgccggttccggcagcgacacgttgcaggctttccagcagcagacgtttcgggaacaagccgtaacaggatttagtcgcctgtttaatcacttcatgtgccgttgaacggtgcgcgccctggaatccgccgataatcagtgtcctcccttccgggcgctgcacaatgctaaacgtcagtgccgcaagcagcgtgtcctccatataaaggaacatgttggcttcgccttcgcgctcagatttgccgatggaaccgagctccacccggaaggcttcaccgtctttgccggtgaaccgggtgatggtattgccggtcgcgctcagaaaggtattgcgcaggcgggcgttttccaggcttttgacgaaggcgtagtgatcaaccagtgcgtcagctcgtccggctgaatgcagaccgagatacagatacggcttgtgaattttactcggtagtttggtctgcacctggaaagcttcctgccagagggtttccgccgccatcttattcagcactttcagggtatcgagcggatgcagtaaggtgcgaacggcatatttgacccggaacatccggtcgcgccacagattgtccggtacgcacgctccgctcagaagatcgacaaacaggctgaatccgctggttttttttgtggtttgttcaacgggatatgcttcgctgagggactcggtttcattgagagggatatgttccatgatctgtttccgctgccagaaataatcacggtatcacctgatttttaaacctctttacaacgttgttttaaagagaagcgaggttatctgaataagtgcaggctgtttagctgccgtttattctggctgactgaaagcgtctcactgtcttacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaacatgaaaattattactactttttgcctcgccagccttttttctgtcaacgcgtttgccctgaccggtaacgatgcgaccaccaaacccgatctttactacctgaaaaacgatcaggcgattaacagcctggcgctgcttccgcccccgcctgcggtgggcagtatcgcttttctaaacgatcaggccatgtatgaacagggtcgtctgctgcgctcaacagaacgtggaaaactggcggcagaagatgccaacctgagtgccggtggcgtggcgaacgccttctcgggtgccttcggttcgccaatcaccgccaaagacagcccggaactgcataaactgctgaccaatatgattgaagatgcgggtgatctggcgacacgctccgccaaagaaaagtatatgcgcattcgtccgtttgccttctacggtgtgccgacctgtaacaccaccgagcaggataagctgtcgaaaaacggttcgtatccctccggtcacacctcaattggctgggccaccgcgctggtgctcaccgaaattaacccgcagcgccaggaccaaatcctgcaacgcggtttcgatttaggccagagccgggtaatttgcggctaccactggcaaagtgatgtcgatgcagcgcgcatcgtcggttccgccgtagtggctaccctgcacactaaccctgctttccagcaacaactgcaaaaagccaaagaagaatttgctaaacagcatccgtaaatattgggcctccccgacctggtcttcgtaaaagccggggagtctgtaatgccacatctctcattgatactaaatttctgaaaattgcttttttgcaacggtcactaattgttaactccccttcacaggcataacgtcattcctcattttaaaaaggagtttcattatggctagccccgcatatctctggctatacgatgccaatggcgcactactttatggcggttctgaggttttaagccgtgaaggcgcgatcgagatccaaagcttcacgcatggtctttccgtacccttcgatggtaataccggtcggctgacatctacccgtgttcatcaaaccatgggactggtaaaagagttcgataaatctactccctacctctaccgtgccgtcgccaccagcgaaaagctgcaaaaggcggtgattaaatggtatcgcattaatgcggctggtatggaggaagagtttttgaatatgacaatggaaggggtgcgcatacttaacatcaatcctcatatgcacaatttcaaacacgctgacggacaggccagtatgcccacggaatccattggccttgggtatcagaaaatcacatggttgtacctcgacggacatatcagttttaccgatcagtggaactccagcatttacgcataaacggagacagggatgatttcatgcagagtggattttaatcgtctttatgagggcggaaaaattgcaaaactttattgtgctggtgtggggatcttccctgtattttcaggacttggttcactgctcaacaggcagggatgttcagatttaaaaaatggtgccattccgccaggaaagtactggattgttgatcgccctgccggcggtttgaattcgtggattgtacaaatgagaaaggagtggaaaacgggtaacgattacgattcatggtttgcgctttatagacaggattctcttatcgatgattggacaaatataggcgccagg

SEQ ID NO: 60 - DNA, 4004 bp, Artificial Sequence
  source: /note=“Description of Artificial Sequence Synthetic polynucleotide”
gattgttctggaacgtaaatactccggcgtagacacccgctggaaacatgacgatcagataggttccgtgccgttctcggttaccggcggtctggattatgaaaccatgaccgaacgtcgtttcggctatgagaacttcaacagtgaaggcgatctgggtgtgaaaggcgacgaacgccgtaatgaaaaaaacgtgatgtggaacctcgatccctatctgcaaaccacgtggaacctgacatcgcgctggacactcgatgccggtgcgcactacagcaccgtgagtttcgactcgcaggattattacattaccggcagcaacccggatgacagcggttcgcgccgttatcataaactgctgccgatggcgtccctgaactatgccgtgacgcctgcactgaacacctatatttctgccggtcgtggttttgaaacgccgaccattaatgaactgtcttaccgcaccgacggtaaatcgggcctgaatctggggctggaaccttccaccagtaccaccgtggagctgggcagcaaatggcgcgtgggcaacggtttggtgaccgccgccgtcttccagactgatactgatgacgagctgattgtggcacaaagtaccggcggtcggtcgagctacaccaacgccggtaagacacgccgtcgggggctggaactgtcattagatcagcagattgaagaaaactggcgcgtaaaaatggcctggacgctgctcgatgcgaccttccgcaacgagacctgtggcgcgggtgattgcacacccgcgggcaaccgtctgccgggtattgcgcgcaacatgggctacgcttctctggagtgggcgccggttgagggatggcacgcgggtgccgatatccgctacatgagcgacattgaagtcaacgacgaaaacagtgaacaggcaccggcatataccgtcgccagcgtgaatgcgggttaccgcttcaactggaacaacgtgacgctcgacttgtttacccgtgtcgacaatttgttcgacagaaattatgtcgggtctgtgattgtcaatgaaggcaacggccgttatttcgaaccggcacctggcagaaactacggtggcggagctacgctttcttatagtttcgaataatacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaacatggaaacgaaagcctcgttatcacgcattgtcgtcataattaccgctttgttcgccgcgttaagcgggatttaccttcttgccggtggtatctggctggcaaaattaggaggttctctttattacatcattgccggtgttatttcgctggttactgcgtggctgctttaccgtcgccgctcttcggcattattgctctatgccatcttcctgttcggcaccactgtctgggctgtatgggaagtgggcacagacttctgggcactgacgccgcgtctggacgtcaccttcttcctgggcctgtggatcctgctgcccgtggtttataaccagatgctggcgaaaaacgcctttgcacgcggcgcgctggcagtttctctgctgttcaccgtgatcgtgctgggctacgccatctttaacgacccgcaggtgattaacggcaccatcaaagcggcggattctgctccggcaaaatctgagtccggcatccctgatggcgactggccggcgtatggtcgtactcagggcggtacccgttactcgccactgaaccagatcaacgataaaaacgtcagcaagctcgacgtggcctggactttccgtaccggtgacctgaagaccccgaacgatccgggcgaaatcactgacgaagtcacgccaatcaaaatcggcgacatgctttatctgtgtacgccgcatcagaagctgtttgctctggatgccgcaaccggtaaagagaagtggaagttcgatcctgagctgaaacccaacccaaccttccagcacgtgacctgtcgtggtgtgtcttatcatgagaccacaccagccgcagaaggcaacgccaccaacggcgcggcgcctgctgtctgttcccgtcgtatcattctgcctgtcaacgatggccgtctgtttgcgctggacgctgaaaccggcgcacgttgcccggcatttggcaacaacggtgagctgaacctgcaaggcaacatgccttatgcaaccccaggccactacgagccaacttcaccacctatcatcaccaaatctgtgatcatcgtggcgggtgcggttaccgataactactcaaaccgcgagccttccggcgtgatccgtggttttgatgttgagaccggtaaactgctgtgggccttcgatccgggtgcggctgagccgaacaaaatccctgaggatggtcagcatttcacgccgaactccccgaactcatgggcacctgcggcttatgatgacaaactggatctggtttacctgccaatcggcgtggcaacccctgatatctggggcggcaaccgtactccggaaatggaacgtttcgcgagcggcctgctggcgctgaatgcgaccaccggtaaactggcctggttctatcagactgtgcatcacgacctgtgggatatggacgtgccagcgcagccaacgctggctgatatcactgacaagagcggcaacaaagttccggcgatttatgtaccgaccaaaaccggtaacatcttcgtgctggatcgccgtgacggtaagctgattgttgatgcgcctgagaaacctgttccgcaaggcgcggcgaaaggcgaccatgtgtctccgacccagccattctccaaactgactttccgtcctgaagccaaactgaccggtaaagacatgtggggcgctacgatctacgaccaactgatgtgtcgtgtgatcttccacaaactgcgttatgaaggcaccttcacgccgccatccgagcagggcactctggtgttcccgggcaacctcggtatgttcgagtggggcggtatttctgtggatacagaccgtcaggtggctatcgcgaacccgattgcgctgccattcgtgtctaaactgatcccacgcggtccgggcaaccctatcgagccagatgcgaacgataaaggcggttccggtactgagactggcattcagccgcagtacggcgtgccatttggcgtgacgctgaatccgttcctgtctccgctgggcttcccatgtaaacagcctgcatggggttacatttccggtgttgacctgaaaaccaacgatatcgtgtggaaaaaacgtatcggtaccgtgcgcgacagctcaccattaccgctgccgttcaaaatgggtatgccaatgctgggcgcaccggtttctaccgccggtaacgtgttcttcatcgcggcaaccgcagataactacctgcgcgcgttcaacatgagcaacggtgacaaactgtgggaagcacgtctgccagcaggcggccaggcaaccccgatgacttactccgtcaatggcaaacagtacgttgttatcgcggcaggcggtcacggttcgtttggcaccaaactgggcgattacatcatcgcttacgcactgccagacgctgatgctaaataattagcggtgttataagttctgaaatctgaaaggccgcgaaagcggccttttttacaactgctgcgcgggtaacattatctgtcacttgctgatgttcattaggttttcctcaggcacccaacctgattctccagatttgttgcatgcccagaaccagccgttgagctttttctccagcgtgaggacttcccccggagcaaccgataattcatatgccgtataattctcagtgcaaattccccggtccggagcactaagggtaaatatttgctggggcgcccatcctgcattgcctgaactctgacgga

SEQ ID NO: 61 - DNA, 3976 bp, Artificial Sequence
  source: /note=“Description of Artificial Sequence Synthetic polynucleotide”
gattgttctggaacgtaaatactccggcgtagacacccgctggaaacatgacgatcagataggttccgtgccgttctcggttaccggcggtctggattatgaaaccatgaccgaacgtcgtttcggctatgagaacttcaacagtgaaggcgatctgggtgtgaaaggcgacgaacgccgtaatgaaaaaaacgtgatgtggaacctcgatccctatctgcaaaccacgtggaacctgacatcgcgctggacactcgatgccggtgcgcactacagcaccgtgagtttcgactcgcaggattattacattaccggcagcaacccggatgacagcggttcgcgccgttatcataaactgctgccgatggcgtccctgaactatgccgtgacgcctgcactgaacacctatatttctgccggtcgtggttttgaaacgccgaccattaatgaactgtcttaccgcaccgacggtaaatcgggcctgaatctggggctggaaccttccaccagtaccaccgtggagctgggcagcaaatggcgcgtgggcaacggtttggtgaccgccgccgtcttccagactgatactgatgacgagctgattgtggcacaaagtaccggcggtcggtcgagctacaccaacgccggtaagacacgccgtcgggggctggaactgtcattagatcagcagattgaagaaaactggcgcgtaaaaatggcctggacgctgctcgatgcgaccttccgcaacgagacctgtggcgcgggtgattgcacacccgcgggcaaccgtctgccgggtattgcgcgcaacatgggctacgcttctctggagtgggcgccggttgagggatggcacgcgggtgccgatatccgctacatgagcgacattgaagtcaacgacgaaaacagtgaacaggcaccggcatataccgtcgccagcgtgaatgcgggttaccgcttcaactggaacaacgtgacgctcgacttgtttacccgtgtcgacaatttgttcgacagaaattatgtcgggtctgtgattgtcaatgaaggcaacggccgttatttcgaaccggcacctggcagaaactacggtggcggagctacgctttcttatagtttcgaataaaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctatggaaacgaaagcctcgttatcacgcattgtcgtcataattaccgctttgttcgccgcgttaagcgggatttaccttcttgccggtggtatctggctggcaaaattaggaggttctctttattacatcattgccggtgttatttcgctggttactgcgtggctgctttaccgtcgccgctcttcggcattattgctctatgccatcttcctgttcggcaccactgtctgggctgtatgggaagtgggcacagacttctgggcactgacgccgcgtctggacgtcaccttcttcctgggcctgtggatcctgctgcccgtggtttataaccagatgctggcgaaaaacgcctttgcacgcggcgcgctggcagtttctctgctgttcaccgtgatcgtgctgggctacgccatctttaacgacccgcaggtgattaacggcaccatcaaagcggcggattctgctccggcaaaatctgagtccggcatccctgatggcgactggccggcgtatggtcgtactcagggcggtacccgttactcgccactgaaccagatcaacgataaaaacgtcagcaagctcgacgtggcctggactttccgtaccggtgacctgaagaccccgaacgatccgggcgaaatcactgacgaagtcacgccaatcaaaatcggcgacatgctttatctgtgtacgccgcatcagaagctgtttgctctggatgccgcaaccggtaaagagaagtggaagttcgatcctgagctgaaacccaacccaaccttccagcacgtgacctgtcgtggtgtgtcttatcatgagaccacaccagccgcagaaggcaacgccaccaacggcgcggcgcctgctgtctgttcccgtcgtatcattctgcctgtcaacgatggccgtctgtttgcgctggacgctgaaaccggcgcacgttgcccggcatttggcaacaacggtgagctgaacctgcaaggcaacatgccttatgcaaccccaggccactacgagccaacttcaccacctatcatcaccaaatctgtgatcatcgtggcgggtgcggttaccgataactactcaaaccgcgagccttccggcgtgatccgtggttttgatgttgagaccggtaaactgctgtgggccttcgatccgggtgcggctgagccgaacaaaatccctgaggatggtcagcatttcacgccgaactccccgaactcatgggcacctgcggcttatgatgacaaactggatctggtttacctgccaatcggcgtggcaacccctgatatctggggcggcaaccgtactccggaaatggaacgtttcgcgagcggcctgctggcgctgaatgcgaccaccggtaaactggcctggttctatcagactgtgcatcacgacctgtgggatatggacgtgccagcgcagccaacgctggctgatatcactgacaagagcggcaacaaagttccggcgatttatgtaccgaccaaaaccggtaacatcttcgtgctggatcgccgtgacggtaagctgattgttgatgcgcctgagaaacctgttccgcaaggcgcggcgaaaggcgaccatgtgtctccgacccagccattctccaaactgactttccgtcctgaagccaaactgaccggtaaagacatgtggggcgctacgatctacgaccaactgatgtgtcgtgtgatcttccacaaactgcgttatgaaggcaccttcacgccgccatccgagcagggcactctggtgttcccgggcaacctcggtatgttcgagtggggcggtatttctgtggatacagaccgtcaggtggctatcgcgaacccgattgcgctgccattcgtgtctaaactgatcccacgcggtccgggcaaccctatcgagccagatgcgaacgataaaggcggttccggtactgagactggcattcagccgcagtacggcgtgccatttggcgtgacgctgaatccgttcctgtctccgctgggcttcccatgtaaacagcctgcatggggttacatttccggtgttgacctgaaaaccaacgatatcgtgtggaaaaaacgtatcggtaccgtgcgcgacagctcaccattaccgctgccgttcaaaatgggtatgccaatgctgggcgcaccggtttctaccgccggtaacgtgttcttcatcgcggcaaccgcagataactacctgcgcgcgttcaacatgagcaacggtgacaaactgtgggaagcacgtctgccagcaggcggccaggcaaccccgatgacttactccgtcaatggcaaacagtacgttgttatcgcggcaggcggtcacggttcgtttggcaccaaactgggcgattacatcatcgcttacgcactgccagacgctgatgctaaataattagcggtgttataagttctgaaatctgaaaggccgcgaaagcggccttttttacaactgctgcgcgggtaacattatctgtcacttgctgatgttcattaggttttcctcaggcacccaacctgattctccagatttgttgcatgcccagaaccagccgttgagctttttctccagcgtgaggacttcccccggagcaaccgataattcatatgccgtataattctcagtgcaaattccccggtccggagcactaagggtaaatatttgctggggcgcccatcctgcattgcctgaactctgacgga

SEQ ID NO: 62 - DNA, 2997 bp, Artificial Sequence
  source: /note=“Description of Artificial Sequence Synthetic polynucleotide”
gtcaggcgttcagtaaagacgctgtgctctcatgcaattcatcaagcaaggtataacgcttgcggtattcagaacgtttcttgctggcaatctcttccatcggtttgcgcggcagttccagtggaacgtgatataaggctgaagatgcgggcgtggcactgatggaatcccagaattcgttataactggcgacaaacacgttcttcttgcggtgacggtaacgcaggctgcggaatacgtgaccggtatcgctgacggcaagaatgcgctgcacgccggttccggcagcgacacgttgcaggctttccagcagcagacgtttcgggaacaagccgtaacaggatttagtcgcctgtttaatcacttcatgtgccgttgaacggtgcgcgccctggaatccgccgataatcagtgtcctcccttccgggcgctgcacaatgctaaacgtcagtgccgcaagcagcgtgtcctccatataaaggaacatgttggcttcgccttcgcgctcagatttgccgatggaaccgagctccacccggaaggcttcaccgtctttgccggtgaaccgggtgatggtattgccggtcgcgctcagaaaggtattgcgcaggcgggcgttttccaggcttttgacgaaggcgtagtgatcaaccagtgcgtcagctcgtccggctgaatgcagaccgagatacagatacggcttgtgaattttactcggtagtttggtctgcacctggaaagcttcctgccagagggtttccgccgccatcttattcagcactttcagggtatcgagcggatgcagtaaggtgcgaacggcatatttgacccggaacatccggtcgcgccacagattgtccggtacgcacgctccgctcagaagatcgacaaacaggctgaatccgctggttttttttgtggtttgttcaacgggatatgcttcgctgagggactcggtttcattgagagggatatgttccatgatctgtttccgctgccagaaataatcacggtatcacctgatttttaaacctctttacaacgttgttttaaagagaagcgaggttatctgaataagtgcaggctgtttagctgccgtttattctggctgactgaaagcgtctcactgtctaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctatgaaaattattactactttttgcctcgccagccttttttctgtcaacgcgtttgccctgaccggtaacgatgcgaccaccaaacccgatctttactacctgaaaaacgatcaggcgattaacagcctggcgctgcttccgcccccgcctgcggtgggcagtatcgcttttctaaacgatcaggccatgtatgaacagggtcgtctgctgcgctcaacagaacgtggaaaactggcggcagaagatgccaacctgagtgccggtggcgtggcgaacgccttctcgggtgccttcggttcgccaatcaccgccaaagacagcccggaactgcataaactgctgaccaatatgattgaagatgcgggtgatctggcgacacgctccgccaaagaaaagtatatgcgcattcgtccgtttgccttctacggtgtgccgacctgtaacaccaccgagcaggataagctgtcgaaaaacggttcgtatccctccggtcacacctcaattggctgggccaccgcgctggtgctcaccgaaattaacccgcagcgccaggaccaaatcctgcaacgcggtttcgatttaggccagagccgggtaatttgcggctaccactggcaaagtgatgtcgatgcagcgcgcatcgtcggttccgccgtagtggctaccctgcacactaaccctgctttccagcaacaactgcaaaaagccaaagaagaatttgctaaacagcatccgtaaatattgggcctccccgacctggtcttcgtaaaagccggggagtctgtaatgccacatctctcattgatactaaatttctgaaaattgcttttttgcaacggtcactaattgttaactccccttcacaggcataacgtcattcctcattttaaaaaggagtttcattatggctagccccgcatatctctggctatacgatgccaatggcgcactactttatggcggttctgaggttttaagccgtgaaggcgcgatcgagatccaaagcttcacgcatggtctttccgtacccttcgatggtaataccggtcggctgacatctacccgtgttcatcaaaccatgggactggtaaaagagttcgataaatctactccctacctctaccgtgccgtcgccaccagcgaaaagctgcaaaaggcggtgattaaatggtatcgcattaatgcggctggtatggaggaagagtttttgaatatgacaatggaaggggtgcgcatacttaacatcaatcctcatatgcacaatttcaaacacgctgacggacaggccagtatgcccacggaatccattggccttgggtatcagaaaatcacatggttgtacctcgacggacatatcagttttaccgatcagtggaactccagcatttacgcataaacggagacagggatgatttcatgcagagtggattttaatcgtctttatgagggcggaaaaattgcaaaactttattgtgctggtgtggggatcttccctgtattttcaggacttggttcactgctcaacaggcagggatgttcagatttaaaaaatggtgccattccgccaggaaagtactggattgttgatcgccctgccggcggtttgaattcgtggattgtacaaatgagaaaggagtggaaaacgggtaacgattacgattcatggtttgcgctttatagacaggattctcttatcgatgattggacaaatataggcgccagg

SEQ ID NO: 63 - DNA, 3193 bp, Artificial Sequence
  source: /note=“Description of Artificial Sequence Synthetic polynucleotide”
cttgtttcttgatggctcactggaaggacatctcgatttcaggcgctgtcgtctgggtggtttcgcaggtgggttatttgccattttcgtaccacctgcttcatatatgccacaactgaagcccgattcacctgcggaacctcatgacgcttttgccattactcgggcacagatttcactgctggaacgccttgaaacgcagtccgccgggcgggcaaaaatctgccggacggtcggtgaaattgaagcgtgtattacacagaatgtgctggcgatggtgatgcatatcgaaggggcggaagcactcggcgatgatttctcgcggctggagcgctggtatgaaaaggggctgcgcagcatcggcccgttatggaacttacccaaccagtttggtaccggcgttaagggcgatttcccgggatcaccggataccggagatggcctgacgcccgccgggcttggattgctgcatgaatgtaaccggaaaaggattctgttcgatgtctcgcacatgaacgaaaaagccttctggcagacggcaaaattcagcgatgcgccgctggttgccacccattcaaatgtgcacgcgttatgtccgcaaccgcgtaatctgaccgataaacaactggctgccattgccgaaagcaacggcttcgttggcgttaatttcggcacggcttttctgcgggcggatggaaaacgcaacggcgacacccccatcaccgaaattgttaaacatcttgataaccttgttggtaaactgggggaagaaaatgtcggttttggttcggatttcgacggtatcaatgtgccggatacgctcggtgatgtcgccggattaccgttgctgcttcaggctatgtctgatgcgggatacggcgatgcattgatcgaaaaaatcgcgtaccgcaactggctgaaagtattaaagcaaacctggggtgaatagaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctatgtggactaaaccttcattcgaagacctgcgtttaggcttagaagtgacactgtacatttctaaccgctaagcctttatgcccacggttaactgtgggcatcttccttccctttcccctggttctcacatgcagattatcgtacttggttccgcggcaggcggcggcttcccgcagtggaactgcaattgcagcaactgtcagggtgtgcgtaatggcaccatgaaaacgtccccccgcacgcaatcttcgattgccgtcagcgacaatggcaccgactgggtgctgtgtaacgcctcgccggatatttgccaccagattgccgccacgccggaactgataaaacaagacgttttacgcggcaccgccattggttccattatcctgactgacagccagattgatcactgcaccggcctgctgaatttgcgtgaaggctgtccgcatcaggtgtggtgtacgccggaagtccacgaagacctcaccaccggtttcccgattttcaccatgctttctcactggaatggcggcctgcaacaccacgctatcaggccggagaaccgcttctccgttgccgtctgcccgaatcttacattcactgccattccgctgctgagcaacgcgccaccgtattcgaaataccgcggcaaaccgctccccggccacaatatcgcgctctttattgaagacacaaaaaccggcacctcgctgctgtacgcaccgggtctgggcgaaccggacgatgaactgctgaaatggctgcataaagccgattgcctgctgattgacggcacgctgtggcaggacaacgagctggcgaccaccggcgtcggccgcaataccggcaaagacatgggccatctggcgcttgccgaagaacaagggctgatcgccctgctgtcgtcacttccggcaaaacgcaaaattctcatccatattaataataccaacccgatcctcaatgaatcctctgccgagcggcaggcgctgacgcaacaaaacatcgaagtcagccgggacgggatgcgcatcgaactgtagggcaaaacgaccatgagcatatcgacaacacagacgtcaccgatgacgccgcaagaatttgaacaggcgctgcgtgccaaaggcgcgttttatcacatccatcatccctaccatattgcgatgcataacggtcaggcgacccgcgagcaaattcagggctgggtggcgaaccgtttctattatcagaccagcattccgctgaaagacgcggcgattatggccaactgcccggatgcgcaaacccgccgtaaatgggtgcagcgtattctcgatcacgacggacatggcggcagtgaaggcggtatcgaagcctggctgcgtctgggcgaagcggtggggttagaccgcgatgtgctgctttcagaagaaagggtgttaccgggggtgcgttttgcggtcgatgcctacgtcaattttgcccgccgcgccgtctggcaggaagccgcgtgcagctcgctcaccgaactgttcgccccgcaaatccatcaggcgcgtctcgacacctggccacagcattacacatggattgaggaagaaggttacggttatttccgcagccgcctgagccaggctaaccgcgacgtcgaacacggcctgcaactggccctggagtattgcgataccgtcgaaaaacaacagcgcatgctggaaatcctgcaattcaaactcgatattttgtggagcatgctcgattccatgagcatggcctacgaactgaaccgcccgccgtaccacagcgtgacgcagcaggcggtctggcataaaggaagactcctgtgatcaccattaccgaacactacacgccgatgtttcgtcgcggctaccgcatgcagtttgagaaaacgcaggactgccatgtgattttgtatccggaagggatggcgaaactcaacgacagtgcgaccttcattttacaactggtggatggcgggcggacaattgccaatattattgatgaactgaatgcccgctttccgcaggccggtggcgtgaatgacgacgtcaaagacttctttgctcaggcccatgcccaaaagtggattatcttccgtgaacctgct

SEQ ID NO: 64 - DNA, 4410 bp, Artificial Sequence
  source: /note=“Description of Artificial Sequence Synthetic polynucleotide”
tgggaagccccggagagaaagagccaaatgctcattgatgatttttgaaagttcgcgttgttcgtccggcaatatggcgttgaacactgcgtgaacctaccctgttcacgcataatgatatgtttatttacttgatatttccgctctttattactttcgaaagtgatatttccggcgcgggaatcccaataaaaaaagcctgaatttcttcaggcccgatctttgccatcctgatgacagattactggaagtaaatatcgccgttttcggctttgaggatcttgccgtcggtgtcagtgatcagtgcgtagctgccgccgatataagtccagtggctacccgcatccggtgccggcaggtgacgttcctgccagccgacgatctcatacggcttggtacggtacagcgcagggacctgatcgccgatggaatacaatttggaatcatcgtagaacgatttgatctcgtgtgagtttgacgcggcaaacgccgtgttggccaggggtaacaacgatgaaacaaccagggctgataacataacaattttagaatgacgcatagattatcccgttaaaaccatcaacttaatgcctgcctcggtgatatgccgccagtccgttgtatgagcagtaaagccgaagcctgtgctttttctgtaggacaggataataacgagaaagtgagtgcaggctgggagatttttttgtgttacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaacatgcgcaatgaacggttttttagctggtcgttcatgaccgggcttgtcacggctgtcatcgggctggcttatctggtgctgggcgtctggctggcggccctcggcggctcaccctggtatgtgctttttggcagcggatatctgctcagcggcatttttatcgcgcgccgccatgccagcgggatctggctttatctgctgacatttctgctttgctgcgtctggtcggtatgggaagtcggactggatggctggcaactgatgccgcgtttgtttgtgatggcgttattaggcgtctggtgcagcctgccgttgatcacccgtcaggtgatggcaacgcggggcaatcaccgcaccggtacgttcgccgggctggtgtatgtggtggcgatcgtgggtattttttacagcggctggcaggtgacagggtcacgttttgtccatcgtcagcctgttccggcgcaatcgggtgatatacaggccacgtcgccggaaagcaacgactggcgctattacggcaggacagaagccggacaacgttattcgccgctgacacagattacacccgctaatgtcagtcagcttaaacccgcctgggaatttcacaccggcgatgtgatgagaaagggtgaggataaagacggacgggaatttaatttcgaagtcacaccggtcaaagtcgggaactcgctgtttatctgtacgccgcaccgcgaagtgattgcgctaaacgccaccaccggcgcgcagcgatggaaattcgatcctaaatctgacacctcggccaatgagtatctggcatgtcgcggcgtggcatacagccagtcagccggtgataaagtgtgtccggaaaaaatcatcgccaccaccagtgaggcgcggatggtggcgctgaatgcacagaccggcgaaccctgttcatcctttggtcagaacgggtttgtcagcctgaccgaccatatgggcgacgtgccgccgggcttccacttcattacctcgcagccgatggtgatggatggccgcatcgtgctgggcggctggatttatgacaaccagtctacgggtgaaccttccggcgtcgtccgggctttcgatgtgaacaccggccagcttgcatgggcgtgggatatgggccgggatccgcaaaatgcgccgctcaaaccgggtgaagtgtatacccggggcacgccaaacggctggggaacctataccggtgatccgaaactggggctggtttatattccgctcggcaatgcgacgccggattattacggtgccggacgccgtccgtttgacgaaaaatattcgagttcgctcgtcgcgctggatattcacaccggcgaggaacgctggcacttccagactgtgcatcatgatgtctgggactttgacttgccgatcggaccgacactggtggatttgccgtcaccggaggggataaccgtgcctgcgctggtgcagaccaccaaaatgggacagcttttcctgctcgaccggcgtaccggcaaaccgctggcgcaggtaaacgaaaaaccggtgaatacctctccctctttgccgggcgaacatctgtcgccaacgcagccggattctgtcggcatgcccagtctctctccgccagatctgaaagaaaccgacgcgtggggcgcgacaccgattgatcagttgtattgccgcatccagttcaaaagtgcccgctatcaggggcagttcaccccgccagcggaaggtaaatccattgcttatccggcctttgacggcgtgatggactggtacggcgcttcggtggatccgatccgccatgtgctgattgccaataccagttacatcccgttcacgatggaagtgaaaaagtcagccgatgcgatcaaagaagggctgatgcacaaatgggccggatggggcagcaaccagccttatccaaaacccaaagagttttcggttggcccgcaatatggcacgccgtgggcggcgatcgtcaaaccgtggctgagttttctgcaggcaccctgtaatgcgccgccgtggggaaaactggttgcggttgatctgaccacccgaaaaatcgcctgggaaagaccggcaggcacgacccgggatatgaacatttttggcacgcataccaacgtgccattgccgaccgggatttttatgatgggcggtaacatcattacccaaagtggcctgattttcaccggcgcaacggcagacaactatttccgcgcattcgacgaaacgacgggtaacgaactctggcgagcgcgacttccggcgggcgggcaggcgacgccgatgacatataccggcgatgatggccgccagtttgtggtgattgccgccggcggacacggcgggctggggacgacgtccggtgatgcgctggtggcgtatgcattaccggccagatagggttggcactcaaacgcaggatattcagaaccgtctgatgatgcccacaggcggcgcagcatggtctatttttaagatcccttcttttttaaataaggagagtttatgaacaacaaaacaatcccgacatccctgaatgaacctcatgttgatgcgtatccgaccccgccgtttgagcatcagaaacagccattcccggggctggccagcaaaatgaacccggtacccgatcacggcgagaaaacctacaaaggcagtgcccggctggaaggccgtaaggcgctgattaccggcggtgactccggtattggccgtgcggtggcgattgccttcgcccgtgaaggtgcacaggtcgccatcaattatctgccggacgaagaggccgatgccaaagaagtgatcgacctgctgtgggccgaagggcggaaagtgattgccattccgggggatattcgcgacgagaaattctgtcagcaactggtgaaggaagcggaagagaaactcggcgggctggatctgctcgtcaataacgccggtcgtcagcagttctgtgattcgattaaggatctgaccaccgaagccttcgacgccacgttcaaaactaacgtgtacgccatgttctggataaccaaagcggcgctggaattcattccgcgaggcggtgcgatcattaatacgacttctgttcaggcgttttcaccgagtgataatttgctcgattactcctccaccaaggcctcgatcatggccttcaccaaagggctggccaaacagctggcgggggacggaattcgggtgaacggtgttgcgccggggccgtactggacgccgttgcaaatttccggcggacagcctcaggaaaaaatcgaatcctttggtcagcaggcaccgctgaaacgtccgggacagccagcggaaatcgccccgctgtacgtgacgctggcctcaaatgaaaacagttatgcctcagggcaggtctggtgttctgatggcggtaccggcacggtctgattaccggtcggaaacgccaaagcccgtcatattgacgggcttttttcatgcaggcagatatccggcgttttttgccgacatcggcacgtcataaataattacgttgctaat

SEQ ID NO: 65 - DNA, 4533 bp, Artificial Sequence
  source: /note=“Description of Artificial Sequence Synthetic polynucleotide”
tgggaagccccggagagaaagagccaaatgctcattgatgatttttgaaagttcgcgttgttcgtccggcaatatggcgttgaacactgcgtgaacctaccctgttcacgcataatgatatgtttatttacttgatatttccgctctttattactttcgaaagtgatatttccggcgcgggaatcccaataaaaaaagcctgaatttcttcaggcccgatctttgccatcctgatgacagattactggaagtaaatatcgccgttttcggctttgaggatcttgccgtcggtgtcagtgatcagtgcgtagctgccgccgatataagtccagtggctacccgcatccggtgccggcaggtgacgttcctgccagccgacgatctcatacggcttggtacggtacagcgcagggacctgatcgccgatggaatacaatttggaatcatcgtagaacgatttgatctcgtgtgagtttgacgcggcaaacgccgtgttggccaggggtaacaacgatgaaacaaccagggctgataacataacaattttagaatgacgcatagattatcccgttaaaaccatcaacttaatgcctgcctcggtgatatgccgccagtccgttgtatgagcagtaaagccgaagcctgtgctttttctgtaggacaggataataacgagaaagtgagtgcaggctgggagatttttttgtgtttaaaaacgtgaccacgagcattaatgaacgctgcgaaatgtggcgtttatttattcaaaaagtatcttctttcataaaaagtgctaaatgcagtagccgcaaaattgggataagtcccatggaatacggctgttttcgctgcaatttttaactttttcgtaaaaaaagatgcttctttgagcgaacgatcaaaatatagcgcttaccgacaaaaaattattctcattagaaaatagtttgtgtaatacttgtaacgctacatggagattaacttaatctagagggttttataatgcgcaatgaacggttttttagctggtcgttcatgaccgggcttgtcacggctgtcatcgggctggcttatctggtgctgggcgtctggctggcggccctcggcggctcaccctggtatgtgctttttggcagcggatatctgctcagcggcatttttatcgcgcgccgccatgccagcgggatctggctttatctgctgacatttctgctttgctgcgtctggtcggtatgggaagtcggactggatggctggcaactgatgccgcgtttgtttgtgatggcgttattaggcgtctggtgcagcctgccgttgatcacccgtcaggtgatggcaacgcggggcaatcaccgcaccggtacgttcgccgggctggtgtatgtggtggcgatcgtgggtattttttacagcggctggcaggtgacagggtcacgttttgtccatcgtcagcctgttccggcgcaatcgggtgatatacaggccacgtcgccggaaagcaacgactggcgctattacggcaggacagaagccggacaacgttattcgccgctgacacagattacacccgctaatgtcagtcagcttaaacccgcctgggaatttcacaccggcgatgtgatgagaaagggtgaggataaagacggacgggaatttaatttcgaagtcacaccggtcaaagtcgggaactcgctgtttatctgtacgccgcaccgcgaagtgattgcgctaaacgccaccaccggcgcgcagcgatggaaattcgatcctaaatctgacacctcggccaatgagtatctggcatgtcgcggcgtggcatacagccagtcagccggtgataaagtgtgtccggaaaaaatcatcgccaccaccagtgaggcgcggatggtggcgctgaatgcacagaccggcgaaccctgttcatcctttggtcagaacgggtttgtcagcctgaccgaccatatgggcgacgtgccgccgggcttccacttcattacctcgcagccgatggtgatggatggccgcatcgtgctgggcggctggatttatgacaaccagtctacgggtgaaccttccggcgtcgtccgggctttcgatgtgaacaccggccagcttgcatgggcgtgggatatgggccgggatccgcaaaatgcgccgctcaaaccgggtgaagtgtatacccggggcacgccaaacggctggggaacctataccggtgatccgaaactggggctggtttatattccgctcggcaatgcgacgccggattattacggtgccggacgccgtccgtttgacgaaaaatattcgagttcgctcgtcgcgctggatattcacaccggcgaggaacgctggcacttccagactgtgcatcatgatgtctgggactttgacttgccgatcggaccgacactggtggatttgccgtcaccggaggggataaccgtgcctgcgctggtgcagaccaccaaaatgggacagcttttcctgctcgaccggcgtaccggcaaaccgctggcgcaggtaaacgaaaaaccggtgaatacctctccctctttgccgggcgaacatctgtcgccaacgcagccggattctgtcggcatgcccagtctctctccgccagatctgaaagaaaccgacgcgtggggcgcgacaccgattgatcagttgtattgccgcatccagttcaaaagtgcccgctatcaggggcagttcaccccgccagcggaaggtaaatccattgcttatccggcctttgacggcgtgatggactggtacggcgcttcggtggatccgatccgccatgtgctgattgccaataccagttacatcccgttcacgatggaagtgaaaaagtcagccgatgcgatcaaagaagggctgatgcacaaatgggccggatggggcagcaaccagccttatccaaaacccaaagagttttcggttggcccgcaatatggcacgccgtgggcggcgatcgtcaaaccgtggctgagttttctgcaggcaccctgtaatgcgccgccgtggggaaaactggttgcggttgatctgaccacccgaaaaatcgcctgggaaagaccggcaggcacgacccgggatatgaacatttttggcacgcataccaacgtgccattgccgaccgggatttttatgatgggcggtaacatcattacccaaagtggcctgattttcaccggcgcaacggcagacaactatttccgcgcattcgacgaaacgacgggtaacgaactctggcgagcgcgacttccggcgggcgggcaggcgacgccgatgacatataccggcgatgatggccgccagtttgtggtgattgccgccggcggacacggcgggctggggacgacgtccggtgatgcgctggtggcgtatgcattaccggccagatagggttggcactcaaacgcaggatattcagaaccgtctgatgatgcccacaggcggcgcagcatggtctatttttaagatcccttcttttttaaataaggagagtttatgaacaacaaaacaatcccgacatccctgaatgaacctcatgttgatgcgtatccgaccccgccgtttgagcatcagaaacagccattcccggggctggccagcaaaatgaacccggtacccgatcacggcgagaaaacctacaaaggcagtgcccggctggaaggccgtaaggcgctgattaccggcggtgactccggtattggccgtgcggtggcgattgccttcgcccgtgaaggtgcacaggtcgccatcaattatctgccggacgaagaggccgatgccaaagaagtgatcgacctgctgtgggccgaagggcggaaagtgattgccattccgggggatattcgcgacgagaaattctgtcagcaactggtgaaggaagcggaagagaaactcggcgggctggatctgctcgtcaataacgccggtcgtcagcagttctgtgattcgattaaggatctgaccaccgaagccttcgacgccacgttcaaaactaacgtgtacgccatgttctggataaccaaagcggcgctggaattcattccgcgaggcggtgcgatcattaatacgacttctgttcaggcgttttcaccgagtgataatttgctcgattactcctccaccaaggcctcgatcatggccttcaccaaagggctggccaaacagctggcgggggacggaattcgggtgaacggtgttgcgccggggccgtactggacgccgttgcaaatttccggcggacagcctcaggaaaaaatcgaatcctttggtcagcaggcaccgctgaaacgtccgggacagccagcggaaatcgccccgctgtacgtgacgctggcctcaaatgaaaacagttatgcctcagggcaggtctggtgttctgatggcggtaccggcacggtctgattaccggtcggaaacgccaaagcccgtcatattgacgggcttttttcatgcaggcagatatccggcgttttttgccgacatcggcacgtcataaataattacgttgctaat

SEQ ID NO: 66 - DNA, 6053 bp, Rahnella aquatilis
cagttgcagtttcagcggcaccgtttcgcccggctgtaatgaccacggcggcgtggccagatacactgataaggttttacgctgtttgaattccatcaccggcatattattgcggtcaaccatgtccatccggcttccgcgcagcgagcgcgctgccgcgacgttctgtgaggacaactgctgatccagtggcacacccagccggtaattcagtgtcagtttcactacatcctgattatccccatcctgtccggcctgatgctcggccatcaccgtcacgatcggcaccggggtgtaattgacgccaaacgacactgccgacggattactctgccgcgtgccgttgccgaacaaatccacctgattaccgaaatactgctcataactcagcgaagcgcctaactggcggtagaaaggtaaatagcctttggtggtaatgtcgtagccacgcgcctgacgccgcagttgcgcagacgtgctgctgtcggctttataggaggaaaacggctgataataattggtagaaaagcgcaggtaatctgtccccgcctccgcgcccagactgccgcgcgtcagctgatggtcgaagtcgttatccacaaacgcgttataccccagcatccacgtaccggtattaaagcgctggccgaggccgaaattaccaaccgtggcatccgtctcgctttgcagaccgatctggctgtaaaccagattggtttcagactggtacagcgggctgaataaccgaccggaactgccgtcaagattatggccggtcatgtccagcgaggtgctgacggtgcccagcggcgaaagcaaactgctggtttctttttgcaccagtttcgccgcctgactcagtgccagcgtttccgcctgcacccgtaagggatcatcgctgtccgccgtcgtggttcccagctctttcagagcggtggcaagctgtttttccatggccgtgggcccggtattgttctgtatgtactggctgccgaggctcggcaacatcccggcgggttgtgccggcgttccgttattaaagacggaactttggtcactggcgggcagaaaaccgttgaggttataaccgggaacgccgtattcgccagatcctaccgtcagcgcactggcgcttacagcatagaaggcggctccccctgcaagcagcaaagaggcgcaggtgtttcctgcaaatcgcttcaataaatcagaaaaaaaatctgtgacttcagtcataccttacacatttcatgagtaaggtatgatcttaccgtacagtcagcataattcaaccttcacgccacgaacggccacacaccgccttatcgccagtcaaatcatgctcattcatttccagagtgaatgcagggctaatttgttgccacatcgcaaaaaatatcgcctaaaattacatcaaagatatcaatcacacacatttaataacatttttaacaaacgaactatttatctttacattctattttcatttatccttagcaggttaacgattatcccgatgccacccgactgccctggcagagtgcgtccggaccttaaagtgatatgtacaaacagggaacataaatgagcaaaaaaaccttgcccggcgtgacccgcagacaaattctctcattcacggccgcctccgcagtattgggtgcagccaaagccgcaaatgcggtgacgctcaaaggcacgcccgtgtggagcccctttgacgccagcccgccgccgcacatcgagaccgatggctgggtgttcttcaccgaagccgaagccgccgccatggaagccattgttgaccgtctggtgcccgccgatgatctgagcgtgggcggaaaagacgccggatgcgcggtatttattgaccgccagctggccggattctacggcaccgcctcccggctttatatgcaaggccctttccaggatggcacgccggaacaaggcgatcagtcgtcgctggttccgcaacagcgctaccgtcttggcctggccgcattagataaatacacccggtcacagcatcagaagttattcaaagatctgcccggtgagcagcaggatcacatcctgtcaggtctcgagagcggaaaaattgcccttgaagggatcgactctaaaatgttctttgccattgcactgaagaacacgatggaaggtttctttgctgacccgatttacggcggtaaccgcaatatggtgtcgtggaaaatgctgggcttccccggtgcacgttacgactaccgcgactatatcggcaagcacaatcagaaactcgatctcgaaccgctgagtatttccggcggtgacgcatggaaaataaaaagctaagggcagaacacgattatggcaaaaaaattacctaaaaccgacgtggtggtgatcgggctcggctgggcaggttcgatcatcgccaatgagttgtgcgacgaagggctgaatgtggtggcgattgaacgcggcccgtggcgtgataccgcccgtgattttaacgtgtcgacggtgacagatgagttacgctacagttcccggcaggaactgatgttacgtacccgccagaacaccattaccatccgcaacaacccggcgcaaaccgcgttgccgatgcgcgaatggggttcttttcacccgggaaatggcaccggcggcgcaggcaaccactgggcggggattactttccgctttcagccggatgaattccgcctgaaaagtcatctgaccgagcgttacggcgctaacgccatcccggaagagttggttctgcaagactggggcaccgactgggatgaaatggaaccgcattacgcctctttcgaacgtctggccggggtttccggtaaagccagcaacgtcaaaggggaacatcatgaaggcggtaacccgtatgaagggatgcgctcgattgaatatccgacccgcccgctcgatcagccctatggcccgacattgtttgctgaagcggcgcgcaatatgggttacaaagcgttcccggtgccctcctccctgatttccgagccttatactaacccgctcggggtgaaaatggggccgtgcaccttctgcggtttctgcacaaacttcggctgcgcgaactactccaaggccagcgccatcaccaccgtattaccggcgctgatccgcaaggagaatttcgaagcccgcactaactgtgaagtgatgcaggtgctgaccgactctaccggcaaacgcgccacgggcgtggtctatatcgattcttcgggcgatgaatgggaacaaccggctgacctggtgattgtgagtgcgtttaccttcgaaaacgtccgcctgatgctactttccggcatcggcaaaccctatgatccggtcacgttaagcggcaccactggccgtaattatgcctatcagaccgcgaacggcgtacagctcttctttgatgacaaaaactttaacccgttcatcggcgcaggcgcggttggaatgggcatcgatgatttcaataacgataactttgaccacagcggcctgggcttctttggcggtggcagtatccgcgtaacgcccatcggcggagcgcccatcggctaccgccctgttccgccgggaacgccgaaatggggcagcgaatggaaaaaagccacagtcgccaactatctcagttcgatgtcgatcggttgtgaagccagtagctacaccactaaaaccaactatctgtcgcttgatccgaactataaagaccgtctggggcgtccgctgctgcgcgtcacttttgacttcccggcaaatgacctgaaaatggccgcctattgcaccggtaaagtagccgaaatcgccaaggcgatgaatcctcgtcaattggtggccaccccgatgaaaggtcactggaacggtacgccgtatcagtcttcgcacgtcgtcggcgggtttgtgatgggtgcggatccgtccacgagttcggtgaataaacatctgcaggtctgggatgtgcctaatctgtttgtggtcggcgcgtctgcctttccgcaaaacccgggctataacccgaccggaaccgtcggcgcactggcgtttaaagccgcccatgcaatccgtaactactatctgaaaaaaccgggagagatgatcgcatgaaaaagttaacgtcattctcactcggtttagtggctttgagcatcagcggactgagccacgcggcgggcgatggctctttcgaacaggttgaacgggggcgttacctggcgaccctgggcgactgcgccgcctgtcataccgcctccacgccggacgccaaaccttttgccggcggggtgccgattgaaacgccatttggccgtttagtcggtgcaaacattacaccggatacgcaaaccggtatcggcagatggagtttcgatgatttccagcgcgcaatgtcggagggcatcggtcacggcggaaaacgtctttacggcgcgatgccattcacggcgtacacgaaagtgacacgcgaagataacgccgccatctgggcgtatctgcaaactttgcaaccggtgcataatgaaattgaaaccaaccagttgcccttcccgttcaacgtgcgtaccagtctgatgggctggaactggctgaacttcaacaaaggcgaatttaaaccgcagatggataagtcggcgcaatggaatcgcggtgcttacatcgttgaaggtctgggacactgcggtacctgtcatacgccgaagaacctgctcggcggcgacaaagacagtgaattcctgcaagggaccgtgatagaaggctgggtcgcgccggatatcaccagcaacaaacacaccggtgtcggtaactggacgcaggaagatctgatgcaatatctgaaaaccggttctaaccgtttcgatattgcttccggtccgatggcggaagaagtcaccaattcgtcccgtcactggacggatgatgatcttaaggccgtggcggtttatctgaaagacagcggacatgacagcggtaacaacgcccctgagccagtaaaagctgacgataaggcgatgatagctggcaagaatatttatgctgatcgttgttctgcctgccataccccaaccggtatgggccaggaaggtttgttccctcgtctggccgattcaccacttatcaaccagaccgacgccacttcacttatgcgcgtagtactggcgggcagccgtcctgtggcaacggacagcaaaccgaccgcgccatcaatgccatcgtttgatgccaccatgagcgatgccgatgtggctaatgtgctgacgtatatccgtaacagctggggaaatgccgccgcgccggtttctgcctcagacgtgaaagacttacgcgcagaactgaaaaaataagcggaaagcctgaaaacataaaggccgcgaaagcggtctttttcatgaatgcagccggggaaatcaatgccggggaggcatctgcataatctggactaacaaatgatcattattctggcttaactgcaccaggtgtttgcattctacttttattgctgtgagttgttcttccgtcagcgagtaaggcagattgatatcaatactgtacatattttgctgctcagccagttctatcagccggacaatattggtctcgtcgctgacctgggtggaaaccacctgtaaggccagtgcacgcaaagcttcctgcctggatttttcttttttcgaacgggatttcagaaccataccaaaaaatggcatcacgccataaatagcatcaacaaaactccatttctttttgcaggatgcggacaggtattccatatagtcaaaggatactttttgactgcgcgcatcgggtaccaggcggttttcattcatcccctcttcctcttcctggtcagggtcaatcaattatttaatcaatgtataacgccgaagatattcatctatataatggcataattcactgacactttgccagtcgcttgcggtgctttgcgtaacggaaactgacaacggcaatatttcagaatttgaccaaagtattttgcggttatggaaaataccgcgcaaaactgacatgttctattacgctagcacgat

SEQ ID NO: 67 - DNA, 5569 bp, Rahnella aquatilis
agtgaagacagacaatgatctttgataactgatgctaatttaatttgagttttggtaaataggaaatttataactatctgttttaaaaggatttaaaatctattctatgattatttaaaaccaccacattagccaccgcacagttcgcatggaatttattcatttattttagatgtagcagtgatattataaatttgtaccccaaaataataataattaacccaaacttaacatcaggcatgtgagagtcgtaattctatgctccttcaaaaaaaaacaacccgtcgcaagtttttacttgggtcgctgatggctctgccggtcggcaccataatgatgaaaggattgtcggcggcacaagctgcggaaatggctgccccggatttactcgattataaacccatatttttcagtaccgatgagtggcagtttattatggccgctgctgaccgcctgatccctgctggtggcaaaggtaaagcaccgggcgcactcgaaaccaacgtgccgatttttatcgaccagcaaatgcacggtgattttggtgatgaaatttacatgcagggcccgttcaatgtgcatgctccggccacaatggggtatcagatcccgttccgtccgcagcaaatctacaaaaccggcattcgcctggcgaacgcctggtgtcagcaaaaccaccagaaagccttccatgacctcgccgctcaggacaaagacaacgttctgacgcagttgcagaaaaatggcattcagtttgcggatcttggtgaagagaacctagtggcttcgcaattcttcagtgaactgctttcagataccaaacacggttatctggcagacccgatctacggcggaaacaaagggatgaaagcatggatagccatcggcttccccggcgcccgcgccagtttcaccgaatgggttaagcagcacaatgtcccttatccgctgggacctgtgagcctgcaaggcgcgcgtgcctgagtgcccgccctattaccgtttctgatgaatgacaggattaaagatggcaaaagtaactaaaccggaagtcgacgttgttgttgtcggcctcggctgggctggctcactgatgagtattgagctggcaatggcgggcctgaccgtgcgcgcgcttgagcgtggcggcgatcgtggctatgaagaatttgcttacccgaaaccggcggacgaatatgcctacgcggtacgcaataaagtcatggcgaccccggcagaagccgccgtcaccgtgcgttacaacatgcgtgaaactgcccttccgacccgtaaatggggtgcatttgcgccgggtaccggcgttggtggttccggtctgcactggacagcagtattgattcgcccgacgccgactgatttaaaactcaaaacctacgctgacgaggcttacaaaccgggcatcttgcaggaagatatgcggatcatggatttcccattcacatgggatgaaattgagccgtattacactaagtttgagcacatctgcggccagtccggcaaaaccggcaatcttcgtggtcagattatggaaggcggtgatccgttcgaagggccccgctccgagccctatcccctgcctgcactggaagatacgcttaacagcagtatgtttgctgaagtgaccaaaaagatgggctatcacccgttcccgaatccgtcagcctgtgtctcccgcgcctggactaacccttacggcaaccaaattgcaccctgtaactactgtggttattgcagtaaatatccgtgcctgaactactcgaaagcctcaccgcagacggcggtcatggatgcacttaaacgtatggataacttctcctatgaagtgcatgccaacgtgctgaaagtcgaactgcatgatgataagaaaaccgcgaaaggcgtgatctacatggatgcagacggcaacgagtgtttccagcccgccaaaatcgtggtgctgagcagcttccagttctgtaacgtgcgcctgatgctgctgtccggcatcggcaaaccttacaacccgatcactgaagaaggtgtgattggccgcaactatgcattcctgagtaacggcggctctacgctgttcttcaaagacaaaaacttcaatccgttcgctaccgcaggtgccaccggccagatgttcaacgacatctctccgggcaacttcgacggccctgcgctgggctttatcggcggcgcgaaaatccacagttcgcaggccaccggcacgccaatcagcacctcgctgccaaaaggcacgcctgcgtggggcaccggctggaaagaaggcatggaagaatggtacggccattcaatgaaaatcagcattaccactacctgccagtcttatcgcgatatttatctggatctcgatccgaattataaagatgaatatggatatccgctgctgcgcatgaccttcgactggaaacagaacgaactcaaattgcagcaatatctgaaaggtatcgttggcaatatcactaaagagctgaacccggacagctacagcgaaagtttcctgccgatggacgcgcactttgacctgaccaaatacgtttccacgcacaacgtgggcggcgcggtgatgggtgacaacccgaaaacttctgcgctgaataagttcctgcaaagctgggatgtgcataacgtctttgtgccgggcggtaatgccttcccgcaaaacttccaggccaacccgaccgataccattggtgcgatcaccctgatggcggcgcaggccattaaagatcagtacctgaaaaatcctggtccactggttcaggcataagcggagatgaacacgatgaaactcaaaagtttatttatcgctaacgccttgctgctgggtgcaggttttatggcgaatgcgcaggcagccacgaatgcacaagccgacaacgtcgcgctcataaaacaaggtgagtatgtggcgcgcctgggcgactgcggcgcgtgtcatacggtggctggcaaaccggcgttttcaggcgggctggcgatcaactcgaatctgggcaccatttattcgaccaacattacgccggataaagaccacggcattggcggttataccgaagcccagttctcggatgcggtgcgtaaaggcgtgctgccggatggcacccgtttgtatccggcgatgccttaccctgattacgccaaaatcagcgatgaggatatgcacgcgctctacgtttactttatgcagggcgtaaaaccgagcgcggaacaaccgccggaaacggatctgagcttcccgttcagccagcgctggggcatgcgcttctggaactgggcgttcgcgtctgacaaaccattccagccaatcggcggtgcgtccgcacaggttaaccgcggcgcttatctggtggaaagcctgggccactgcggcagttgccacactcctcgcggcttcggcatgaatgaaaaagcactcgacagcagcgactctgattttctggcgggtggcagcctcaataactgggacgtcccgtcgctgcgcggcgtagcgcactggaacgaacaggagatcgtcgattatctgggcaaagggcgtaacgacaaagcggcggtaggcggcgaaatgacgtctgtggtggaaaatagcaccgcccatatgaccgatgaagacctcaaagccattgcggcatacatcaagttcctgggcggtaatccaccggttccggcacaagattcgcagaaagccagcgcgaccgaagccagactgacggcagcgaaaaatctcaccgaaggcgagcgtctgtatctggataactgtggtgcctgccactttgtgactggcaaaggcgcaccaggcgtgttcccgcagctggatcaggcaacgattgtgaacgcgaaagacccgacaggactgatccacaccattctcgcgggtgcacagcaaccttcaacggcacgtgcaccgtcgacactggtcatgccgggctttgcccaccgtctgactgacgaagaagcagcacaattgtcgaccttcatccgtcagggttggagcaacaacgcaccggctgtcacagcaaaagacgtcagtgaagtgagaaaaactctggctcactaaacgttctgctcatcaacaaacggctttcctcacggaaggccgtttttatgtcatcttctcaaagggacgcctcgccgaacaccacgatgcgattgcggccgctttgcttcgcctgatacatcgcagcatcagctttcgccacacaagcttcggctttatcatcctcactacccataaaatagatgccgacgctgaccgttaccggcgcggggagagtgccgggaaattctgacctatgatgttgtgtcacgttgatacgaaaacgctctgccatcgtacgaacctcctcttcagaggcaccgatcaccagtgcggaatattcctcaccgccaatgcgtgaaggaatatcttgatcacgcagggaatgcgaaagtgaccaggcaacatatttaatgacctcatcgccctgaatgtgtccgtaggtatcattgaagcgtttgaaataatcaatgtcagccaccattaccgccagccttacccctgattgttttttatctaaataagttttcattgcatcgtaataataccggcgattatagagattagtcagcggatcgcgaatagaagactcataagaaatctgatatttcatcaatgttttcttgtaaaccatagaaatgtcgtatagtaaaacaatcacgacaaagaacgtcgataacacctcaaaaaaacgcgcgtaataccagaccagcgaatgaatataaatgctgctatgtaaaatagatacgttgcctaaataggaaatgcagctcaatccgatagcaagccagaaggtatttttaagccgggtcacagaaataatcaggacggtggccagtgcccagataccgccaataatataccccatttttccagaccaaagctggctataatcacgcccgtctgccgttacaatctccatgccgaacagcggatttttactggaaagaaaataggcaaagatcacagccaggcagcttgcggccagcacaatgctcagcacccgacgatggctgatgcacagagtatccctgcgctgatacaggaaagcagtgagcaaaaatacagcggccatcgagatactgcgaaagacccagtaaatagccagatcattgacggagccttcaagcactgccccggtcaggaagatccccggataatagagcagcttgtaaaccagatagaatgccgacaacgagtacgcaaaggataatacagccaggtagagtttcttgcgattcgccatgtattgcatcagcatgaaaaaggcgataagggtatgcagcacaaataaaatggcggtagtgacgggaaatattgagactacagcaggcgtttcatgaaaaaaatagcgatgagacatcaccgtaacaggaaccaaaacaatcaatatgtataagaaaaacactaacatatttaacttacacttatctttaacatcaaataacgtcatatcacgcatccctctccgttgggaataagcctctgtcctgcaaggcggcgtga

SEQ ID NO: 68 - DNA, 3769 bp, Rahnella aquatilis
cggtattccggtggtagagatgatggattgcgtatcgccttgcgtggatctggcggtcggtttcaacaactttgaagccgcacggcagatgacccagcaaattatcgcgcgcggccatcgtcatgtggtgtatttcggcgcacgtcaggacgaacgtacccttattaaacagcagggttacgagcaggcgatgcgtgaatccggtctggagccttacagtgtgatgaccagccgctcttccagttacagtggtggtgctgagttattgcgacaggcgcaaaaagactggccacaaatcgacagtattttctgtaccaacgatgaccttgcggcaggtgcattctttgaatgccagcgtcagggactgaaaattccttcacaaatggcgattgccggttttcatggacacaacatgggacaaacgatggagccaccgctcgccagcgtcctgacgccgcgtgaaaaaatggggcaaatctccgccgaacgcctgctggcgagacttcgcggcgaagaagtggtgccgagaatggtggatgtagggtttacgatttctgcgggcggaagtatctgatttctttgcgccttcccctctcacgaggggatggctgggatggggtgttggttttgcggatgacagcgctgccattaagcccccctcccgacttccccggtctaaacatcccccttcgcaggggaaagtcttacttctgtaccgtcttcaaagccgcgatacagcgatccaccacaccttcaaacttgtgatcgatatttacgcgcaacacgtccggctcgtcttcgccgggttcttcgagtgcttcaaactggctgcgcagcaaatcggtcggcatgaagtgtccggcgcgtgctttcagacgttcggcaatcacctcaaaactgcctttcatgtaaataaataccatgccttcgttgtctttacgcagggcgtcgcggtaacggcgcttcagtgcggaacagacaatgatgccggtttcatttttatggcgcaggctgtaagccgcgtcgttgaggcgtaatagccacggtgcacgatcatcgtcgttcagtggcgtgccgctggccatcttttcaatattggcgcggggatgaagatcatcgccgtcgataaatttcgcgttaatttcacgggccagtgccgcgccgacggtggatttaccgctgcctgacacgcccatcagaatgatgctttgtcctgccataagtatgatctctatcccaaaatgaacattttgactgcgtagtgagtcctaatattagcatgttaccggtatcatgataccggtaacaaatggagatgtgactaatatcacaaaggaatgcactgctgttttagcggtcgcggtctgtacccgttgcactgttaattcctacaacatcttgaaaactaaaaataatgcgtcacccggtttttagccatacctgagtgacagcataaggggaagaatatgccattagttattgtagcggtgggtgtcgcactgctgttgttacttatgatccgatttaagcttaatggcttcattgcactgattcttgtcgcgctggcggtggggattatgcagggaatgccggtcgataaagttatcacctcaatcaaaaacggggtgggtggcacgcttggcagtcttgcgctgatcatgggcttcggtgccatgctcggtaaaatgctggcggattgcggtggtgcacaacgtatcgcgaccacccttatcgaaaagtttggccgtgagcacattcagtgggcgattgtgctgacaggtttcatcgtcgggtttgccctgttctatgaagttggcttcgtgctgatgttgccactggtcttcactgtggcggcagcagcgcgtctgcctttgctgtatgtcggcgttccgatggcggccgcgttatccgtcacacacggattcctgcctccgcaccctggcccgacggcaattgcgacgattttccatgcggatatgggtaaaacgctgttgtttggctcactgctggcggtgccgaccgttattctggccggtcctgtgtatgcgcgtttcctgaaaggtatcgacaaaccggtgccggaaggtctgtttaacccgaaaaccttcacggaagaagagatgccgggctttggcgtcagtgtcgccacctccctggtgccggtcattctgatggcgtttcgcgcgctctgcgaaatgatcctgccgaaaggccatccggtgctggcgtatgctgaattcttcggcgacccggtgatggcaacgctgattgcggtgctgatcgctatcttcacctttggtctgaaccgtggccgcaagatggaagatgtgatggcgactgtgaccgactccatcaaaatcatcgcgatgatgttgttaattatcggcggtggcggggcgttcaaacaggtgctggtggacagcggtattgaaaaatacatcgcggctctgatgcacggcagcaacctgtcgccaatcctgctggcctggtctatcgcagcggttctgcgtatcgcgttgggttctgccaccgtggccgcgattacggcgggcggtattgccgcaccactgatcgccacgaccggcgtcagtcctgaactgatggtgatcgccgtcggttccggcagcgtgattttctcgcacgtcaacgatccgggcttctggctgttcaaggaatatttcaacctgagcattgtcgaaaccttcaaatcctggtcggtgctggaaaccatcatttccctgtgcggtctggcgggatgcctgctgctgtcgatggtcgtctgaaggcaaacggatagcgtaaaaaaggcgagccagatggctcgcctttttcatttcttctgtcgctaaatcttcagatacgccctgatcccatcgaggaacatctgcgtcgagagcatgaccagaatcaggcccatcaggcgttcgagggcgctgacgcctttatcacccagcaggcgcaggaacaggtttgagagcaacaggattgccatcgacatgccccacgcgataaacagcgccagcgttaagtgactcatctggtttggatactggtgcgaaagcagcatcagcgtggcgagaatcgacggaccggcgaccagcgggatcgccatcgggacgaggaagggttcttcaccggcaggcagtccgctgctgcttttctcttccgacgggaaaatcattttgatggcgatcaggaacaggataataccgccggaaatcgacacagtttcagtgcgcagattcagaaacgacaggattttttcaccggcgaacaggaagatcagcatcaggatgagtgcgatcagcatttcgcggatcaacaccacacggcgacgtttcgggtcgagatgttttaataccgacataaaaatcggtaaattacccagcggatccataattaaaaacagcaaaatagttgctgaaatcatttcggtcataactacctcagaaaaaatgccagttatatacccgtcatacttcaagtcgcagatgcgttaactgcgctcactcacgccggtcacttagttgtctaagctccccaggcttcctgagcttgtcgcgttactcgcagggccagcgcaagcgctgttcaaaagcggaaacttttgtcctgcaactcgaattattgagggtataagttgcgctgctattaaaagccgcactattgattaaattcacttgccacttctactacattttgtaaggtggtagacccgcgcgttaatggggcgcagtgcatcaaaacaggcactcactgagtgcaaacg

SEQ ID NO: 69 - DNA, 5145 bp, Artificial Sequence
  source: /note=“Description of Artificial Sequence Synthetic polynucleotide”
ttcatttccagagtgaatgcagggctaatttgttgccacatcgcaaaaaatatcgcctaaaattacatcaaagatatcaatcacacacatttaataacatttttaacaaacgaactatttatctttacattctattttcatttatccttagcaggttaacgattatcccgatgccacccgactgccctggcagagtgcgtccggaccttaaagtgatatgtacaaacagggaacataaatgagcaaaaaaaccttgcccggcgtgacccgcagacaaattctctcattcacggccgcctccgcagtattgggtgcagccaaagccgcaaatgcggtgacgctcaaaggcacgcccgtgtggagcccctttgacgccagcccgccgccgcacatcgagaccgatggctgggtgttcttcaccgaagccgaagccgccgccatggaagccattgttgaccgtctggtgcccgccgatgatctgagcgtgggcggaaaagacgccggatgcgcggtatttattgaccgccagctggccggattctacggcaccgcctcccggctttatatgcaaggccctttccaggatggcacgccggaacaaggcgatcagtcgtcgctggttccgcaacagcgctaccgtcttggcctggccgcattagataaatacacccggtcacagcatcagaagttattcaaagatctgcccggtgagcagcaggatcacatcctgtcaggtctcgagagcggaaaaattgcccttgaagggatcgactctaaaatgttctttgccattgcactgaagaacacgatggaaggtttctttgctgacccgatttacggcggtaaccgcaatatggtgtcgtggaaaatgctgggcttccccggtgcacgttacgactaccgcgactatatcggcaagcacaatcagaaactcgatctcgaaccgctgagtatttccggcggtgacgcatggaaaataaaaagctaagggcagaacacgatttgctcccctcgtcccgacacttccagatcgccatagcgcacagcgcctcgagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaatggtacccttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgacagcccatgcaatccgtaactactatctgaaaaaaccgggagagatgatcgcatgaaaaagttaacgtcattctcactcggtttagtggctttgagcatcagcggactgagccacgcggcgggcgatggctctttcgaacaggttgaacgggggcgttacctggcgaccctgggcgactgcgccgcctgtcataccgcctccacgccggacgccaaaccttttgccggcggggtgccgattgaaacgccatttggccgtttagtcggtgcaaacattacaccggatacgcaaaccggtatcggcagatggagtttcgatgatttccagcgcgcaatgtcggagggcatcggtcacggcggaaaacgtctttacggcgcgatgccattcacggcgtacacgaaagtgacacgcgaagataacgccgccatctgggcgtatctgcaaactttgcaaccggtgcataatgaaattgaaaccaaccagttgcccttcccgttcaacgtgcgtaccagtctgatgggctggaactggctgaacttcaacaaaggcgaatttaaaccgcagatggataagtcggcgcaatggaatcgcggtgcttacatcgttgaaggtctgggacactgcggtacctgtcatacgccgaagaacctgctcggcggcgacaaagacagtgaattcctgcaagggaccgtgatagaaggctgggtcgcgccggatatcaccagcaacaaacacaccggtgtcggtaactggacgcaggaagatctgatgcaatatctgaaaaccggttctaaccgtttcgatattgcttccggtccgatggcggaagaagtcaccaattcgtcccgtcactggacggatgatgatcttaaggccgtggcggtttatctgaaagacagcggacatgacagcggtaacaacgcccctgagccagtaaaagctgacgataaggcgatgatagctggcaagaatatttatgctgatcgttgttctgcctgccataccccaaccggtatgggccaggaaggtttgttccctcgtctggccgattcaccacttatcaaccagaccgacg

SEQ ID NO: 70 - DNA, 5218 bp, Artificial Sequence
  source: /note=“Description of Artificial Sequence Synthetic polynucleotide”
  modified_base (42)..(42): a, c, t, g, unknown or other
cacaacaactaataacaggaactttccgtgcgcaattatatngatctaaattcagccagtgaagacagacaatgatctttgataactgatgctaatttaatttgagttttggtaaataggaaatttataactatctgttttaaaaggatttaaaatctattctatgattatttaaaaccaccacattagccaccgcacagttcgcatggaatttattcatttattttagatgtagcagtgatattataaatttgtaccccaaaataataataattaacccaaacttaacatcaggcatgtgagagtcgtaattctatgctccttcaaaaaaaaacaacccgtcgcaagtttttacttgggtcgctgatggctctgccggtcggcaccataatgatgaaaggattgtcggcggcacaagctgcggaaatggctgccccggatttactcgattataaacccatatttttcagtaccgatgagtggcagtttattatggccgctgctgaccgcctgatccctgctggtggcaaaggtaaagcaccgggcgcactcgaaaccaacgtgccgatttttatcgaccagcaaatgcacggtgattttggtgatgaaatttacatgcagggcccgttcaatgtgcatgctccggccacaatggggtatcagatcccgttccgtccgcagcaaatctacaaaaccggcattcgcctggcgaacgcctggtgtcagcaaaaccaccagaaagccttccatgacctcgccgctcaggacaaagacaacgttctgacgcagttgcagaaaaatggcattcagtttgcggatcttggtgaagagaacctagtggcttcgcaattcttcagtgaactgctttcagataccaaacacggttatctggcagacccgatctacggcggaaacaaagggatgaaagcatggatagccatcggcttccccggcgcccgcgccagtttcaccgaatgggttaagcagcacaatgtcccttatccgctgggacctgtgagcctgcaaggcgcgcgtgcctgagtgcccgccctattaccgtttctgatgaatgacaggattaaagtgctcccctcgtcccgacacttccagatcgccatagcgcacagcgcctcgagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaatggtacccttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgacagcggagatgaacacgatgaaactcaaaagtttatttatcgctaacgccttgctgctgggtgcaggttttatggcgaatgcgcaggcagccacgaatgcacaagccgacaacgtcgcgctcataaaacaaggtgagtatgtggcgcgcctgggcgactgcggcgcgtgtcatacggtggctggcaaaccggcgttttcaggcgggctggcgatcaactcgaatctgggcaccatttattcgaccaacattacgccggataaagaccacggcattggcggttataccgaagcccagttctcggatgcggtgcgtaaaggcgtgctgccggatggcacccgtttgtatccggcgatgccttaccctgattacgccaaaatcagcgatgaggatatgcacgcgctctacgtttactttatgcagggcgtaaaaccgagcgcggaacaaccgccggaaacggatctgagcttcccgttcagccagcgctggggcatgcgcttctggaactgggcgttcgcgtctgacaaaccattccagccaatcggcggtgcgtccgcacaggttaaccgcggcgcttatctggtggaaagcctgggccactgcggcagttgccacactcctcgcggcttcggcatgaatgaaaaagcactcgacagcagcgactctgattttctggcgggtggcagcctcaataactgggacgtcccgtcgctgcgcggcgtagcgcactggaacgaacaggagatcgtcgattatctgggcaaagggcgtaacgacaaagcggcggtaggcggcgaaatgacgtctgtggtggaaaatagcaccgcccatatgaccgatgaagacctcaaagccattgcggcatacatcaagttcctgggcggtaatccaccggttccggcacaagattcgcagaaagccagcgcgaccgaagccagactgacggcagcgaaaaatctcaccgaaggcgagcgtctgtatctggataactgtggtgcctgccactttgtgactggcaaaggcgcaccaggcgtgttcccgcagctggatcaggcaacgattgtgaacg

SEQ ID NO: 71 - DNA, 5236 bp, Artificial Sequence
  source: /note=“Description of Artificial Sequence Synthetic polynucleotide”
aaattccttcacaaatggcgattgccggttttcatggacacaacatgggacaaacgatggagccaccgctcgccagcgtcctgacgccgcgtgaaaaaatggggcaaatctccgccgaacgcctgctggcgagacttcgcggcgaagaagtggtgccgagaatggtggatgtagggtttacgatttctgcgggcggaagtatctgatttctttgcgccttcccctctcacgaggggatggctgggatggggtgttggttttgcggatgacagcgctgccattaagcccccctcccgacttccccggtctaaacatcccccttcgcaggggaaagtcttacttctgtaccgtcttcaaagccgcgatacagcgatccaccacaccttcaaacttgtgatcgatatttacgcgcaacacgtccggctcgtcttcgccgggttcttcgagtgcttcaaactggctgcgcagcaaatcggtcggcatgaagtgtccggcgcgtgctttcagacgttcggcaatcacctcaaaactgcctttcatgtaaataaataccatgccttcgttgtctttacgcagggcgtcgcggtaacggcgcttcagtgcggaacagacaatgatgccggtttcatttttatggcgcaggctgtaagccgcgtcgttgaggcgtaatagccacggtgcacgatcatcgtcgttcagtggcgtgccgctggccatcttttcaatattggcgcggggatgaagatcatcgccgtcgataaatttcgcgttaatttcacgggccagtgccgcgccgacggtggatttaccgctgcctgacacgcccatcagaatgatgctttgtcctgccataagtatgatctctatcccaaaatgaacattttgactgcgtagtgagtcctaatattagcatgttaccggtatcatgataccggtaacaaatggagatgtgactaatatcacaaaggaatgcactgctgttttagcggtcgcggtctgtacccgttgcactgttaattcctacaacatcttgaaaactaaaaataatgcgtcacccggtttttagccatacctgagtgacagcataaggggaagaattgctcccctcgtcccgacacttccagatcgccatagcgcacagcgcctcgagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaatggtacccttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgacaaggcaaacggatagcgtaaaaaaggcgagccagatggctcgcctttttcatttcttctgtcgctaaatcttcagatacgccctgatcccatcgaggaacatctgcgtcgagagcatgaccagaatcaggcccatcaggcgttcgagggcgctgacgcctttatcacccagcaggcgcaggaacaggtttgagagcaacaggattgccatcgacatgccccacgcgataaacagcgccagcgttaagtgactcatctggtttggatactggtgcgaaagcagcatcagcgtggcgagaatcgacggaccggcgaccagcgggatcgccatcgggacgaggaagggttcttcaccggcaggcagtccgctgctgcttttctcttccgacgggaaaatcattttgatggcgatcaggaacaggataataccgccggaaatcgacacagtttcagtgcgcagattcagaaacgacaggattttttcaccggcgaacaggaagatcagcatcaggatgagtgcgatcagcatttcgcggatcaacaccacacggcgacgtttcgggtcgagatgttttaataccgacataaaaatcggtaaattacccagcggatccataattaaaaacagcaaaatagttgctgaaatcatttcggtcataactacctcagaaaaaatgccagttatatacccgtcatacttcaagtcgcagatgcgttaactgcgctcactcacgccggtcacttagttgtctaagctccccaggcttcctgagcttgtcgcgttactcgcagggccagcgcaagcgctgttcaaaagcggaaacttttgtcctgcaactcgaattattgagggtataagttgcgctgctattaaaagccgcactattgattaaattcacttgccacttctactacattttgtaaggtggtagacccgcgcgttaatggggcgcagtgcatcaaaacaggcactcactgagtgcaaacgcgcgttcaagccgttttaagcagttatcaccctcagggcaggacagatattatgaaaaatgtaggtttcgttggctggcgtgga

SEQ ID NO: 72 - protein, 339 aa, Klebsiella variicola
MAIFDGHNDLLLNLWLHHREDPVSAFFVGIENGHLDYPRIRQGGLAGGLFALFVPPQEYIARVAPQYASEAWDPLAILWQQLAILKAICAHDASRARLCLSAADIERCRQDNALAMVAHIEGAGGFDAQGEDLQAFYRAGVRSIGPFWNIANRFGCGVTGAFPGSPDSGPGLTREGIALIAQANALKMQIDVSHMNEQAFWDTAHHSTAPLVATHSNAHALCPQPRNLTDRQLRAIRDSGGVVGVNFGNAFLRADGRRDSDTPLTTIVRHIDYLINIMGEDHVALGSDFDGITLPDELGDVAGLPRLINALRDNGYDQLVLDKLLWNNWLRVLKKVWQQ

SEQ ID NO: 73 - protein, 308 aa, Klebsiella variicola
MFIKVLGSAAGGGFPQWNCNCANCQGLRNGTIQASARTQSSIIVSDNGKEWVLCNASPDISQQIAHTPELNKPGVLRGTSIGGIILTDSQIDHTTGLLSLREGCPHQVWCTPEVHQDLSTGFPVFTMLRHWNGGLVHHPIAPQQPFTVDACPDLQFTAVPIASNAPPYSPYRDRPLPGHNVALFIENRRNGQTLFYAPGLGEPDEALLPWLQKADCLLIDGTVWQDDELQAAGVGRNTGRDMGHLALSDEHGMMALLASLPAKRKILIHINNTNPILNEQSPQRQALTQQGIEVSWDGMAITLQDTAC

SEQ ID NO: 74 - protein, 251 aa, Klebsiella variicola
MLITDTLSPQAFAEALRAKGAFYHIHHPYHIAMHNGEATREQIQGWVANRFYYQTTIPLKDAAIMANCPDAQTRRKWVQRILDHDGSHGEDGGIEAWLRLGEAVGLSRDDLLSERHVLPGVRFAVDAYLNFARRACWQEAACSSLTELFAPQIHQSRLDSWPQHYPWIKEEGYFYFRSRLSQANRDVEHGLALAKTYCDSAEKQNRMLEILQFKLDILWSMLDAMTMAYALQRPPYHTVTDKAAWHTTRLV

SEQ ID NO: 75 - protein, 92 aa, Klebsiella variicola
MQKTSIVAFRRGYRLQWEAAQESHVILYPEGMAKLNETAAAILELVDGRRDVAAIIAMLNERFPEAGGVDDDVVEFLQIACQQKWITCREPE

SEQ ID NO: 76 - protein, 380 aa, Klebsiella variicola
MSQNKPAVNPPLWLLAELTYRCPLQCPYCSNPLDFARQEKELTTEQWIEVFRQARAMGSVQLGFSGGEPLTRKDLPELIRAARDLGFYTNLITSGIGLTESKLDAFSEAGLDHIQISFQASDEVLNAALAGNKKAFQQKLAMARAVKARDYPMVLNFVLHRHNIDQLDKIIELCIELEADDVELATCQFYGWAFLNREGLLPTREQIARAEQVVADYRQKMAASGNLTNLLFVTPDYYEERPKGCMGGWGSIFLSVTPEGTALPCHSARQLPVAFPSVLEQSLESIWYDSFGFNRYRGYDWMPEPCRSCDEKEKDFGGCRCQAFMLTGSADNADPVCSKSPHHHKILEARREAACSDIKVSQLQFRNRTRSQLIYKTREL

SEQ ID NO: 77 - protein, 251 aa, Klebsiella variicola
MMSSEKTNNSRRDFLVKSMALIPTVVIGGAGAGAIGVATSATAQAAPASEPASGNTAAASDWKPQFFNDREWAFINAAVARLIPADELGPGAKEAGVPEFIDRQLNTPYATGSIWYMQGPFNPDVPKEMGYQLPLVPKQIYNLGIADAEAWCQDKYHKTFAELSNEQQDEALGLWESGKAEFKQLPASLFFTYLLQNTREGFFSDPIHGGNKGMVGWTLINFPGARADFMDWVERGERYPFPPVSINGERA

SEQ ID NO: 78 - protein, 438 aa, Klebsiella variicola
MPLIIVAIGVALLLLLMIRFKMNGFIALVLVALAVGLMQGMPLDKVIVSIKNGVGGTLGSLALIMGFGAMLGKLLADCGGAQRIATTLINKFGKKHIQWAVVLTGFTVGFALFYEVGFVLMLPLVFTIAASARIPLLYVGVPMAAALSVTHGFLPPHPGPTAIATIFHADMGKTLLYGTILAIPTVILAGPVFARFLKGIDKPIPEGLHNPKVFTEEEMPGFGVSVWTSLVPVILMAMRAVAEMILPKGHAFLPIAEFFGDPVMATLIAVLIALFTFGLNRGRSMEQINDTLTSSIKIIAMMLLIIGGGGAFKQVLVDSGMDKYIASIMHESNMSPLFMAWSIAAVLRIALGSATVAAITAGGIAAPLIATTGVSPELMVIAVGSGSVIFSHVNDPGFWLFKEYFNLTIGETIRSWSVLETIISVCGLVGCLLLGMVV

SEQ ID NO: 79 - protein, 796 aa, Klebsiella variicola
MAETKSQQSRLLVTLTALFAAFCGLYLLIGGAWLVVLGGSWYYPIAGLVMLGVTVMLLRGKRAALWLYAALLLATMIWGVWEVGFDFWALTPRSDILVFFGIWLILPFVWRRLSVPSAGAVGALVVALLISGGMLTWAGFNDPQEVNGTLSADATPAAPISNVADGDWPAYGRNQEGQRFSPLKQINADNVKNLKEAWVFRTGDLKQPNDPGEITNEVTPIKVGDTLFLCTAHQRLFALDAATGKEKWHFDPQLNADPSFQHVTCRGVSYHEAKADNAPADVVADCPRRIILPVNDGRLFAVNADNGKLCETFANKGILNLQTNMPVTTPGMYEPTSPPIITDKTIVIAGAVTDNFSTREPSGVIRGFDVNTGKLLWAFDPGAKDPNAIPSDEHHFTLNSPNSWAPAAYDAKLDLVYLPMGVTTPDIWGGNRTPEQERYASSIVALNATTGKLAWSYQTVHHDLWDMDMPSQPTLADIEVNGKTVPVVYAPAKTGNIFVLDRRNGELVVPAPEKPVPQGAAKGDYVAKTQPFSDLSFRPKKDLTGADMWGATMFDQLVCRVIFHQMRYEGIFTPPSEQGTLVFPGNLGMFEWGGISVDPNRQVAIANPMALPFVSKLIPRGPGNPMEPPKDAKGSGTESGVQPQYGVPYGVTLNPFLSPFGLPCKQPAWGYISALDLKTNEVVWKKRIGTPQDSLPFPMPVKLPFTMGMPMLGGPISTAGNVLFIGATADNYLRAYNMSNGEKLWEARLPAGGQATPMTYEVNGKQYVVISAGGHGSFGTKMGDYIVAYALPDDAK

SEQ ID NO: 80 - protein, 438 aa, Rahnella aquatilis
MPLVIVAVGVALLLLLMIRFKLNGFIALILVALAVGIMQGMPVDKVITSIKNGVGGTLGSLALIMGFGAMLGKMLADCGGAQRIATTLIEKFGREHIQWAIVLTGFIVGFALFYEVGFVLMLPLVFTVAAAARLPLLYVGVPMAAALSVTHGFLPPHPGPTAIATIFHADMGKTLLFGSLLAVPTVILAGPVYARFLKGIDKPVPEGLFNPKTFTEEEMPGFGVSVATSLVPVILMAFRALCEMILPKGHPVLAYAEFFGDPVMATLIAVLIAIFTFGLNRGRKMEDVMATVTDSIKIIAMMLLIIGGGGAFKQVLVDSGIEKYIAALMHGSTLSPILLAWSIAAVLRIALGSATVAAITAGGIAAPLIATTGVSPELMVIAVGSGSVIFSHVNDPGFWLFKEYFNLSIVETFKSWSVLETIISLCGLAGCLLLSMVV

SEQ ID NO: 81 - protein, 802 aa, Rahnella aquatilis
MRNERFFSWSFMTGLVTAVIGLAYLVLGVWLAALGGSPWYVLFGSGYLLSGIFIARRHASGIWLYLLTFLLCCVWSVWEVGLDGWQLMPRLFVMALLGVWCSLPLITRQVMATRGNHRTGTFAGLVYVVAIVGIFYSGWQVTGSRFVHRQPVPAQSGDIQATSPESNDWRYYGRTEAGQRYSPLTQITPANVSQLKPAWEFHTGDVMRKGEDKDGREFNFEVTPVKVGNSLFICTPHREVIALNATTGAQRWKFDPKSDTSANEYLACRGVAYSQSAGDKVCPEKIIATTSEARMVALNAQTGEPCSSFGQNGFVSLTDHMGDVPPGFHFITSQPMVMDGRIVLGGWIYDNQSTGEPSGVVRAFDVNTGQLAWAWDMGRDPQNAPLKPGEVYTRGTPNGWGTYTGDPKLGLVYIPLGNATPDYYGAGRRPFDEKYSSSLVALDIHTGEERWHFQTVHHDVWDFDLPIGPTLVDLPSPEGITVPALVQTTKMGQLFLLDRRTGKPLAQVNEKPVNTSPSLPGEHLSPTQPDSVGMPSLSPPDLKETDAWGATPIDQLYCRIQFKSARYQGQFTPPAEGKSIAYPAFDGVMDWYGASVDPIRHVLIANTSYIPFTMEVKKSADAIKEGLMHKWAGWGSNQPYPKPKEFSVGPQYGTPWAAIVKPWLSFLQAPCNAPPWGKLVAVDLTTRKIAWERPAGTTRDMNIFGTHTNVPLPTGIFMMGGNIITQSGLIFTGATADNYFRAFDETTGNELWRARLPAGGQATPMTYTGDDGRQFVVIAAGGHGGLGTTSGDALVAYALPAR

SEQ ID NO: 82 - protein, 799 aa, Rahnella aquatilis
METKASLSRIVVIITALFAALSGIYLLAGGIWLAKLGGSLYYIIAGVISLVTAWLLYRRRSSALLLYAIFLFGTTVWAVWEVGTDFWALTPRLDVTFFLGLWILLPVVYNQMLAKNAFARGALAVSLLFTVIVLGYAIFNDPQVINGTIKAADSAPAKSESGIPDGDWPAYGRTQGGTRYSPLNQINDKNVSKLDVAWTFRTGDLKTPNDPGEITDEVTPIKIGDMLYLCTPHQKLFALDAATGKEKWKFDPELKPNPTFQHVTCRGVSYHETTPAAEGNATNGAAPAVCSRRIILPVNDGRLFALDAETGARCPAFGNNGELNLQGNMPYATPGHYEPTSPPIITKSVIIVAGAVTDNYSNREPSGVIRGFDVETGKLLWAFDPGAAEPNKIPEDGQHFTPNSPNSWAPAAYDDKLDLVYLPIGVATPDIWGGNRTPEMERFASGLLALNATTGKLAWFYQTVHHDLWDMDVPAQPTLADITDKSGNKVPAIYVPTKTGNIFVLDRRDGKLIVDAPEKPVPQGAAKGDHVSPTQPFSKLTFRPEAKLTGKDMWGATIYDQLMCRVIFHKLRYEGTFTPPSEQGTLVFPGNLGMFEWGGISVDTDRQVAIANPIALPFVSKLIPRGPGNPIEPDANDKGGSGTETGIQPQYGVPFGVTLNPFLSPLGFPCKQPAWGYISGVDLKTNDIVWKKRIGTVRDSSPLPLPFKMGMPMLGAPVSTAGNVFFIAATADNYLRAFNMSNGDKLWEARLPAGGQATPMTYSVNGKQYVVIAAGGHGSFGTKLGDYIIAYALPDADAK

SEQ ID NO: 83 - protein, 245 aa, Rahnella aquatilis
MKIITTFCLASLFSVNAFALTGNDATTKPDLYYLKNDQAINSLALLPPPPAVGSIAFLNDQAMYEQGRLLRSTERGKLAAEDANLSAGGVANAFSGAFGSPITAKDSPELHKLLTNMIEDAGDLATRSAKEKYMRIRPFAFYGVPTCNTTEQDKLSKNGSYPSGHTSIGWATALVLTEINPQRQDQILQRGFDLGQSRVICGYHWQSDVDAARIVGSAVVATLHTNPAFQQQLQKAKEEFAKQHP

SEQ ID NO: 84 - protein, 232 aa, Rahnella aquatilis
MRKSLVALLLFTLGSTFAVQATEEAKPFITSQELDLTQYLPAPPADDSAQTQAELKELLQIQATRTPEQEKAAIADAQENVWRFADVMGPGFDAEKLPKTAALFERIVATEDVVDDHAKKAFNRPRPYMLDEQIHPLLKKSKSGSWPSGHSTIGYLMATVLGEMVPEKRNALFARASGYAENRLVAGFHYRSDTVMSRTGAALIAQKMEEQPDFKTEFDAAKAELRTQLGLK

SEQ ID NO: 85 - protein, 345 aa, Rahnella aquatilis
MNTIPHPPVFDGHNDLLLRLWLHDAADPAALFLDGSLEGHLDFRRCRLGGFAGGLFAIFVPPASYMPQLKPDSPAEPHDAFAITRAQISLLERLETQSAGRAKICRTVGEIEACITQNVLAMVMHIEGAEALGDDFSRLERWYEKGLRSIGPLWNLPNQFGTGVKGDFPGSPDTGDGLTPAGLGLLHECNRKRILFDVSHMNEKAFWQTAKFSDAPLVATHSNVHALCPQPRNLTDKQLAAIAESNGFVGVNFGTAFLRADGKRNGDTPITEIVKHLDNLVGKLGEENVGFGSDFDGINVPDTLGDVAGLPLLLQAMSDAGYGDALIEKIAYRNWLKVLKQTWGE

SEQ ID NO: 86 - protein, 303 aa, Rahnella aquatilis
MQIIVLGSAAGGGFPQWNCNCSNCQGVRNGTMKTSPRTQSSIAVSDNGTDWVLCNASPDICHQIAATPELIKQDVLRGTAIGSIILTDSQIDHCTGLLNLREGCPHQVWCTPEVHEDLTTGFPIFTMLSHWNGGLQHHAIRPENRFSVAVCPNLTFTAIPLLSNAPPYSKYRGKPLPGHNIALFIEDTKTGTSLLYAPGLGEPDDELLKWLHKADCLLIDGTLWQDNELATTGVGRNTGKDMGHLALAEEQGLIALLSSLPAKRKILIHINNTNPILNESSAERQALTQQNIEVSRDGMRIEL

SEQ ID NO: 87 - protein, 255 aa, Rahnella aquatilis
MSISTTQTSPMTPQEFEQALRAKGAFYHIHHPYHIAMHNGQATREQIQGWVANRFYYQTSIPLKDAAIMANCPDAQTRRKWVQRILDHDGHGGSEGGIEAWLRLGEAVGLDRDVLLSEERVLPGVRFAVDAYVNFARRAVWQEAACSSLTELFAPQIHQARLDTWPQHYTWIEEEGYGYFRSRLSQANRDVEHGLQLALEYCDTVEKQQRMLEILQFKLDILWSMLDSMSMAYELNRPPYHSVTQQAVWHKGRLL

SEQ ID NO: 88 - protein, 95 aa, Rahnella aquatilis
MITITEHYTPMFRRGYRMQFEKTQDCHVILYPEGMAKLNDSATFILQLVDGGRTIANIIDELNARFPQAGGVNDDVKDFFAQAHAQKWIIFREPA

SEQ ID NO: 89 - protein, 377 aa, Rahnella aquatilis
MNLLKPAVKPPLWLLAELTYRCPLQCPYCSNPLDFAKQEKELTTAQWIKVFEEAREMGAVQIGFSGGEPLVRKDLPELIRAARDLGFYTNLITSGIGLTEKKIDAFAEAGLDHIQISFQASDETLNAALAGNAKAFRQKLVMAKAVKAHGYPMVLNFVLHRHNIDQIDKIIDLSIELEADDVELATCQFYGWAQLNREGLLPTREQIARAEQVVHQYREKMAGTGNLANLLFVTPDYYEERPKGCMGGWGAIFLSVTPEGMALPCHSARQLPVEFPSVLENTLQEIWYDSFGFNKYRGFDWMPEPCRSCSEKEKDFGGCRCQAFMLTGNADNADPVCSKSEHHGKILAAREQANCTNIQINQLQFRNRVNSQLIFKG

SEQ ID NO: 90 - protein, 587 aa, Rahnella aquatilis
MAKKLPKTDVVVIGLGWAGSIIANELCDEGLNVVAIERGPWRDTARDFNVSTVTDELRYSSRQELMLRTRQNTITIRNNPAQTALPMREWGSFHPGNGTGGAGNHWAGITFRFQPDEFRLKSHLTERYGANAIPEELVLQDWGTDWEEMEPHYASFERLAGVSGKASNVKGEHHEGGNPYEGMRSIEYPTRPLDQPYGPTLFAQAARNMGYKAFPVPSSLISEPYTNPLGVKMGPCTFCGFCTNFGCANYSKASAITTVLPALIRKENFEARTNCEVMQVLTDSTGKRATGVVYIDSSGDEWEQPADLVIVSAFTFENVRLMLLSGIGKPYDPVTLSGTTGRNYAYQTANGVQLFFDDKNFNPFIGAGAVGMGIDDFNNDNFDHSGLGFFGGGSIRVTPIGGAPIGYRPVPPGTPKWGSEWKKATVANYLSSMSIGCEASSYTTKTNYLSLDPNYKDRLGRPLLRVTFDFPENDLKMAAYCTGKVAEIAKAMNPRQLVASPMKGHWNGTPYQSSHVVGGFVMGADPSTSSVNKHLQVWDVPNLFVVGASAFPQNPGYNPTGTVGALAFKAAHAIRHYYLKKPGEMIA

SEQ ID NO: 91 - protein, 587 aa, Rahnella aquatilis
MAKKLPKTDVVVIGLGWAGSIIANELCDEGLNVVAIERGPWRDTARDFNVSTVTDELRYSSRQELMLRTRQNTITIRNNPAQTALPMREWGSFHPGNGTGGAGNHWAGITFRFQPDEFRLKSHLTERYGANAIPEELVLQDWGTDWDEMEPHYASFERLAGVSGKASNVKGEHHEGGNPYEGMRSIEYPTRPLDQPYGPTLFAEAARNMGYKAFPVPSSLISEPYTNPLGVKMGPCTFCGFCTNFGCANYSKASAITTVLPALIRKENFEARTNCEVMQVLTDSTGKRATGVVYIDSSGDEWEQPADLVIVSAFTFENVRLMLLSGIGKPYDPVTLSGTTGRNYAYQTANGVQLFFDDKNFNPFIGAGAVGMGIDDFNNDNFDHSGLGFFGGGSIRVTPIGGAPIGYRPVPPGTPKWGSEWKKATVANYLSSMSIGCEASSYTTKTNYLSLDPNYKDRLGRPLLRVTFDFPANDLKMAAYCTGKVAEIAKAMNPRQLVATPMKGHWNGTPYQSSHVVGGFVMGADPSTSSVNKHLQVWDVPNLFVVGASAFPQNPGYNPTGTVGALAFKAAHAIRNYYLKKPGEMIA

SEQ ID NO: 92 - protein, 589 aa, Rahnella aquatilis
MAKVTKPEVDVVVVGLGWAGSLMSIELAMAGLTVRALERGGDRGYEEFAYPKPADEYAYAVRNKVMATPAEAAVTVRYNMRETALPTRKWGAFAPGTGVGGSGLHWTAVLIRPTPTDLKLKTYADEAYKPGILQEDMRIMDFPFTWDEIEPYYTKFEHICGQSGKTGNLRGQIMEGGDPFEGPRSEPYPLPALEDTLNSSMFAEVTKKMGYHPFPNPSACVSRAWTNPYGNQIAPCNYCGYCSKYPCLNYSKASPQTAVMDALKRMDNFSYEVHANVLKVELHDDKKTAKGVIYMDADGNECFQPAKIVVLSSFQFCNVRLMLLSGIGKPYNPITEEGVIGRNYAFLSNGGSTLFFKDKNFNPFATAGATGQMFNDISPGNFDGPALGFIGGAKIHSSQATGTPISTSLPKGTPAWGTGWKEGMEEWYGHSMKISITTTCQSYRDIYLDLDPNYKDEYGYPLLRMTFDWKQNELKLQQYLKGIVGNITKELNPDSYSESFLPMDAHFDLTKYVSTHNVGGAVMGDNPKTSALNKFLQSWDVHNVFVPGGNAFPQNFQANPTDTIGAITLMAAQAIKDQYLKNPGPLVQA

SEQ ID NO: 93 - protein, 438 aa, Rahnella aquatilis
MPLVIVAVGVALLLLLMIRFKLNGFIALILVALAVGIMQGMPVDKVITSIKNGVGGTLGSLALIMGFGAMLGKMLADCGGAQRIATTLIEKFGREHIQWAIVLTGFIVGFALFYEVGFVLMLPLVFTVAAAARLPLLYVGVPMAAALSVTHGFLPPHPGPTAIATIFHADMGKTLLFGSLLAVPTVILAGPVYARFLKGIDKPVPEGLFNPKTFTEEEMPGFGVSVATSLVPVILMAFRALCEMILPKGHPVLAYAEFFGDPVMATLIAVLIAIFTFGLNRGRKMEDVMATVTDSIKIIAMMLLIIGGGGAFKQVLVDSGIEKYIAALMHGSNLSPILLAWSIAAVLRIALGSATVAAITAGGIAAPLIATTGVSPELMVIAVGSGSVIFSHVNDPGFWLFKEYFNLSIVETFKSWSVLETIISLCGLAGCLLLSMVV